(19) United States
(12) Patent Application Publication
(10) Pub. No.: US 2026/0286299 A1
(43) Pub. Date: Sep. 24, 2026

(54) COMPOSITIONS AND METHODS FOR MULTIPLEX ENGINEERING OF IMMUNE CELLS

(71) Applicant: Be Biopharma, Inc., Cambridge, MA (US)

(72) Inventors: Siyi Huang, Lexington, MA (US); Anja Fides Hohmann, Somerville, MA (US); Richard A. Morgan, Moultonborough, NH (US); Xuqing Zhang, Quincy, MA (US); Alexis Benoit Hubaud, Melrose, MA (US)

(21) Appl. No.: 19/484,651

(22) PCT Filed: May 15, 2024
(86) PCT No.: PCT/US2024/029548

§ 371 Date: Nov. 14, 2025

Related U.S. Application Data
(60) Provisional application No. 63/502,645, filed on May 16, 2023.

(51) Int. Cl. C12N 5/0781; C12N 9/22; C12N 15/11

(52) U.S. Cl. CPC C12N 5/0635; C12N 9/226; C12N 15/111; C12N 2310/20; C12N 2510/00

(57) ABSTRACT

The present disclosure provides methods and compositions for culturing and engineering of B lineage cell populations at multiple target loci to express one or more transgenes.

### CROSS-REFERENCE TO RELATED APPLICATIONS

This application claims priority to U.S. Provisional Application No. 63/502,645, filed on May 16, 2023, the entirety of which is incorporated herein by reference.

### BACKGROUND

Cell therapy is an emerging from of treatment that comprises administration of viable human cells to a subject to act as “living drugs.” In particular, certain cell therapies make use of engineered immune cells, to combat disease. Culturing and methods of administration of these engineered adaptive immune cells are critical both in clinical and pre-clinical settings for treatment in a patient population.

### SUMMARY OF THE INVENTION

The present disclosure encompasses, among other things, compositions, methods, and administration of said compositions comprising modified immune cells (e.g., B lineage cells). The present disclosure also encompasses, among other things, culturing methods and compositions for multiplex engineering of immune cells. Among other things, the present disclosure provides a recognition that one or more modifications of B lineage cells (e.g., through genetic engineering and/or alternative culturing methods) may provide improved properties, e.g., reduced immune response upon or after administration to a subject. In some embodiments, the present disclosure provides culturing methods and compositions for engineering B lineage cells sourced from a donor, wherein the engineered B lineage cells produce a reduced immune response in a subject that is not the donor as compared to a reference (e.g., untreated subject, healthy subject, subject treated with non-engineered or alternatively engineered B lineage cells, etc.).

In some embodiments, the present disclosure provides methods of genetically modifying B lineage cell populations, the method comprising steps of: (a) isolating primary B cells so that a primary B cell population is obtained; (b) activating the primary B cell population; and (c) during or after the activating step, multiplex engineering the primary B cell population such that a first expression cassette comprising a first transgene integrates into a first target locus, and cither (i) a second expression cassette comprising a second transgene integrates into a second target locus, or (ii) endogenous gene expression at the second target locus is disrupted without integration of a transgene, thereby generating a genetically modified B lineage cell population. In some embodiments, the method further comprises a step of expanding an engineered B lineage cell population.

Among other things, in one aspect, the present disclosure provides methods of differentiating an engineered B lineage cell population into a population of plasmablasts. In some embodiments, a differentiation step comprises contacting an engineered B lineage cell population with culture media comprising: (a) IL-2, (b) IL-6, (c) IL-10, and/or (d) IL-15.

Among other things, in one aspect, the present disclosure provides methods of differentiating an engineered B lineage cell population into a population of plasma cells. In some embodiments, a differentiation step comprises contacting a population of plasmablasts with culture media comprising: (a) IL-6, (b) IL-15, and/or (c) IFN-alpha-2-beta (IFNα-2β).

In some embodiments, the present disclosure provides methods of engineering B lineage cell populations. In some embodiments, the method further comprises a step of transferring an engineered B lineage cell population to cell culture media without human or bovine serum for about 24 hours. In some embodiments, the step of engineering further comprises introducing an expression cassette comprising a transgene into the primary B cell population.

In some embodiments, multiplex engineering of B lineage cell populations comprises simultaneously contacting primary B lineage cell populations with both the first and second expression cassettes. In some other embodiments, multiplex engineering of B lineage cell populations comprises sequentially contacting primary B lineage cell populations with a first prior to a second expression cassette. In some embodiments, sequential contact comprises contacting primary B lineage cell populations with a first expression contact on day 3 of the culture process, followed by contacting the cell populations with a second expression cassette on day 4 of the culture process, wherein the culture process has a day 1 start date. In some embodiments, multiplex engineering of B lineage cell populations comprises contacting primary B lineage cell populations with both a first and second expression cassette over a time range of about 24 to about 144 hours.

In some embodiments, an expression cassette comprises: (a) a 5′ homology arm that is at least 95% identical to a sequence 5′ to a double-stranded break site, and (b) a 3′ homology arm that is at least 95% identical to a sequence 3′ to a double-stranded break site. In some embodiments, a 5′ homology arm comprises or is: (i) a length about 450-850 base pairs in size, (ii) a PAM site or absence of a PAM site, and/or (iii) symmetrical or asymmetrical in length with the 3′ homology arm. In some embodiments, a 3′ homology arm comprises or is: (i) a length about 450-850 base pairs in size, (ii) a PAM site or absence of a PAM site, and/or (iii) symmetrical or asymmetrical in length with the 5′ homology arm.

In some embodiments, a step of engineering comprises contacting the primary B cell population with a targeted nuclease capable of introducing double-stranded breaks. In some embodiments, a targeted nuclease is or comprises a CRISPR-associated (Cas) protein, zinc finger nuclease (ZFN), transcription activator-like effector-based nuclease (TALEN), or meganuclease. In some embodiments, a Cas protein is or comprises Cas9, Cas12a, or Cas13a, or a variant thereof. In some embodiments, a Cas protein is complexed with a guide RNA (gRNA). In some embodiments, a gRNA is a single guide RNA (sgRNA).

In some embodiments, a step of engineering further comprises electroporation of the primary B cell population, and/or (b) transduction of the primary B cell population with an expression cassette. In some embodiments, electroporation is performed on a composition comprising: (a) a Cas protein complexed with the gRNA, and (b) a primary B cell population.

In some embodiments, a B lineage cell population is contacted with media that further comprises one or more of the following: XL413, M3814, nocodazole, and/or LAH4.

In some embodiments, the present disclosure provides populations of genetically modified B lineage cells comprising a transgene, wherein a transgene is expressed from an endogenous CCR5 locus. In some embodiments, cells express a transgene and at least partially disrupt expression of endogenous CCR5.

In some embodiments, the present disclosure provides populations of genetically modified B lineage cells comprising a transgene, wherein the transgene is expressed from an endogenous JCHAIN locus. In some embodiments, cells express a transgene without disrupting expression of endogenous JCHAIN.

In some embodiments, the present disclosure provides populations of genetically modified B lineage cells comprising a transgene, wherein a transgene is expressed from an endogenous IgH locus. In some embodiments, cells express a transgene without disrupting expression of endogenous IgH.

In some embodiments, the present disclosure provides populations of genetically modified B lineage cells comprising a transgene, wherein the transgene is expressed from an endogenous B2M locus. In some embodiments, cells express a transgene and at least partially disrupt expression of endogenous B2M.

In some embodiments, the present disclosure provides populations of engineered B lineage cells comprising a transgene sequence, wherein the transgene is integrated in at least 10% of the cells. In some embodiments, a transgene is expressed from an endogenous CCR5 locus.

In some embodiments, multiplex engineering of B lineage cell populations comprises simultaneously contacting primary B lineage cell populations with the first and second expression cassette. In some embodiments, multiplex engineering of B lineage cell populations comprises sequentially contacting primary B lineage cell populations with the first expression cassette, then the second expression cassette. In some embodiments, sequential contact comprises contact with the first expression cassette on day 3 of the culture process and contact with the second expression cassette on day 4 of the culture process wherein the culture process has a day 1 start date. In some embodiments, the time range of contact between the first and second expression cassettes in about 24 hours to about 144 hours.

In some embodiments, the present disclosure provides a pharmaceutical composition comprising one or more B lineage cells selected from a population of genetically modified B lineage cells. In some embodiments, a pharmaceutical composition comprises one or more B lineage cells selected from a population of genetically modified B lineage cells and further comprises one or more pharmaceutically acceptable excipients. In some embodiments, a method of administering a pharmaceutical composition, wherein the pharmaceutical composition comprises: (a) one or more B lineage cells selected from a population of genetically modified B lineage cells of any aspect or embodiment described herein, and (b) one or more pharmaceutically acceptable excipients, wherein the pharmaceutical composition is administered to a subject.

In some embodiments, the present disclosure provides methods of treating a disease, disorder, or condition in a subject, the method comprising administering to the subject a therapeutically effective amount of a pharmaceutical composition, thereby treating the disease, disorder, or condition in the subject. In some embodiments, a pharmaceutical composition is administered intravenously. In some embodiments, a pharmaceutical composition is administered to an adult subject. In some embodiments, a pharmaceutical composition is administered to a pediatric subject. In some embodiments, a subject has hemophilia B.

In some embodiments, the present disclosure provides methods of characterizing a population of genetically modified B lineage cells, the method comprising assessing one or more of (a)-(g) using an assay: (a) presence of a CD38 marker, (b) presence of a CD138 marker, (c) presence of a CD27 marker in at least 50% of live, singlet cells, (d) secretion of at least 0.5 pg/cell/day of IgG, (e) secretion of at least 2.5 pg/cell/day of IgM, (f) secretion of at least 0.5 pg/cell/day of IgD, and (g) secretion of at least 0.1 pg/cell/day of a FIX protein. In some embodiments, an assay comprises or is one or more of: fluorescence-activated cell sorting (FAC-sort), Western Blot, flow cytometry, enzyme-linked immunosorbent spot assay (ELISpot), quantitative polymerase chain reaction (qPCR), reverse transcriptase PCR (RT-PCR), RT-qPCR, Meso Scale Discovery (MSD), and enzyme-linked immunosorbent assay (ELISA). In some embodiments, one or more B lineage cells selected from a population of genetically modified B lineage cells engraft within bone marrow of a subject. In some embodiments, a method of monitoring engraftment of genetically modified B lineage cells within a subject, comprising one or more of: bioluminescence, ELISpot, flow cytometry, qPCR, RT-PCR, RT-qPCR, and enzyme-linked immunosorbent assay.

In some embodiments, multiplex engineering of B lineage cell populations comprises engineering of a first target locus selected from CCR5, JCHAIN, and IGH. In some embodiments, multiplex engineering of B lineage cell populations comprises engineering of a second target locus selected from B2M, BCMA, CD19, and CD20. In some embodiments, the first target locus is CCR5 and the second target locus is CD19, wherein endogenous gene expression at the second target locus is disrupted without integration of a transgene. In some embodiments, the first target locus is CCR5 and the second target locus is B2M, wherein endogenous gene expression at the second target locus is disrupted without integration of a transgene. In some embodiments, the first target locus is JCHAIN and the second target locus is B2M, wherein endogenous gene expression at the second target locus is disrupted without integration of a transgene. In some embodiments, the first target locus is IGH and the second target locus is B2M, wherein endogenous gene expression at the second target locus is disrupted without integration of a transgene. In some embodiments, the first target locus is CCR5 and the second target locus is B2M, wherein a second expression cassette comprising a second transgene integrates into the second target locus. In some embodiments, the first target locus is JCHAIN and the second target locus is B2M, wherein a second expression cassette comprising a second transgene integrates into the second target locus. In some embodiments, the first target locus is IGH and the second target locus is B2M, wherein a second expression cassette comprising a second transgene integrates into the second target locus. In some embodiments, the first transgene integrating into the first target locus is a BiTE. In some embodiments, the first transgene integrating into the first target locus is selected from Factor IX, SMPD1, and blinatumomab. In some embodiments, the second transgene integrating into the second target locus is selected from HLA-A, HLA-B, HLA-C, HLA-E, and HLA-G.

In some embodiments, the present disclosure provides methods of genetically modifying a B lineage cell population, a method comprising the steps of: (a) isolating primary B cells so that a primary B cell population is obtained; (b) activating the primary B cell population; and (c) during or after the activating step, engineering the primary B cell population to reduce expression of one or more endogenous target genes; thereby generating a genetically modified B lineage cell population. In some embodiments, one or more endogenous target genes is NLRC5, CD58, RFX5, TAP2, and/or TAPBP.

In some embodiments, the present disclosure provides a method for reducing expression comprising knockout of one or more endogenous target genes. In some embodiments, a step of engineering comprises integrating one or more transgenes. In some embodiments, integration of one or more transgenes reduces expression of one or more endogenous target genes. In some embodiments, methods and embodiments described herein comprise a transgene further comprising a sequence encoding HLA-E, HLA-G, CD47, PDL1, FIX, SMPD1, and/or bispecific dual-scFv. In some embodiments, a step of engineering comprises integrating a transgene into an endogenous NLRC5, RFX5, CD58, TAP2, TAPBP, CCR5, B2M, CD19, JCHAIN, AAVS1, TRAC, and/or GSH locus.

In some embodiments, a step of engineering further comprises introducing a donor construct comprising a transgene into a primary B cell population. In some embodiments, a donor construct comprises: (a) a 5′ homology arm that is at least 95% identical to a sequence 5′ to a double-stranded break site; and (b) a 3′ homology arm that is at least 95% identical to a sequence 3′ to a double-stranded break site. In some embodiments, a donor construct comprises a transgene sequence of any one of SEQ ID NOs: 34-39. In some embodiments, a donor construct comprises homology arm sequences of any one of SEQ ID NOs: 10-18.

In some embodiments, the present disclosure provides a population of genetically modified B lineage cells comprising a knockout modification at one or more endogenous gene loci. In some embodiments, a knockout modification is at an endogenous NLRC5, B2M, CD58, RFX5, TAP2, and/or TAPBP gene locus. In some embodiments, a transgene comprises a sequence encoding a HLA-E or HLA-G protein. In some embodiments, a transgene further comprises a 2A peptide or IRES sequence 3′ of the sequence encoding a HLA-E protein. In some embodiments, a transgene further comprises a sequence encoding a second protein 3′ of the 2A peptide or IRES sequence.

### BRIEF DESCRIPTION OF THE DRAWINGS

The drawings are for illustration purposes only, not for limitation.

FIG. 1A shows an exemplary schematic for multiplexed genome editing at two separate genetic loci. FIG. 1B shows assessment of editing efficiency for certain guide RNAs alone or in multiplex, targeting either CCR5 or B2M as indicated. Guide RNAs were administered in a 1:1:1 ratio for (gRNA targeting CCR5):(gRNA targeting B2M):(Cas9). FIG. 1C shows assessment of editing efficiency for certain guide RNAs alone or in multiplex, targeting either CCR5 or B2M as indicated. Guide RNAs were administered in a 3.58:1:1 ratio for (gRNA targeting CCR5):(gRNA targeting B2M):(Cas9).

FIG. 2 shows assessment of engineered B lineage cells. Cells were multiplex engineered for disruptive expression of GFP from the CCR5 locus and disruptive expression of an immune modulator (HLA-E) from the B2M locus.

FIG. 3A shows assessment of engineered B lineage cells. Cells were multiplex engineered for disruptive expression of GFP from the CCR5 locus and disruptive expression of an immune modulator (HLA-E) from the B2M locus. FIG. 3B and FIG. 3C show assessment of engineered B lineage cells. Cells were multiplex engineered for disruptive expression of GFP-BiTE (Blinatumomab) from the IgH locus and disruptive expression of an immune modulator (HLA-E) from the B2M locus.

FIG. 4 shows assessment of engineered B lineage cells. Cells were multiplex engineered for disruptive expression of either a BiTE (Blinatumomab) or Factor IX (FIX) from the CCR5 locus, combined with disruptive expression of an immune modulator (HLA-E) from the B2M locus.

FIG. 5 shows assessment of engineered B lineage cells. Cells were multiplex engineered for disruptive expression of either a BiTE (Blinatumomab) or Factor IX (FIX) from the CCR5 locus, combined with disruptive expression of an immune modulator (HLA-E) from the B2M locus.

FIG. 6A shows assessment of simultaneous and sequential (Seq) editing of B lineage cell populations to produce multiplex engineered B lineage cell populations. Cells were either engineered with guide RNA/Cas9 RNPs through Seq 1 protocol (Cas9 RNP complexed with gRNA targeting CCR5 and AAV with GFP donor template on day 3 of culture process, then Cas9 RNP complexed with gRNA targeting B2M and AAV HLA-E donor template on day 4 of culture process), Seq 2 protocol (Cas9 RNP complexed with gRNA targeting B2M and AAV with HLA-E donor template on day 3 of culture process, then Cas9 RNP complexed with gRNA targeting CCR5 and AAV GFP donor template on day 4 of culture process), Simultaneous day 3 (simultaneous administration of both RNPs and AAVs with donor templates on day 3 of culture process), and Simultaneous day 4 (simultaneous administration of both RNPs and AAVs with donor templates on day 4 of culture process). FIG. 6B demonstrates potential translocation events at CCR5 and B2M loci and assessment of such translocation events in multiplex and single engineered B lineage cell populations. Culture timing is described with a day 1 start date.

FIG. 7 shows multiplex engineering to express transgenes at multiple loci. An expression cassette comprising a MND promoter and luciferase (LUC) transgene was inserted at CCR5 locus, and an expression cassette comprising a EF1a promoter and HLA-E transgene was inserted at B2M locus (top panel). Percentage of HDR at CCR5 locus was assessed via ddPCR (left panel) and luminescence was measured using Steady-Glo (middle panel). HLA-E integration was shown by percentage of Day 13 engineered B lineage cells of HLA-E+HLA-ABC-phenotype.

FIG. 8 shows administration of B lineage cells engineered to express indicated expression constructs from a CCR5 and/or B2M locus. Engineered B lineage cells were administered to hCD34-NSG-IL15 humanized and NOG-IL6 mice and monitored over 6 weeks. Luminescence was measured through whole-body imaging of treated mice at the 6-week time point.

FIG. 9A shows B lineage cells engineered as indicated to comprise a MND promoter and bicistronic HLA-E transgene and luciferase transgene (“Biscis HLA-E”) at a CCR5 locus and a EF1a promoter and GFP transgene at a B2M locus as compared to control with a MND promoter and GFP transgene alone at a CCR5 locus. Percentage of GFP-positive cells (left panel) and percentage of HLA-E-positive HLA-ABC-negative cells within GFP-positive cells (right panel) were assessed. Comparison with the control (engineered at CCR5 locus only) and relative percentage of cells that were HLA-E positive (HLA-E+) and/or HLA-ABC positive (HLA-ABC+) are also shown (right panel). FIG. 9B shows assessment of primed T cell activation and naïve T cell proliferation after co-culture with autologous or allogeneic engineered B lineage cells as compared to activated B cells (day 2 B cells) and B lineage cells engineered to knock out B2M expression (D13 B2M−/− engineered B lineage cells). cells. FIG. 9C shows assessment of various B lineage cell populations in co-culture with NK cells, including assessment of real-time killing and competitive survival rates. Culture timing is described with culture start date as day 0.

FIG. 10A shows assessment of B lineage cells engineered as indicated to comprise a MND promoter and a bicistronic Factor IX (FIX) transgene and GFP transgene at a CCR5 locus (Control) or comprise a MND promoter and a bicistronic FIX transgene and HLA-E transgene at a CCR5 locus and a MND promoter and GFP transgene at a B2M locus (Bicis HLA-E). Percentage integration at CCR5 was assessed (left panel), as well as percentage of GFP-positive within live cells (middle panel). Percentage of FIX-HLA-E-positive HLA-ABC-negative within GFP+ cells for the Day 13 Bicis HLA-E engineered B lineage cells were also assessed (right panel). FIG. 10B shows assessment of killing of Control, B2M−/− engineered B lineage cells, or Bicis HLA-E by HLA mismatched NK cells (left panel), competitive survival of cells of different phenotype when Bicis HLA-E engineered B lineage cells were co-cultured with or without HLA mismatched NK cells (middle panel), and FIX activity by capture chromogenic assay (right panel).

FIG. 11A demonstrates assessment of various conditions for editing and/or integration at CCR5 and TAP using CRISPR/Cas9, with integration of bicistronic LUC transgene and GFP transgene at CCR5 alone or in combination with knockout of TAP or in combination with knockout of both B2M and CD58. Various ratios of CCR5 to TAP gRNA were tested (1:1 and 3.58:1) as well as ratios of B2M and CD58 gRNAs (1:1). Percentage reduction in HLA-ABC was assessed for indicated engineered B lineage cell populations. FIG. 11B demonstrates assessment of conditions for multiplexed gene editing with CRISPR/Cas9 at CCR5 and TAP. FIG. 11C demonstrates assessment of conditions for gene editing with CRISPR/Cas9 at CCR5 (with bicistronic LUC transgene and GFP transgene) alone or in combination with TAP2 and TAPBP. As indicated, a 1:1 ratio of gRNAs was used for TAP2 and TAPB2. Percentage reduction in HLA-ABC was assessed for indicated engineered B lineage cell populations. FIG. 11D demonstrates assessment of conditions for multiplexed gene editing with CRISPR/Cas9 at CCR5, TAP, and TAPBP loci. FIG. 11E demonstrates assessment of various conditions for editing and/or integration at CCR5 and NLRC5 using CRISPR/Cas9, with integration of bicistronic LUC transgene and GFP transgene at CCR5 alone or in combination with knockout of NLRC5. Various ratios of CCR5 to NLRC5 gRNA were tested (1:1 and 3.58:1). FIG. 11F demonstrates assessment of conditions for multiplexed gene editing with CRISPR/Cas9 at CCR5 and NLRC5. FIG. 11G demonstrates assessment of various conditions for editing and/or integration at CCR5, B2M, and/or CD58 using CRISPR/Cas9, with integration of bicistronic LUC transgene and GFP transgene at CCR5 alone or in combination with knockout of B2M and CD58. Various ratios of B2M to CD58 gRNA were tested (1:1). FIG. 11H demonstrates assessment of B lineage cells edited at indicated loci.

FIG. 12A shows assessment of B lineage cells engineered as indicated to comprise a MND promoter and a GFP transgene at CCR5, knockout of B2M, and knockout of CD58 (B2M/CD58 DKO) along with a Control with only CCR5 integration. Percentage of GFP-positive cells from the Control and B2M/CD58 DKO (left panel), and editing efficiency at B2M and CD58 locus of the B2M.CD58 DKO (right panel) were assessed. FIG. 12B shows assessment CD8 T cell activation and proliferation after co-culture with autologous or allogeneic D2 B cells, Control D13 engineered B lineage cells, D13 B2M−/− engineered B lineage cells, and B2M/CD58 DKO as well as competitive survival of cells of different phenotype when B2M/CD58 DKO engineered B lineage cells were co-cultured with autologous or allogeneic primed T cells. FIG. 12C shows measurement of real time target engineered B lineage cells killing by HLA mismatched NK cells and NK degranulation after exposure to allogeneic engineered B lineage cells for 4 hours.

FIG. 13A shows assessment of B lineage cells engineered as indicated to comprise FIX-GFP transgene at CCR5 (Control), or FIX-GFP transgene at CCR5 and knockout of CD58 (CD58 KO). Measurement of integration efficiency at CCR5, GFP-positive cells, and percentage of CD58-positive cells was assessed for the Control and CD58 KO. FIG. 13B shows assessment of primed CD8 T-cell activation after co-culture with autologous or allogeneic D2 B cells, Control D13 engineered B lineage cells, D13 B2M−/− engineered B lineage cells, and CD58 KO and FIX activity of indicated engineered B lineage cell populations.

FIG. 14A shows assessment of B lineage cells engineered as indicated to comprise a GFP transgene at CCR5 either alone (Control) or in combination with knockout of TAP2 (TAP2 KO), knockout of TAP2 and TAPBP (TAP2/TAPBP DKO), knockout of NLRC5 (NLRC5 KO), knockout of RFX5 (RFX5 KO), or knockout of B2M (B2M KO). Non-engineered day 2 activated B cells were also assessed. Measurement of MHC-I expression, MHC-II expression, and HLA-E was conducted for indicated engineered B lineage cell populations.

FIG. 14B shows assessment of primed T-cell activation by indicated autologous or allogeneic engineered B lineage cell populations. FIG. 14C shows real-time target engineered B lineage cells killing by allogeneic NK cells (left panels and top right panel), NK degranulation 4 hours post exposure to allogeneic engineered B lineage cells targets (bottom middle panel), and percentage killing of GFP-positive cells by allogeneic NK cells (bottom right panel). Culture timing is described with culture start date as day 0.

FIG. 15A shows assessment of B lineage cells engineered as indicated to comprise a FIX-GFP transgene at CCR5 (Control), a FIX-GFP transgene at CCR5 and a knockout of B2M (B2M KO), a FIX-GFP transgene at CCR5 and knockout of NLRC5 (NLRC5 KO), a FIX-GFP transgene at CCR5 and knockout of CD58 (CD58 KO), a FIX-GFP transgene at CCR5 and knockout of NLRC5 and CD58 (NLRC5/CD58 DKO), a bicistronic FIX transgene and HLA-E transgene at CCR5 and a GFP transgene at B2M (Bicis HLA-E), or a FIX transgene at CCR5 (Control′). Integration efficiency at CCR5 and percentage of GFP-positive cells were assessed. FIG. 15B shows assessment of indicated B lineage cells for HLA-ABC levels and percentage of CD58-positive cells. FIG. 15C shows assessment of engineering efficiency for Bicis HLA-E. FIG. 15D shows assessment of indicated engineered B lineage cell populations for NK cell resistance. FIG. 15E shows assessment of indicated engineered B lineage cell populations for T-cell resistance.

FIG. 16 shows assessment of B lineage cells engineered as indicated to comprise a FIX transgene at CCR5 (Control), a FIX transgene at CCR5 and a knockout of B2M (B2M KO), a FIX transgene at CCR5 and a knockout of NLRC5 (NLRC5 KO), or a FIX transgene at CCR5 and knockout of B2M and CD58 (B2M/CD58 DKO). Integration efficiency, FIX secretion, and FIX activity were assessed for indicated engineered B lineage cell populations.

FIG. 17 shows assessment of B lineage cell populations engineered as indicated to comprise a FIX transgene at CCR5 (Control) or a bicistronic FIX transgene and HLA-E transgene at CCR5 and a GFP transgene at B2M (Bicis HLA-E). Integration efficiency, FIX secretion, and FIX activity were assessed for indicated engineered B lineage cell populations.

FIG. 18 shows assessment of various gRNAs targeting a NLRC5 locus in B lineage cells as described herein with a CCR5 guide as control in two donors.

FIG. 19A shows the cutting efficiency on day 5. FIG. 19B shows assessment of cell differentiation of D13 B lineage cells as determined by plasma cell (PC) and plasmablast (PB) %. FIG. 19C shows HLA-ABC, HLA-DR/DP/DQ, HLA-E expression levels on day 7 and day 13 B lineage cells. FIG. 19D demonstrates assessment of percentage of HDR and FIX secretion for B lineage cell populations engineered with indicated gRNAs at day 7 and day 13 of cell culture.

FIG. 20A shows a schematic demonstrating various single editing and multiplex editing strategies for integration of HLA-E, blinatumomab (Blina), FIX, or combinations thereof. FIG. 20B shows assessment of indicated engineered B lineage cell populations.

FIG. 21A shows assessment of various indicated engineered B lineage cell populations comprising a LUC transgene at CCR5 (Control), a LUC transgene at CCR5 and a knockout of B2M (B2M KO), a bicistronic HLA-E transgene and LUC transgene (comprising a 2A peptide) at CCR5 and a knockout of B2M (Bicis HLA-E), a LUC transgene at CCR5 and a knockout of NLRC5, or a LUC transgene at CCR5 and knockout of B2M and CD58 (B2M/CD58 DKO). B lineage cell populations were engineered and cultured as described herein to comprise engineered plasma cell preparations. Engineered plasma cell preparations were administered to hCD34-NSG-IL15 and NOG-IL6 mice and were assessed 7 weeks post-transfer. FIG. 21B shows whole-body luminescence imaging of treated mice. FIG. 21C shows assessment of totality of engraftment (AUC measurement). Persistence of engrafted B lineage cells in indicated mouse models was assessed for Control and Bicis HLA-E engineered B lineage cell preparations.

FIG. 22A shows assessment of various indicated engineered B lineage cell populations comprising a LUC transgene at CCR5 (Control), a LUC transgene at CCR5 and a knockout of B2M (B2M KO), a bicistronic HLA-E transgene and LUC transgene (comprising a 2A peptide) at CCR5 and a knockout of B2M (Bicis HLA-E), a LUC transgene at CCR5 and a knockout of NLRC5, or a LUC transgene at CCR5 and knockout of B2M and CD58 (B2M/CD58 DKO). B lineage cell populations were engineered and cultured as described herein to comprise engineered plasma cell preparations. Engineered plasma cell preparations were administered to NOG-IL15 mice and were assessed 7 weeks post-transfer. FIG. 22B shows whole-body luminescence imaging of treated mice.

FIG. 23 shows percentage of HLA-ABC-negative and “Don't eat me” signal (HLA-E or HLA-G) positive expressing B lineage cell populations after either multiplex engineering with a promoter (EF1a) or promoterless HLA-E construct compared to a bicistronic HLA-E construct or multiplex engineering with a promoterless HLA-E with without ILT2 KO.

FIG. 24A shows percentage of living B lineage cells that were either plasmablasts or plasma cells after cither undergoing simultaneous or sequential multiplex engineering to knock out both B2M and CD58 loci and knock in GFP at CCR5 locus. These results were compared to a control with just knock in GFP at CCR5. FIG. 24B shows percentage of GFP expression in B lineage cells that underwent the aforementioned conditions. FIG. 24C shows percentage of CD58 expression among the same set of B lineage cell populations. FIG. 24D shows percentage of HLA-ABC expression among the same set of B lineage cell populations.

FIG. 25A shows in vitro bioluminescence intensity (BLI) for various indicated engineered B lineage cell populations comprising a LUC transgene at CCR5 (Control), a bicistronic HLA-E transgene and LUC transgene (comprising a 2A peptide) at CCR5 and a knockout of B2M (Bicis HLA-E), a LUC transgene at CCR5 and a knockout of NLRC5 (NLRC5 KO), or a LUC transgene at CCR5 and knockout of B2M and CD58 (B2M/CD58 DKO). These B lineage cells were dosed into NOG-IL15 mice with or without NK transfer alongside with K562 as a control. FIG. 25B and FIG. 25C show bioluminescence images of mice four hours (FIG. 25B) or 14 days (FIG. 25C) after they were dosed with engineered B lineage cells. FIG. 25D shows percentage of BLI of mice 14 days after they were dosed with B lineage cell populations or K562 in mice under NK pressure relative to mice without NK cells.

FIG. 26A shows bioluminescence images of NOG-IL6 mice 5 weeks after they were dosed with B lineage cell populations comprising a LUC transgene at CCR5 (Control), a LUC transgene at CCR5 and a knockout of B2M (B2M KO), a bicistronic HLA-E transgene and LUC transgene (comprising a 2A peptide) at CCR5 and a knockout of B2M (Bicis HLA-E), a LUC transgene at CCR5 and a knockout of NLRC5 (NLRC5 KO), or a LUC transgene at CCR5 and knockout of B2M and CD58 (B2M/CD58 DKO). FIG. 26B shows bioluminescence images of huCD34-NSG-IL15 mice 36 days after they were dosed with the same B lineage cell populations as FIG. 26A. FIG. 26C and FIG. 26D show measurement of whole body BLI were at timepoints 1, 3, 7, 14, 21, 27, and 36 days in NOG-IL6 mice (FIG. 26C) and hCD34-NSG-IL15 mice (FIG. 26D) after administration of indicated B lineage cell populations.

FIG. 27 shows percentage integration (homology directed repair, HDR, top left), Factor IX (FIX) secretion (top right), and FIX activity on D7 or D13 of B cell culture process from two different donor B lineage cell populations (LKP230152 and LKP230157) that comprise a FIX transgene at CCR5 (Control), a FIX transgene at CCR5 and a knockout of B2M (B2M KO), a FIX transgene at CCR5 and a knockout of NLRC5 (NLRC5 KO), a FIX transgene at CCR5 and a knockout of RFX5 (RFX5 KO) or a FIX transgene at CCR5 and knockout of B2M and CD58 (B2M/CD58 DKO) compared to a RNP alone control.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Activation: As used herein, the term “activation” refers to the state of a cell, for example a B cell that has been sufficiently stimulated to induce detectable cellular proliferation or has been stimulated to exert its effector function. Activation can also be associated with induced cytokine production, cell signaling, differentiation, and/or antigen processing and presentation.

Administration: As used herein, the term “administration” typically refers to the administration (e.g., of a composition or treatment) to a subject or system (e.g., that is or comprises one or more cells, tissues, organisms, etc), for example to achieve delivery of an agent that is, is included in, or is otherwise delivered or generated by, such composition or treatment. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent” (or “biological agent” or “therapeutic agent”), refers to a molecule that may be expressed, released, secreted or delivered to a target by a modified cell described herein. An agent includes, but is not limited to, a nucleic acid, an antibiotic, an anti-inflammatory agent, an antibody or fragments thereof, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Alloantigen: The term “alloantigen”, as used herein, refers to an antigen associated with allorecognition and/or graft rejection (e.g., an antigen against which a rejection immune response is directed). In general, alloantigens are agents that are present in or on tissue from one individual (e.g., a donor individual) of a particular species, but not in or on tissue from another individual (e.g., a recipient individual, for example who is genetically different from the donor individual) of the species, so that transfer of tissue from the donor individual to the recipient individual risks and/or results in a rejection immune response. In general, an antigen may be or include any chemical entity such as, for example, a small molecule, a nucleic acid, a polypeptide, a carbohydrate, a lipid, etc. In some embodiments, an alloantigen is or comprises a polypeptide. A variety of polypeptides are known in the art whose amino acid sequences can vary between and among individuals of the same species such that they might act as alloantigens.

Allogeneic: As used herein, the term “allogeneic” refers to any material (e.g., a population of cells) derived from a different animal of the same species.

Allorecognition: The term “allorecognition”, as used herein, typically refers to an immune response mounted by the immune system of an individual (i.e., a recipient) who receives a tissue graft from another individual (i.e., a donor, who for example is genetically distinct from the recipient individual) of the same species, which immune response involves recognition of an alloantigen on the grafted tissue. Typically, allorecognition involves T cell recognition of the alloantigen. In many embodiments, T cells recognize an alloantigen peptide, for example, encoded by a polymorphic gene whose sequence differs between the donor and recipient individuals.

Amelioration: As used herein, refers to the prevention, reduction or palliation of a state, or improvement of the state of a subject. Amelioration includes, but does not require complete recovery or complete prevention of a disease, disorder or condition (e.g., radiation injury).

Antigen: As used herein, the term “antigen” or “Ag” refers to a molecule that is capable of provoking an immune response. This immune response may involve either antibody production, activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Antibody agent: As used herein, the term “antibody agent” (interchangeably referred to herein as “antibody”) refers to a polypeptide that may be expressed, released, secreted, or delivered to a target by a modified cell described herein. The polypeptide includes canonical immunoglobulin sequence elements sufficient to confer specific binding to a particular target antigen. In some embodiments, an antibody agent comprises of an antibody. As is known in the art, antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody agent tetramers comprises two heavy chain-light chain dimers in which the heavy and light chains are linked to one another by a single disulfide bond; two other disulfide bonds connect the heavy chain hinge regions to one another, so that the dimers are connected to one another and a tetramer is formed. Antibody agents are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present disclosure (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody agent”, whether such polypeptide is naturally produced (e.g., generated by an organism reacting to an antigen), or produced by recombinant engineering, chemical synthesis, or other artificial system or methodology. In some embodiments, an antibody agent is polyclonal. In some embodiments, an antibody agent is monoclonal. In some embodiments, an antibody agent has constant region sequences that are characteristic of mouse, rabbit, primate, or human antibodies. In some embodiments, antibody agent sequence elements are humanized, primatized, chimeric, etc, as is known in the art. Moreover, the term “antibody agent”, as used herein, can refer in appropriate embodiments (unless otherwise stated or clear from context) to any of the art-known or developed constructs or formats for utilizing antibody structural and functional features in alternative presentation. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.].

Autologous: As used herein, the term “autologous” refers to any material derived from an individual to which it is later to be re-introduced into the same individual.

Biologically active: As used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Biomarker: The term “biomarker” is used herein, consistent with its use in the art, to refer to a to an entity, event, or characteristic whose presence, level, degree, type, and/or form, correlates with a particular biological event or state of interest, so that it is considered to be a “marker” of that event or state. To give but a few examples, in some embodiments, a biomarker may be or comprise a marker for a particular disease state, or for likelihood that a particular disease, disorder or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be or comprise an entity of any chemical class, and may be or comprise a combination of entities. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is detected outside of cells (e.g., is secreted or is otherwise generated or present outside of cells, e.g., in a body fluid such as blood, urine, tears, saliva, cerebrospinal fluid, etc. In some embodiments, a biomarker may be or comprise a genetic or epigenetic signature. In some embodiments, a biomarker may be or comprise a gene expression signature.

Bispecific antibody: As used herein, refers to a bispecific binding agent in which at least one, and typically both, of the binding moieties is or comprises an antibody component. A variety of different bi-specific antibody structures are known in the art. In some embodiments, each binding moiety in a bispecific antibody that is or comprises an antibody component includes VH and/or VL regions; in some such embodiments, such VH and/or VL regions are those found in a particular monoclonal antibody. In some embodiments, where the bispecific antibody contains two antibody component-binding moieties, each includes VH and/or VL regions from different monoclonal antibodies. In some embodiments, a bispecific antibody contains two antibody component binding moieties, wherein one of the two antibody component binding moieties includes an immunoglobulin molecule having VH and/or VL regions that contain CDRs from a first monoclonal antibody, and one of the two antibody component binding moieties includes an antibody fragment (e.g., Fab, F(ab′), F(ab′)2, Fd, Fv, dAB, scFv, etc.) having VH and/or VL regions that contain CDRs from a second monoclonal antibody. In some embodiments, a bispecific antibody can include Fc moieties, tags (e.g., His tags), or other elements to aid in circulation, purification, characterization, etc.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in an engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with a second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, such manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Endogenous: As used herein “endogenous” refers to any material from or produced inside a particular organism, cell, tissue or system.

Excipient: As used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, monocytes, macrophages, and/or dendritic cells. In one embodiment, monocytes, macrophages, or dendritic cells that are expanded ex vivo increase in number relative to the number originally present in a culture. In another embodiment, monocytes, macrophages, or dendritic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion,” as used interchangeably herein, refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Functional: As used herein, a “functional” biological molecule is a biological molecule in a form in which it exhibits a property and/or activity by which it is characterized.

Gene product or expression product: As used herein, the term “gene product” or “expression product” generally refers to an RNA transcribed from a gene (pre- and/or post-processing) or a polypeptide (pre- and/or post-modification) encoded by an RNA transcribed from a gene.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Immune cell: As used herein, the term “immune cell,” refers to a cell that is involved in an immune response, e.g., promotion of an immune response. Examples of immune cells include, but are not limited to, macrophages, monocytes, dendritic cells, neutrophils, eosinophils, mast cells, platelets, large granular lymphocytes, Langerhans' cells, natural killer (NK) cells, T-lymphocytes, plasma cells, plasmablasts, or B-lymphocytes. A source of immune cells (e.g., macrophages, monocytes, or dendritic cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, refers to a substance and/or entity that has been (1) separated from at least some of the components with which it was associated when initially produced (whether in nature and/or in an experimental setting) and/or otherwise previously associated, and/or (2) designed, produced, prepared, and/or manufactured by the hand of man. In some embodiments, a substance may be considered to be “isolated” if it is (or has been caused to be) free of or separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of other components (e.g., components with which it was previously associated). In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of a substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of presence or absence of a marker may vary depending upon a particular marker. In some embodiments, detection of a marker is highly specific in that it reflects a high probability that such tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. Those skilled in the art will appreciate that, in many embodiments, a useful marker need not distinguish with 100% accuracy.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid (e.g., a transgene), a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, an payload may be or comprise an agent in isolated or pure form; in some embodiments, such agent may be in crude form.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a response to treatment may refer to a beneficial alteration in a subject's condition that occurs as a result of or correlates with treatment. In some embodiment, such alteration may be or comprise stabilization of a condition (e.g., prevention of deterioration that would have taken place in the absence of a treatment), amelioration of symptoms of a condition, and/or improvement in prospects for cure of a condition, etc. In some embodiments, the term “response” may refer to a response of a particular system or components thereof (e.g., of a particular cell, tissue, organism, or subject). Those skilled in the art will be aware of technologies available to assess a response of interest.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample may be a “crude” sample in that it has been subjected to relatively little processing and/or is complex in that it includes components of relatively varied chemical classes.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Significant: As used herein, the term “significant” typically refers to the context wherein the difference or relationship between two variables (e.g., sequence identity, protein production, spatiotemporal conditions, etc.) are certain and exist. Significance can be statistically measured (e.g., statistically significant) by various mathematical formulas and models as understood by one skilled in the art. These methods include, but are not limited to, student t-test, two-tailed test, analysis of variance (ANOVA), etc. Furthermore, significance can impart differences within structure and chemistry between two different entities. For example, a sample molecule may be compared to a reference molecule, and exhibits a structural difference from said reference molecule that is significant, e.g., in the presence or absence or in the level of one or more biological or chemical moieties as compared to the reference entity.

Source: The term “source” as used herein, typically refers to a context in which an agent of interest (e.g., that may be or comprise a carbohydrate, a lipid, a nucleic acid, a metal, polypeptide, a small molecule, or a combination thereof) may be found in nature, or from which such agent can be or has been obtained (e.g., isolated). In some embodiments, a source may be or comprise a biological source (e.g., an organism, tissue, or cell, or sample thereof); in some embodiments, a source may be an environmental source. In some embodiments, a source may be or comprise a primary sample from an organism (e.g., which may be or comprise a tissue or fluid of such organism, and/or may be or comprise cell(s) of such organism). In some embodiments, an organism may be or comprise a prokaryotic organism (e.g., a bacterium) or a eukaryotic organism (e.g., a fungus or yeast, an insect, a mammal, a plant, a reptile, etc.). In some embodiments, an infectious agent such as a virus or phage may be considered an organism for purposes of this disclosure, and in particular with respect to being a source. In some embodiments, a source may be or comprise an engineered source, such as a cell line or culture, an in vitro system, etc.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a discase, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Substantially purified: As used herein, the term “substantially purified”, for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by.

Target Locus: As used herein, the term “target locus” may refer to a specific site or location on a chromosome of interest. For example, a target locus may be a site to be “engineered” or “altered” by the hand of man. In some embodiments described and/or utilized herein, an engineered polynucleotide comprises homology to a target locus in order to allow further alterations at a specific site (e.g., CCR5 as a target locus, whose homologous sequence may be part of a guide RNA to result in incorporation of an edit via CRISPR/Cas-mediated gene editing). In some embodiments, target locus may interchangeably refer to a target gene of interest for manipulation by man. In some embodiments, such target locus manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols).

Target site: As used herein, the term “target site” or “target sequence” refers to a genomic nucleic acid sequence that defines a portion of a nucleic acid to which a binding molecule may specifically bind under conditions sufficient for binding to occur.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more biological or chemical moieties as compared to the reference entity. In some embodiments, a variant also differs functionally from its reference molecule. In some embodiments, a variant differs structurally but performs the same or similar function as its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a variant polypeptide or nucleic acid is a truncated form of the reference polypeptide or nucleic acid. In some embodiments, a variant polypeptide that is a truncated form of the reference polypeptide may demonstrate comparable, identical, or greater levels of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

The present disclosure encompasses culturing and engineering in order to produce highly viable and productive (e.g., multiplex integration of one or more desired payloads) B lineage cell populations for administration into a subject population.

### Cell Therapy

Cell-based therapeutics (cell therapies) are an emerging class of medicine that make use of innate cellular machinery to combat discase. Unlike many traditional treatment methods, cell therapies make use of cellular localization, migration, and proliferation within the body, which can translate to improved biodistribution and targeted delivery of therapeutics. Cell therapies also benefit from cellular ability to sense and respond to various extrinsic signals within a subject, including, e.g., small molecules, other cells, physical forces, and/or marker proteins. Cellular persistence in vivo also enables cell therapies to survive, differentiate, function, etc. within a subject over extended time periods. These innate qualities may also lead to improved safety and efficacy for cell therapies as compared to other biologics or pharmaceutical compounds, providing long-lived, specifically targeted, adjustable, and/or responsive treatment for disease.

Cell-based therapies may have potential applications for a broad range of diseases, including those that have proved intractable or difficult to manage with traditional treatment options. Diseases that have been targeted for cell-based treatment include, e.g., various cancers, autoimmune diseases, central nervous system (CNS) diseases, neurodegenerative disorders, and cardiovascular diseases, among others. Cellular therapeutics offer an alternative over other treatment options for diseases where highly specific targeting (e.g., to a particular tissue type, area of the body, etc.) and/or longer-term treatment efficacy (e.g., enabling lower dosage frequency, single treatment options, etc.) are highly favored or necessary.

Cell therapies may employ a number of different cell types, which are typically modified to provide a therapeutic effect (e.g., transgene expression, reprogrammed cellular targeting, etc.). Although many cell types have potential to provide some form of therapeutic effect, recent therapies have heavily favored adaptive immune cells such as T lymphocytes and B lymphocytes (referred to interchangeably herein as T cells and B cells, respectively). For example, chimeric antigen receptor T (CAR-T) cells have been engineered to treat various cancers through recognition of one or more tumor cell markers, leading to cytotoxic destruction of tumor cells. CAR-T cells are also being adapted to treat infectious diseases (e.g., HIV) through recognition of other targeted antigens. Recent engineering efforts are focused on enhancing CAR-T receptor functionality, reducing innate immune response to CAR-T, and developing allogeneic therapies that make use of donor cells.

### B Lineage Cell Therapies

While cell-based therapies present an exciting new avenue for treatment of diseases, there are numerous challenges including achievement of safe, specific, and long-term therapeutic changes within targeted cells or tissues while reducing off-target effects. Furthermore, immune tolerance of these cell-based therapies is essential in order to obviate any deleterious side effects (See Jeske et al. 2021, incorporated by reference herein in its entirety). In order to address these challenges, engineering of B lineage cells have also been an area of development for cell-based therapies, due to the natural role of B lineage cells in antibody production within the body while minimizing inflammation. Antibody-based treatments are an established, well-studied form of treatment for a number of diseases, including cancer, autoimmune diseases, and infectious diseases. Monoclonal antibodies can be produced to target antigens within the body, which is valuable for treatment of diseases where such antibodies cannot be induced through natural processes (e.g., self-antigens for cancer and/or autoimmune diseases, antigens that fail to elicit natural immune response through infection and/or vaccination, etc.). Current antibody therapies require frequent administration and are costly to produce. Researchers have attempted to address these issues through use of gene therapy, which makes use of various techniques (e.g., viral vectors, CRISPR/Cas9 editing) to deliver antibody payloads to endogenous cells in the body, leading to persistent antibody production within a subject. However, these approaches can result in low levels of antibody expression and a counter-active response by the subject's own immune system.

Furthermore, due to their minimal impact on a subject's native immune system and capacity to continually produce antibodies, B lineage cells are a highly desirable cell-based target to secrete other payloads including, but not limited to, enzymes, complement proteins, cytokines, cytokine receptors, chimeric antigen receptors (CARs), anti-fibrotic molecules, anti-thrombotic molecules, antigens, both wild type and variant proteins, coagulation factors, glucose response elements, and fragments of antibodies, antigens, and proteins. Recent reports have described successful engineering of B lineage cells, for example, producing human B-cell activating factor (hBAFF) in a murine model. Excitingly, this work not only demonstrates successful production of hBAFF, but also engraftment in the bone marrow and viability of these engineered B lineage cells up to sixty days post-engraftment (See, Cheng et al. 2022, incorporated herein by reference in its entirety). B lincage cells are an attractive option for development of cell-based therapies and may potentially offer improved therapeutic effects (e.g., payload delivery, targeting, long-term payload expression, reduced auto-immune response, etc.) as compared to traditional therapies (e.g., antibody-based therapeutics, other cell therapies, etc.).

In some embodiments, a B lineage cell is a cell that expresses one or more B cell receptors (BCR) on a cell membrane. In some embodiments, a B lineage cell is a modified version or variant of a cell that expresses one or more B cell receptors (BCRs) on a cell membrane. In some embodiments, a B lineage cell is a naïve or memory B cell. In some embodiments, a B lineage cell is a cell derived from a naïve B cell (e.g., activated B lineage cell, plasmablast, plasma cell) or a variant thereof. In some embodiments, a B lineage cell is an activated B lineage cell. In some embodiments, a B lineage cell is a plasmablast. In some embodiments, a B lineage cell is a plasma cell.

In some embodiments, a B lineage cell population comprise naïve B cells. In some embodiments, naïve B cell populations are used as reference cell populations. In some embodiments, naïve B cell populations express CD19 (CD19⁺). In some embodiments, expression of CD19 in naïve B cell populations is used as a reference to assist in characterization of other B lineage cell populations. In some embodiments, naïve B cell populations express CD20 (CD20⁺). In some embodiments, expression of CD20 in naïve B cell populations is used as a reference to assist in characterization of other B lineage cell populations. In some embodiments, naïve B cell populations express low amounts of CD27 (CD27^{lo}). In some embodiments, expression of CD27 in naïve B cell populations are as a reference to assist in characterization of other B lineage cell populations. In some embodiments, naïve B cell populations express low amounts of CD38 (CD38^{lo}). In some embodiments, expression of CD38 in naïve B cell populations are used as a reference to assist in characterization of other B lineage cell populations. In some embodiments, naïve B cell populations express low amounts of CD138 (CD138^{lo}). In some embodiments, expression of CD138 in naïve B cell populations are used as a reference to assist in characterization of other B lineage cell population.

In some embodiments, a B lineage cell population comprises activated B lineage cells. In some embodiments, activated B lineage cell populations are used as reference cell populations. In some embodiments, activated B lineage cell populations are compared to a reference cell population (e.g., naïve B cell population). In some embodiments, activated B lineage cell populations express lower amounts of CD19 (CD19^{lo}) as compared to a reference cell population (e.g., naïve B cell populations). In some embodiments, activated B lineage cell populations express different (e.g., higher or lower) amounts of CD19 as compared to a reference cell population (e.g., differentiated B cell populations, etc.). In some embodiments, activated B lineage cell populations express lower amounts of CD20 (CD20^{lo}) as compared to a reference cell population (e.g., naïve B cell populations). In some embodiments, activated B lineage cell populations express different (e.g., higher or lower) amounts of CD20 as compared to a reference cell population (e.g., differentiated B cell populations, etc.). In some embodiments, activated B lineage cell populations express higher amounts of CD27 (CD27^{hi}) as compared to a reference cell population (e.g., naïve B cell populations). In some embodiments, activated B lineage cell populations express different (e.g., higher or lower) amounts of CD27 as compared to a reference cell population (e.g., differentiated B cell populations, etc.). In some embodiments, activated B lineage cell populations express lower amounts of CD38 (CD38^{lo}) as compared to a reference cell population (e.g., naïve B cell populations). In some embodiments, activated B lineage cell populations express different (e.g., higher or lower) amounts of CD38 as compared to a reference cell population (e.g., differentiated B cell populations, etc.). In some embodiments, activated B lineage cell populations express lower amounts of CD138 (CD138^{lo}) as compared to a reference cell population (e.g., naïve B cell populations). In some embodiments, activated B lineage cell populations express different (e.g., higher or lower) amounts of CD138 as compared to a reference cell population (e.g., differentiated B cell populations, etc.).

In some embodiments, a B lineage cell population may comprise plasmablast cells. In some embodiments, plasmablast cell populations are used as reference cell populations. In some embodiments, plasmablast cell populations are compared to a reference cell population (e.g., naïve B cell population). In some embodiments, plasmablast cell populations express lower amounts of CD19 (CD19^{lo}) as compared to a reference cell population (e.g., naïve B cell populations). In some embodiments, plasmablast cell populations express different (e.g., higher or lower) amounts of CD19 as compared to a reference cell population (e.g., activated cell populations, plasma cell populations, etc.). In some embodiments, plasmablast cell populations express lower amounts of CD20 (CD20^{lo}) as compared to a reference cell population (e.g., naïve B cell populations). In some embodiments, plasmablast cell populations express different (e.g., higher or lower) amounts of CD20 as compared to a reference cell population (e.g., activated cell populations, plasma cell populations, etc.). In some embodiments, plasmablast cell populations express higher amounts of CD27 (CD27^{hi}) as compared to a reference cell population (e.g., naïve B cell populations). In some embodiments, plasmablast cell populations express different (e.g., higher or lower) amounts of CD27 as compared to a reference cell population (e.g., activated cell populations, plasma cell populations, etc.). In some embodiments, plasmablast cell populations express higher amounts of CD38 (CD38^{hi}) as compared to a reference cell population (e.g., naïve B cell populations). In some embodiments, plasmablast cell populations express different (e.g., higher or lower) amounts of CD38 as compared to a reference cell population (e.g., activated cell populations, plasma cell populations, etc.). In some embodiments, plasmablast cell populations express lower amounts of CD138 (CD138^{lo}) as compared to a reference cell population (e.g., naïve B cell populations). In some embodiments, plasmablast cell populations express different (e.g., higher or lower) amounts of CD138 as compared to a reference cell population (e.g., activated cell populations, plasma cell populations, etc.).

In some embodiments, a B lineage cell population comprises plasma cells. In some embodiments, plasma cell populations are used as reference cell populations. In some embodiments, plasma cell populations are compared to a reference cell population (e.g., naïve B cell population). In some embodiments, plasma cell populations express lower amounts of CD19 (CD19^{lo}) as compared to a reference cell population (e.g., naïve B cell populations). In some embodiments, plasma cell populations express different (e.g., higher or lower) amounts of CD19 as compared to a reference cell population (e.g., activated cell populations, plasmablast populations, etc.). In some embodiments, plasma cell populations express lower amounts of CD20 (CD20^{lo}) as compared to a reference cell population (e.g., naïve B cell populations). In some embodiments, plasma cell populations express different (e.g., higher or lower) amounts of CD20 as compared to a reference cell population (e.g., activated cell populations, plasmablast populations, etc.). In some embodiments, plasma cell populations express higher amounts of CD27 (CD27^{hi}) as compared to a reference cell population (e.g., naïve B cell populations). In some embodiments, plasma cell populations express different (e.g., higher or lower) amounts of CD27 as compared to a reference cell population (e.g., activated cell populations, plasmablast populations, etc.). In some embodiments, plasma cell populations express higher amounts of CD38 (CD38^{hi}) as compared to a reference cell population (e.g., naïve B cell populations). In some embodiments, plasma cell populations express different (e.g., higher or lower) amounts of CD38 as compared to a reference cell population (e.g., activated cell populations, plasmablast populations, etc.). In some embodiments, plasma cell populations express higher amounts of CD138 (CD138^{hi}) as compared to a reference cell population (e.g., naïve B cell populations). In some embodiments, plasma cell populations express different (e.g., higher or lower) amounts of CD138 as compared to a reference cell population (e.g., activated cell populations, plasmablast populations, etc.).

### Allogeneic Therapy

Allogencic therapy provides an appealing option for therapies involving administration (e.g., transplant, treatment, etc.) of one or more cells (e.g., cells, tissues, organs, etc.) from a single source (referred to interchangeably herein as a donor) to a recipient that is not genetically identical to the donor. Unlike autologous therapy methods, which require a donor that is genetically identical (or substantially genetically identical) to a recipient (e.g., donor and recipient are the same individual), allogeneic therapies are described in the art as potentially “universal” therapies that can provide treatment options (e.g., cell therapies, transplants, etc.) from a single source that are administered to multiple patients. Allogeneic therapies are desirable because, among other things, they can reduce manufacturing costs and complications by treating donor cells (e.g., cells, tissues, organs, etc.) to prepare therapies compatible for administration to multiple patients. One challenge recognized by those in the art is the preparation of allogeneic therapies that are capable of administration to multiple non-donor subjects without eliciting an immune response in a recipient. Current therapies are often administered in combination with one or more alternative therapies, e.g., immunosuppressants, to reduce the risk of a recipient immune response. The present disclosure provides a recognition that, among other things, certain modifications (e.g., genetic modifications) to one or more donor cells (e.g., donor B lineage cells, engineered B lineage cells) can provide improved allogeneic properties (e.g., reduced immune response in recipient) while providing one or more therapeutic benefits (e.g., expression and/or secretion of therapeutic transgene). In some embodiments, improved allogeneic properties may comprise reduced immune response in a recipient through evasion of one or more recipient immune response pathways, including, e.g., T cell recognition (e.g., CD8+ T cells, CD4+ T cells) and natural killer (NK) cell recognition.

In some embodiments, the present disclosure provides a recognition that B lineage cells (e.g., plasma cells, plasmablasts) may be engineered to produce improved allogeneic properties (referred to interchangeably herein as allogeneicity). In some embodiments, B lineage cell populations are engineered to reduce levels of one or more endogenous proteins (e.g., MHC-I proteins, MHC-II proteins, proteins involved in formation of immune synapses, etc.) in order to provide improved allogeneic properties.

Among other things, the present disclosure provides a recognition that modification of one or more endogenous genes (e.g., through modification of corresponding mRNA and/or protein expression) can provide improve allogeneic properties. For example, it has been reported in the art that certain signals (e.g., CD58) are associated with formation of immune synapses by natural killer (NK) cells and cytotoxic T cells, which are necessary to induce target cell killing. In some embodiments, modification (e.g., disruption, knockout) of CD58 expression in B lineage cells may improve resistance to NK cells and T cells (e.g., reduce target cell killing or cell activation).

It has also been reported in the art that certain signals (e.g., TAP1, TAP2, TAPBP, NLRC5) are associated with formation of endogenous MHC-I complexes on the surface of B lineage cells. Among other things, the present disclosure provides an insight that, in some embodiments, modification (e.g., disruption, knockout) of expression of certain signals (e.g., TAP1, TAP2, TAPBP, NLRC5) may improve resistance to T-cell response. In some embodiments, modification (e.g., disruption, knockout) of expression of certain signals (e.g., TAP1, TAP2, TAPBP, NLRC5) may improve resistance to T-cell response without increasing NK cell response. In some embodiments, modification (e.g., disruption, knockout) of expression of certain signals (e.g., TAP1, TAP2, TAPBP, NLRC5) may improve resistance to T-cell response while maintaining comparable NK cell response. In some embodiments, modification (e.g., disruption, knockout) of expression of certain signals (e.g., TAP1, TAP2, TAPBP, NLRC5) may reduce expression and/or formation of endogenous MHC-I. In some embodiments, modification (e.g., disruption, knockout) of expression of certain signals (e.g., TAP1, TAP2, TAPBP, NLRC5) may reduce expression and/or formation of endogenous MHC-I while maintaining comparable levels of endogenous HLA-E. In some embodiments, modification (e.g., disruption, knockout) of expression of certain signals (e.g., TAP1, TAP2, TAPBP, NLRC5) may reduce expression and/or formation of endogenous MHC-I while reducing levels of endogenous HLA-E. In some embodiments, modification (e.g., disruption, knockout) of expression of certain signals (e.g., TAP1, TAP2, TAPBP, NLRC5) may reduce expression and/or formation of endogenous MHC-I while increasing levels of endogenous HLA-E.

Without wishing to be bound by any particular theory, the present disclosure also provides an insight that certain signals (e.g., RFX5) are associated with immune response (e.g., T-cell response and NK cell response) to certain cell therapies (e.g., B lineage cell therapies). In some embodiments, modification (e.g., disruption, knockout) of expression of certain signals (e.g., RFX5) may reduce expression and/or formation of endogenous MHC-I while maintaining comparable levels of endogenous HLA-E. In some embodiments, modification (e.g., disruption, knockout) of expression of certain signals (e.g., RFX5) may reduce expression and/or formation of endogenous MHC-I while increasing levels of endogenous HLA-E. In some embodiments, modification (e.g., disruption, knockout) of expression of certain signals (e.g., RFX5) may reduce expression and/or formation of endogenous MHC-II. In some embodiments, modification (e.g., disruption, knockout) of expression of certain signals (e.g., RFX5) may reduce expression and/or formation of endogenous MHC-I and MHC-II. In some embodiments, modification (e.g., disruption, knockout) of expression of certain signals (e.g., RFX5) may reduce expression and/or formation of endogenous MHC-I and MHC-II while maintaining comparable levels of endogenous HLA-E. In some embodiments, modification (e.g., disruption, knockout) of expression of certain signals (e.g., RFX5) may reduce expression and/or formation of endogenous MHC-I and MHC-II while increasing levels of endogenous HLA-E.

### Cell Engineering

Production of engineered cells for various uses including, e.g., cell therapies is an active area of development. Genomic and epigenomic modifications, synthetic biology, and application of biomaterials may be employed to generate engineered cells with desired properties for therapeutic applications. Selection of an appropriate method for generating engineered cells is typically dependent upon the desired output and effects of cell therapy and requires optimization for different cell types, transgenes of interest, etc. It is understood in the art that engineering methods that are effective for a particular cell type may be less effective (or not viable) for another cell type. Furthermore, engineering methods may vary depending upon whether a therapy requires cellular localization, expression of an endogenous or exogenous protein, removal of an endogenous protein, etc.

In some embodiments, cell engineering comprises use of one or more genome editing tools as described herein.

### Genome Editing

Genome editing tools can alter a cellular genome to produce a desired therapeutic effect, e.g., expression of a therapeutic protein. Targeted nucleases (e.g., Cas proteins, TALENS, ZFNs, etc.), viral vectors (e.g., AAV, lentiviral, adenoviral, etc.), recombinases (e.g., Cre recombinase, Flp recombinase, PhiC31 integrase, etc.), and other tools may be employed to provide genetic modifications. Gene editing efficiency may vary depending on, e.g., cell type, desired function, and ease of delivery. Accordingly, editing methods often require extensive optimization to provide engineered cells with intended functionality for therapeutic applications.

### B Lineage Cell Engineering

Various B lineage cell engineering techniques are described in the art, including, e.g., CRISPR/Cas9, AAV, lentiviral, and recombinase-based methods. These methods are generally employed to introduce a payload (e.g., expression cassette, transgene, etc.) into naïve B cells in order to express a protein of interest. A payload may be designed for episomal expression, integration into a specific target locus (e.g., endogenous target gene locus), or integration into a non-specific locus (e.g., endogenous random or non-target gene locus). Payloads may be designed such that an endogenous target gene locus continues to produce functional protein and/or fulfill its natural function (non-disruptive integration). Payloads may also be designed to intentionally disrupt an endogenous target gene locus to produce lowered or non-detectable levels of functional protein and/or some amount of non-functional protein (also referred to interchangeably herein as disruptive integration). The present disclosure provides for methods for multiplex integration of more than one payload at one or more target locus.

The methods for integration of a payload (e.g., expression cassette comprising one or more transgenes) into an endogenous target locus may comprise site-specific cleavage with a targeted nuclease (e.g., Cas protein, including Cas9), followed by integration of a transgene (e.g., SMPD1, Factor IX, blinatumomab, etc.) through an endogenous repair pathway (e.g., homologous recombination, homology-directed repair, etc.). In some embodiments, methods for integration of an expression cassette comprising a transgene comprise site-specific cleavage at a target locus (e.g., CCR5) with a guide RNA/Cas9 complex, followed by integration of a transgene at the target locus through homologous recombination.

In some embodiments, a method of B lineage cell engineering is or comprises administration of a ribonucleoprotein (RNP) to a cell population. In some embodiments, a method of B lineage cell engineering is or comprises administration of a composition comprising a Cas protein complexed with guide RNA (gRNA) to a cell population. In some embodiments, a method of B lineage cell engineering is or comprises administration of a composition comprising a Cas9/guide RNA complex a cell population. In some embodiments, a method of B lineage cell engineering is or comprises administration of a composition comprising a Cas9/guide RNA complex a cell population. In some embodiments, a method of B lineage cell engineering is or comprises administration of a composition comprising a payload (e.g., expression cassette comprising one or more transgenes) of interest to a cell population. In some embodiments, a method of B lineage cell engineering is or comprises administration of a composition comprising a payload (e.g., expression cassette comprising one or more transgenes) to a cell population through use of a viral vector. In some embodiments, a method of B lineage cell engineering is or comprises administration of a composition comprising a payload (e.g., expression cassette comprising one or more transgenes) encapsulated within an AAV capsid (e.g., AAV2, AAV3, AAV5, AAV6, AAV8, etc.) to a cell population. In some embodiments, a method of B lineage cell engineering is or comprises administration of a composition comprising a transgene encapsulated within an AAV capsid (e.g., AAV2, AAV3, AAV5, AAV6, AAV8, etc.) in combination with or in addition to administration of a composition comprising a Cas9/gRNA complex.

In some embodiments, a method of B lineage cell engineering comprises a step of electroporation to facilitate cellular uptake of one or more engineering components. In some embodiments, a method of B lineage cell engineering comprises a step of electroporation to facilitate cellular uptake of a Cas9/gRNA complex. In some embodiments, a method of B lincage cell engineering comprises a step of electroporation to facilitate cellular uptake of a Cas9/gRNA complex and a payload. In some embodiments, a method of B lineage cell engineering may comprise a step of electroporation to facilitate cellular uptake of a Cas9/gRNA complex and a payload (e.g., expression cassette comprising one or more transgenes) encapsulated in an AAV capsid (AAV2, AAV3, AAV5, AAV6, AAV8, etc.). In some embodiments, a method of B lineage cell engineering comprises a step of electroporation to facilitate cellular uptake of a Cas9/gRNA complex and a payload (e.g., expression cassette comprising one or more transgenes) encapsulated in an AAV6 capsid.

In some embodiments, a method of B lineage cell engineering comprises a step of transfection. In some embodiments, a method of B lineage cell engineering comprises a step of nucleofection.

In some embodiments, a method of B lineage cell engineering comprises a step of viral transduction to facilitate cellular uptake of a payload (e.g., expression cassette comprising one or more transgenes). In some embodiments, a method of B lineage cell engineering comprises a step of viral transduction to facilitate cellular uptake of a payload (e.g., transgene) encapsulated in an AAV capsid (AAV2, AAV3, AAV5, AAV6, AAV8, etc.). In some embodiments, a method of B lineage cell engineering comprises a step of viral transduction to facilitate cellular uptake of a payload (e.g., expression cassette comprising one or more transgenes) encapsulated in an AAV6 capsid. In some embodiments, a method of B lineage cell engineering comprises one or more steps of: (i) electroporation to facilitate cellular uptake of a Cas9/gRNA complex; and (ii) viral transduction to facilitate cellular uptake of a payload encapsulated in an AAV capsid.

In some embodiments, a method of B lineage cell engineering comprises integration into multiple target loci. In some embodiments, a method of B lineage cell engineering comprises integration of a payload (transgenes, expression cassettes, etc.) into a first target locus (e.g., safe harbor locus, endogenous gene locus, etc.) and disruption (e.g., knockout of endogenous protein expression, RNA production, etc.) of a second target locus (e.g., safe harbor locus, endogenous gene locus, etc.)). In some embodiments, a method of B lineage cell engineering comprises integration of a payload (e.g., transgenes, expression cassettes, etc.) into a CCR5 target locus and disruption (e.g., knockout of endogenous protein expression, RNA production, etc.) of a B2M target locus.

In some embodiments, a method of B lineage cell engineering comprises integration of one or more payloads (e.g., transgenes, expression cassettes, etc.) into multiple target loci (e.g., safe harbor loci, endogenous gene loci, combinations thereof, etc.), also referred to interchangeably herein as multiplex engineering. In some embodiments, a method of B lineage cell engineering comprises integration of one or more payloads (e.g., transgenes, expression cassettes, etc.) into multiple, distinct target loci (e.g., safe harbor loci, endogenous gene loci, combinations thereof, etc.). In some embodiments, a method of B lineage cell engineering comprises non-disruptive integration of one or more payloads (e.g., transgenes, expression cassettes, etc.) into multiple, distinct target loci (e.g., safe harbor loci, endogenous gene loci, combinations thereof, etc.). In some embodiments, a method of B lineage cell engineering comprises disruptive integration of one or more payloads (e.g., transgenes, expression cassettes, etc.) into multiple, distinct target loci (e.g., safe harbor loci, endogenous gene loci, combinations thereof, etc.). In some embodiments, a method of B lineage cell engineering comprises simultaneous integration of one or more payloads (e.g., transgenes, expression cassettes, etc.) into multiple, distinct target loci (e.g., safe harbor loci, endogenous gene loci, combinations thereof, etc.).

In some embodiments, a method of B lineage cell engineering comprises sequential integration of a first payload (e.g., transgene, expression cassette, etc.) into a first target locus (e.g., safe harbor locus, endogenous gene locus, etc.), followed by integration of a second payload (e.g., transgene, expression cassette, etc.) into a second target locus (e.g., safe harbor locus, endogenous gene locus, etc.). In some embodiments, a method of B lineage cell engineering comprises sequential integration of a first payload (e.g., transgene, expression cassette, etc.) into a first target locus (e.g., safe harbor locus, endogenous gene locus, etc.) at a first timepoint (e.g., day 2 or 3 in culture process), followed by integration of a second payload (e.g., transgene, expression cassette, etc.) into a second target locus (e.g., safe harbor locus, endogenous gene locus, etc.) at a second timepoint (e.g., day 3 or 4 in culture process). In some embodiments, a first step of culturing cells is described as day 1 of a culture process. In some embodiments, a first step of culturing cells is described as day 0 of a culture process.

In some embodiments, a method of B lineage cell engineering comprises integration of one or more payloads (e.g., transgenes, expression cassettes, etc.) into multiple target loci (e.g., safe harbor loci, endogenous gene loci, combinations thereof, etc.) to produce multiplex engineered B lineage cell populations. In some embodiments, a method of B lineage cell engineering comprises simultaneous integration of one or more payloads (e.g., transgenes, expression cassettes, etc.) into multiple, distinct target loci (e.g., safe harbor loci, endogenous gene loci, combinations thereof, etc.) to produce simultaneously multiplex engineered B lineage cell populations. In some embodiments, a method of B lineage cell engineering comprises sequential integration of a first payload (e.g., transgene, expression cassette, etc.) into a first target locus (e.g., safe harbor locus, endogenous gene locus, etc.), followed by integration of a second payload (e.g., transgene, expression cassette, etc.) into a second target locus (e.g., safe harbor locus, endogenous gene locus, etc.) to produce sequentially multiplex engineered B lineage cell populations.

In some embodiments, a method for multiplex engineering of B lineage cells may comprise site-specific cleavage with one or more targeted nuclease (e.g., Cas protein, including Cas9), followed by integration of one or more transgenes (e.g., SMPD1, Factor IX, blinatumomab, etc.) through an endogenous repair pathway (e.g., homologous recombination, homology-directed repair, etc.). In some embodiments, a method for multiplex engineering of B lineage cells may comprise two or more expression cassettes, each comprising one or more transgenes which comprise site-specific cleavage at each target locus (e.g., CCR5, B2M) with one or more guide RNA/Cas9 complexes, followed by integration of one or more transgenes at each target locus through homologous recombination. In some embodiments, a method for multiplex engineering of B lineage cells may comprise simultaneous introduction of two or more expression cassettes each comprising one or more transgenes which comprise site-specific cleavage at each target locus (e.g., CCR5, B2M) with one or more guide RNA/Cas9 complexes, followed by integration of one or more transgenes at each target locus through homologous recombination. In some other embodiments, a method for multiplex engineering of B lineage cells may comprise introduction of two or more expression cassettes each comprising one or more transgenes which comprise site-specific cleavage at a target locus (e.g., CCR5, B2M) with one or more guide RNA/Cas9 complexes occurs within a time period under 24 hours. In some embodiments, a method for multiplex engineering of B lineage cells may comprise sequential introduction of two or more expression cassettes each comprising one or more transgenes which comprise site-specific cleavage at a target locus (e.g., CCR5, B2M) with one or more guide RNA/Cas9 complexes, followed by integration of one or more transgenes at each target locus through homologous recombination. In some embodiments, a time period may exist between sequential introduction of expression cassettes. In some embodiments, a time period between each sequential introduction of expression cassettes may be about 24 hours to about 144 hours.

In some embodiments, a method of B lineage cell engineering is or comprises administration of one or more ribonucleoproteins (RNPs) to a cell population. In some embodiments, a method of multiplex B lineage cell engineering is or comprises administration of one or more compositions, wherein each comprise of a Cas protein complexed with guide RNA (gRNA) to a cell population. In some embodiments, a method of multiplex B lineage cell engineering is or comprises administration of one or more compositions, wherein each comprising a Cas9/guide RNA complex a cell population. In some embodiments, a method of multiplex B lineage cell engineering is or comprises administration of one or more composition, wherein each comprising a Cas9/guide RNA complex a cell population. In some embodiments, a method of multiplex B lineage cell engineering is or comprises administration of one or more compositions, wherein each comprising a payload (e.g., expression cassette comprising one or more transgenes) of interest to a cell population. In some embodiments, a method of multiplexB lineage cell engineering is or comprises administration of one or more compositions, wherein each comprising a payload (e.g., expression cassette comprising one or more transgenes) to a cell population through use of a viral vector. In some embodiments, a method of multiplex B lineage cell engineering is or comprises administration of one or more compositions, wherein each comprising a payload (e.g., expression cassette comprising one or more transgenes) encapsulated within an AAV capsid (e.g., AAV2, AAV3, AAV5, AAV6, AAV8, etc.) to a cell population. In some embodiments, a method of multiplex B lineage cell engineering is or comprises administration of one or more compositions, wherein each comprising a transgene encapsulated within an AAV capsid (e.g., AAV2, AAV3, AAV5, AAV6, AAV8, etc.) in combination with or in addition to administration of a composition comprising a Cas9/gRNA complex.

In some embodiments, a method of multiplex B lineage cell engineering comprises a step of electroporation to facilitate cellular uptake of one or more engineering components. In some embodiments, a method of multiplex B lineage cell engineering may comprise more than one step of electroporation to facilitate cellular uptake of one or more engineering components. In some embodiments, a method of multiplex B lineage cell engineering comprises one or more steps of electroporation to facilitate cellular uptake of one or more Cas9/gRNA complexes. In some embodiments, a method of multiplex B lineage cell engineering comprises one or more steps of electroporation to facilitate cellular uptake of one or more Cas9/gRNA complexes and one or more payloads. In some embodiments, a method of multiplex B lineage cell engineering may comprise one or more steps of electroporation to facilitate cellular uptake of one or more Cas9/gRNA complexes and one or more payloads (e.g., expression cassette comprising one or more transgenes) encapsulated in an AAV capsid (AAV2, AAV3, AAV5, AAV6, AAV8, etc.). In some embodiments, a method of multiplex B lineage cell engineering comprises one or more steps of electroporation to facilitate cellular uptake of one or more Cas9/gRNA complexes and one or more payloads (e.g., expression cassette comprising one or more transgenes) each encapsulated in an AAV6 capsid.

In some embodiments, a method of multiplex B lineage cell engineering comprises one or more steps of viral transduction to facilitate cellular uptake of one or more payloads (e.g., expression cassette comprising one or more transgenes). In some embodiments, a method of multiplex B lineage cell engineering comprises one or more steps of viral transduction to facilitate cellular uptake of one or more payload (e.g., transgene) each encapsulated in an AAV capsid (AAV2, AAV3, AAV5, AAV6, AAV8, etc.). In some embodiments, a method of multiplex B lineage cell engineering comprises one or more steps of viral transduction to facilitate cellular uptake of one or more payloads (e.g., expression cassette comprising one or more transgenes) each encapsulated in an AAV6 capsid. In some embodiments, a method of multiplex B lineage cell engineering comprises one or more steps of: (i) electroporation to facilitate cellular uptake of a Cas9/gRNA complex; and (ii) viral transduction to facilitate cellular uptake of a payload encapsulated in an AAV capsid. In some other embodiments, a method of multiplex B lineage cell engineering may comprise one or more additional repeats of the following one or more steps: (i) electroporation to facilitate cellular uptake of a Cas9/gRNA complex; and (ii) viral transduction to facilitate cellular uptake of a payload encapsulated in an AAV capsid.

In some embodiments, a method of cell engineering is particularly effective for one type of cell (e.g., T cell) and less effective for another type of cell (e.g., B cell). In some embodiments, a method of B lineage cell engineering provides improved genome editing efficiency in B lineage cells as compared to other cell types (e.g., T cell). In some embodiments, a method of B cell engineering provides at least about 10%, 15%, 20%, 25%, 30%, 35%, 40%, 45%, 50%, 55%, 60%, 65%, 70%, 75%, 80%, 85%, 90%, 95%, or 99% editing efficiency. In some embodiments, a method of B lineage cell engineering provides at least about 1E6, 1.5E6, 2E6, 2.5E6, 3E6, 3.5E6, 4E6, 4.5E6, 5E6, 5.5E6, 6E6, 6.5E6, 7E6, 7.5E6, 8E6, 8.5E6, 9E6, 9.5E6, 1E7, 1.5E7, 2E7, 2.5E7, 3E7, 3.5E7, 4E7, 4.5E7, or 5E7 edited B lineage cells.

In some embodiments, a method of B lineage cell engineering comprises a step of editing activated B cells. In some embodiments, a method of B lineage cell engineering comprises a step of editing B lineage cells after an activation step of about 1, 2, 3, 4, or 5 day(s). In some embodiments, a method of B lineage cell engineering comprises a step of editing B lineage cells after an activation step of 2 days. In some embodiments, a method of B lineage cell engineering comprises a step of editing B lineage cells after an activation step of about 1, 2, 3, 4, or 5 day(s) and expanding edited B lineage cells in the activation media for an additional period of about 1, 2, 3, 4, 5, 6, 7, or 8 days. In some embodiments, a method of B lineage cell engineering comprises a step of editing B lineage cells after an activation step of 2 days and expanding edited B lineage cells in the activation media for an additional 6 days. In some embodiments, a method of B lineage cell engineering comprises one or more additional steps of editing B lineage cells after an activation step of about 1, 2, 3, 4, or 5 day(s). In some embodiments, there exist a time period between each subsequent editing step. In some embodiments, this time period is between 24 to 144 hours.

### Payloads

Various methods described herein may be used for generation of engineered cells (e.g., B lineage cell populations, etc.) comprising one or more payloads. In some embodiments, engineered cells (e.g., B lineage cell populations, etc.) comprise a polynucleotide sequence encoding one or more payloads. In accordance with various aspects of the present disclosure, any of a variety of payloads may be used (e.g., those with a therapeutic or monitoring purpose), alone or in combination. In some embodiments, a payload is or comprises a polynucleotide sequence encoding a peptide or polypeptide. In some embodiments, a payload is or comprises one or more transgenes. In some embodiments, a payload is or comprises one or more homology arm sequences. In some embodiments, a payload is or comprises a transgene flanked by one or more homology sequences.

In some embodiments, a payload may comprise a sequence for polycistronic expression (including, e.g., a 2A peptide, intronic sequence, or internal ribosomal entry site (IRES)). In some embodiments, 2A peptides are small (e.g., approximately 18-22 amino acids) peptide sequences enabling co-expression of two or more discrete protein products within a single coding sequence. In some embodiments, 2A peptides allow co-expression of two or more discrete protein products regardless of arrangement of protein coding sequences. In some embodiments, 2A peptides are or comprise viral sequences (e.g., foot-and-mouth diseases virus (F2A), equine Rhinitis A virus, porcine teschovirus-1 (P2A), or Thosea asigna virus (T2A)). In some embodiments, a 2A peptide is P2A. In some embodiments, a 2A peptide is T2A. In some embodiments, a 2A peptide is Furin-P2A.

In some embodiments, a payload may be or comprise one or more nucleic acid sequences encoding a reporter gene (e.g., a fluorescent or luminescent reporter).

In some embodiments, a payload may be or comprise a polynucleotide sequence, which comprises an expression cassette. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.). In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination in order to promote expression of a transgene in a cell population. In some embodiments, an expression cassette comprises one or more polynucleotide sequence elements (e.g., promoters, enhancers, transgenes, termination elements, homology arms, biomarkers, signal peptide sequences, internal ribosome entry site elements, self-cleaving peptide sequences, ubiquitous chromatin opening element, etc.) in a particular configuration and/or combination in order to promote expression of a transgene in an engineered B lineage cell population.

In some embodiments, an expression cassette comprises one or more polynucleotide sequences encoding one or more promoters (e.g., MND, CMV, SFFV, FEEK I, EF-1a, etc.). In some embodiments, an expression cassette comprises one or more polynucleotide sequences encoding one or more exogenous promoters. In some embodiments, an expression cassette comprises one or more polynucleotide sequences encoding one or more endogenous promoters. In some embodiments, an expression cassette does not comprise one or more promoters. In some embodiments, an expression cassette does not comprise one or more exogenous promoters. In some embodiments, an expression cassette does not comprise one or more endogenous promoters.

In some embodiments, an expression cassette comprises one or more polynucleotide sequences encoding a translation initiation site (e.g., Kozak consensus sequence, ribosomal binding site, etc.). In some embodiments, an expression cassette comprises one or more exogenous polynucleotide sequences encoding a translation initiation site. In some embodiments, an expression cassette comprises one or more endogenous polynucleotide sequences encoding a translation initiation site. In some embodiments, an expression cassette does not comprise one or more polynucleotide sequences encoding a translation initiation site. In some embodiments, an expression cassette does not comprise one or more exogenous polynucleotide sequences encoding a translation initiation site. In some embodiments, an expression cassette does not comprise one or more endogenous polynucleotide sequences encoding a translation initiation site.

In some embodiments, an expression cassette comprises one or more polynucleotide sequences encoding one or more enhancers (e.g., WPRE, beta-globin etc.). In some embodiments, an expression cassette comprises one or more polynucleotide sequences encoding one or more exogenous enhancers. In some embodiments, an expression cassette comprises one or more polynucleotide sequences encoding one or more endogenous enhancers. In some embodiments, an enhancer may be viral (e.g., WPRE, etc.) or non-viral. In some embodiments, an expression cassette does not comprise one or more enhancers. In some embodiments, an expression cassette does not comprise one or more exogenous enhancers. In some embodiments, an expression cassette does not comprise one or more endogenous enhancers.

In some embodiments, an expression cassette comprises one or more polynucleotide sequences encoding one or more terminators (e.g., polyA, including, e.g., BGH polyA, SV40 polyA, etc.). In some embodiments, an expression cassette comprises one or more polynucleotide sequences encoding one or more exogenous terminators. In some embodiments, an expression cassette comprises one or more polynucleotide sequences encoding one or more endogenous terminators. In some embodiments, an expression cassette does not comprise one or more terminators. In some embodiments, an expression cassette does not comprise one or more exogenous terminators. In some embodiments, an expression cassette does not comprise one or more endogenous terminators.

In some embodiments, an expression cassette comprises a polynucleotide sequence encoding a transgene (e.g., SMPD1, Factor IX, blinatumomab, etc.) or variant thereof. In some embodiments, an expression cassette comprises a polynucleotide sequence encoding a transgene (e.g., SMPD1, Factor IX, blinatumomab, etc.) or variant thereof for expression of a peptide or polypeptide (e.g., acid sphingomyelinase, Factor IX, blinatumomab, etc.) or variant thereof.

### Transgenes

In some embodiments, a transgene is a corrective gene chosen to improve one or more signs and/or symptoms of a disease, disorder, or condition. In some embodiments, transgenes are functional versions of disease associated genes (i.e., gene isoform(s) which are associated with the manifestation or worsening of a disease, disorder or condition) found in a subject. In some embodiments, one or more transgenes are optimized versions of disease-associated genes found in a subject (e.g., codon optimized or expression-optimized variants). In some embodiments, transgenes are variants of disease-associated genes found in a subject (e.g., a functional gene fragment or variant thereof). In some embodiments, a transgene is a gene that causes expression of a peptide that is normally expressed in one or more healthy tissues.

In some embodiments, a transgene is a gene that causes expression of an altered protein with a gain- or loss-of-function mutation. In some embodiments, a transgene is a gene that causes expression of a fusion protein. In some embodiments, a transgene is a gene that causes expression of an antibody agent. In some embodiments, a transgene is a gene that causes expression of a multispecific antibody. In some embodiments, a transgene is a fragment of an antibody, antigen, or protein. In some embodiments, a transgene is a gene that causes expression an enzyme (e.g., for enzyme replacement therapy). In some embodiments, a transgene is a gene that causes expression of a cytokine. In some embodiments, a transgene is a gene that causes expression of a cytokine receptor. In some embodiments, a transgene is a gene that causes expression of a chimeric antigen receptor (CAR). In some embodiments, a transgene is a gene that causes expression of an anti-thrombotic molecule. In some embodiments, a transgene is a gene that causes expression of a coagulation factor. In some embodiments, a transgene is a gene that causes expression of a glucose response element. In some embodiments, a transgene is a gene that causes expression of a nanobody. In some embodiments, a transgene is a gene that causes expression of sphingomyelin phosphodiesterase 1 (SMPD1), acid sphingomyelinase (ASM), Factor IX, blinatumomab, HLA (e.g., HLA-A, HLA-B, HLA-C, HLA-E, HLA-G etc.), B2M, and/or BCMA, or a variant thereof. Inn some embodiments, a transgene may comprise one or more antibody agents (e.g., antibody, BiTE, etc.). In some embodiments, a transgene may comprise one or more protein fusions. In some embodiments, a transgene may comprise a B2M fusion. In some embodiments, a transgene may comprise a HLA fusion. In some embodiments, a transgene may comprise a B2M/HLA-E single-chain fusion. In some embodiments, a transgene may comprise one or more proteins linked.

In some embodiments, a transgene is or comprises a gene encoding a functional nucleic acid. In some embodiments, a therapeutic agent is or comprises an agent that has a therapeutic effect upon a host cell or subject (including, e.g., a ribozyme, guide RNA (gRNA), antisense oligonucleotide (ASO), miRNA, siRNA, and/or shRNA). For example, in some embodiments, a therapeutic agent promotes a biological process to treat a medical condition, e.g., at least one symptom of a disease, disorder, or condition.

In some embodiments, transgene expression in a subject results substantially from integration at a target locus. In some embodiments, 75% or more (e.g., 80% or more, 85% or more, 90% or more, 95% or more, 99% or more, 99.5% or more) of total transgene expression in a subject is from transgene integration at a target locus. In some embodiments, 25% or less (e.g., 20% or less, 15% or less, 10% or less, 5% or less, 1% or less, 0.5% or less, 0.1% or less) of total transgene expression in a subject is from a source other than transgene integration at a target locus (e.g., episomal expression, integration at a non-target locus).

In some embodiments, a transgene is or comprises a sequence having at least 80%, 85%, 90%, 95%, 99%, or 100% identity to a corresponding wild-type reference nucleotide sequence (e.g., a wild-type gene sequence). In some embodiments, a transgene is or comprises a sequence having a least 80%, 85%, 90%, 95%, 99%, or 100% identity to a portion of a corresponding wild-type reference nucleotide sequence (e.g., a wild-type gene sequence).

### Homology Arms

In some embodiments, a payload (e.g., expression cassette) may comprise one or more flanking polynucleotide sequences with significant sequence homology to a target locus (e.g., homology arms). In some embodiments, homology arms flank a polynucleotide sequence encoding a payload (e.g., one homology arm is 5′ to a payload (also referred to herein as a 5′ homology arm) and one homology arm is 3′ to a payload (also referred to herein as a 3′ homology arm). In some embodiments, homology arms direct site-specific integration of a payload.

In some embodiments, homology arms are between 50 and 1000 nt in length. In some embodiments, homology arms are between 100 and 500 nt in length. In some embodiments, homology arms are between 50 and 250 nt in length. In some embodiments, homology arms are between 50 and 850 nt in length. In some embodiments, homology arms are between 300 and 1000 nt in length. In some embodiments, homology arms are between 800 and 1500 nt in length. In some embodiments, homology arms are at least 350 nt in length. In some embodiments, homology arms are at least 500 nt in length. In some embodiments, homology arms are less than 1500 nt in length. In some embodiments, homology arms arc 1000 nt in length. In some embodiments, homology arms are at least 50 nt in length. In some embodiments, homology arms are at least 600 nt in length. In some embodiments, homology arms are of the same length. In some embodiments, homology arms are not of the same length. In some embodiments, homology arms have at least 70% sequence homology to a target locus. In some embodiments, homology arms have at least 80% sequence homology to a target locus. In some embodiments, homology arms have at least 90% sequence homology to a target locus. In some embodiments, homology arms have at least 95% sequence homology to a target locus. In some embodiments, homology arms have at least 99% sequence homology to a target locus. In some embodiments, homology arms have 100% sequence homology to a target locus. In some embodiments, homology arms have at least 70% sequence identity to a target locus. In some embodiments, homology arms have at least 80% sequence identity to a target locus. In some embodiments, homology arms have at least 90% sequence identity to a target locus. In some embodiments, homology arms have at least 95% sequence identity to a target locus. In some embodiments, homology arms have at least 99% sequence identity to a target locus. In some embodiments, homology arms have 100% sequence identity to a target locus.

In some embodiments, constructs comprising homology arms provide rates of target site integration of at least 5%. In some embodiments, constructs comprising homology arms provide rates of target site integration of 1%, 2%, 5%, 10%, 15%, 20%, 25%, 30%, 35%, 40%, 45%, 50%, 55%, 60%, 65%, 70%, 75%, 80%, 85%, 90% or more. In some embodiments, constructs comprising homology arms provide rates of target site integration of 30% or more. In some embodiments, constructs comprising homology arms provide rates of target site integration of 35% or more.

### Multiplex Integration

In some embodiments, methods and compositions disclosed herein direct integration of two or more payloads (e.g., transgenes) at one or more target loci (e.g., an endogenous gene). In some embodiments, compositions provided herein direct integration of two or more payloads at one or more target loci in a specific cell type (e.g., naïve B cells, B lineage cell populations, etc.). In some embodiments, methods provided herein direct integration of two or more payloads (e.g., expression cassette comprising a transgene encoding SPMD1) at a target locus in a specific cell type (e.g, naïve B cells, B lineage cell populations, etc.). In some embodiments, a payload is or comprises a transgene or variant thereof.

In some embodiments, methods and compositions disclosed herein direct integration of one or more payloads (e.g., transgenes) at one or more target loci. In some embodiments, methods and compositions disclosed herein direct integration of one or more payloads (e.g., transgenes) at two or more target loci. In some embodiments, methods and compositions disclosed herein direct integration of one or more payloads (e.g., transgenes) at two or more target loci. In some embodiments, methods and compositions disclosed herein direct integration of one or more payloads (e.g., transgenes) at four or more target loci. In some embodiments, methods and compositions disclosed herein direct integration of one or more payloads (e.g., transgenes) at five or more target loci. In some embodiments, methods and compositions disclosed herein direct integration of one or more payloads (e.g., transgenes) at two target loci. In some embodiments, methods and compositions disclosed herein direct integration of one or more payloads (e.g., transgenes) at three target loci. In some embodiments, methods and compositions disclosed herein direct integration of one or more payloads (e.g., transgenes) at four target loci. In some embodiments, methods and compositions disclosed herein direct integration of one or more payloads (e.g., transgenes) at five target loci.

In some embodiments, methods and compositions disclosed herein direct integration of one or more payloads (e.g., transgenes) at one or more target loci, optionally in combination with disruption (e.g., reduced expression, knockout, etc.) at one or more target loci. In some embodiments, methods and compositions disclosed herein direct integration of one or more payloads (e.g., transgenes) at two or more target loci, optionally in combination with disruption (e.g., reduced expression, knockout, etc.) at one or more target loci. In some embodiments, methods and compositions disclosed herein direct integration of one or more payloads (e.g., transgenes) at three or more target loci, optionally in combination with disruption (e.g., reduced expression, knockout, etc.) at one or more target loci. In some embodiments, methods and compositions disclosed herein direct integration of one or more payloads (e.g., transgenes) at four or more target loci, optionally in combination with disruption (e.g., reduced expression, knockout, etc.) at one or more target loci. In some embodiments, methods and compositions disclosed herein direct integration of one or more payloads (e.g., transgenes) at two or more target loci, optionally in combination with disruption (e.g., reduced expression, knockout, etc.) at five or more target loci. In some embodiments, methods and compositions disclosed herein direct integration of one or more payloads (e.g., transgenes) at one or more target loci, optionally in combination with disruption (e.g., reduced expression, knockout, etc.) at two or more target loci. In some embodiments, methods and compositions disclosed herein direct integration of one or more payloads (e.g., transgenes) at two or more target loci, optionally in combination with disruption (e.g., reduced expression, knockout, etc.) at two or more target loci. In some embodiments, methods and compositions disclosed herein direct integration of one or more payloads (e.g., transgenes) at three or more target loci, optionally in combination with disruption (e.g., reduced expression, knockout, etc.) at two or more target loci. In some embodiments, methods and compositions disclosed herein direct integration of one or more payloads (e.g., transgenes) at three or more target loci, optionally in combination with disruption (e.g., reduced expression, knockout, etc.) at three or more target loci. In some embodiments, methods and compositions disclosed herein direct integration of one or more payloads (e.g., transgenes) at four or more target loci, optionally in combination with disruption (e.g., reduced expression, knockout, etc.) at three or more target loci.

In some embodiments, methods and compositions provided herein direct integration of a payload at a target locus that is considered a safe-harbor site (e.g., CCR5, AAVS1). In some embodiments, methods and compositions provided herein direct integration of a payload at a target locus that is considered a tissue biomarker and/or immune regulator (e.g., B2M). In some embodiments, methods and compositions provided herein direct integration of one or more payloads at one or more target loci simultaneous (<24 hours). In some embodiments, methods and compositions provided herein direct integration of one or more payloads at the same target locus. In some embodiments, methods and compositions provided herein direct integration of one or more payloads at two or more different target loci. In some embodiments, methods and compositions provided herein direct integration of two or more payloads at one or more target loci by simultaneous introduction of two or more expression cassettes. In some embodiments, methods for multiplex integration may involve simultaneous introduction of two or more expression cassettes each comprising one or more transgenes which comprise site-specific cleavage at each target locus (e.g., CCR5, B2M) with one or more guide RNA/Cas9 complexes, followed by integration of one or more transgenes at each target locus through homologous recombination. In some other embodiments, introduction of two or more expression cassettes each comprising one or more transgenes which comprise site-specific cleavage at a target locus (e.g., CCR5, B2M) with one or more guide RNA/Cas9 complexes occurs within a time period under 24 hours. In some embodiments, methods for multiplex integration may involve sequential introduction of one or more expression cassettes each comprising one or more transgenes which comprise site-specific cleavage at a target locus (e.g., CCR5, B2M) with one or more guide RNA/Cas9 complexes, followed by integration of one or more transgenes at each target locus through homologous recombination and then followed by a time period before additional introduction of one or more expression cassettes. In some other embodiments, a time period may exist between sequential introduction of two or more expression cassettes. In some embodiments, a time period between each sequential introduction of expression cassettes may be 24-144 hours. In some embodiments, a target locus is selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, a target locus is selected from one or more of CD19, CD20, IGH, B2M, CCR5, JCHAIN, PAX5, IRF4, IRF8, BACH2, EZH2, XBP1, CARD11, PRDM1, BAFF, TAP1, TAP2, TAPBP, NLRC5, RFX5, BCMA, and CD58.

In some embodiments, methods and compositions disclosed herein direct multiplex integration at a safe harbor locus (e.g., CCR5) and a B2M locus. In some embodiments, methods and compositions disclosed herein direct multiplex integration at a safe harbor locus (e.g., CCR5) and a CD58 locus, methods and compositions disclosed herein direct multiplex integration at a safe harbor locus (e.g., CCR5), a B2M locus, and a CD58 locus. In some embodiments, methods and compositions disclosed herein direct integration at a safe harbor locus (e.g., CCR5) and disruption (e.g., knockout, reduced expression, etc.) at a B2M locus. In some embodiments, methods and compositions disclosed herein direct multiplex integration at a safe harbor locus (e.g., CCR5) and a CD58 locus. In some embodiments, methods and compositions disclosed herein direct integration at a safe harbor locus (e.g., CCR5) and disruption (e.g., knockout, reduced expression, etc.) at a CD58 locus. In some embodiments, methods and compositions disclosed herein direct multiplex integration at a safe harbor locus (e.g., CCR5), a B2M locus, and a CD58 locus. In some embodiments, methods and compositions disclosed herein direct integration at a safe harbor locus (e.g., CCR5), disruption (e.g., knockout, reduced expression, etc.) at a B2M locus, and disruption (e.g., knockout, reduced expression, etc.) at a CD58 locus.

In some embodiments, methods and compositions disclosed herein direct multiplex integration at a CCR5 locus and a B2M locus. In some embodiments, methods and compositions disclosed herein direct multiplex integration at a CCR5 locus and a CD58 locus, methods and compositions disclosed herein direct multiplex integration at a CCR5 locus, a B2M locus, and a CD58 locus. In some embodiments, methods and compositions disclosed herein direct integration at a CCR5 locus and disruption (e.g., knockout, reduced expression, etc.) at a B2M locus. In some embodiments, methods and compositions disclosed herein direct multiplex integration at a CCR5 locus and a CD58 locus. In some embodiments, methods and compositions disclosed herein direct integration at a CCR5 locus and disruption (e.g., knockout, reduced expression, etc.) at a CD58 locus. In some embodiments, methods and compositions disclosed herein direct multiplex integration at a CCR5 locus, a B2M locus, and a CD58 locus. In some embodiments, methods and compositions disclosed herein direct integration at a CCR5 locus, disruption (e.g., knockout, reduced expression, etc.) at a B2M locus, and disruption (e.g., knockout, reduced expression, etc.) at a CD58 locus.

In some embodiments, methods and compositions disclosed herein direct multiplex integration at an endogenous gene locus (e.g., JCHAIN) and a B2M locus. In some embodiments, methods and compositions disclosed herein direct multiplex integration at an endogenous gene locus (e.g., JCHAIN) and a CD58 locus, methods and compositions disclosed herein direct multiplex integration at an endogenous gene locus (e.g., JCHAIN), a B2M locus, and a CD58 locus. In some embodiments, methods and compositions disclosed herein direct integration at an endogenous gene locus (e.g., JCHAIN) and disruption (e.g., knockout, reduced expression, etc.) at a B2M locus. In some embodiments, methods and compositions disclosed herein direct multiplex integration at an endogenous gene locus (e.g., JCHAIN) and a CD58 locus. In some embodiments, methods and compositions disclosed herein direct integration at an endogenous gene locus (e.g., JCHAIN) and disruption (e.g., knockout, reduced expression, etc.) at a CD58 locus. In some embodiments, methods and compositions disclosed herein direct multiplex integration at an endogenous gene locus (e.g., JCHAIN), a B2M locus, and a CD58 locus. In some embodiments, methods and compositions disclosed herein direct integration at an endogenous gene locus (e.g., JCHAIN), disruption (e.g., knockout, reduced expression, etc.) at a B2M locus, and disruption (e.g., knockout, reduced expression, etc.) at a CD58 locus.

In some embodiments, methods and compositions disclosed herein direct multiplex integration at a safe harbor locus (e.g., CCR5) and a TAP2 locus. In some embodiments, methods and compositions disclosed herein direct multiplex integration at a safe harbor locus (e.g., CCR5) locus and a TAPBP locus. In some embodiments, methods and compositions disclosed herein direct multiplex integration at a safe harbor locus (e.g., CCR5) locus, a TAP2 locus, and a TAPBP locus. In some embodiments, methods and compositions disclosed herein direct integration at a safe harbor locus (e.g., CCR5) locus and disruption (e.g., knockout, reduced expression, etc.) at a TAP2 locus. In some embodiments, methods and compositions disclosed herein direct integration at a safe harbor locus (e.g., CCR5) locus and disruption (e.g., knockout, reduced expression, etc.) at a TAPBP locus. In some embodiments, methods and compositions disclosed herein direct integration at a safe harbor locus (e.g., CCR5) locus, disruption (e.g., knockout, reduced expression, etc.) at a TAP2 locus, and disruption (e.g., knockout, reduced expression, etc.) at a TAPBP locus.

In some embodiments, methods and compositions disclosed herein direct multiplex integration at a CCR5 locus and a TAP2 locus. In some embodiments, methods and compositions disclosed herein direct multiplex integration at a CCR5 locus and a TAPBP locus. In some embodiments, methods and compositions disclosed herein direct multiplex integration at a CCR5 locus, a TAP2 locus, and a TAPBP locus. In some embodiments, methods and compositions disclosed herein direct integration at a CCR5 locus and disruption (e.g., knockout, reduced expression, etc.) at a TAP2 locus. In some embodiments, methods and compositions disclosed herein direct integration at a CCR5 locus and disruption (e.g., knockout, reduced expression, etc.) at a TAPBP locus. In some embodiments, methods and compositions disclosed herein direct integration at a CCR5 locus, disruption (e.g., knockout, reduced expression, etc.) at a TAP2 locus, and disruption (e.g., knockout, reduced expression, etc.) at a TAPBP locus.

In some embodiments, methods and compositions disclosed herein direct multiplex integration at an endogenous gene locus (e.g., JCHAIN) and a TAP2 locus. In some embodiments, methods and compositions disclosed herein direct multiplex integration at an endogenous gene locus (e.g., JCHAIN) locus and a TAPBP locus. In some embodiments, methods and compositions disclosed herein direct multiplex integration at an endogenous gene locus (e.g., JCHAIN) locus, a TAP2 locus, and a TAPBP locus. In some embodiments, methods and compositions disclosed herein direct integration at an endogenous gene locus (e.g., JCHAIN) locus and disruption (e.g., knockout, reduced expression, etc.) at a TAP2 locus. In some embodiments, methods and compositions disclosed herein direct integration at an endogenous gene locus (e.g., JCHAIN) locus and disruption (e.g., knockout, reduced expression, etc.) at a TAPBP locus. In some embodiments, methods and compositions disclosed herein direct integration at an endogenous gene locus (e.g., JCHAIN) locus, disruption (e.g., knockout, reduced expression, etc.) at a TAP2 locus, and disruption (e.g., knockout, reduced expression, etc.) at a TAPBP locus.

In some embodiments, methods and compositions disclosed herein direct multiplex integration at a safe harbor locus (e.g., CCR5) and a NLRC5 locus. In some embodiments, methods and compositions disclosed herein direct integration at a safe harbor locus (e.g., CCR5) and disruption (e.g., knockout, reduced expression, etc.) at a NLRC5 locus.

In some embodiments, methods and compositions disclosed herein direct multiplex integration at a CCR5 locus and a NLRC5 locus. In some embodiments, methods and compositions disclosed herein direct integration at a CCR5 locus and disruption (e.g., knockout, reduced expression, etc.) at a NLRC5 locus.

In some embodiments, methods and compositions disclosed herein direct multiplex integration at an endogenous gene locus (e.g., JCHAIN) and a NLRC5 locus. In some embodiments, methods and compositions disclosed herein direct integration at an endogenous gene locus (e.g., JCHAIN) and disruption (e.g., knockout, reduced expression, etc.) at a NLRC5 locus.

In some embodiments, methods and compositions disclosed herein direct multiplex integration at a safe harbor locus (e.g., CCR5) and a RFX5 locus. In some embodiments, methods and compositions disclosed herein direct multiplex integration at a safe harbor locus (e.g., CCR5), a RFX5 locus, and a CD58 locus. In some embodiments, methods and compositions disclosed herein direct integration at a safe harbor locus (e.g., CCR5) and disruption (e.g., knockout, reduced expression, etc.) at a RFX5 locus. In some embodiments, methods and compositions disclosed herein direct integration at a safe harbor locus (e.g., CCR5) and disruption (e.g., knockout, reduced expression, etc.) at a RFX5 locus and disruption (e.g., knockout, reduced expression, etc.) at a CD58 locus. In some embodiments, methods and compositions disclosed herein direct integration at a safe harbor locus (e.g., CCR5) and integration at RFX5 and disruption (e.g., knockout, reduced expression, etc.) at a CD58 locus.

In some embodiments, methods and compositions disclosed herein direct multiplex integration at a CCR5 locus and a RFX5 locus. In some embodiments, methods and compositions disclosed herein direct multiplex integration at a CCR5 locus, a RFX5 locus, and a CD58 locus. In some embodiments, methods and compositions disclosed herein direct integration at a CCR5 locus and disruption (e.g., knockout, reduced expression, etc.) at a RFX5 locus. In some embodiments, methods and compositions disclosed herein direct integration at a CCR5 locus and disruption (e.g., knockout, reduced expression, etc.) at a RFX5 locus and disruption (e.g., knockout, reduced expression, etc.) at a CD58 locus. In some embodiments, methods and compositions disclosed herein direct integration at a CCR5 locus and integration at RFX5 and disruption (e.g., knockout, reduced expression, etc.) at a CD58 locus.

In some embodiments, methods and compositions disclosed herein direct multiplex integration at an endogenous gene locus (e.g., JCHAIN) and a RFX5 locus. In some embodiments, methods and compositions disclosed herein direct multiplex integration at an endogenous gene locus (e.g., JCHAIN), a RFX5 locus, and a CD58 locus. In some embodiments, methods and compositions disclosed herein direct integration at an endogenous gene locus (e.g., JCHAIN) and disruption (e.g., knockout, reduced expression, etc.) at a RFX5 locus. In some embodiments, methods and compositions disclosed herein direct integration at an endogenous gene locus (e.g., JCHAIN) and disruption (e.g., knockout, reduced expression, etc.) at a RFX5 locus and disruption (e.g., knockout, reduced expression, etc.) at a CD58 locus. In some embodiments, methods and compositions disclosed herein direct integration at an endogenous gene locus (e.g., JCHAIN) and integration at RFX5 and disruption (e.g., knockout, reduced expression, etc.) at a CD58 locus.

In some embodiments, disruption (e.g., knockout, reduced expression, etc.) of an endogenous target gene in B lineage cell populations comprises use of a nuclease (e.g., Cas nuclease, ZFN, TALEN, etc.). In some embodiments, disruption (e.g., knockout, reduced expression, etc.) of an endogenous target gene in B lineage cell populations comprises use of one or more RNAi agents. In some embodiments, disruption (e.g., knockout, reduced expression, etc.) of an endogenous target gene in B lineage cell populations comprises use of siRNA. In some embodiments, disruption (e.g., knockout, reduced expression, etc.) of an endogenous target gene in B lineage cell populations comprises use of shRNA. In some embodiments, disruption (e.g., knockout, reduced expression, etc.) of an endogenous target gene in B lineage cell populations comprises use of miRNA.

In some embodiments, methods and compositions disclosed herein direct multiplex integration of one or more payloads at one or more target locus at a higher rate when compared to methods and compositions not including methods described herein. In some embodiments, methods and compositions that lead to increased multiplex integration include, but are not limited to, serum vacation, etc. In some embodiments, methods and compositions that lead to increased multiplex integration may include addition of small peptides, inhibitors (e.g., XL413, M3814, etc.), anti-mitotic agents (e.g., nocodazole), and/or AAV integration enhancers (e.g., LAH4). In some embodiments, methods and compositions described herein provide rates of target site integration of 1%, 2%, 5%, 10%, 15%, 20%, 25%, 30%, 35%, 40%, 45%, 50%, 55%, 60%, 65%, 70%, 75%, 80%, 85%, 90% or more.

In some embodiments, methods and compositions disclosed herein direct multiplex integration of a payload, which provides higher rates of engraftment of an engineered B lineage cell population when administered to a subject. In some embodiments, a subject is a clinical species (e.g., human). In some embodiments, a subject is a non-clinical species (e.g., mouse model).

In some embodiments, the methods disclosed herein comprise measuring and/or monitoring engraftment of B lineage cells engineered with the methods and compositions described herein. In some embodiments, a sample is collected from a subject administered with engineered B lineage cells described herein. In some embodiments, sample collection is or comprises venipuncture. In some embodiments, sample collection is or comprises tissue collection. In some embodiments, levels of protein (e.g., Factor IX, SMPD1, blinatumomab, etc.) arc measured from sample collection. In some embodiments, lipids are assessed from a collected sample.

In some embodiments, engraftment of one or more B lineage cells of a population of genetically modified B lineage cells described herein is measured in a non-clinical species (e.g., hIL6-/NOG mice). In some embodiments, engraftment is measured by use of bioluminescence. In some embodiments, engraftment is measured by use of ELISpot. In some embodiments, levels of plasma IgG are determined to measure engraftment. In some embodiments, levels of plasma IgM are determined to measure engraftment. In some embodiments, levels of a transgene (e.g., SMPD1, Factor IX, blinatumomab, etc.) are determined to measure engraftment. In some embodiments, engraftment of one or more B lineage cells of a population of genetically modified B lineage cells described herein occurs in a non-clinical species (e.g., hIL6-/NOG mice) with or without preconditioning the non-clinical subject (e.g., chemotherapy, immunosuppressive treatment, etc.) (Cheng et al. 2022). In some embodiments, engraftment of one or more B lineage cells of a population of genetically modified B lineage cells described herein occurs in a clinical species (e.g., human subject) with or without preconditioning the clinical subject (e.g., chemotherapy, immunosuppressive treatment, etc.).

### B Lineage Cell Culture Methods

Various methods for culturing and long-term maintenance of B lineage cells in vitro are described in the art (See, Rawlings et al. 1995, Rawlings et al 1997, and Fluckinger et al. 1998, each of which is incorporated by reference herein in its entirety). B lineage cell culturing conditions can significantly affect normal human B lineage development and for production of mature Ig-secreting B cells. Without wishing to be bound by any theory, it is generally thought that activation of B lineage cells ex vivo may be necessary for later genome editing approaches that make use of homology-directed repair (HDR), as required DNA repair proteins are present during the G2/S phases of the cell cycle (See, Rogers and Cannon 2021, incorporated herein by reference in its entirety).

### Activation of B Lineage Cells

Various technologies for activation of B lineage cells in vitro have been described (also interchangeably referred to as “activated B lineage cells”). Traditionally, B lineage cell activation and proliferation in vitro employed CD40L-expressing feeder cell layer systems. Such feeder cell systems were described as difficult to standardize and often unreliable for providing consistent levels of activation and proliferation of B lineage cells. Recent advances have shifted to protocols for in vitro activation and proliferation of B lineage cells in specific culture systems comprising cytokines or other components in the absence of feeder cells (See Jourdan et al., 2009 and Hartweger et al., 2019, each of which is incorporated herein by reference in its entirety).

In some embodiments, methods for B lineage cell activation comprise contacting cells with media comprising one or more components of the present disclosure. In some embodiments, methods for B lineage cell activation comprise contacting cells with media comprising one or more cytokines and/or oligonucleotides (e.g., multimeric human CD40L, IL-2, IL-10, IL-15, IL-21 and/or CpG). In some embodiments, methods for B cell activation comprise contacting cells with media comprising at least about 5 ng/ml, 10 ng/ml, 15 ng/ml, 20 ng/ml, 25 ng/ml, 30 ng/mL, 35 ng/mL, 40 ng/mL, 45 ng/mL, 50 ng/mL, 55 ng/ml, 60 ng/mL, 65 ng/mL, 70 ng/mL, 75 ng/mL, 80 ng/mL, 85 ng/mL, 90 ng/mL, 95 ng/ml, 100 ng/mL, 150 ng/mL, 200 ng/mL, or 500 ng/mL of one or more cytokines and/or oligonucleotides (e.g., multimeric human CD40L, IL-2, IL-10, IL-15, IL-21 and/or CpG). In some embodiments, methods for B lineage cell activation comprise contacting cells with media comprising at least about 0.1 μg/mL, 0.2 μg/mL, 0.3 μg/mL, 0.4 μg/mL, 0.5 μg/mL, 0.6 μg/mL, 0.7 μg/mL, 0.8 ug/mL, 0.9 μg/mL, 1 μg/mL, 1.5 μg/mL, 2 μg/mL, 2.5 μg/mL, 3 μg/mL, 3.5 μg/mL, 4 μg/mL, 4.5 ug/mL, or 5 μg/mL of one or more cytokines and/or oligonucleotides (e.g., multimeric human CD40L, IL-2, IL-10, IL-15, IL-21 and/or CpG).

In some embodiments, methods for B lineage cell activation comprise a step of contacting cells with media comprising one or more components of the present disclosure for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of gene editing. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of B lineage cell expansion for at least about 1, 2, 3, 4, 5, 6, 7, or 8 days.

In some embodiments, methods described herein result in an activated B lineage cell population.

### Culturing Methods (Serum, Plasma, and Recombinant Protein Variation)

The present application provides, amongst other things, methods that result in enhanced genetic integration in B lineage cell populations. The application of these methods may further lead to increased engraftment of an engineered B lineage cell population in a subject (e.g., a human subject). Increased engraftment of an engineered B lineage cell population in a subject has larger ramification for cell therapy, such as treatment of disorders including, without limitation, hemophilia B and Niemann Pick Disease type B. Such methods to enhance genetic integration and increase engraftment include, but are not limited to, serum variation methods described herein.

In some embodiments, B lineage cells are isolated and cultured in media comprising one or more cytokines and/or oligonucleotides (e.g., CD40L, IL-2, IL-10, IL-15, IL-21 and/or CpG) for at least about 1, 2, 3, 4, or 5 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days. In some embodiments, methods for B lineage cell activation comprise a step of contacting cells with media comprising one or more cytokines and/or oligonucleotides (e.g., CD40L, IL-2, IL-10, IL-15, IL-21 and/or CpG) for at least 2 days, followed by a step of gene editing.

In some embodiments, a B lineage cell population is contacted with cell culture media that varies in serum, plasma, and/or recombinant proteins (e.g., lacks or is substantially free of serum, plasma, and/or recombinant proteins) from initial media conditions during or after gene engineering. In some embodiments, cell culture medium varies in (e.g., lacks or is substantially free of) one or more of: human serum, bovine serum, horse serum, newborn calf serum, goat serum, rabbit serum, porcine serum, or chicken serum. In some embodiments, a B lineage cell population undergoes a wash in base media that varies in serum (e.g., human serum, bovine serum, horse serum, newborn calf serum, goat serum, rabbit serum, porcine serum, chicken serum, or a combination thereof) from initial media conditions and lacks any added cytokines and/or oligonucleotides during or immediately after gene engineering. In some other embodiments, a B lineage cell population is contacted with cell culture media that varies in plasma (e.g., lacks plasma or is substantially free of plasma) from initial media conditions during or after gene engineering. In some such embodiments, cell culture medium varies in (e.g., lacks or is substantially free of) one or more of: human plasma, bovine plasma, horse plasma, newborn calf plasma, goat plasma, rabbit plasma, porcine plasma, or chicken plasma. In some such embodiments, a B lineage cell population undergoes a wash in base media that varies in plasma (e.g., human plasma, bovine plasma, horse plasma, newborn calf plasma, goat plasma, rabbit plasma, porcine plasma, chicken plasma, or a combination thereof) from initial media conditions and lacks any added cytokines and/or oligonucleotides during or immediately after gene engineering. In some other embodiments, a B lineage cell population is contacted with cell culture media that varies in plasma (e.g., lacks plasma or is substantially free of plasma) from initial media conditions during or after gene engineering. In some such embodiments, cell culture medium varies in (e.g., lacks or is substantially free of) one or more of: human recombinant proteins, bovine recombinant proteins, horse recombinant proteins, newborn calf recombinant proteins, goat recombinant proteins, rabbit recombinant proteins, porcine recombinant proteins, or chicken recombinant proteins. In some such embodiments, a B lineage cell population undergoes a wash in base media that varies in recombinant proteins (e.g., human recombinant proteins, bovine recombinant proteins, horse recombinant proteins, newborn calf recombinant proteins, goat recombinant proteins, rabbit recombinant proteins, porcine recombinant proteins, chicken recombinant proteins, or a combination thereof) from initial media conditions and lacks any added cytokines and/or oligonucleotides during or immediately after gene engineering.

In some embodiments, after gene engineering described herein, a B lineage cell population is contacted with media that varies in serum, plasma, and/or recombinant proteins (e.g., lacks or is substantially free of serum, plasma, and/or recombinant proteins) from initial media conditions, and then incubated for at least 1, 2, 3, 4, or 5 days. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for at least about 1 hour, 2 hours, 3 hours, 4 hours, 5 hours, 6 hours, 7 hours, 8 hours, 9 hours, 10 hours, or 12 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for at least 12 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for at least 24 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for at least 48 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for at least 72 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for no more than 72 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions, and then incubated for about 24 hours. In some embodiments, a B lineage cell population, post-gene engineering, is contacted with media that varies in serum, plasma, and/or recombinant proteins (e.g., lacks or is substantially free of serum, plasma, and/or recombinant proteins) from initial media conditions and comprises one or more cytokines and/or oligonucleotides (e.g., CD40L, IL-2, IL-10, IL-15, IL-21 and/or CpG).

In some embodiments, a B lineage cell population, after incubation, are spun down, supernatant removed, and re-plated in media with initial serum, plasma, and/or recombinant proteins conditions (e.g., human serum, bovine serum, horse serum, newborn calf serum, goat serum, rabbit serum, porcine serum, chicken serum, human plasma, bovine plasma, horse plasma, newborn calf plasma, goat plasma, rabbit plasma, porcine plasma, chicken plasma, human recombinant proteins, bovine recombinant proteins, horse recombinant proteins, newborn calf recombinant proteins, goat recombinant proteins, rabbit recombinant proteins, porcine recombinant proteins, chicken recombinant proteins, or a combination thereof). In some embodiments, B lineage cells are re-plated in media comprising one or more cytokines and/or oligonucleotides (e.g., CD40L, IL-2, IL-10, IL-15, IL-21 and/or CpG) and expanded for at least 1, 2, 3, 4, or 5 days.

Differentiation of B Cells into Plasmablasts

Plasmablasts are short-lived, rapidly produced effector cells that are primarily present in an early antibody response and are one potential product of terminal B cell differentiation. Plasmablasts are capable of secreting antibodies, including IgM subtype antibodies, in order to mount an immediate response to certain antigens in the body. Differentiation of B cells into plasmablasts in vitro may be promoted through use of certain signaling molecules, including one or more cytokines (e.g., IL-2, IL-6, IL-10, and/or IL-15). A variety of methods for differentiation of B cells into plasmablasts are known, but not limited to, those outlined in WO/2018/170150, incorporated herein by reference in its entirety. Plasmablasts produced by such methods may be characterized as cells that are CD27⁺/CD38⁺/CD138⁻.

In some embodiments, methods for B cell differentiation into plasmablasts comprise contacting cells with media comprising one or more components. In some embodiments, methods for B cell differentiation into plasmablasts comprise contacting activated B lincage cells with media comprising one or more cytokines (e.g., IL-2, IL-6, IL-10, and/or IL-15). In some embodiments, methods for B cell differentiation into plasmablasts comprise contacting activated B lineage cells with media comprising at least about 0.5 ng/ml, 1 ng/ml, 1.5 ng/mL, 2 ng/mL, 2.5 ng/mL, 3 ng/ml, 3.5 ng/mL, 4 ng/mL, 4.5 ng/mL, 5 ng/mL, 10 ng/ml, 15 ng/mL, 20 ng/mL, 25 ng/ml, 30 ng/mL, 35 ng/ml, 40 ng/ml, 45 ng/ml, 50 ng/ml, 55 ng/mL, 60 ng/mL, 65 ng/mL, 70 ng/mL, 75 ng/mL, 80 ng/ml, 85 ng/ml, 90 ng/ml, 95 ng/ml, or ng/ml of one or more cytokines (e.g., IL-2, IL-6, IL-10, and/or IL-15).

In some embodiments, methods for B cell differentiation into plasmablasts comprise a step of contacting cells with media comprising one or more components of the present disclosure for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least about 1, 2, 3, or 4 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days. In some embodiments, methods for B cell differentiation into plasmablasts comprise a step of contacting activated B lineage cells with media comprising one or more cytokines (e.g., IL-2, IL-6, IL-10, and/or IL-15) for at least 3 days, followed by a step of plasmablast expansion.

Differentiation of Plasmablasts into Plasma Cells

Although plasmablasts secrete more antibodies than naïve B cells, they are shorter-lived and secrete fewer antibodies than plasma cells (PCs). Long-lived plasma cells (LLPCs, used interchangeably throughout with plasma cells) localize to bone marrow in the body and are capable of secreting high levels of antibodies and surviving for decades in the absence of proliferation (See, Hammerland et al., 2017 and Khodadadi et al., 2019, both of which incorporated herewith in their entirety). Differentiation of plasmablasts to long-lived plasma cells can be triggered by certain events in the body, including, e.g., activity of transcription factors Blimp-1/PRDM1 and IRF4. Differentiation of plasmablasts to plasma cells in vitro may be promoted through use of certain signaling molecules, including one or more cytokines (e.g., IL-6, IL-15, and/or IFNα-2β). A variety of methods for differentiation of plasmablasts into plasma cells are known, but not limited to, those outlined in Jourdan et al. 2019 and WO/2018/170150 (each of which incorporated herein by reference in its entirety). Plasma cells produced by such methods may be characterized as cells that are CD27⁺/CD38⁺/CD138⁺.

In some embodiments, methods for plasmablast differentiation into plasma cells comprise contacting cells with media comprising one or more cytokines (e.g., IL-6, IL-15, and/or IFNα-2β). In some embodiments, methods for plasmablast differentiation into plasma cells comprise contacting plasmablasts with media comprising one or more cytokines (e.g., IL-6, IL-15, and/or IFNα-2β). In some embodiments, methods for plasmablast differentiation into plasma cells comprise contacting plasmablasts with media comprising at least about 0.5 ng/mL, 1 ng/mL, 1.5 ng/mL, 2 ng/mL, 2.5 ng/ml, 3 ng/ml, 3.5 ng/mL, 4 ng/ml, 4.5 ng/ml, 5 ng/mL, 10 ng/mL, 15 ng/mL, 20 ng/mL, 25 ng/ml, 30 ng/mL, 35 ng/mL, 40 ng/ml, 45 ng/mL, 50 ng/ml, 55 ng/mL, 60 ng/mL, 65 ng/mL, 70 ng/mL, 75 ng/mL, 80 ng/mL, 85 ng/ml, 90 ng/ml, 95 ng/ml, or ng/ml of one or more cytokines (e.g., IL-6, IL-15, and/or IFNα-2β).

In some embodiments, methods for plasmablast differentiation into plasma cells comprise a step of contacting cells with media comprising one or more components of the present disclosure for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least about 1, 2, 3, or 4 days. In some embodiments, methods for plasmablast differentiation into plasma cells comprise a step of contacting plasmablasts with media comprising one or more cytokines (e.g., IL-6, IL-15, and/or IFNα-2β) for at least 3 days. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of cell isolation. In some embodiments, methods for plasmablast differentiation into plasma cells comprise a step of contacting plasmablasts with media comprising one or more cytokines (e.g., IL-6, IL-15, and/or IFNα-2β) for at least 3 days, followed by a step of administration to a subject.

### Engineered Cell Preparations

The present disclosure describes engineered cell preparations comprising populations of cells modified to perform one or more desired functions. In some embodiments, engineered cell preparations are compositions comprising genetically modified immune cell populations (e.g., B cell, T cell). In some embodiments, engineered cell preparations are compositions comprising genetically modified B lineage cell populations (e.g., B cell, plasmablast, plasma cell). In some embodiments, engineered cell preparations are compositions comprising genetically modified plasmablast cell populations. In some embodiments, engineered cell preparations are compositions comprising genetically modified plasma cell populations.

In some embodiments, engineered cell preparations are genetically modified to express one or more payloads (e.g., transgene) of interest. In some embodiments, engineered cell preparations are genetically modified to express one or more transgenes from one or more expression cassettes (e.g., comprising additional polynucleotide sequence elements).

In some embodiments, engineered cell preparations comprise genetically modified cells that express one or more transgene of interest (e.g., therapeutic protein, antibody, etc.). In some embodiments, engineered cell preparations comprise genetically modified cells that express one or more transgenes of interest (e.g., therapeutic protein, antibody, etc.) from one or more endogenous gene loci. In some embodiments, engineered cell preparations comprise genetically modified cells that express one or more transgenes of interest (e.g., therapeutic protein, antibody, etc.) from one or more endogenous gene locus under control of an endogenous promoter. In some embodiments, engineered cell preparations comprise genetically modified cells that express one or more transgene of interest (e.g., therapeutic protein, antibody, etc.) from one or more endogenous gene loci under control of an exogenous promoter. In some embodiments, engineered cell preparations comprise genetically modified cells that express one or more transgenes of interest (e.g., therapeutic protein, antibody, etc.) from one or more endogenous gene loci without disrupting endogenous gene expression and/or function. In some embodiments, engineered cell preparations comprise genetically modified cells that express one or more transgenes of interest (e.g., therapeutic protein, antibody, etc.) from one or more endogenous gene loci and partially or fully disrupt endogenous gene expression and/or function.

In some embodiments, engineered cell preparations are genetically modified to express one or more transgene from one or more expression cassettes, wherein each expression cassette further comprises one or more polynucleotide sequences encoding one or more promoters. In some embodiments, engineered cell preparations are genetically modified to express one or more transgenes from one or more expression cassettes, wherein each expression cassette further comprises one or more polynucleotide sequences encoding one or more enhancers. In some embodiments, engineered cell preparations are genetically modified to express one or more transgenes from one or more expression cassettes, wherein each expression cassette further comprises one or more polynucleotide sequences encoding one or more terminators. In some embodiments, engineered cell preparations are genetically modified to express one or more transgenes from one or more expression cassettes, wherein each expression cassette further comprises one or more polynucleotide sequences encoding one or more homology arms. In some embodiments, engineered cell preparations are genetically modified to express one or more transgenes from one or more expression cassettes, wherein each expression cassette further comprises one or more polynucleotide sequences encoding one or more promoters, one or more enhancers, one or more terminators, and/or one or more homology arms. In some embodiments, engineered cell preparations are genetically modified to express one or more transgenes, or variant thereof, from one or more expression cassettes, wherein each expression cassette further comprises one or more polynucleotide sequences encoding a MND promoter, a WPRE enhancer, a BGH polyA, a 5′ homology arm, and a 3′ homology arm. In some embodiments, engineered cell preparations are genetically modified to express one or more transgenes, or variant thereof, from one or more expression cassettes, wherein each expression cassette further comprises one or more polynucleotide sequences encoding a MND promoter, a BGH polyA, a 5′ homology arm, and a 3′ homology arm. In some embodiments, engineered cell preparations are genetically modified to express one or more transgenes, or variant thereof, from one or more expression cassettes, wherein each expression cassette further comprises one or more polynucleotide sequences encoding a MND promoter, a WPRE enhancer, a SV40 polyA, a 5′ homology arm, and a 3′ homology arm. In some embodiments, engineered cell preparations are genetically modified to express one or more transgenes, or variant thereof, from one or more expression cassette, wherein each expression cassette further comprises one or more polynucleotide sequences encoding a MND promoter, a SV40 polyA, a 5′ homology arm, and a 3′ homology arm. In some embodiments, engineered cell preparations are genetically modified to express one or more transgenes, or variant thereof, from one or more expression cassettes, wherein each expression cassette further comprises one or more polynucleotide sequences encoding a EF-1a promoter, a WPRE enhancer, a BGH polyA, a 5′ homology arm, and a 3′ homology arm. In some embodiments, engineered cell preparations are genetically modified to express one or more transgenes, or variant thereof, from one or more expression cassettes, wherein each expression cassette further comprises one or more polynucleotide sequences encoding a EF-1a promoter, a BGH polyA, a 5′ homology arm, and a 3′ homology arm. In some embodiments, engineered cell preparations are genetically modified to express one or more transgene, or variant thereof, from one or more expression cassettes, wherein one or more expression cassette further comprises one or more polynucleotide sequences encoding a EF-1a promoter, a SV40 polyA, a 5′ homology arm, and a 3′ homology arm.

### Production

The present disclosure describes production of certain engineered B lineage cell preparations. As disclosed herein, in some embodiments, a B lineage cell preparation comprises both engineered and non-engineered cells. In some embodiments, an engineered B lineage cell population comprises plasmablasts. In some embodiments, an engineered B lineage cell population comprises plasma cells. In some embodiments, an engineered B lineage cell population comprises long-lived plasma cells. In some embodiments, an engineered B lineage cell population comprises plasmablasts, plasma cells, and/or long-lived plasma cells and/or any mixtures or combinations thereof disclosed herein.

### Plasmablast Preparation

In some embodiments, an engineered B lineage cell preparation comprises plasmablasts. As understood in the art, plasmablasts are rapidly produced and short-lived effector cells of the early antibody response. Plasmablasts can be generated from activated B lineage cells using methods described herein. In some embodiments, engineered plasmablasts may be generated from engineered activated B lineage cells.

In some embodiments, plasmablast cell populations may be contacted with media comprising one or more components of the present disclosure (e.g., IL-6, IL-15, and/or IFNα-2β) in order to initiate differentiation into plasma cell populations. In some embodiments, methods for plasmablast cell population differentiation into a plasma cell population comprise contacting plasmablast cell population with media comprising one or more cytokines (e.g., IL-6, IL-15, and/or IFNα-2β). In some embodiments, methods for a plasmablast cell population differentiation into a plasma cell population comprise contacting plasmablast cell population with media comprising at least about 0.5 ng/ml, 1 ng/ml, 1.5 ng/ml, 2 ng/mL, 2.5 ng/ml, 3 ng/ml, 3.5 ng/mL, 4 ng/mL, 4.5 ng/mL, 5 ng/mL, 10 ng/mL, 15 ng/mL, 20 ng/mL, 25 ng/ml, 30 ng/mL, 35 ng/mL, 40 ng/mL, 45 ng/mL, 50 ng/ml, 55 ng/mL, 60 ng/ml, 65 ng/mL, 70 ng/mL, 75 ng/mL, 80 ng/ml, 85 ng/mL, 90 ng/mL, 95 ng/mL, or ng/mL of one or more cytokines (e.g., IL-6, IL-15, and/or IFNα-2β).

In some embodiments, methods for plasmablast cell population differentiation into a plasma cell population comprise a step of contacting cells with media comprising one or more components of the present disclosure for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least about 1, 2, 3, or 4 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days. In some embodiments, methods for plasmablast cell population differentiation into a plasma cell population comprise a step of contacting plasmablast cell population with media comprising one or more cytokines (e.g., IL-6, IL-15, and/or IFNα-2β) for at least 3 days, followed by a step of cell isolation. In some embodiments, methods for plasmablast cell population differentiation into a plasma cell population comprise a step of contacting plasmablasts with media comprising one or more cytokines (e.g., IL-6, IL-15, and/or IFNα-2β) for at least 3 days, followed by a step of administration to a subject.

### Plasma Cell Preparation

In some embodiments, an engineered B lineage cell preparation comprises a plasma cell population. In some embodiments, a plasma cell population comprises a predetermined engineered plasma cell precursors (e.g., plasma blasts) that, upon administration to a subject, further differentiates into a mature plasma cell population. As understood in the art, a mature plasma cell population comprises quiescent, non-dividing cells of the humoral immune response that are capable of secreting large amounts of antibodies. In some embodiments, a mature plasma cell population can comprise short-lived plasma cells and/or long-lived plasma cells (LLPCs) and/or any combination thereof.

### Characterization

The present disclosure provides various methods for characterization of engineered cell populations (e.g., B lineage cell populations). In some embodiments, methods disclosed herein are used to segregate and/or characterize naïve B cell subpopulations within a B lineage cell population. In some embodiments, naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing naïve B cell subpopulations are employed during each step of a controlled cooling method. In some embodiments, methods of characterization are employed to segregate and/or characterize engineered naïve B cell subpopulations. In some embodiments, engineered naïve B cell subpopulations are characterized through one or more of flow cytometry, fluorescence-activated cell sorting (FACS), magnetic-activated cell sorting (MACS), and/or affinity chromatography. In some embodiments, one or more methods of characterizing engineered naïve B cell subpopulations are employed during each step of a controlled cooling method.

In some embodiments, methods disclosed herein are used to segregate and/or characterize activated B lineage cell subpopulations within a B lineage cell population. In some embodiments, activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing activated B lineage cell subpopulations are employed during each step of a controlled cooling method. In some embodiments, methods disclosed herein are used to segregate and/or characterize engineered activated B lineage cell subpopulations. In some embodiments, engineered activated B lineage cell subpopulations are characterized through one or more of flow cytometry, fluorescence-activated cell sorting (FACS), magnetic-activated cell sorting (MACS), and/or affinity chromatography. In some embodiments, one or more methods of characterizing engineered activated B lineage cell subpopulations are employed during each step of a controlled cooling method.

In some embodiments, methods disclosed herein are used to segregate and/or characterize plasmablast cell subpopulations within a B lineage cell population. In some embodiments, plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing plasmablast cell subpopulations are employed during each step of a controlled cooling method. In some embodiments, methods disclosed herein are used to segregate and/or characterize engineered plasmablast cell subpopulations. In some embodiments, engineered plasmablast cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasmablast cell subpopulations are employed during each step of a controlled cooling method.

In some embodiments, methods disclosed herein are used to segregate and/or characterize plasma cell precursor subpopulations within a B lineage cell population. In some embodiments, plasma cell precursor subpopulations are characterized through one or more of flow cytometry, fluorescence-activated cell sorting (FACS), magnetic-activated cell sorting (MACS), and/or affinity chromatography. In some embodiments, one or more methods of characterizing plasma cell precursor subpopulations are employed during each step of a controlled cooling method. In some embodiments, methods disclosed herein are used to segregate and/or characterize engineered plasma cell precursor subpopulations. In some embodiments, engineered plasma cell precursor subpopulations are characterized through one or more of flow cytometry, fluorescence-activated cell sorting (FACs), magnetic-activated cell sorting (MACs), and/or affinity chromatography. In some embodiments, one or more methods of characterizing engineered plasma cell precursor subpopulations are employed during each step of a controlled cooling method.

In some embodiments, methods disclosed herein are used to segregate and/or characterize plasma cell subpopulations within a B lineage cell population. In some embodiments, plasma cell subpopulations may be characterized through one or more of flow cytometry, fluorescence-activated cell sorting (FACS), magnetic-activated cell sorting (MACS), and/or affinity chromatography. In some embodiments, one or more methods of characterizing plasma cell subpopulations are employed during each step of a controlled cooling method. In some embodiments, methods disclosed herein are used to segregate and/or characterize engineered plasma cell subpopulations. In some embodiments, engineered plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered plasma cell subpopulations are employed during each step of a controlled cooling method.

In some embodiments, methods disclosed herein are used to segregate and/or characterize short-lived plasma cell populations within a B lineage cell population. In some embodiments, short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing short-lived plasma cell subpopulations are employed during each step of a controlled cooling method. In some embodiments, methods disclosed herein are used to segregate and/or characterize engineered short-lived plasma cell subpopulations. In some embodiments, engineered short-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered short-lived plasma cell subpopulations are employed during each step of a controlled cooling method.

In some embodiments, methods disclosed herein may be used to segregate and/or characterize long-lived plasma cell subpopulations with a B lineage cell population. In some embodiments, long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing long-lived plasma cell subpopulations are employed during each step of a controlled cooling method. In some embodiments, methods disclosed herein are used to segregate and/or characterize engineered long-lived plasma cell subpopulations. In some embodiments, engineered long-lived plasma cell subpopulations are characterized through one or more of flow cytometry, fluorescence-activated cell sorting (FACS), magnetic-activated cell sorting (MACS), and/or affinity chromatography. In some embodiments, one or more methods of characterizing engineered long-lived plasma cell subpopulations are employed during each step of a controlled cooling method.

### Methods of Treatment

The present disclosure, among other things, provides methods of treating a disease, disorder, or condition (e.g., a disease, disorder, or condition described herein) in a subject comprising administering a pharmaceutical composition described herein. In some embodiments, a therapeutically effective amount of a pharmaceutical composition described herein is administered to a subject having a disease or disorder. Pharmaceutical compositions described herein can be for use in the manufacture of a medicament for treating a disease, disorder, or condition (e.g., a disease, disorder, or condition described herein) in a subject.

Pharmaceutical compositions described herein can comprise one or more B lineage cells selected from a population of genetically modified B lineage cells described herein. In some embodiments, B lineage cell populations are engineered to express or comprise one or more payloads. In some embodiments, a payload is or comprises an expression cassette. In some embodiments, an expression cassette comprising a transgene (e.g., SMPD1, Factor IX, blinatumomab, etc.).

A subject to be treated with methods described herein can be a mammal, e.g., a primate, e.g., a human (e.g., a patient having, or at risk of having, a disease, disorder, or condition described herein. In some embodiments, a subject can be an adult subject. In some embodiments, engineered B lineage cells are administered to a pediatric subject.

Administration of pharmaceutical compositions described herein may be carried out in any convenient manner (e.g., injection, ingestion, transfusion, inhalation, implantation, or transplantation). In some embodiments, a pharmaceutical composition described herein is administered by injection or infusion. Pharmaceutical compositions described herein may be administered to a subject intravenously transarterially, subcutaneously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection. In some embodiments, a pharmaceutical composition described herein is administered directly to central nervous system (CNS) tissue.

In some embodiments, a pharmaceutical composition described herein is administered at a pharmaceutically suitable dosage to a subject. In some embodiments, a pharmaceutical composition described herein is administered monthly. In some embodiments, a pharmaceutical composition described herein is administered once every other month. In some embodiments, a pharmaceutical composition described herein is administered once every three months. In some embodiments, a pharmaceutical composition described herein is administered once every six months. In some embodiments, a pharmaceutical composition described herein is administered once a year.

In some embodiments, the methods disclosed herein comprise measuring and/or monitoring treatment. In some embodiments, a sample is collected from a subject treated with a pharmaceutical composition described herein. In some embodiments, sample collection is or comprises venipuncture. In some embodiments, sample collection is or comprises tissue collection. In some embodiments, levels of protein (e.g., SMPD1, Factor IX, blinatumomab, etc.) are measured from sample collection. In some embodiments, lipids are assessed from a collected sample.

In some embodiments, engraftment of one or more B lineage cells of a population of genetically modified B lineage cells described herein is measured in a non-clinical species (e.g., hIL6-/NOG mice). In some embodiments, engraftment is measured by use of bioluminescence. In some embodiments, engraftment is measured by use of ELISpot. In some embodiments, levels of plasma IgG are determined to measure engraftment. In some embodiments, levels of plasma IgM are determined to measure engraftment. In some embodiments, levels of a transgene (e.g., SMPD1, Factor IX, blinatumomab, etc.) are determined to measure engraftment. In some embodiments, engraftment of one or more B lineage cells of a population of genetically modified B lineage cells described herein occurs in a non-clinical species (e.g., hIL6-/NOG mice) with or without preconditioning the non-clinical subject (e.g., chemotherapy, immunosuppressive treatment, etc.) (Cheng et al. 2022). In some embodiments, engraftment of one or more B lineage cells of a population of genetically modified B lineage cells described herein occurs in a clinical species (e.g., human subject) with or without preconditioning the human (e.g., chemotherapy, immunosuppressive treatment, etc.).

### EXAMPLES

The following examples are provided so as to describe to the skilled artisan how to make and use methods and compositions described herein, and are not intended to limit the scope of the present disclosure.

### Example 1: Materials and Methods

The present Example demonstrates exemplary materials and methods that may be implemented for preparation, generation, and analysis of certain cell populations, including, but not limited to, B lineage cell populations. Cell populations can comprise or be B lineage cell populations. B lineage cell populations comprise or be naïve B cells. B lineage cell populations comprise or be activated B lineage cells. B lineage cell populations may comprise or be plasmablast. B lineage cell populations may comprise or be plasma cells.

### Inference of CRISPR Editing (ICE)

Cells were collected 72 hours after electroporation from non-engineered control (reference template) and Ribonucleoprotein (RNP)-only engineered conditions. The Maxwell DNA extraction kit was used to extract genomic DNA and normalized to 16.5 ng/mL after quantitation on the NanoDrop Spectrometer. Primers specific to the CRISPR sgRNA site were designed such that the target cut site was ~200 bp downstream the sequencing primer and ~600 bp upstream of the amplicon terminus. PCR amplification was performed using Q5 HiFi Hot start 2× MasterMix and the recommended thermalcycler program. A no-template control was included. Amplicon sizes were checked using the Lonza FlashGel system and shipped for Sanger Sequencing. Analysis of the Sanger sequences traces was performed using the Synthego ICE analytical method run off an internal server.

ICE Forward primer for CCR5:
(SEQ ID NO: 1)
GCAGCAAACCTTCCCTTCACTAC
ICE Reverse primer for CCR5:
(SEQ ID NO: 2)
AGGATTCCCGAGTAGCAGATGAC
ICE Sequencing primer for CCR5:
(SEQ ID NO: 3)
GGGTGGAACAAGATGGATTATC
ICE Forward primer for B2M:
AGGACCTTCTCTGAGCTGTC
ICE Reverse and Sequencing primer for B2M:
GCCCTAAACTTTGTCCCGAC

Droplet Digital Polymerase Chain Reaction (ddPCR)

ddPCR enables quantification of targeted integration efficiency as follows: copies/μL of HDR-edited allele (FAM-positive droplets)/copies/μL of a reference allele (HEX-positive cells)*100=% Targeted integration

For the integration site amplicon, one primer lies outside of a homology arm and the other lies inside the transgene. Thus, an amplicon is only generated when a genomic integration event has occurred. The reference amplicon is ideally a similar size and generated from either a distal region on the same locus or a reference gene. Amplicon primers were designed using the IDT PrimerQuest tool, and ddPCR probes were designed according to the following rules:
1) Designed a probe labeled with FAM inside the integration site amplicon sequence and in proximity to one of the two primers used for the integration site amplicon.
2) Designed probe labeled with HEX inside the reference amplicon sequence and in proximity to one of the two primers used for the reference amplicon
5) Included two controls: no-template control (excludes contamination), unedited cells (confirms specificity)

Reaction mix (including replicates) and ddPCR Supermix for Probes (no dUTP, BioRad) were allowed to equilibrate to room temperature. Droplets were generated using the Automated Droplet Generator QX200 following the manufacturer's instructions (BioRad). Reactions were then run in the C1000 Touch Thermal Cycler program recommended for the amplicon length. Samples were then read on a QX200 Droplet Reader (BioRad) and analyzed on 2D Amplitude (dot plot) for discrimination between negative, single, and double-positive droplets. Thresholding was done by calculating: Average of negative droplet intensity+0.1×(Average of positive droplet intensity−average of negative droplet intensity)=Threshold

% Targeted integration was then calculated using the above equation based on the adjusted thresholding and the copies/uL of FAM and HEX.

The following CCR5 target gene primers and probes were used:

CCR5 ddPCR Forward primer:
(SEQ ID NO: 4)
catcgcattgtctgagtagg
CCR5 ddPCR Reverse primer:
(SEQ ID NO: 5)
CAGTGGATCGGGTGTAAAC
CCR5_ddPCR Probe (FAM):
(SEQ ID NO: 6)
TCGGGAGCCTCTTGCTGGAAAATAGAA
The following CCRL2 reference gene primers and
probes were used:
CCRL2_ddPCR Forward primer:
(SEQ ID NO: 7)
CCACATCAGAAGGAAGACTAC
CCRL2_ddPCR Reverse primer:
(SEQ ID NO: 8)
GCTGTATGAATCCAGGTCC
CCRL2_ddPCR Probe (HEX):
(SEQ ID NO: 9)
TGTTTCCTCCAGGATAAGGCAGCTGT

### Cell Count and Viability

Cells were sampled for the CellacaMx counter after thorough resuspension by gentle pipetting. Cells were gently mixed with the appropriate diluent of AOPI stain, loaded into a Nexcelom count plate, and analyzed for count and viability based on the acquired image and dilution factor.

### Flow Cytometry Analysis

Following resuspension of the cell samples, 2×10⁵ cells were set-aside for flow cytometric analysis. Samples were washed and stained with antibody cocktails focused on immune cell purity (markers: CD19, CD20, CD16, CD56, CD3, CD14, CD45), on B lineage cell phenotyping (markers: CD19, CD20, CXCR4, CD27, CD38, CD138), on HLA phenotyping (HLA-A/B/C, HLA-E, HLA-G), or on Ig profiling (markers: CD38, CD138, IgM, IgG, IgE, IgA). Cells were washed once in PBS, then stained with LIVE/DEAD Fixable Near-IR Dead Cell Stain (Life Technologies) for 10 minutes at room temperature. Cells were washed with Flow Cytometry Staining Buffer (eBioscience) and resuspended in Human TruStain FcX (BioLegend). After 5 minutes of Fc blocking at 4° C., samples were then resuspended in 100 μL of antibody cocktail master mix. Samples were incubated for 20 minutes at 4° C. in the dark, then washed two times before being resuspended in 400 μL staining buffer for acquisition on the flow cytometer. Single color controls for voltage gating and compensation were also prepared. Following compensation as guided by the NovoCyte software prompt, >10,000 cellular events were acquired per sample. Files were saved and imported into FlowJo. Live cell singlets were further gated in this software, then population percentages, total events, and geometric mean fluorescence intensity values were exported, formatted, and statistically analyzed.

For IgM/IgG profiling, intracellular staining was performed using cell fixation and permeabilization. Following the last wash from viability staining, cells were suspended in fixation buffer (4% paraformaldehyde) and incubated at room temperature for 20 minutes. Cells were then washed in Permeabilization Buffer (eBioscience) three times, then resuspended in Ig profiling antibody master mix (dilutions used were manufacturer's recommendation). Samples were incubated 20 minutes at room temperature protected from light, then washed 2 times with permeabilization buffer. Pellets were suspended in staining buffer as for fresh cell staining and run with forward/side-scatter gating adjusted for cell shrinkage after fixation.

ELISA to Detect Human FIX from Engineered B Lineage Cell Population

Engineered B lineage cell population supernatant was harvested from relevant experiments and frozen at −80° C. prior to testing. Samples were then tested for concentrations of human Factor IX (hFIX) in the 96 well FIX ELISA (Abcam-ab108831). Briefly, all kit components, samples, and standards were thawed and allowed to equilibrate to room temperature. 50 μL of sample or standard were added per well of pre-coated plates and then incubated at room temperature for 2 hours. After incubation, plates were washed 5 times with 200 μL of 1× Wash Buffer. Next, 50 μL of 1× biotinylated FIX detector antibody was added into each well and incubated for 1 hour at room temperature. Plates were then washed and 50 μL of 1× SP conjugate was added to each well. After addition of 1× SP conjugate, plates were then incubated for 30 minutes at room temperature. After another round of plate washing, 50 μL of Chromogen Substrate was added per well and incubated for 10 minutes at room temperature. After color develops in the wells, 50 μL of Stop Solution was added to quench. The plate absorbance was read at 450 nm and 570 nm by a Cytation5 Plate Reader. Sample values were interpolated using the standards at known hFIX concentrations.

### Example 2: Simultaneous Multiplex Editing at Two Loci in B Lineage Cell Populations to Express Multiple Proteins

The present Example demonstrates that B lineage cell populations can be engineered simultaneously at multiple, distinct genetic loci to express one or more proteins of interest.

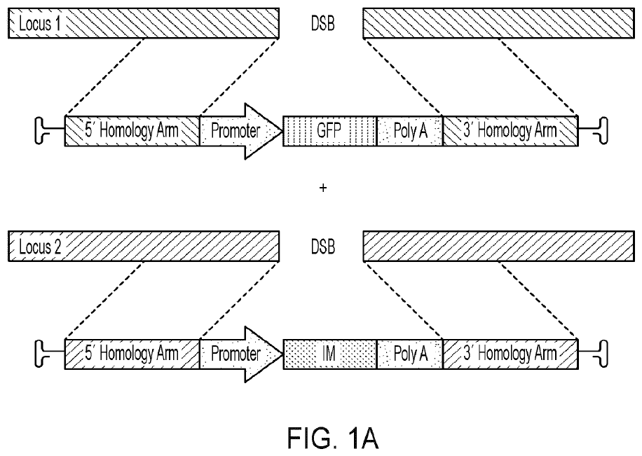

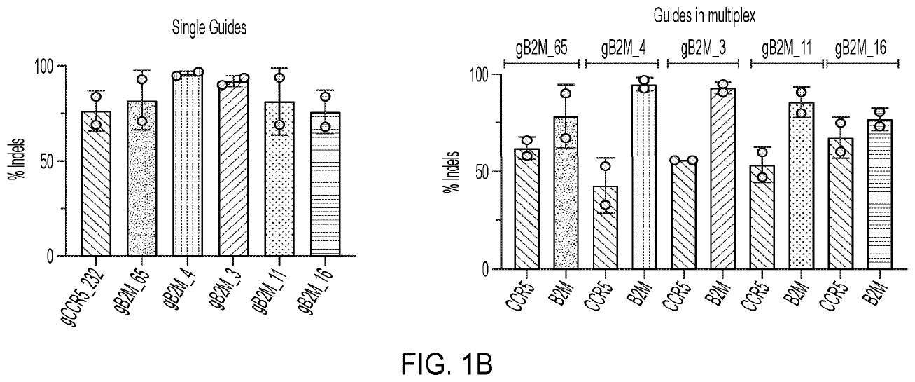

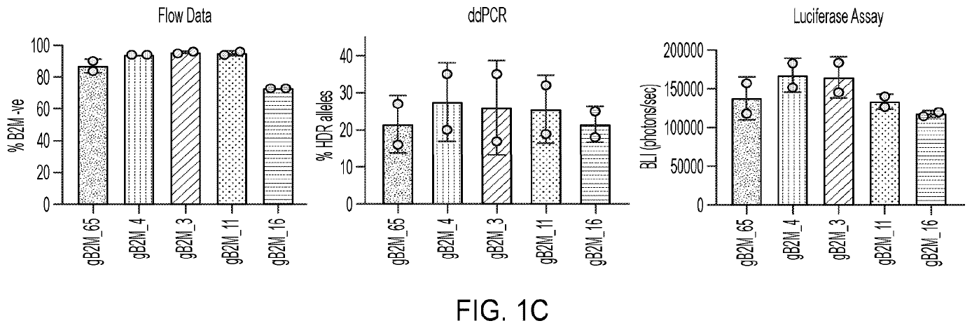

B lineage cells were cultured and engineered with CRISPR/Cas9 as described in the present disclosure and in WO 2018/170150 or US 2018/0282692, the entirety of each of which is incorporated herein by reference. In a multiplexed editing reaction, two guides were combined with Cas9 protein. Optimizing the relative amount of a given set of guides in a multiplexed reaction can ensure optimal editing at both loci is achieved. As is shown in FIG. 1B when the CCR5 guide was combined with one of 5 different B2M guides in a 1:1 sgRNA: sgRNA ratio, DNA editing efficiencies at CCR5 locus dropped compared to editing reactions in which single guides were used. In FIGS. 1B and 1C, gRNA 1 is a CCR5-targeting guide RNA, while gRNA 2 is a B2M-targeting guide RNA.

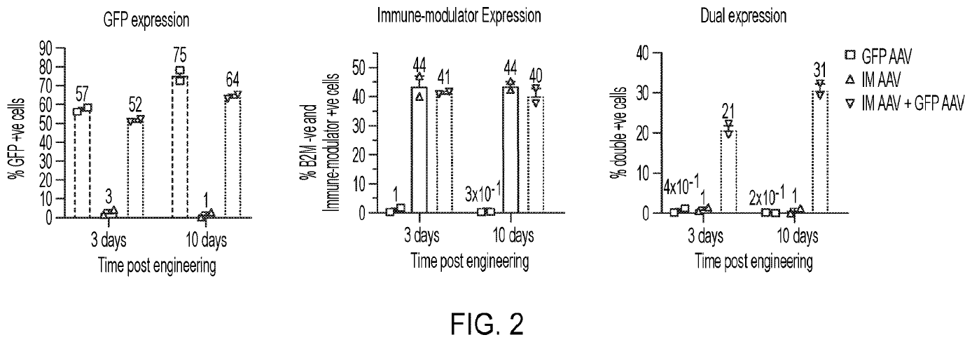

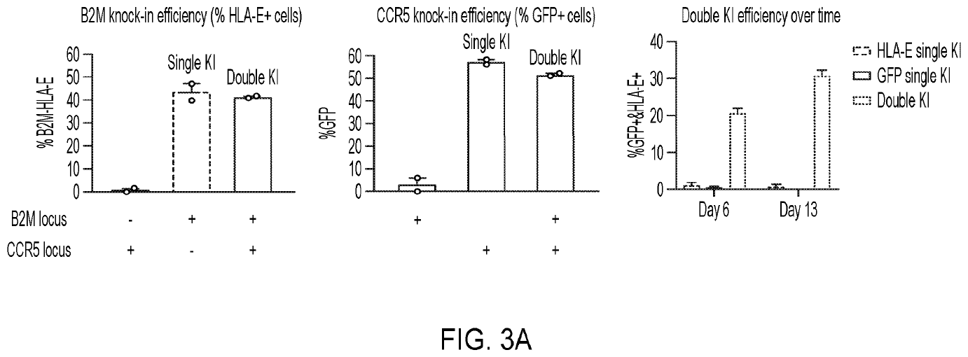

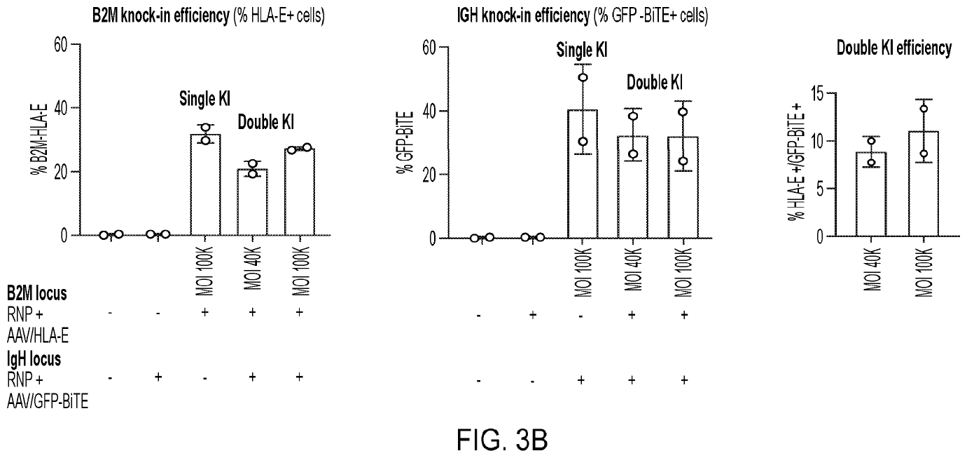

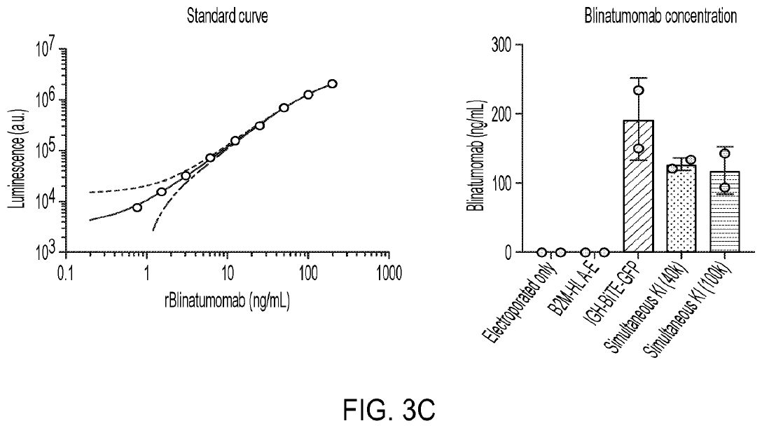

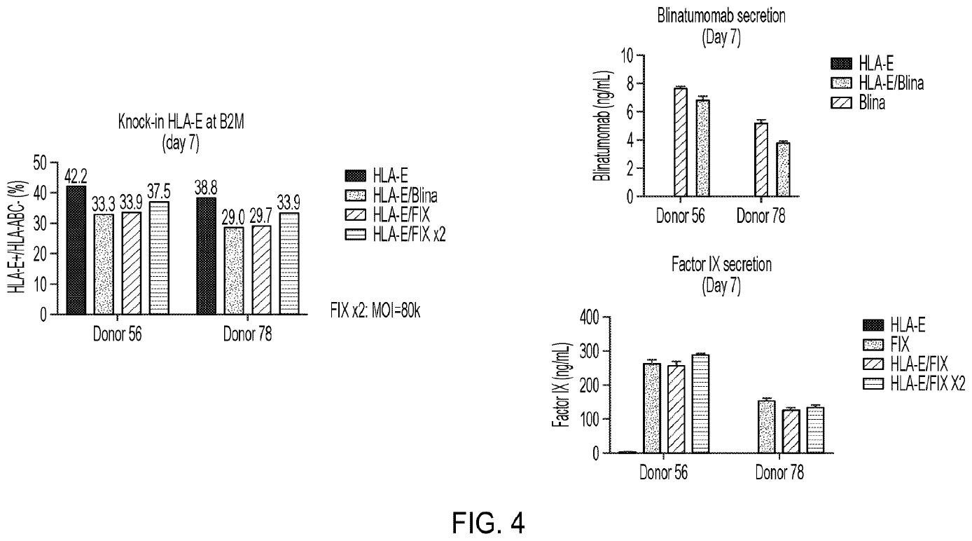

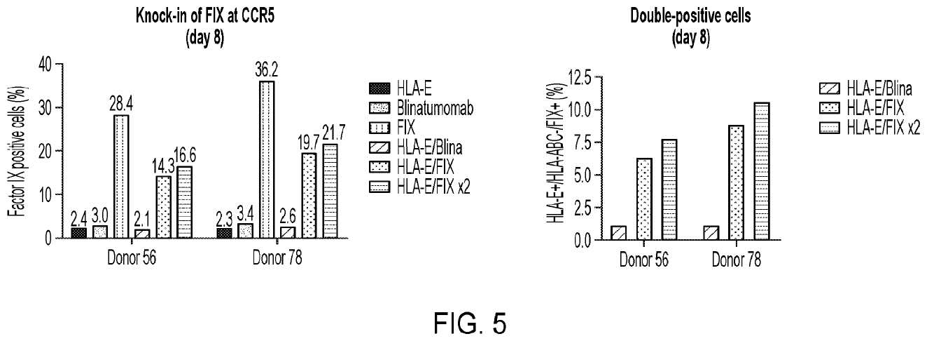

Certain data are presented in FIG. 2, FIG. 3, FIG. 4, and FIG. 5 that demonstrate multiplex engineering of B lineage cell populations may disrupt expression and/or secretion of endogenous gene products (e.g., B2M, CCR5, etc.) while allowing expression of one or more transgenes (e.g., GFP, FIX, one or more HLA proteins, including, e.g., HLA-E, etc.). In some embodiments, multiplex engineered B lineage cell populations may demonstrate comparable IgH and/or IgA expression as compared to a reference (e.g., non-engineered B lineage cell populations). In some embodiments, multiplex engineered B lineage cell populations may demonstrate transgene integration at one or more genetic loci that is comparable to a reference (e.g., transgene integration observed in B lineage cells at a single loci through non-multiplexed editing) (FIG. 3A). In some embodiments, integration efficiency of multiplex engineered B lineage cell populations may be altered through optimization of one or more factors (e.g., guide RNA: Cas9 ratios, target genetic loci, culturing methods, cell media, etc.). In some embodiments, multiplex engineered B lineage cells may preferentially expand within engineered B lineage cell populations.

### Example 3: Sequential Multiplex Editing at Two Loci in B Lineage Cell Populations to Express One or More Proteins

The present Examples demonstrates that B lineage cell populations can be engineered sequentially at multiple, distinct genetic loci to express one or more proteins of interest, including one or more antibody agents.

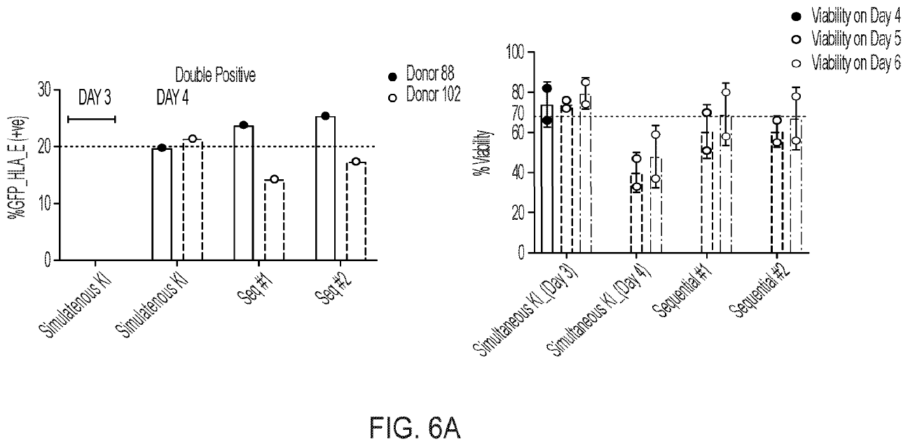

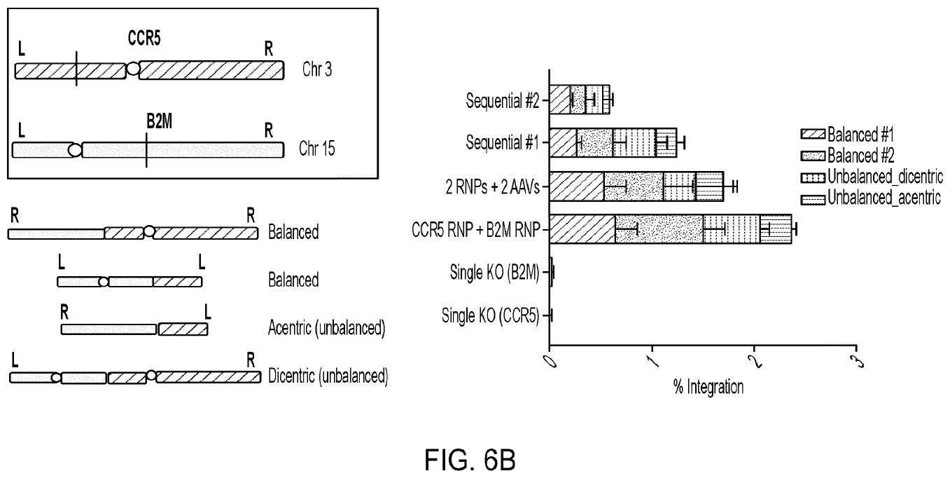

Certain data are presented in FIG. 6, comparing integration efficiency of multiplex engineered B lineage cell populations through simultaneous and sequential engineering at multiple target loci. In some embodiments, sequential multiplex engineered B lineage cell populations may provide comparable integration efficiency as compared to a reference (e.g., non-engineered B lineage cell populations, simultaneous multiplex engineered B lineage cell populations). In some embodiments, sequential multiplex engineered lineage cell populations may provide a reduced number of genetic translocation events as compared to a reference (e.g., simultaneous multiplex engineered B lineage cell populations).

### Example 4: Methods for Production of Engineered B Lineage Cells with Allogeneic Properties

The present Example demonstrates that B lineage cell populations can be engineered to improve one or more allogeneic properties (e.g., reduced immune response). In some embodiments, B lineage cell populations are engineered simultaneously at multiple, distinct genetic loci to express one or more proteins of interest.

B lineage cells were cultured and engineered with CRISPR/Cas9 as described in the present disclosure and in WO 2018/170150 or US 2018/0282692, the entirety of each of which is incorporated herein by reference.

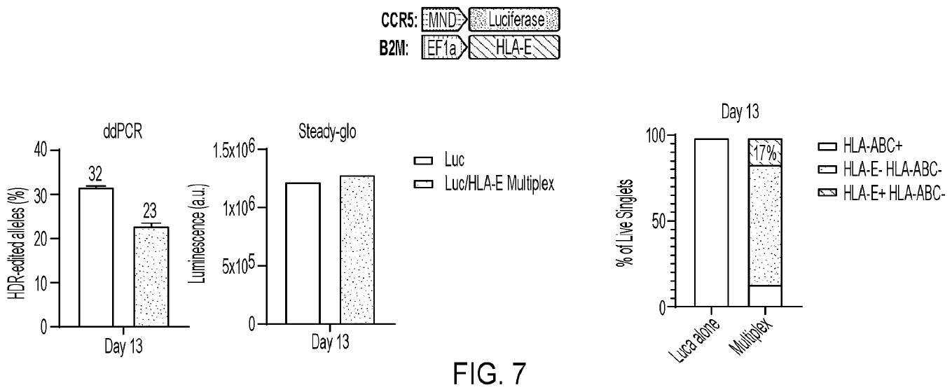

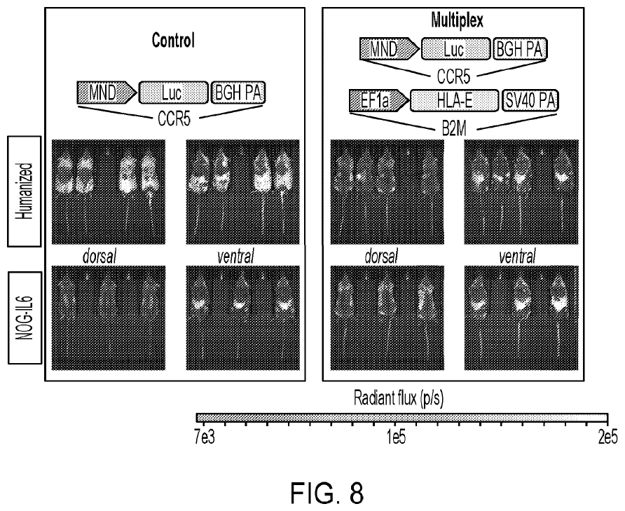

As outlined in FIG. 7, B lineage cells were edited at both CCR5 and B2M loci, expression a MND promoter and LUC transgene at CCR5 and a EF1a promoter and HLA-E at B2M. B lineage cell populations were cultured and differentiated in plasma cell populations as described herein. Multiplex engineered plasma cell populations comprising a MND promoter, LUC transgene, and BGH polyA at a CCR5 locus and comprising a EF1a promoter, HLA-E transgene, and SV40 polyA at a B2M locus were administered to mice (humanized and NOG-IL6 mice). A second engineered plasma cell population (“Control”) was also administered to mice, comprising a MND promoter, LUC transgene, and BGH polyA at a CCR5 locus. Treated mice were monitored for 6 weeks after administration to monitor engraftment and persistence of administered engineered B lineage cell populations (FIG. 8). At the 6-week time point, mice treated with multiplex engineered plasma cell populations demonstrated higher persistence in spleen and bone marrow as compared to mice treated with Control engineered plasma cell populations.

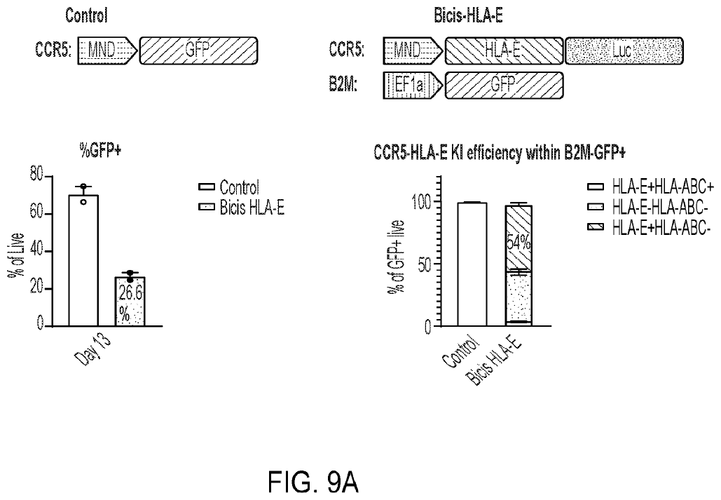

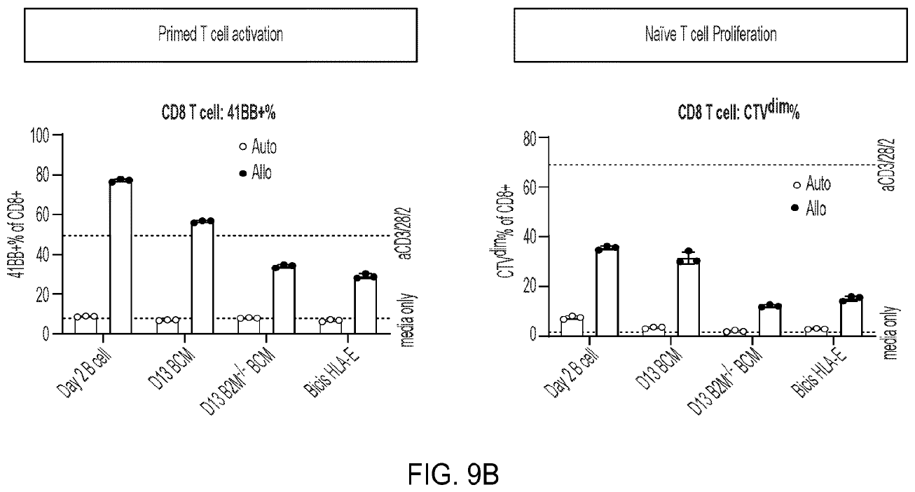

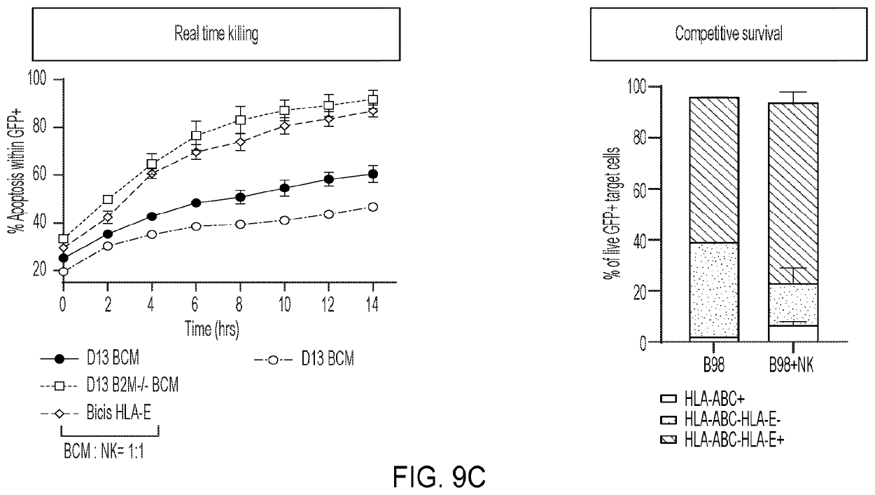

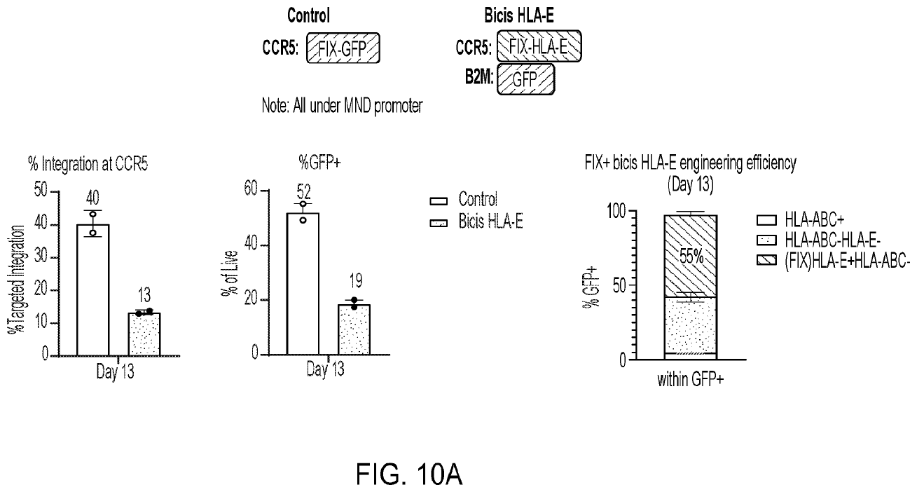

As outlined in FIG. 9A, B lineage cells were engineered at multiple, distinct loci and assessed for percentage of GFP expression cells, percentage of cells expressing HLA-E and/or HLA-ABC cells within the GFP-expressing population. B lineage cells were engineered to comprise an expression cassette including bicistronic HLA-E and LUC at CCR5, and GFP at B2M (“Bicis HLA-E”), an expression cassette including bicistronic HLA-E and LUC at CCR5 only (“Control”), or an expression cassette including bicistronic HLA-E and LUC at CCR5 combined with knock out of B2M expression (“Bicis-HLA-E”). As shown in FIG. 9B, Bicis HLA-E engineered B lineage cells demonstrated reduced T-cell activation and T-cell proliferation. As shown in FIG. 9C, Bicis HLA-E engineered B lineage cells also demonstrated increased resistance to NK cell targeting as compared to B2M KO BCM.

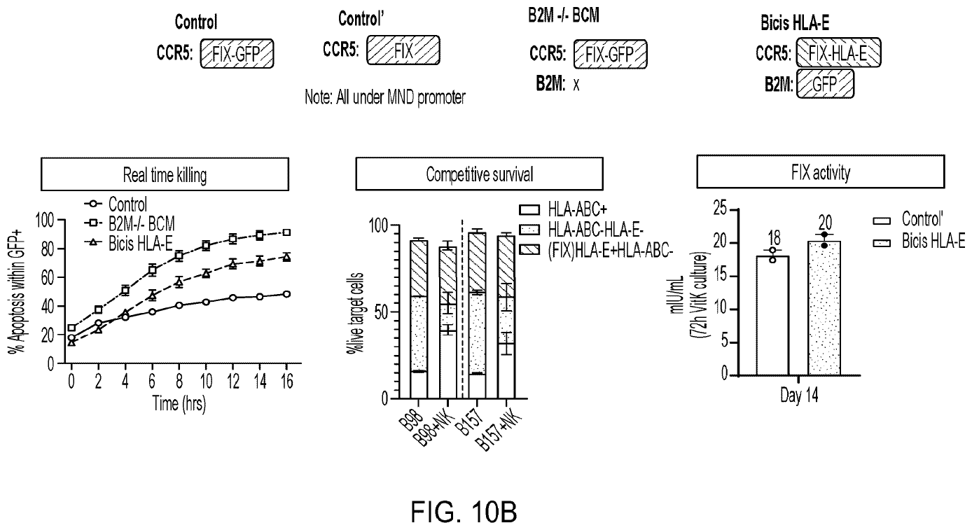

As outlined in FIG. 10A, B lineage cells were engineered at multiple, distinct loci and assessed for integration efficiency at CCR5, percentage of GFP expression cells, and percentage of cells expressing FIX within the GFP-expressing population. B lineage cells were engineered to comprise an expression cassette including bicistronic HLA-E and FIX at CCR5 and a GFP transgene at B2M (“Bicis HLA-E”) or an expression cassette including FIX-GFP only (“Control”). an expression cassette comprising FIX-GFP combined with knockout of B2M (“B2M−/−BCM”), or an expression cassette comprising FIX at CCR5 (“Control′”). As outlined in FIG. 10B, engineered B lineage cells were assessed for real-time killing and competitive survival rates when co-cultured with NK cells. FIX activity was also assessed for Control′ and Bicis HLA-E engineered B lineage cells. Bicis HLA-E engineered B lineage cells were able to produce functional FIX and demonstrated some resistance to NK cell killing.

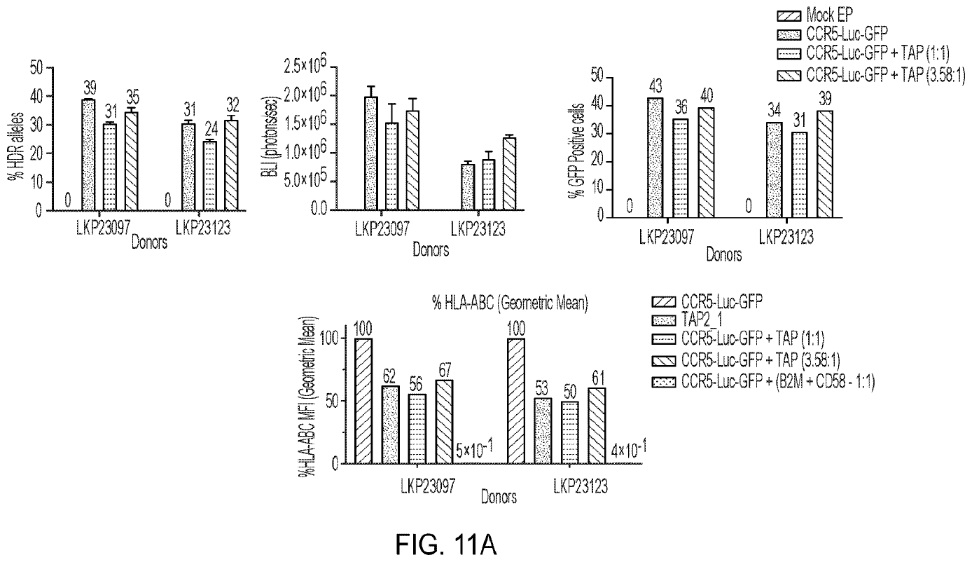

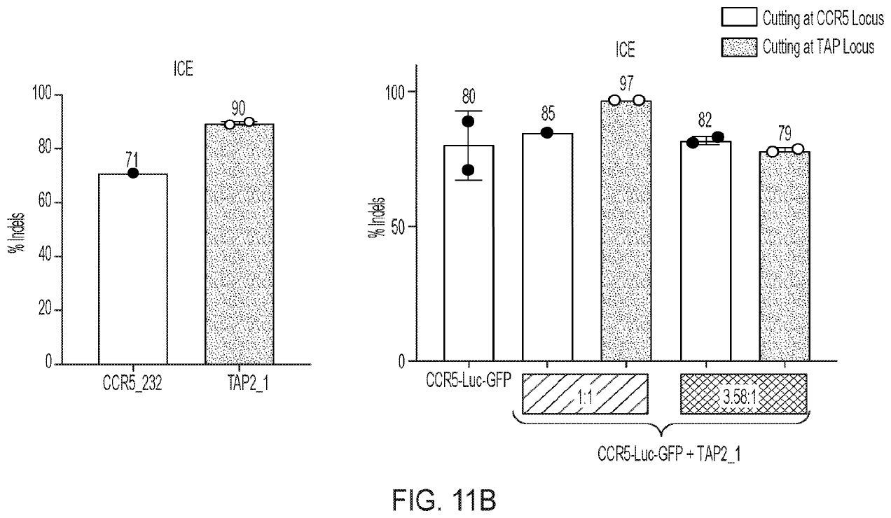

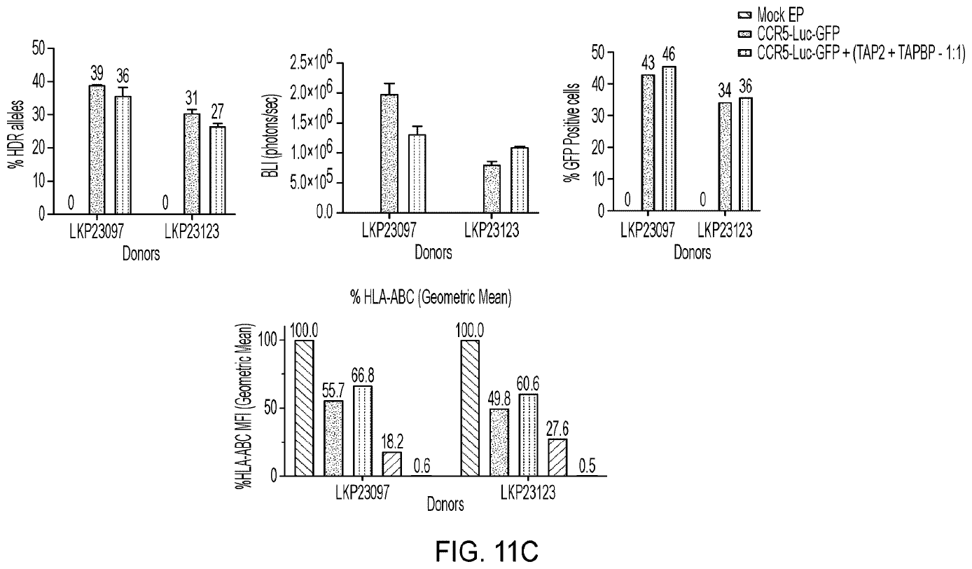

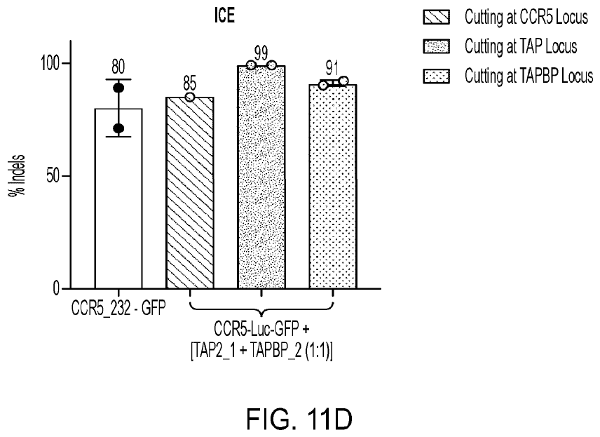

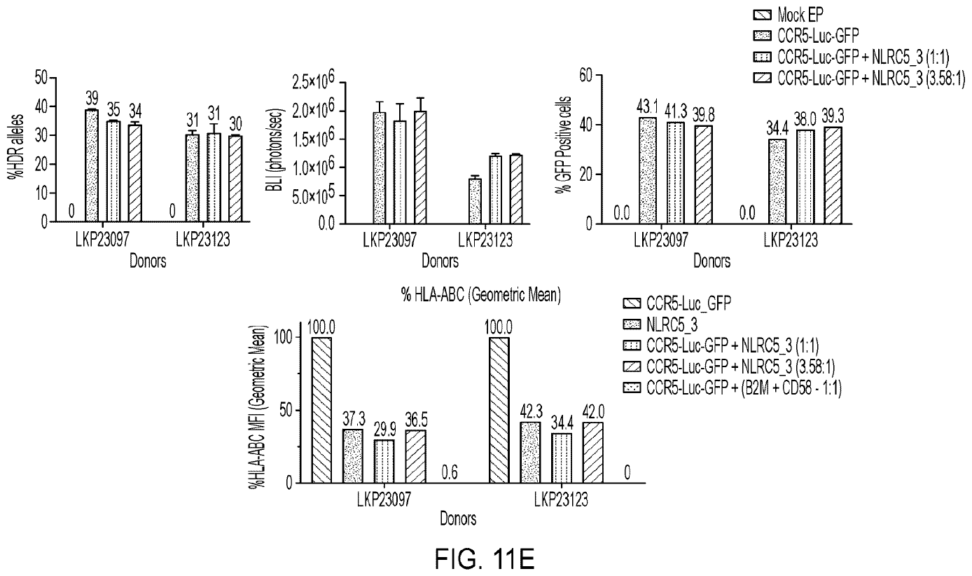

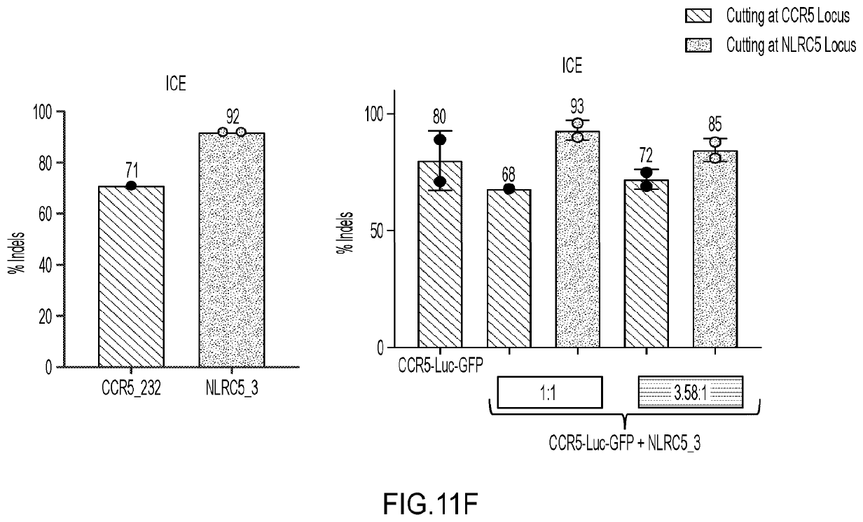

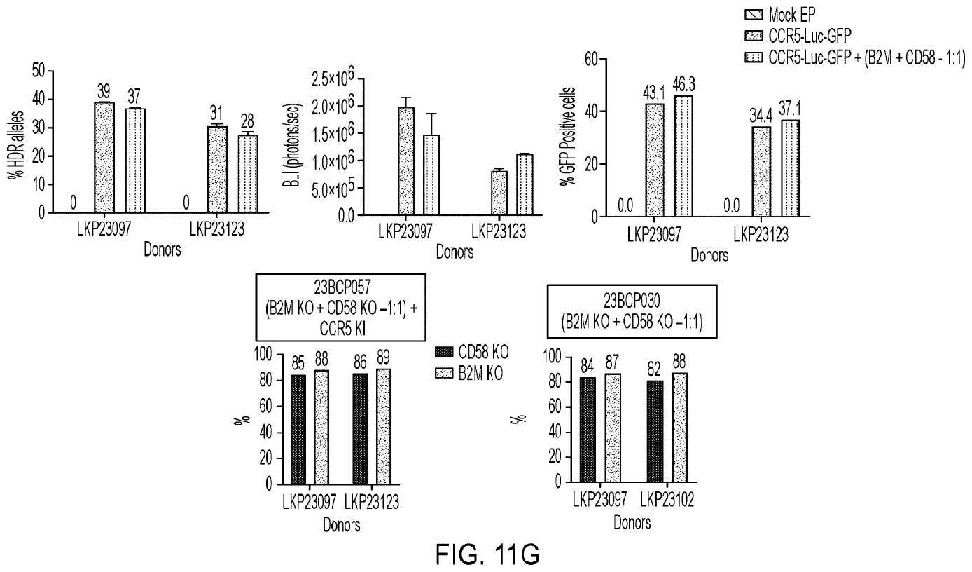

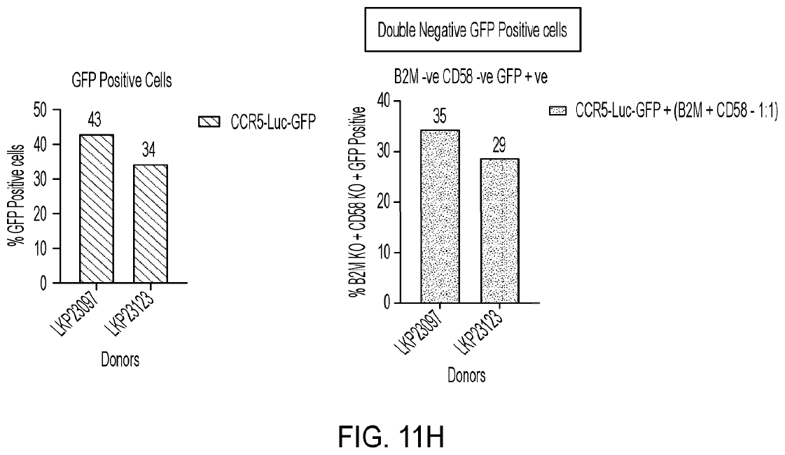

As outlined in FIG. 11A, FIG. 11B, FIG. 11C, FIG. 11D, FIG. 11E, FIG. 11F, FIG. 11G, and FIG. 11H, various conditions for gene editing with CRISPR/Cas9 can produce efficient editing at multiple loci in B lineage cells. In some embodiments, B lineage cells may be edited at two or more sites (e.g., CCR5, TAP, TAP2, TAPBP, etc.). In some embodiments, B lineage cells may be edited at three or more sites (e.g., CCR5, TAP, TAP2, TAPBP, etc.).

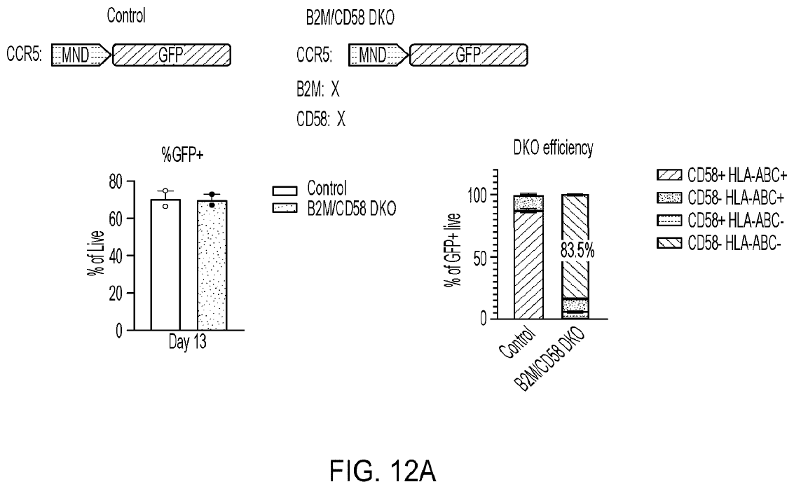

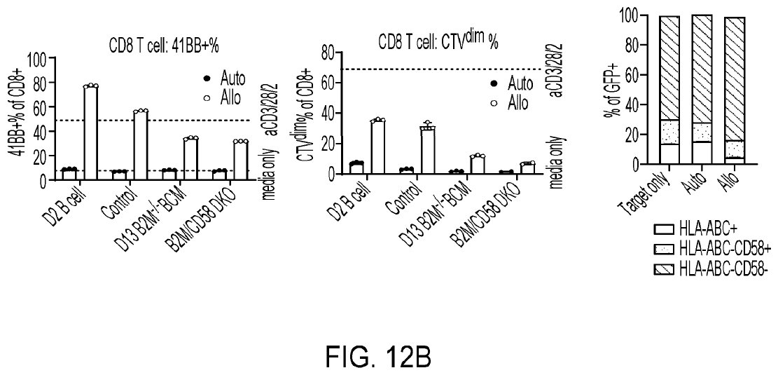

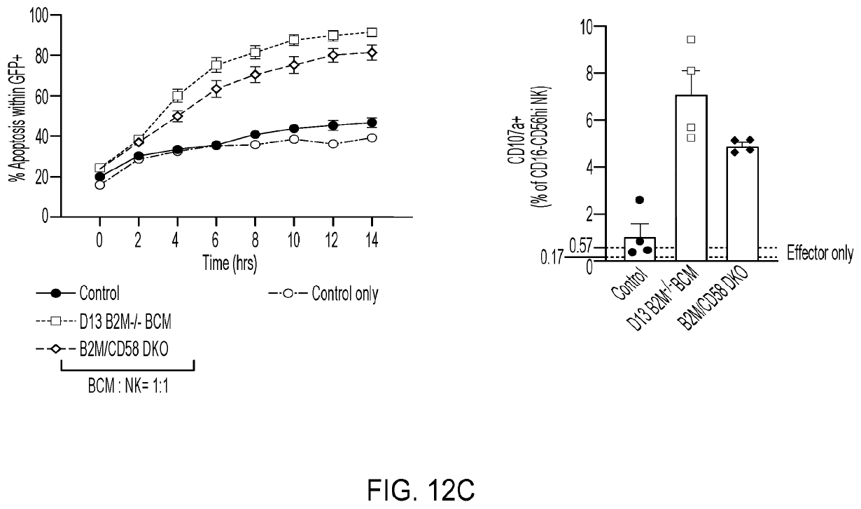

As outlined in FIG. 12A, engineered B lineage cells were assessed for efficiency of integration and/or knockout at one or more loci. Percentage of GFP-positive cells and efficiency of knock out were assessed for B lineage cells engineered to comprise a GFP transgene at CCR5 (Control), and a GFP transgene at CCR5, knockout of B2M, and knockout of CD58 (B2M/CD58 DKO). As outlined in FIG. 12B, B2M/CD58 DKO engineered B lineage cells demonstrated improved T-cell resistance as compared to other methods. As outlined in FIG. 12C, B2M/CD58 DKO engineered B lineage cell populations demonstrated some improved NK cell resistance as compared to other engineered B lineage cell populations.

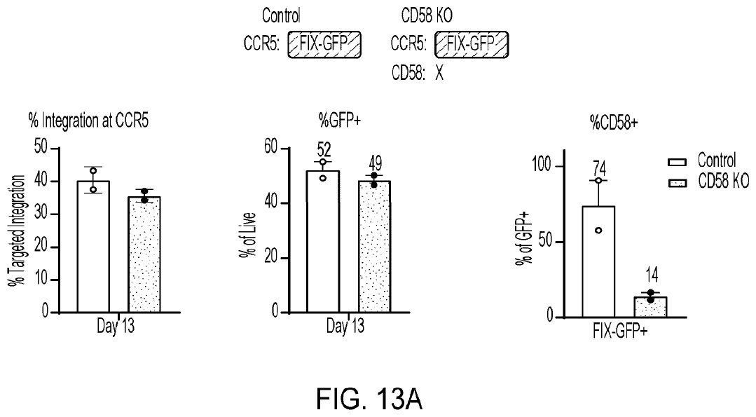

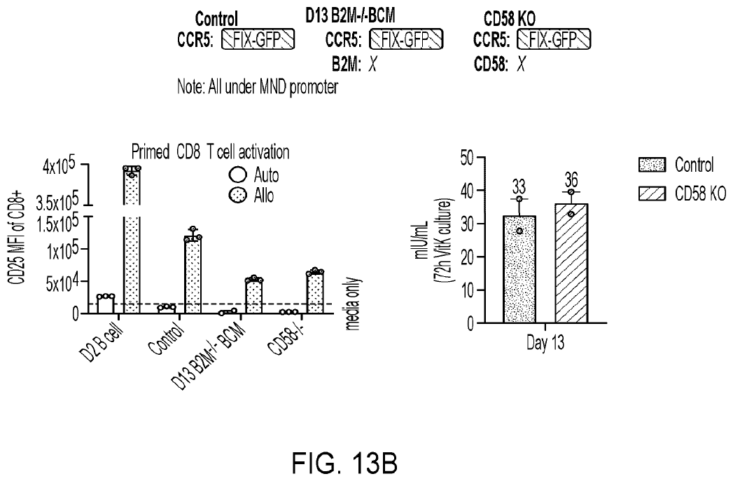

As outlined in FIG. 13A, engineered B lineage cells were assessed for integration efficiency at CCR5, GFP-positive cell percentages, and CD58-positive cell percentages. As outlined in FIG. 13B, engineered B lineage cells were assessed for T-cell resistance. Measurements were conducted for B lineage cells engineered as indicated to comprise FIX-GFP transgene at CCR5 (Control), a FIX-GFP transgene at CCR5 and knockout of CD58 (CD58 KO), or a FIX-GFP transgene at CCR5 and a knockout of B2M (D13 B2M−/−BCM). As outlined in FIG. 13B, Measurement of CD25 MFI among CD8+ cell populations was assessed for indicated engineered B lineage cell populations and Day 2 non-engineered primary B cells. FIX activity was also assessed for Control and CD58 KO engineered B lineage cell populations. Culture timing is described with culture start date as day 0.

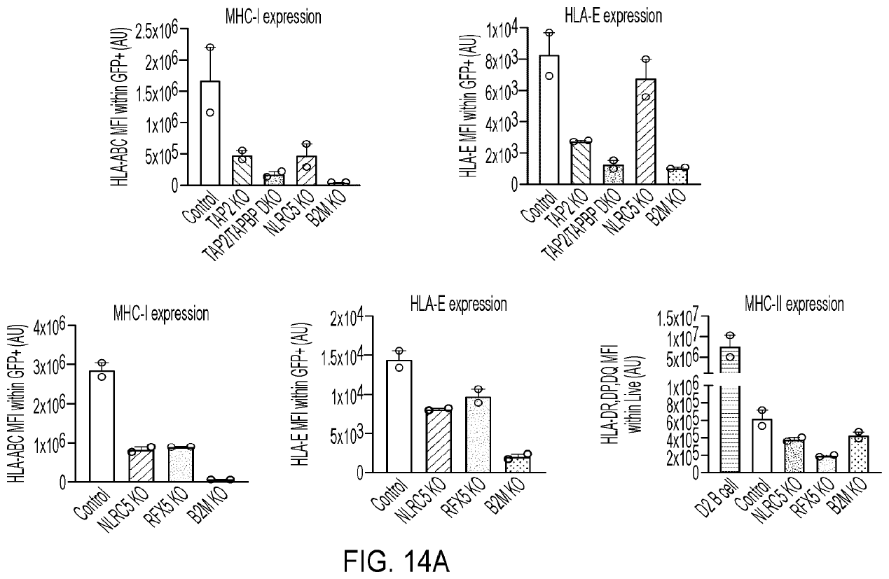

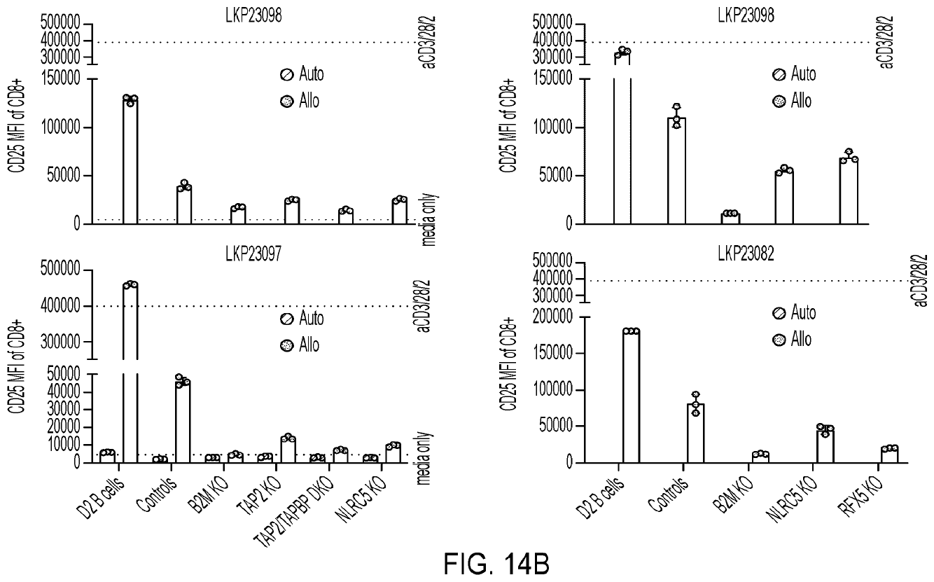

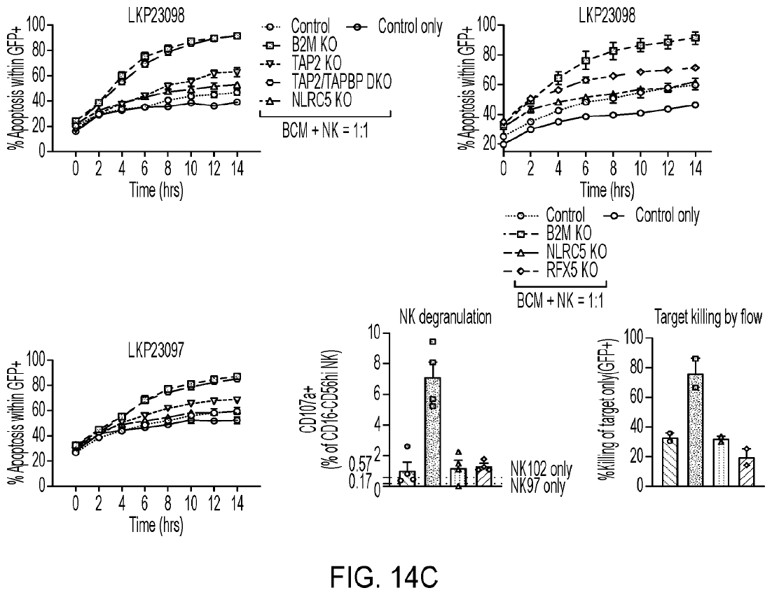

As outlined in FIG. 14A, engineered B lineage cells were assessed for MHC-I, MHC-II, and HLA-E expression. B lineage cells were engineered as indicated to comprise a GFP transgene at CCR5 in combination either alone (Control) or in combination with knockout of TAP2 (TAP2 KO), knockout of TAP2 and TAPBP (TAP2/TAPBP KO), knockout of NLRC5 (NLRC5 KO), knockout of RFX5 (RFX5 KO), or knockout of B2M (B2M KO). Non-engineered day 3 primary B cells were also assessed. As outlined in FIG. 14B, indicated engineered B lineage cell populations were assessed for T-cell resistance. As outlined in FIG. 14C, indicated engineered B lineage cell populations were assessed for NK cell resistance.

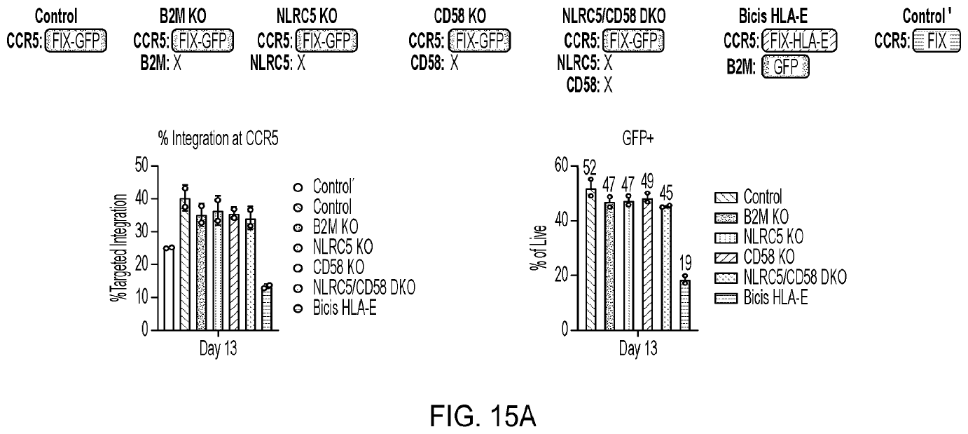

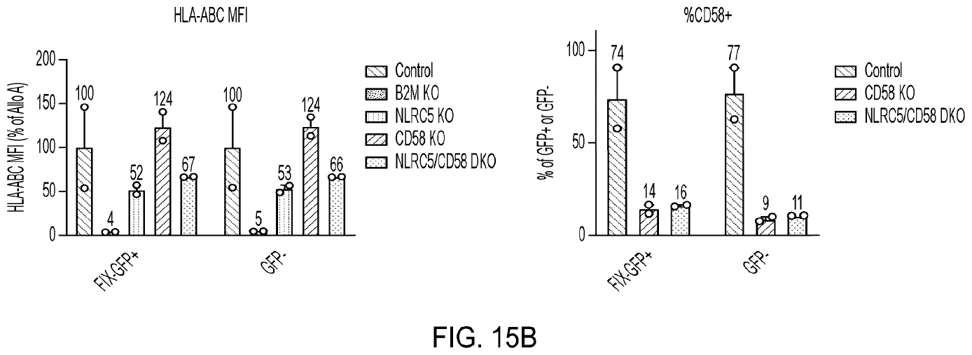

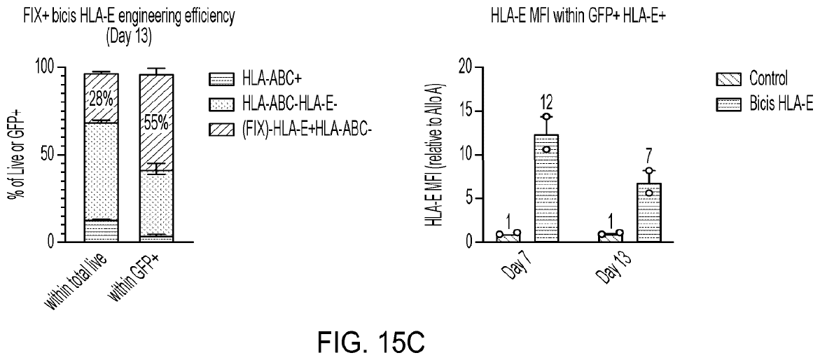

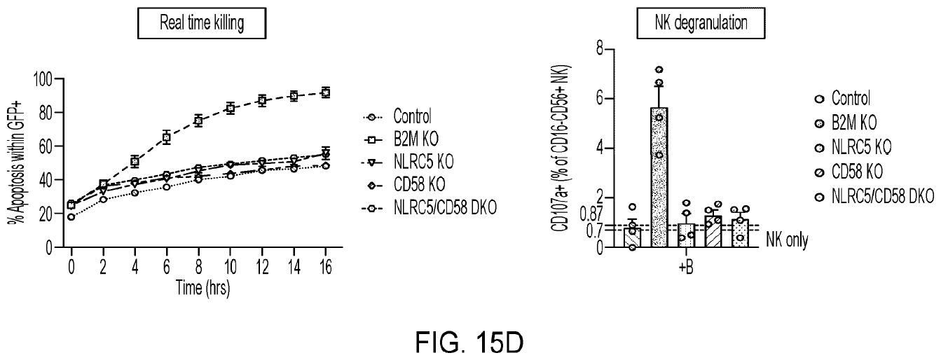

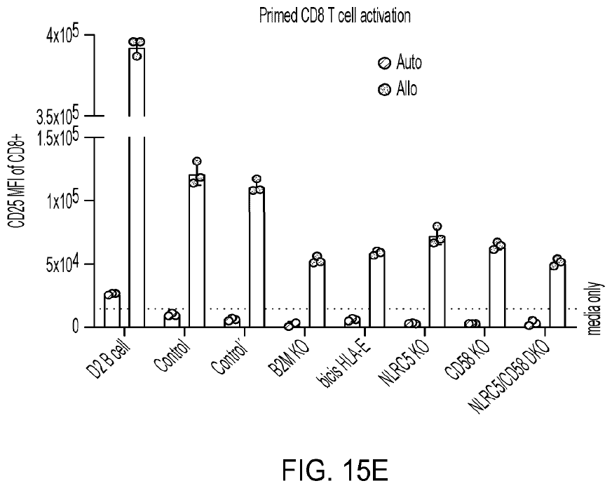

As outlined in FIG. 15A, engineered B lineage cells were assessed for integration efficiency. B lineage cells were engineered as indicated to comprise a FIX-GFP transgene at CCR5 (Control), a FIX-GFP transgene at CCR5 and a knockout of B2M (B2M KO), a FIX-GFP transgene at CCR5 and knockout of NLRC5 (NLRC5 KO), a FIX-GFP transgene at CCR5 and knockout of CD58 (CD58 KO), a FIX-GFP transgene at CCR5 and knockout of NLRC5 and CD58 (NLRC5/CD58 DKO), a bicistronic FIX transgene and HLA-E transgene at CCR5 and a GFP transgene at B2M (Bicis HLA-E), or a FIX transgene at CCR5 (Control′). As outlined in FIG. 15B and FIG. 15C, protein expression and engineering efficiency was assessed for various engineered B cell populations. As indicated in FIG. 15D and FIG. 15E, NK cell resistance and T-cell resistance was also assessed for engineered B lineage cell populations. Certain engineered B lineage cell populations exhibited relatively moderate to high NK cell resistance. Certain engineered B lineage cell populations exhibited moderate to high T-cell resistance.

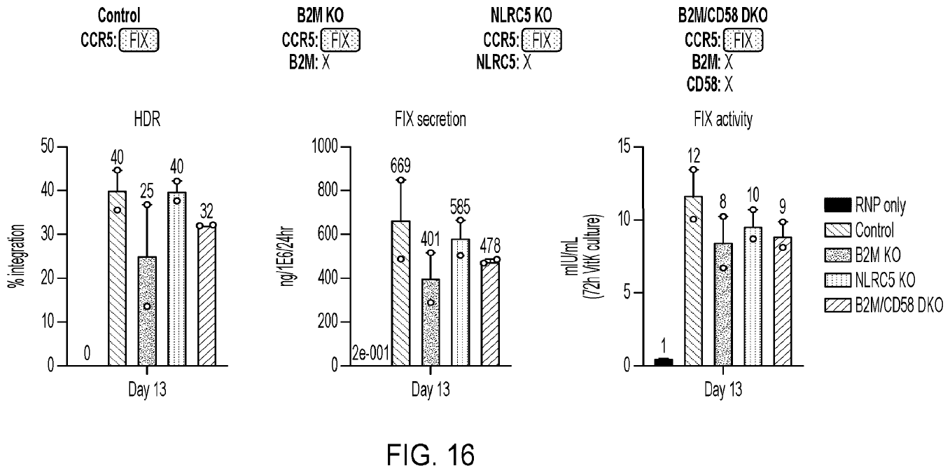

As outlined in FIG. 16, engineered B lineage cells were assessed for integration efficiency, secretion levels, and activity of FIX. B lineage cells were engineered as indicated to comprise a FIX transgene at CCR5 (Control), a FIX transgene at CCR5 and a knockout of B2M (B2M KO), a FIX transgene at CCR5 and a knockout of NLRC5 (NLRC5 KO), or a FIX transgene at CCR5 and knockout of B2M and CD58 (B2M/CD58 DKO).

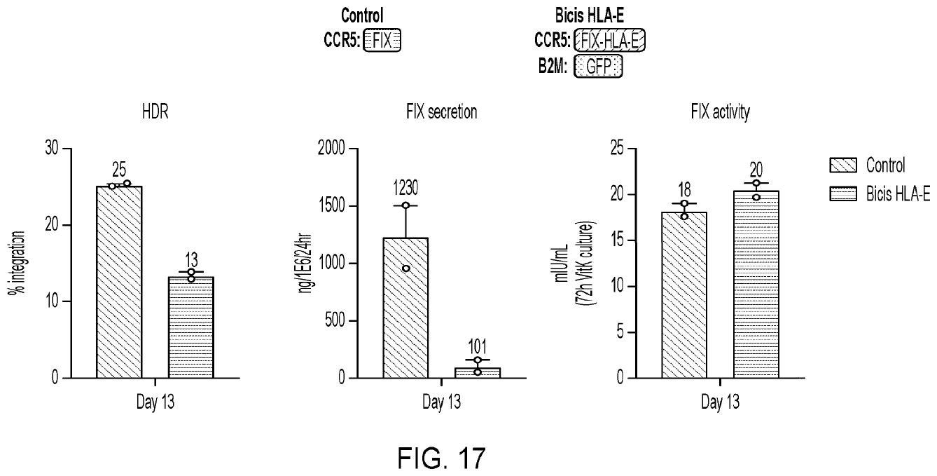

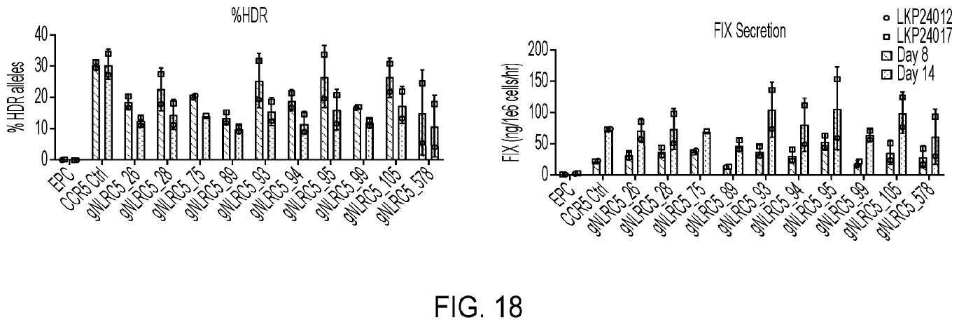

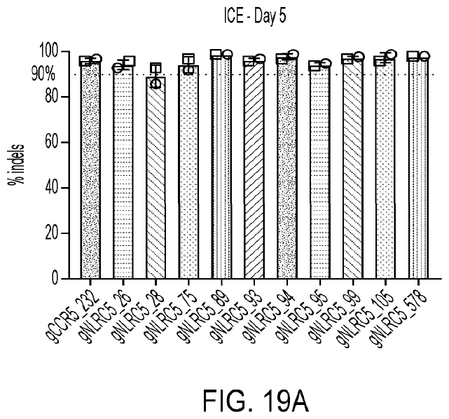

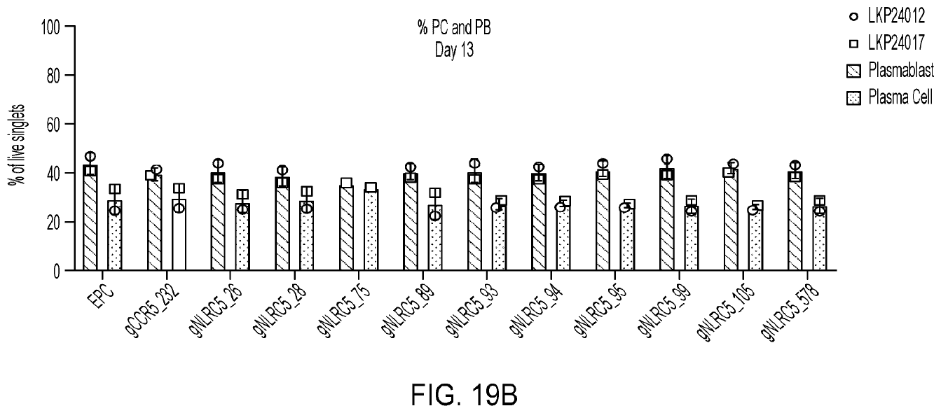

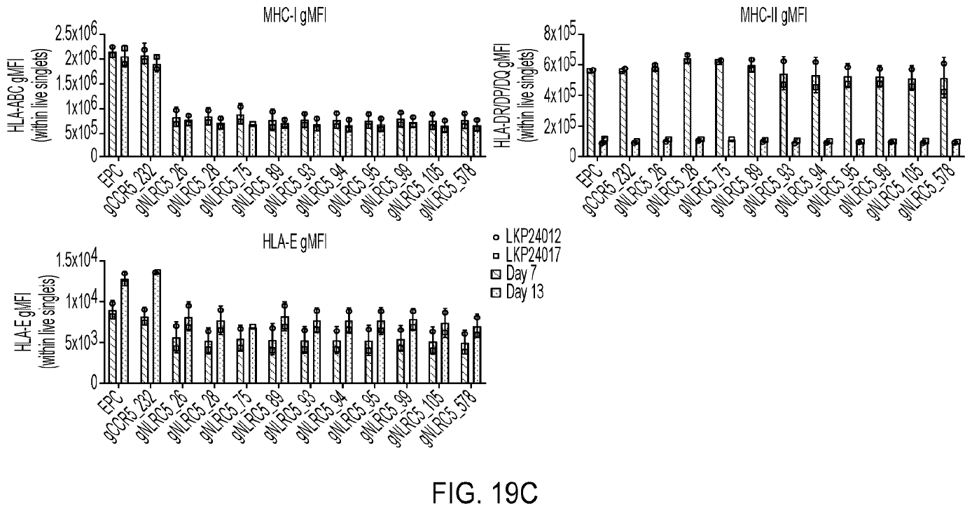

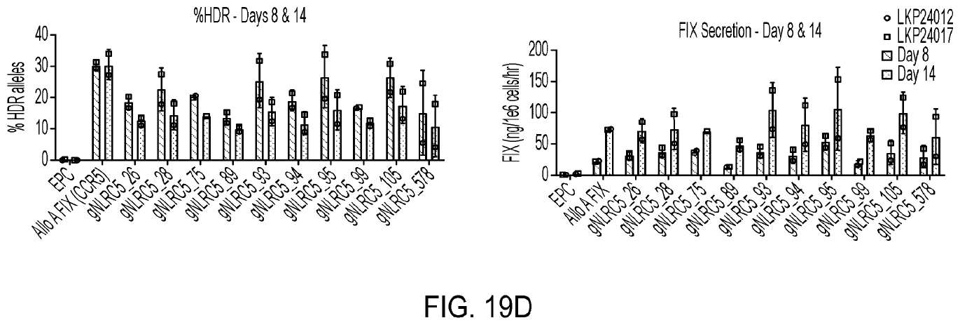

As outlined in FIG. 17, engineered B lineage cells were assessed for integration efficiency, secretion levels, and activity of FIX. B lineage cells were engineered as indicated to comprise a FIX transgene at CCR5 (Control) or a bicistronic FIX transgene and HLA-E transgene at CCR5 and a GFP transgene at B2M (Bicis HLA-E).

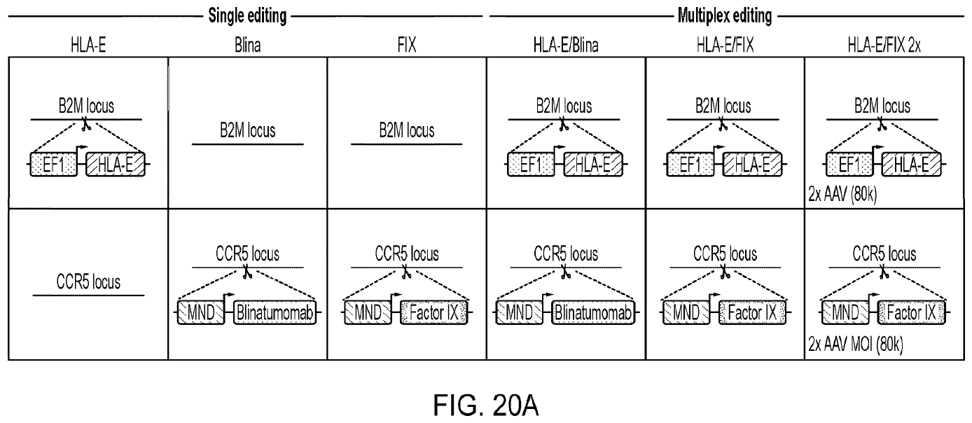

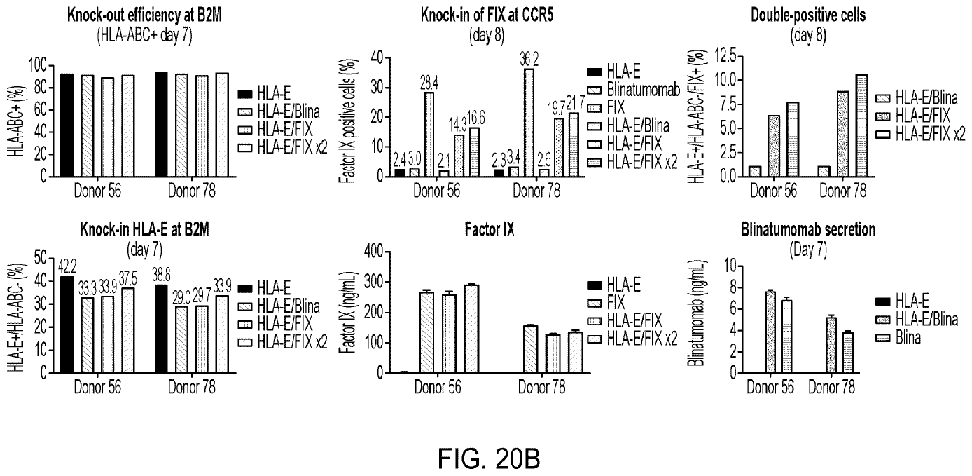

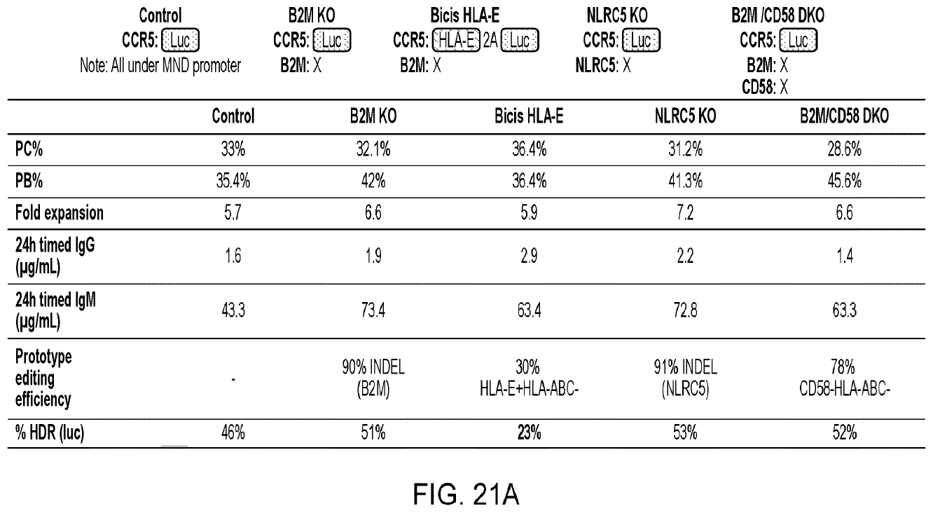

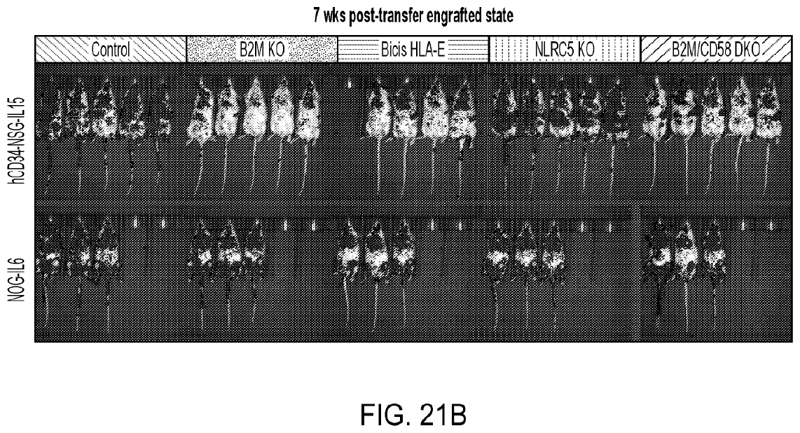

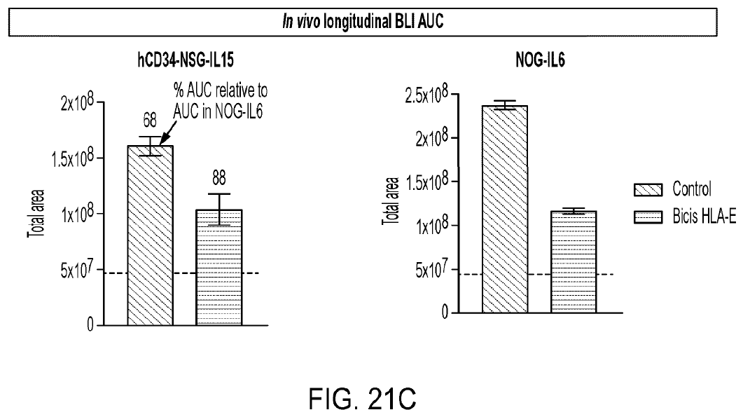

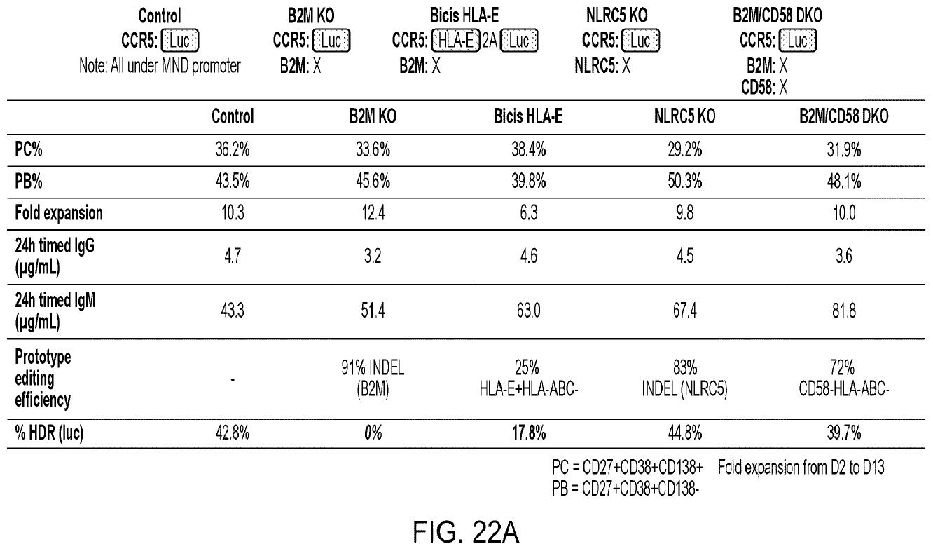

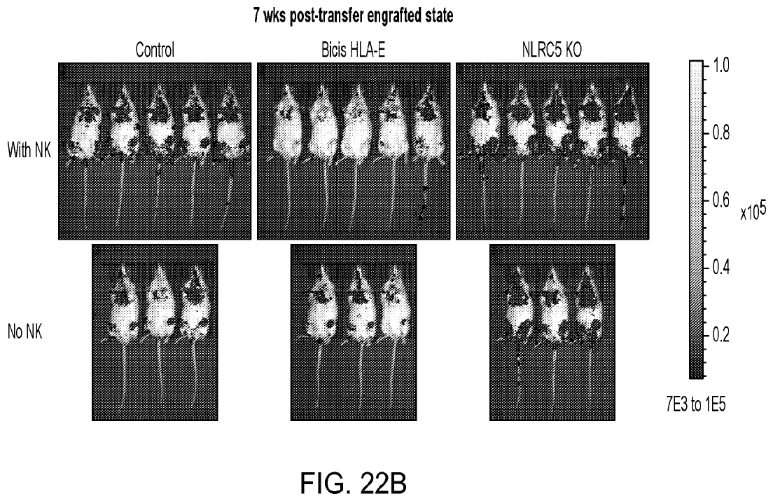

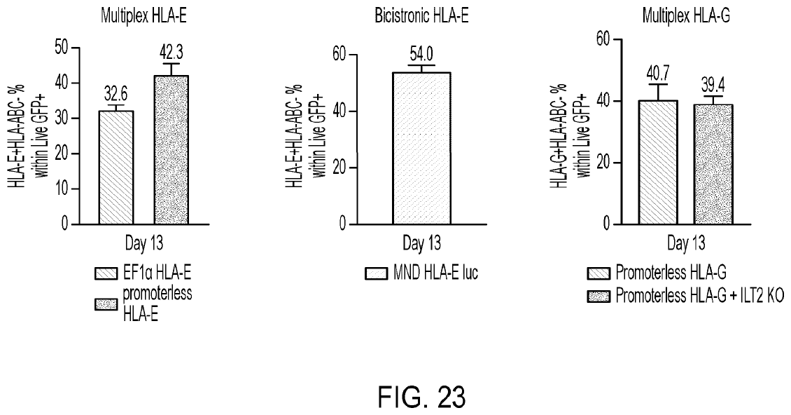

As outlined in FIG. 20A and FIG. 20B, various engineered B lineage cells were prepared through single and multiplex editing and assessed for editing efficiency and transgene expression.

In some embodiments, the present example demonstrates that B lineage cells engineered at multiple, distinct loci can engraft in vivo and exhibit persistent expression (e.g., greater than 6 weeks) in vivo. In some embodiments, B lineage cells engineered at multiple, distinct loci (e.g., CCR5 and B2M) can demonstrate improved engraftment as compared to B lineage cells that are engineered at a single locus (e.g., CCR5).

In some embodiments, B lineage cell populations engineered to reduce or knock out expression of B2M can demonstrate improved allogeneic properties (e.g., reduced immune response, increased persistence in vivo) as compared to a reference B lineage cell population (e.g., unengineered B lineage cell populations, B lineage cell populations that do have reduced B2M expression). In some embodiments, B lineage cells engineered to express one or more transgenes (e.g., HLA-E) can demonstrate improved allogeneic properties (e.g., reduced immune response, increased persistence in vivo) as compared to a reference B lineage cell population (e.g., unengineered B lineage cell populations). In some embodiments, B lineage cells engineered to express one or more transgenes (e.g., HLA-E) and reduce or knock out expression of B2M can demonstrate improved allogeneic properties (e.g., reduced immune response, increased persistence in vivo) as compared to a reference B lineage cell population (e.g., unengineered B lineage cell populations).

### Example 5: Multiplexed Allogeneic Delivery Involving Cultured and Engineered B Lineage Cell Populations Result in Reduced Immunogenic Response, Increased Persistence, and/or Improved Transgene Secretion

The present Example demonstrates that B lineage cell populations cultured and engineered with methods described by the present application (e.g., multiplex) may result in production of engineered B lineage cell populations that elicit reduced immunogenic response and increased persistence in a subject that is different than the donor (e.g., allogenic delivery). In some other embodiments, cultured B lineage cell populations may or may not be further engineered using methods and expression cassettes described herein to produce engineered B lineage cell populations. In some such embodiments, engineered B lineage cell populations may further elicit reduced immunogenic response and increased persistence in a subject that is different than the donor (e.g., allogenic delivery).

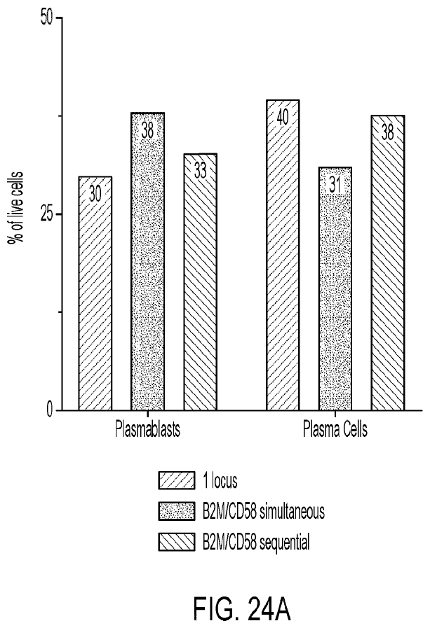

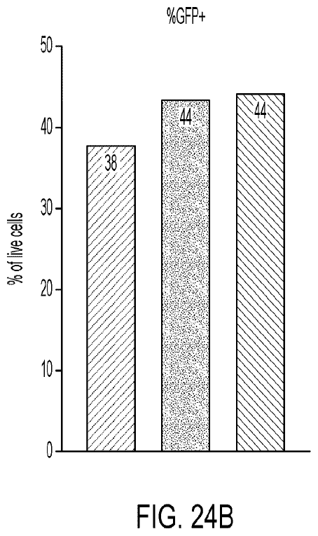

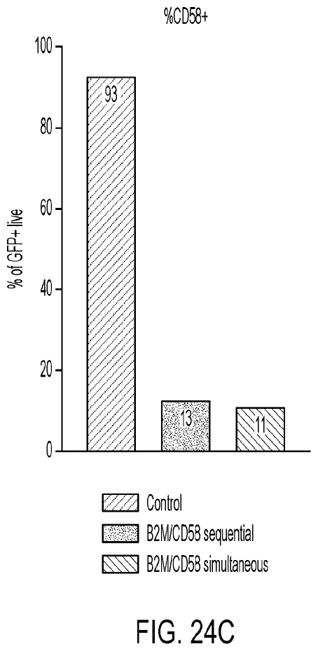

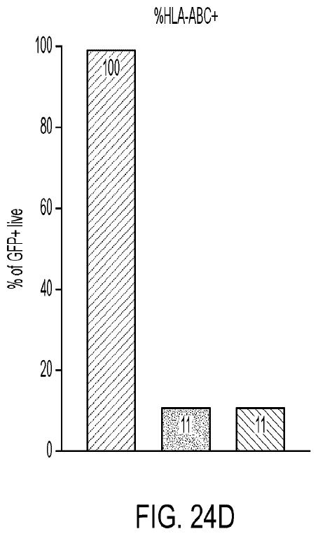

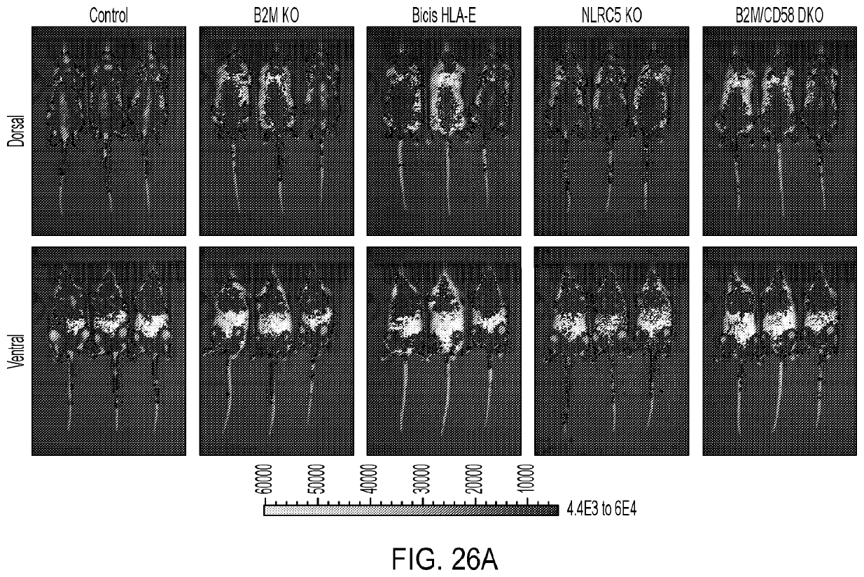

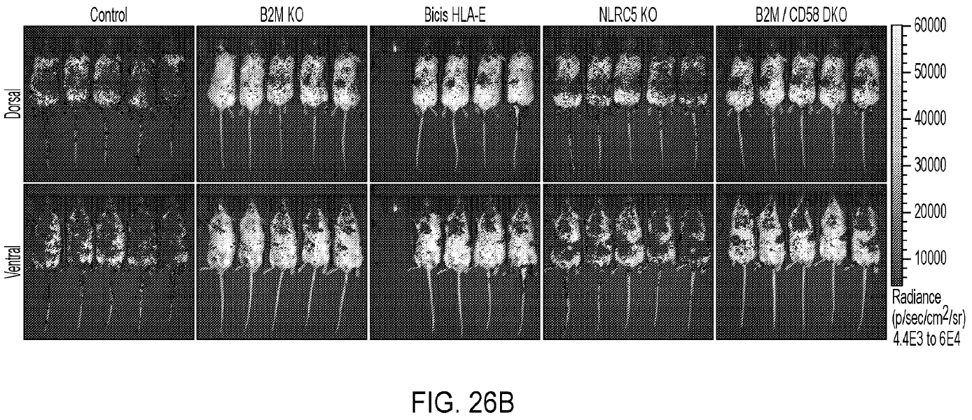

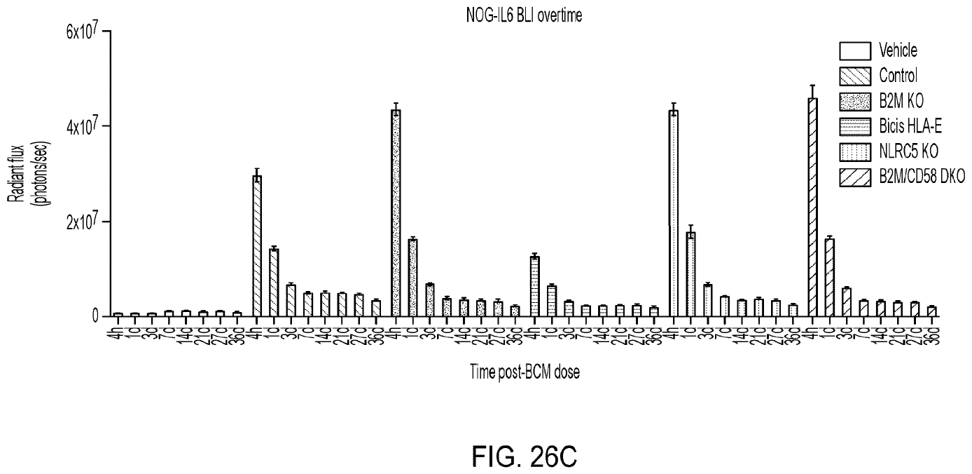

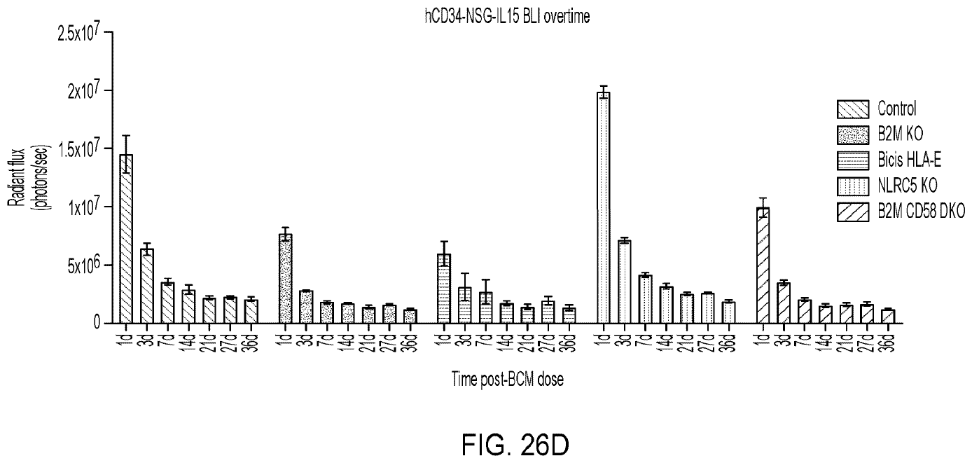

Further, B lineage cell populations were engineered using the methods described herein to have either simultaneous or sequential knockout of B2M and CD58 loci in addition to GFP knock in at CCR5. Percentage of plasmablasts and plasma cells were assessed in comparison to cultured B lineage cell population with GFP knock in at CCR5 alone (Control) at day 13 (FIG. 24A). In addition, percentage of living B lineage cells expressing GFP (FIG. 26B), percentage expressing CD58 (FIG. 24C), and percentage expressing HLA-ABC (FIG. 24D) were measured using flow cytometry.

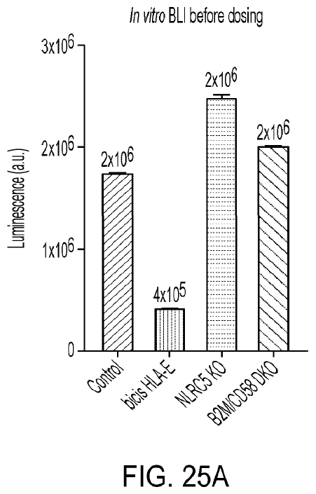

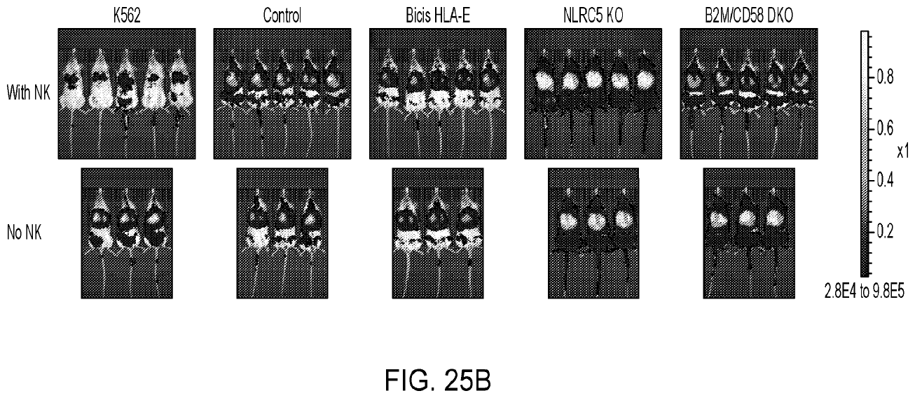

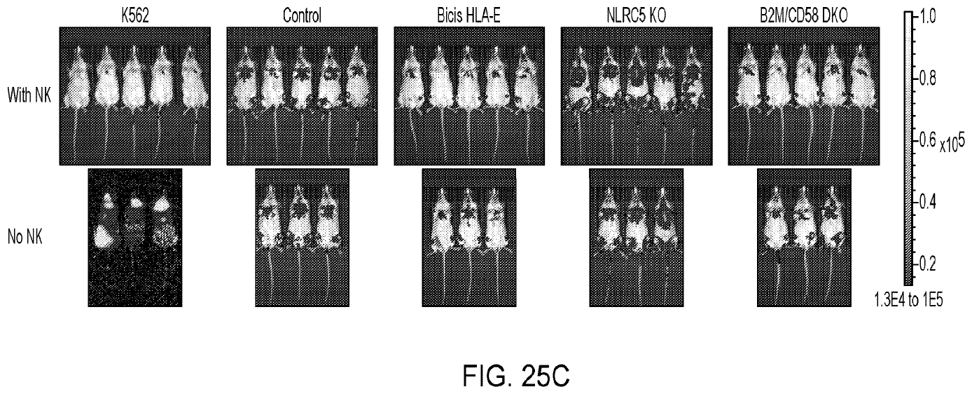

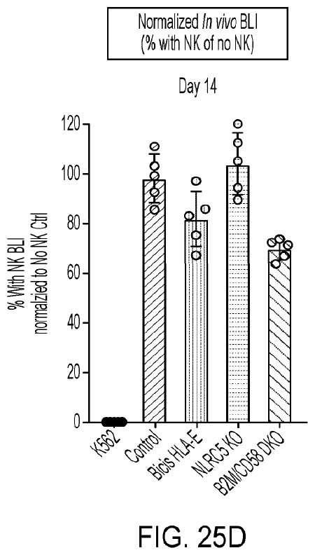

Next, B lineage cell populations were engineered to comprise a LUC transgene at CCR5 (Control), a bicistronic HLA-E transgene and LUC transgene (comprising a 2A peptide) at CCR5 and a knockout of B2M (Bicis HLA-E), a LUC transgene at CCR5 and a knockout of NLRC5 (NLRC5 KO), or a LUC transgene at CCR5 and knockout of B2M and CD58 (B2M/CD58 DKO). Said B lineage cell populations were measured for bioluminescence in vitro (FIG. 25A). Next, engineered B lineage cell populations were administered to an immunocompromised mouse model with or without pre-engraftment of NK cells. Bioluminescence was then measured in these mouse models (FIG. 25B, FIG. 25C, FIG. 25D).

Additional in vivo administration experiments were undertaken involving administration of said B lineage cell populations (Control, B2M KO, Bicis HLA-E, NLRC5 KO, and B2M/CD58 DKO) into either hCD34-NSG-IL15 mouse model or NOG-IL6 mouse model and assessed for bioluminescence over time as outlined in FIG. 26A and FIG. 26B.

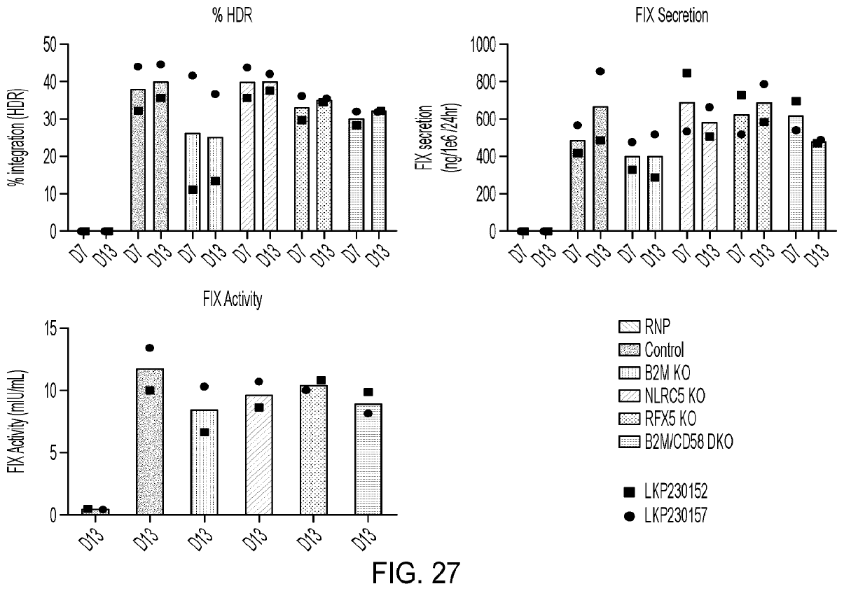

Further, multiplex engineering of two different donor B lineage cell populations using the following conditions: RNP alone, a FIX transgene at CCR5 (Control), a FIX transgene at CCR5 and a knockout of B2M (B2M KO), a FIX transgene at CCR5 and a knockout of NLRC5 (NLRC5 KO), a FIX transgene at CCR5 and a knockout of RFX5 (RFX5 KO) or a FIX transgene at CCR5 and knockout of B2M and CD58 (B2M/CD58 DKO) were assessed for their effects on genetic integration and secretion of a Factor IX (FIX). FIX secretion and activity was measured and assessed using ELISA and capture chromogenic assay as described in Example 1 (FIG. 27).

Among other things, the present disclosure demonstrates that multiplex engineered B lineage cell populations may provide comparable or improved integration efficiency as compared to a reference (e.g., non-engineered B lineage cell populations, alternatively engineered B lineage cell populations). In some embodiments, multiplex engineered lineage cell populations may provide a reduced number of genetic translocation events as compared to a reference (e.g., alternatively engineered B lineage cell populations). In some embodiments, multiplex engineered B lineage cell populations may provide engraftment and/or transgene expression (e.g., SMPD1, FIX, luciferase) that is comparable or not significantly reduced as compared to a reference condition after administration to a subject. In some embodiments, multiplex engineered B lineage cell populations described herein may provide improved engraftment and/or transgene expression as compared to a reference condition. In some embodiments, multiplex engineered B lineage cell populations described herein may engraft and/or express transgene for extended durations (e.g., 36 days). In some embodiments, multiplex engineered B lineage cells are sustained viability and may further comprise of plasmablasts and plasma cells. In some embodiments, multiplexed engineered B lineage cell populations may have reduced immunogenic response when compared to a reference condition (e.g., non-engineered B lineage cell populations or alternatively engineered B lineage cell populations).

### EXEMPLARY SEQUENCES

TABLE 1
Exemplary Homology Arms (HA)

| | | SEQ ID |
|---|---|---|
| Name | Nucleotide Sequence | NO |
| gCCR5_232 5′ | GGTGCTATAGAGCACAAGATTTTATTTGGTGAGATGGTGCTTTCATGAATTC | 10 |
| HA | CCCCAACAGAGCCAAGCTCTCCATCTAGTGGACAGGGAAGCTAGCAGCAAA | |
| | CCTTCCCTTCACTACAAAACTTCATTGCTTGGCCAAAAAGAGAGTTAATTCA | |
| | ATGTAGACATCTATGTAGGCAATTAAAAACCTATTGATGTATAAAACAGTT | |
| | TGCATTCATGGAGGGCAACTAAATACATTCTAGGACTTTATAAAAGATCAC | |
| | TTTTTATTTATGCACAGGGTGGAACAAGATGGATTATCAAGTGTCAAGTCCA | |
| | ATCTATGACATCAATTATTATACATCGGAGCCCTGCCAAAAAATCAATGTG | |
| | AAGCAAATCGCAGCCCGCCTCCTGCCTCCGCTCTACTCACTGGTGTTCATCT | |
| | TTGGTTTTGTGGGCAACATGCTGGTCATCCTCATCCTGATAAACTGCAAAAG | |
| | GCTGAAGAGCATGACTGACATCTACCTGCTCAACCTGGCCATCTCTGACCTG | |
| | TTTTTCCTTCTTACTGTCCCCTTCTGGGCTCACTATGCTGCCGCCCAGTGGGA | |
| | CTTTGGAAATACAATGTGTCAACTCTTGACA | |
| gJCHAIN_2 5′ | tacaaaatacagattttgtgagatatatacaaaatacagattttgtgagatat | 11 |
| HA | atacaaaatacagattttgtgagatatatacaaaatacatattttgtgagata | |
| | tatacaaaataTATATTTAAATCTGGTAGCTTGGTTAATTAAGGAC | |
| | TAGTATAAAAAATTAACATAAGATACACTTGGGACCTTAAAGGACTTTCAT | |
| | TTCTCTCCCCTTCCTTCATCTCTCCTCCTTCACTTACCTTCATTTATTATTCTA | |
| | GGCATGACAAGTGTCAGATGTCTGATTTGAAGACTAATATGAATATATCTA | |
| | GGTAATAAAATGGAAAATCCCTATTTCAACATTCTTAATGGAATCCTTCCTT | |
| | GAAATTATTATTAGTCTTCTGGTTTAAACATTTATATAGTGTAAATGCAAAA | |
| | TAAAAATGATTGTATCAAATTATGGTTTTTATTTGGATGCTTTGCAAGAAAA | |
| | TACTTAAATGTGAGTCATACATTTTTCCAGTTCAGCATTTAAACTTCAGACT | |
| | TCAACCACAGTTGTGATTGTTTTTAGTTTGTTAGCTGCCTGGAGTGTTATTTT | |
| | AAGAAAGCAGAAGCACCATCATTTGCACACT | |
| gJCHAIN_31 5′ | atacatattttgtgagatatatacaaaataTATATTTAAATCTGGTAGCTTGG | 12 |
| HA | TTAATTAAGGACTAGTATAAAAAATTAACATAAGATACACTTGGGACCTTAAA | |
| | GGACTTTCATTTCTCTCCCCTTCCTTCATCTCTCCTCCTTCACTTACCTTCAT | |
| | TTATTATTCTAGGCATGACAAGTGTCAGATGTCTGATTTGAAGACTAATATGA | |
| | ATATATCTAGGTAATAAAATGGAAAATCCCTATTTCAACATTCTTAATGGAAT | |
| | CCTTCCTTGAAATTATTATTAGTCTTCTGGTTTAAACATTTATATAGTGTAAA | |
| | TGCAAAATAAAAATGATTGTATCAAATTATGGTTTTTATTTGGATGCTTTGCA | |
| | AGAAAATACTTAAATGTGAGTCATACATTTTTCCAGTTCAGCATTTAAACTTC | |
| | AGACTTCAACCACAGTTGTGATTGTTTTTAGTTTGTTAGCTGCCTGGAGTGTT | |
| | ATTTTAAGAAAGCAGAAGCACCATCATTTGCACACTCCTTATAGATCACACAC | |
| | CTTAACCCTGACTTTTTTTGCTCCAGTTTTTCAGAAGAAGTGAAGTCAAGATG | |
| | AAGAACCATTTGCTTTT | |
| gJCHAIN_54 5′ | TTTTCTAAAGAAAAAATCATTGGCATTCATAGAAAGGCCAATTTCTCTTAAT | 13 |
| HA | AGTTCAATAAGTGGATTTGATCTTATAAAAAGGCAGGTGTTTCTTTGGAAAT | |
| | GACAGACTCCAACATCAATTTTTTTAAAAATTCTCCCTTTCTTGTCACTATA | |
| | AATAACTTGTTTAGACAGATATACAGTTGGGAATAAGCCTAACACAGTAGA | |
| | AATTGCTGTATGGTGTAGATAAAACAATCATATTATCATATCATTAATTATA | |
| | TTGCTTACTTTCAACTAATATATATTAAAGATTGGAAAATCCCATAAGCTAT | |
| | TCTGTATTGTAGAGCTGCTTATGTCTGAAAGGAGTCATCCCTTGCTGTCATG | |
| | TCAGAGCTGCAAGAACTAATTGATTTTGGATTGAAATGTGTAGTCACATTTT | |
| | GAGACAGCATTTGAGGGGATTGTCTAATACATATATTTGCTTTTCAGCTGTA | |
| | AAAAATGTGATCCTACAGAAGTGGAGCTGGATAATCAGATAGTTACTGCTA | |
| | CCCAGAGCAATATCTGTGATGAAGACAGTGCTACAGAGACCTGCTACACTT | |
| | ATGACAGAAACAAGTGCTACACAGCTGTGGT | |
| gJtolGHM_23 5′ | ATGTGACGCCCGGAGACAGAAGGTCTCTGGGTGGCTGGGTTTTTGTGGGGT | 14 |
| HA | GAGGATGGACATTCTGCCATTGTGATTACTACTACTACTACTACATGGACGT | |
| | CTGGGGCAAAGGGACCACGGTCACCGTCTCCTCAGGTAAGAATGGCCACTC | |
| | TAGGGCCTTTGTTTTCTGCTACTGCCTGTGGGGTTTCCTGAGCATTGCAGGT | |
| | TGGTCCTCGGGGCATGTTCCGAGGGGACCTGGGCGGACTGGCCAGGAGGGG | |
| | ATGGGCACTGGGGTGCCTTGAGGATCTGGGAGCCTCTGTGGATTTTCCGAT | |
| | GCCTTTGGAAAATGGGACTCAGGTTGGGTGCGTCTGATGGAGTAACTGAGC | |
| | CTGGGGGCTTGGGGAGCCACATTTGGACGAGATGCCTGAACAAACCAGGG | |
| | GTCTTAGTGATGGCTGAGGAATGTGTCTCAGGAGCGGTGTC | |
| gCCR5_232 3′ | CTCTATTTTATAGGCTTCTTCTCTGGAATCTTCTTCATCATCCTCCTGACAAT | 15 |
| HA | CGATAGGTACCTGGCTGTCGTCCATGCTGTGTTTGCTTTAAAAGCCAGGACG | |
| | GTCACCTTTGGGGTGGTGACAAGTGTGATCACTTGGGTGGTGGCTGTGTTTG | |
| | CGTCTCTCCCAGGAATCATCTTTACCAGATCTCAAAAAGAAGGTCTTCATTA | |
| | CACCTGCAGCTCTCATTTTCCATACAGTCAGTATCAATTCTGGAAGAATTTC | |
| | CAGACATTAAAGATAGTCATCTTGGGGCTGGTCCTGCCGCTGCTTGTCATGG | |
| | TCATCTGCTACTCGGGAATCCTAAAAACTCTGCTTCGGTGTCGAAATGAGA | |
| | AGAAGAGGCACAGGGCTGTGAGGCTTATCTTCACCATCATGATTGTTTATTT | |
| | TCTCTTCTGGGCTCCCTACAACATTGTCCTTCTCCTGAACACCTTCCAGGAA | |
| | TTCTTTGGCCTGAATAATTGCAGTAGCTCTAACAGGTTGGACCAAGCTATGC | |
| | AGGTGACAGAGACTCTTGGGATGACGCACTGCTGCATCAACCCCATCATCT | |
| | ATGCCTTTGTCGGGGAGAAGTTCAGAAAC | |
| gJCHAIN_2 3′ | AGATCACACACCTTAACCCTGACTTTTTTTGCTCCAGTTTTTCAGAAGAAGT | 16 |
| HA | GAAGTCAAGATGAAGAACCATTTGCTTTTCTGGGGAGTCCTGGCGGTTTTTA | |
| | TTAAGGCTGTTCATGTGAAAGGTATGTGATATTTAGAAAATGATCCCAGAT | |
| | CAAAGGAAAAATATAGGACAGATCTGTTTTTCAGTTTTAAACATTTATGCCT | |
| | ATGCTTTGGTTGGCCAGTTAGCCAGCTACTTTTTCCTAACATGCTTTCATCTT | |
| | TCTATGGCAGTTGTCTTTACATAGATAAGACTCTTTTTATTCTTTTTTCTCTT | |
| | ATCTTACTTACTTGTTTGCCTACTACAAACTAGAGAATAAGAAATTAGCTAA | |
| | CTACAGATAGTCCTGCTTATATGAATGTCCATATTGAATCTTAAAAATCTAT | |
| | AATCCTTCCCTTGAGTCTCTTAAATTAATATTCTGGAGAAATGAAATAATAA | |
| | TTACTGTAAGTACAACATGGAATGCATTGGGATTTTATTTCCCCAAAGAGGC | |
| | TGGTATTGGTTTTAACAGCTTTCCTGGATCTGATCCCACAGATTTATTATGA | |
| | CCCTTTAACAATGTTTAATGTTATACA | |
| gJCHAIN_31 3′ | AGTCCTGGCGGTTTTTATTAAGGCTGTTCATGTGAAAGGTATGTGATATTTA | 60 |
| HA | GAAAATGATCCCAGATCAAAGGAAAAATATAGGACAGATCTGTTTTTCAGT | |
| | TTTAAACATTTATGCCTATGCTTTGGTTGGCCAGTTAGCCAGCTACTTTTTCC | |
| | TAACATGCTTTCATCTTTCTATGGCAGTTGTCTTTACATAGATAAGACTCTTT | |
| | TTATTCTTTTTTCTCTTATCTTACTTACTTGTTTGCCTACTACAAACTAGAGA | |
| | ATAAGAAATTAGCTAACTACAGATAGTCCTGCTTATATGAATGTCCATATTG | |
| | AATCTTAAAAATCTATAATCCTTCCCTTGAGTCTCTTAAATTAATATTCTGG | |
| | AGAAATGAAATAATAATTACTGTAAGTACAACATGGAATGCATTGGGATTT | |
| | TATTTCCCCAAAGAGGCTGGTATTGGTTTTAACAGCTTTCCTGGATCTGATC | |
| | CCACAGATTTATTATGACCCTTTAACAATGTTTAATGTTATACAGGTTGCCT | |
| | ATAAATTGATGACTGAACTTTTGTTATTTTGTTTACTGATCTTTCAAACAACT | |
| | TCAGAAAATGGAATACATTCTGAATT | |
| gJCHAIN_54 3′ | CGTATATGGTGGTGAGACCAAAATGGTGGAAACAGCCTTAACCCCAGATGC | 17 |
| HA | CTGCTATCCTGACTAATTTAAGTCATTGCTGACTGCATAGCTCTTTTTCTTGA | |
| | GAGGCTCTCCATTTTGATTCAGAAAGTTAGCATATTTATTACCAATGAATTT | |
| | GAAACCAGGGCttttttttttttttGGGTGATGTAAAACCAACTCCCTGCCAC | |
| | CAAAATAATTAAAATAGTCACATTGTTATCTTTATTAGGTAATCACTTCTTAA | |
| | TTATATGTTCATACTCTAAGTATCAAAATCTTCCAATTATCATGCTCACCTGA | |
| | AAGAGGTATGCTCTCTTAGGAATACAGTTTCTAGCATTAAACAAATAAACAAG | |
| | GGGAGAAAATAAAACTCAAGGACTGAAAATCAGGAGGTGTAATAAAATGTTCCT | |
| | CGCATTCCCCCCCGCttttttttttttttttGACTTTGCCTTGGAGAGCCAGAG | |
| | CTTCCGCATTTTCTTTACTATTCTTTTTAAAAAAAGTTTCACTGTGTAGAGAAC | |
| | ATATATGCATAAACATAGGTCAATTATATGTCTCCATTAGAAAAATAATAATTG | |
| | GAAAACATGTTCTAGA | |
| gJtolGHM_23 3′ | TGTAGGACTGCAAGATCGCTGCACAGCAGCGAATCGTGAAATATTTTCTTT | 18 |
| HA | AGAATTATGAGGTGCGCTGTGTGTCAACCTGCATCTTAAATTCTTTATTGGC | |
| | TGGAAAGAGAACTGTCGGAGTGGGTGAATCCAGCCAGGAGGGACGCGTAG | |
| | CCCCGGTCTTGATGAGAGCAGGGTTGGGGGCAGGGGTAGCCCAGAAACGG | |
| | TGGCTGCCGTCCTGACAGGGGCTTAGGGAGGCTCCAGGACCTCAGTGCCTT | |
| | GAAGCTGGTTTCCATGAGAAAAGGATTGTTTATCTTAGGAGGCATGCTTACT | |
| | GTTAAAAGACAGGATATGTTTGAAGTGGCTTCTGAGAAAAATGGTTAAGAA | |
| | AATTATGACTTAAAAATGTGAGAGATTTTCAAGTATATTAATTTTTTTAACT | |
| | GTCCAAGTATTTGAAATTCTTATCATTTGATTAACACCCAT | |

TABLE 2
Exemplary Expression Cassettes

| | | SEQ |
|---|---|---|
| | | ID |
| Name | Nucleotide Sequence | NO |
| ATM061 | GCCACCATGCAGAGGGTGAACATGATCATGGCTGAGAGCCCTGGCCTGAT | 19 |
| | CACCATCTGCCTGCTGGGCTACCTGCTGTCTGCTGAGTGCACTGTGTTCCT | |
| | GGACCATGAGAATGCCAACAAGATCCTGAACAGGCCCAAGAGATACAAC | |
| | TCTGGCAAGCTGGAGGAGTTTGTGCAGGGCAACCTGGAGAGGGAGTGCA | |
| | TGGAGGAGAAGTGCAGCTTTGAGGAGGCCAGGGAGGTGTTTGAGAACAC | |
| | TGAGAGGACCACTGAGTTCTGGAAGCAGTATGTGGATGGGGACCAGTGT | |
| | GAGAGCAACCCCTGCCTGAATGGGGGCAGCTGCAAGGATGACATCAACA | |
| | GCTATGAGTGCTGGTGCCCCTTTGGCTTTGAGGGCAAGAACTGTGAGCTG | |
| | GATGTGACCTGCAACATCAAGAATGGCAGATGTGAGCAGTTCTGCAAGA | |
| | ACTCTGCTGACAACAAGGTGGTGTGCAGCTGCACTGAGGGCTACAGGCTG | |
| | GCTGAGAACCAGAAGAGCTGTGAGCCTGCTGTGCCATTCCCATGTGGCAG | |
| | AGTGTCTGTGAGCCAGACCAGCAAGCTGACCAGGGCTGAGGCTGTGTTCC | |
| | CTGATGTGGACTATGTGAACAGCACTGAGGCTGAAACCATCCTGGACAAC | |
| | ATCACCCAGAGCACCCAGAGCTTCAATGACTTCACCAGGGTGGTGGGGG | |
| | GGGAGGATGCCAAGCCTGGCCAGTTCCCCTGGCAAGTGGTGCTGAATGGC | |
| | AAGGTGGATGCCTTCTGTGGGGGCAGCATTGTGAATGAGAAGTGGATTGT | |
| | GACTGCTGCCCACTGTGTGGAGACTGGGGTGAAGATCACTGTGGTGGCTG | |
| | GGGAGCACAACATTGAGGAGACTGAGCACACTGAGCAGAAGAGGAATGT | |
| | GATCAGGATCATCCCCCACCACAACTACAATGCTGCCATCAACAAGTACA | |
| | ACCATGACATTGCCCTGCTGGAGCTGGATGAGCCCCTGGTGCTGAACAGC | |
| | TATGTGACCCCCATCTGCATTGCTGACAAGGAGTACACCAACATCTTCCT | |
| | GAAGTTTGGCTCTGGCTATGTGTCTGGCTGGGGCAGGGTGTTCCACAAGG | |
| | GCAGGTCTGCCCTGGTGCTGCAGTACCTGAGGGTGCCCCTGGTGGACAGG | |
| | GCCACCTGCCTGCTGAGCACCAAGTTCACCATCTACAACAACATGTTCTG | |
| | TGCTGGCTTCCATGAGGGGGGCAGGGACAGCTGCCAGGGGGACTCTGGG | |
| | GGCCCCCATGTGACTGAGGTGGAGGGCACCAGCTTCCTGACTGGCATCAT | |
| | CAGCTGGGGGGAGGAGTGTGCCATGAAGGGCAAGTATGGCATCTACACC | |
| | AAAGTCTCCAGATATGTGAACTGGATCAAGGAGAAGACCAAGCTGACCT | |
| | GA | |
| NO CG SS | GCCACCATGCAGAGGGTGAACATGATCATGGCTGAGAGCCCTGGCCTGAT | 20 |
| | CACCATCTGCCTGCTGGGCTACCTGCTGAGTGCTGAGTGCACAGTGTTCCT | |
| | GGACCATGAGAATGCCAACAAGATCCTGAACAGACCCAAGAGATACAAC | |
| | AGTGGCAAGCTGGAGGAGTTTGTGCAGGGCAACCTGGAGAGGGAGTGCA | |
| | TGGAGGAGAAGTGCAGCTTTGAGGAGGCCAGGGAAGTGTTTGAGAACAC | |
| | AGAGAGAACCACAGAGTTCTGGAAGCAGTATGTGGATGGAGACCAGTGT | |
| | GAGAGCAACCCTTGCCTGAATGGAGGCAGCTGCAAGGATGACATCAACA | |
| | GCTATGAGTGCTGGTGCCCTTTTGGCTTTGAGGGCAAGAACTGTGAGCTG | |
| | GATGTGACCTGCAACATCAAGAATGGAAGGTGTGAGCAGTTCTGCAAGA | |
| | ACTCTGCTGACAACAAAGTGGTGTGTAGCTGCACAGAGGGCTACAGACTG | |
| | GCTGAGAACCAGAAGTCTTGTGAGCCAGCTGTGCCTTTTCCCTGTGGCAG | |
| | AGTCAGTGTGTCCCAGACCAGCAAGCTGACCAGAGCTGAGACAGTGTTCC | |
| | CTGATGTGGACTATGTGAATAGCACAGAGGCTGAGACCATCCTGGACAAC | |
| | ATCACCCAGAGCACCCAGTCCTTCAATGACTTCACCAGAGTTGTGGGAGG | |
| | GGAGGATGCCAAGCCTGGCCAGTTCCCCTGGCAGGTGGTGCTGAATGGCA | |
| | AAGTGGATGCCTTCTGTGGAGGCAGCATTGTGAATGAGAAGTGGATTGTG | |
| | ACAGCTGCCCACTGTGTGGAGACAGGAGTGAAGATCACAGTGGTGGCTG | |
| | GAGAACACAATATTGAGGAGACAGAGCACACTGAGCAGAAGAGGAATGT | |
| | CATCAGAATTATCCCCCACCACAACTACAATGCTGCCATCAACAAGTACA | |
| | ACCATGACATTGCCCTGCTGGAGCTGGATGAGCCTCTGGTGCTGAATAGC | |
| | TATGTGACCCCCATCTGCATTGCTGACAAGGAGTACACCAACATCTTCCT | |
| | GAAGTTTGGCAGTGGCTATGTCAGTGGCTGGGGCAGAGTGTTCCACAAGG | |
| | GCAGATCTGCCCTGGTGCTGCAGTACCTGAGAGTGCCCCTGGTGGACAGA | |
| | GCCACCTGCCTGTTGAGCACCAAGTTCACCATCTACAACAACATGTTCTG | |
| | TGCTGGCTTCCATGAGGGAGGCAGAGACAGCTGCCAGGGAGATTCTGGA | |
| | GGGCCCCATGTGACAGAAGTGGAGGGCACCAGCTTCCTGACAGGCATCA | |
| | TCAGCTGGGGGGAGGAGTGTGCCATGAAGGGCAAGTATGGCATCTACAC | |
| | CAAAGTCAGCAGATATGTGAACTGGATCAAGGAGAAAACCAAGCTGACC | |
| | TGA | |
| NO CG AH | GCCACCATGCAGAGGGTGAACATGATCATGGCAGAGAGCCCTGGCCTGA | 21 |
| | TCACCATCTGCCTGCTGGGCTACCTGCTGTCTGCAGAATGTACAGTGTTTC | |
| | TGGACCATGAGAATGCCAACAAGATCCTGAACAGGCCCAAGAGATACAA | |
| | CTCTGGCAAGCTGGAGGAGTTTGTGCAGGGCAACCTGGAGAGGGAGTGC | |
| | ATGGAGGAGAAGTGCAGCTTTGAGGAGGCCAGGGAAGTGTTTGAGAACA | |
| | CTGAGAGGACCACTGAGTTCTGGAAGCAGTATGTGGATGGAGACCAGTG | |
| | TGAGAGCAACCCTTGCCTGAATGGAGGCAGCTGCAAGGATGACATCAAC | |
| | AGCTATGAGTGCTGGTGCCCATTTGGCTTTGAGGGCAAGAACTGTGAGCT | |
| | GGATGTGACCTGCAACATCAAGAATGGCAGATGTGAGCAGTTCTGCAAG | |
| | AACTCTGCTGACAACAAAGTGGTGTGTAGCTGCACTGAGGGCTACAGACT | |
| | GGCAGAGAATCAGAAGTCCTGTGAACCTGCTGTGCCTTTTCCCTGTGGCA | |
| | GAGTCTCTGTGTCCCAGACCAGCAAGCTGACCAGAGCAGAGACTGTGTTC | |
| | CCAGATGTGGACTATGTGAATAGCACTGAGGCAGAGACCATCCTGGACA | |
| | ACATCACCCAGAGCACCCAGTCCTTCAATGACTTCACCAGAGTTGTGGGA | |
| | GGAGAGGATGCCAAGCCAGGCCAGTTCCCCTGGCAGGTGGTGCTGAATG | |
| | GCAAAGTGGATGCCTTCTGTGGAGGCAGCATTGTGAATGAGAAGTGGATT | |
| | GTGACAGCAGCCCACTGTGTGGAGACTGGAGTGAAGATCACTGTGGTGG | |
| | CAGGAGAACACAATATTGAGGAGACTGAGCACACAGAGCAGAAGAGAA | |
| | ATGTCATCAGGATTATCCCCCACCACAACTACAATGCAGCCATCAACAAG | |
| | TACAACCATGACATTGCCCTGCTGGAGCTGGATGAGCCTCTGGTGCTGAA | |
| | TAGCTATGTGACCCCCATCTGCATTGCAGACAAGGAGTACACCAACATCT | |
| | TCCTGAAGTTTGGCTCTGGCTATGTCAGTGGCTGGGGCAGAGTGTTCCAC | |
| | AAGGGCAGATCTGCCCTGGTGCTGCAGTACCTGAGAGTGCCCCTGGTGGA | |
| | CAGAGCCACCTGCCTGTTGAGCACCAAGTTCACCATCTACAACAACATGT | |
| | TCTGTGCAGGCTTCCATGAGGGAGGCAGAGACAGCTGCCAGGGAGACTC | |
| | TGGAGGACCCCATGTGACTGAAGTGGAGGGCACCAGCTTCCTGACTGGCA | |
| | TCATCAGCTGGGGAGAGGAGTGTGCCATGAAGGGCAAGTATGGCATCTA | |
| | CACCAAAGTCAGCAGATATGTGAACTGGATCAAGGAGAAAACCAAGCTG | |
| | ACCTGA | |
| p00362_SMPD1 | cgcggaacccctatttgtttatttttctaaatacattcaaa | 22 |
| wild-type | tatgtatccgctcatgagacaataaccctgataaatgcttcaataa | |
| | tattgaaaaaggaagagtATGAGCCATATTCAACGGGAAACGTCTTGCTCGAGGCCG | |
| | CGATTAAATTCCAACATGGATGCTGATTTATATGGGTATAAATGGGCTCG | |
| | CGATAATGTCGGGCAATCAGGTGCGACAATCTATCGATTGTATGGGAAGC | |
| | CCGATGCGCCAGAGTTGTTTCTGAAACATGGCAAAGGTAGCGTTGCCAAT | |
| | GATGTTACAGATGAGATGGTCAGACTAAACTGGCTGACGGAATTTATGCC | |
| | TCTTCCGACCATCAAGCATTTTATCCGTACTCCTGATGATGCATGGTTACT | |
| | CACCACTGCGATCCCCGGGAAAACAGCATTCCAGGTATTAGAAGAATATC | |
| | CTGATTCAGGTGAAAATATTGTTGATGCGCTGGCAGTGTTCCTGCGCCGG | |
| | TTGCATTCGATTCCTGTTTGTAATTGTCCTTTTAACAGCGATCGCGTATTTC | |
| | GTCTCGCTCAGGCGCAATCACGAATGAATAACGGTTTGGTTGATGCGAGT | |
| | GATTTTGATGACGAGCGTAATGGCTGGCCTGTTGAACAAGTCTGGAAAGA | |
| | AATGCATAAGCTTTTGCCATTCTCACCGGATTCAGTCGTCACTCATGGTGA | |
| | TTTCTCACTTGATAACCTTATTTTTGACGAGGGGAAATTAATAGGTTGTAT | |
| | TGATGTTGGACGAGTCGGAATCGCAGACCGATACCAGGATCTTGCCATCC | |
| | TATGGAACTGCCTCGGTGAGTTTTCTCCTTCATTACAGAAACGGCTTTTTC | |
| | AAAAATATGGTATTGATAATCCTGATATGAATAAATTGCAGTTTCATTTG | |
| | ATGCTCGATGAGTTTTTCTAActgtcagaccaagtttactcatatatact | |
| | ttagattgatttaaaacttcatttttaatttaaaaggatctaggtgaaga | |
| | tcctttttgataatctcatgaccaaaatcccttaacgtgagttttcgttc | |
| | cactgagcgtcagaccccgtagaaaagatcaaaggatcttcttgagatcc | |
| | tttttttctgcgcgtaatctgctgcttgcaaacaaaaaaaccaccgctac | |
| | cagcggtggtttgtttgccggatcaagagctaccaactctttttccgaag | |
| | gtaactggcttcagcagagcgcagataccaaatactgttcttctagtgta | |
| | gccgtagttaggccaccacttcaagaactctgtagcaccgcctacatacc | |
| | tcgctctgctaatcctgttaccagtggctgctgccagtggcgataagtcg | |
| | tgtcttaccgggttggactcaagacgatagttaccggataaggcgcagcg | |
| | gtcgggctgaacggggggttcgtgcacacagcccagcttggagcgaacga | |
| | cctacaccgaactgagatacctacagcgtgagctatgagaaagcgccacg | |
| | cttcccgaagggagaaaggcggacaggtatccggtaagcggcagggtcgg | |
| | aacaggagagcgcacgagggagcttccagggggaaacgcctggtatcttt | |
| | atagtcctgtcgggtttcgccacctctgacttgagcgtcgatttttgtga | |
| | tgctcgtcaggggggcggagcctatggaaaaacgccagcaacgcggcctt | |
| | tttacggttcctggccttttgctggccttttgctcacatgttctttcctg | |
| | cgttatcccctgattctgtggataaccgtattaccgcctttgagtgagct | |
| | gataccgctcgccgcagccgaacgaccgagcgcagcgagtcagtgagcga | |
| | ggaagcggaagagcgcccaatacgcaaaccgcctctccccgcgcgttggc | |
| | cgattcattaatgcagctggcacgacaggtttcccgactggaaagcgggc | |
| | agtgagcgcaacgcaattaatgtgagttagctcactcattaggcacccca | |
| | ggctttacactttatgcttccggctcgtatgttgtgtggaattgtgagcg | |
| | gataacaatttcacacaggaaacagctatgaccatgattacgaattgcct | |
| | gcaggcagctgcgcgctcgctcgctcactgaggccgcccgggcaaagccc | |
| | gggcgtcgggcgacctttggtcgcccggcctcagtgagcgagcgagcgcg | |
| | cagagagggagtggccaactccatcactaggggttcctatcgatggcgcg | |
| | cctccagatatcatacactcgagcaactttgtatagaaaagttgggtgct | |
| | atagagcacaagattttatttggtgagatggtgctttcatgaattccccc | |
| | aacagagccaagctctccatctagtggacagggaagctagcagcaaacct | |
| | tcccttcactacaaaacttcattgcttggccaaaaagagagttaattcaa | |
| | tgtagacatctatgtaggcaattaaaaacctattgatgtataaaacagtt | |
| | tgcattcatggagggcaactaaatacattctaggactttataaaagatca | |
| | ctttttatttatgcacagggtggaacaagatggattatcaagtgtcaagt | |
| | ccaatctatgacatcaattattatacatcggagccctgccaaaaaatcaa | |
| | tgtgaagcaaatcgcagcccgcctcctgcctccgctctactcactggtgt | |
| | tcatctttggttttgtgggcaacatgctggtcatcctcatcctgataaac | |
| | tgcaaaaggctgaagagcatgactgacatctacctgctcaacctggccat | |
| | ctctgacctgtttttccttcttactgtccccttctgggctcactatgctg | |
| | ccgcccagtgggactttggaaatacaatgtgtcaactcttgacagaacag | |
| | ttggaacagcagaatatgggccaaacaggatatctgtggtaagcagttcc | |
| | tgccccggctcagggccaagaacagttggaacagcagaatatgggccaaa | |
| | caggatatctgtggtaagcagttcctgccccggctcagggccaagaacag | |
| | atggtccccagatgcggtcccgccctcagcagtttctagagaaccatcag | |
| | atgtttccagggtgccccaaggacctgaaatgaccctgtgccttatttga | |
| | actaaccaatcagttcgcttctcgcttctgttcgcgcgcttctgctcccc | |
| | gagctctatataagcagagctcgtttagtgaaccgtcagatccaagtttg | |
| | tacaaaaaagcaggctgccaccatgccccgctacggagcgtcactccgcc | |
| | agagctgccccaggtccggccgggagcagggacaagacgggaccgccgga | |
| | gcccccggactcctttggatgggcctggtgctggcgctggcgctggcgct | |
| | ggcgctggcgctggctctgtctgactctcgggttctctgggctccggcag | |
| | aggctcaccctctttctccccaaggccatcctgccaggttacatcgcata | |
| | gtgccccggctccgagatgtctttggggggggaacctcacctgcccaatc | |
| | tgcaaaggtctattcaccgccatcaacctcgggctgaagaaggaacccaa | |
| | tgtggctcgcgtgggctccgtggccatcaagctgtgcaatctgctgaaga | |
| | tagcaccacctgccgtgtgccaatccattgtccacctctttgaggatgac | |
| | atggtggaggtgtggagacgctcagtgctgagcccatctgaggcctgtgg | |
| | cctgctcctgggctccacctgtgggcactgggacattttctcatcttgga | |
| | acatctctttgcctactgtgccgaagccgccccccaaaccccctagcccc | |
| | ccagccccaggtgcccctgtcagccgcatcctcttcctcactgacctgca | |
| | ctgggatcatgactacctggagggcacggaccctgactgtgcagacccac | |
| | tgtgctgccgccggggttctggcctgccgcccgcatcccggccaggtgcc | |
| | ggatactggggcgaatacagcaagtgtgacctgcccctgaggaccctgga | |
| | gagcctgttgagtgggctgggcccagccggcccttttgatatggtgtact | |
| | ggacaggagacatccccgcacatgatgtctggcaccagactcgtcaggac | |
| | caactgcgggccctgaccaccgtcacagcacttgtgaggaagttcctggg | |
| | gccagtgccagtgtaccctgctgt | |
| | gggtaaccatgaaagcacacctgtcaatagcttccctccccccttcattgagggcaaccactcctcccgctggctctatgaa | |
| | gcgatggccaaggcttgggagccctggctgcctgccgaagccctgcgcaccctcagaattggggggttctatgctctttccc | |
| | cataccccggtctccgcctcatctctctcaatatgaatttttgttcccgtgagaacttctggctcttgatcaactccacgga | |
| | tcccgcaggacagctccagtggctggtgggggagcttcaggctgctgaggatcgaggagacaaagtgcatataattggcc | |
| | acattcccccagggcactgtctgaagagctggagctggaattattaccgaattgtagccaggtatgagaacaccctggctgc | |
| | tcagttctttggccacactcatgtggatgaatttgaggtcttctatgatgaagagactctgagccggccgctggctgtagcc | |
| | ttcctggcacccagtgcaactacctacatcggccttaatcctggttaccgtgtgtaccaaatagatggaaactactccggga | |
| | gctctcacgtggtcctggaccatgagacctacatcctgaatctgacccaggcaaacataccgggagccataccgcactgg | |
| | cagcttctctacagggctcgagaaacctatgggctgcccaacacactgcctaccgcctggcacaacctggtatatcgcatg | |
| | cggggcgacatgcaacttttccagaccttctggtttctctaccataagggccacccaccctcggagccctgtggcacgccc | |
| | tgccgtctggctactctttgtgcccagctctctgcccgtgctgacagccctgctctgtgccgccacctgatgccagatggga | |
| | gcctcccagaggcccagagcctgtggccaaggccactgttttgctagacccagctttcttgtacaaagtggctgtgccttct | |
| | agttgccagccatctgttgtttgcccctcccccgtgccttccttgaccctggaaggtgccactcccactgtcctttcctaat | |
| | aaaatgaggaaattgcatcgcattgtctgagtaggtgtcattctattctggggggtggggggggcaggacagcaagggggag | |
| | gattgggaagagaatagcaggcatgctggggactctattttataggcttcttctctggaatcttcttcatcatcctcctgac | |
| | aatcgataggtacctggctgtcgtccatgctgtgtttgctttaaaagccaggacggtcacctttggggtggtgacaagtgtg | |
| | atcacttgggtggtggctgtgtttgcgtctctcccaggaatcatctttaccagatctcaaaaagaaggtcttcattacacct | |
| | gcagctctcattttccatacagtcagtatcaattctggaagaatttccagacattaaagatagtcatcttggggctggtcct | |
| | gccgctgcttgtcatggtcatctgctactcgggaatcctaaaaactctgcttcggtgtcgaaatgagaagaagaggcacagg | |
| | gctgtgaggcttatcttcaccatcatgattgtttattttctcttctgggctccctacaacattgtccttctcctgaacacct | |
| | tccaggaattctttggcctgaataattgcagtagctctaacaggttggaccaagctatgcaggtgacagagactcttgggat | |
| | gacgcactgctgcatcaaccccatcatctatgcctttgtcggggagaagttcagaaaccaactttattatacatagttgatg | |
| | gccggccgcttcgagttaattaatccaaccggttaccgcctaggatcgatagatctaggaacccctagtgatggagttggcc | |
| | actccctctctgcgcgctcgctcgctcactgaggccgggcgaccaaaggtcgcccgacgcccgggctttgcccgggggcctc | |
| | agtgagcgagcgagcgcgcagctgcctgcaggcagcttggcactggccgtcgttttacaacgtcgtgactgggaaaaccctg | |
| | gcgttacccaacttaatcgccttgcagcacatccccctttcgccagctggcgtaatagcgaagaggcccgcaccgatcgccc | |
| | ttcccaacagttgcgcagcctgaatggcgaatggcgcctgatgcggtattttctccttacgcatctgtgcggtatttcacac | |
| | cgcatacgtcaaagcaaccatagtacgcgccctgtagcggcgcattaagcgcggcgggtgtggtggttacgcgcagcgtgac | |
| | cgctacacttgccagcgccctagcgcccgctcctttcgctttcttcccttcctttctcgccacgttcgccggctttccccgt | |
| | caagctctaaatcgggggctccctttagggttccgatttagtgctttacggcacctcgaccccaaaaaacttgatttgggtg | |
| | atggttcacgtagtgggccatcgccctgatagacggtttttcgccctttgacgttggagtccacgttctttaatagtggact | |
| | cttgttccaaactggaacaacactcaaccctatctcgggctattcttttgatttataagggattttgccgatttcggcctat | |
| | tggttaaaaaatgagctgatttaacaaaaatttaacgcgaattttaacaaaatattaacgtttacaattttatggtgcactc | |
| | tcagtacaatctgctctgatgccgcatagttaagccagccccgacacccgccaacacccgctgacgcgccctgacgggcttg | |
| | tctgctcccggcatccgcttacagacaagctgtgaccgtctccgggagctgcatgtgtcagaggttttcaccgtcatcaccg | |
| | aaacgcgcgagacgaaagggcctcgtgatacgcctatttttataggttaatgtcatgataataatggtttcttagacgtcag | |
| | gtggcacttttcggggaaatgtg | |
| p00690 SMPD1 | cgcggaacccctatttgtttatttttctaaatacattcaaatatgtatccgctcatgagacaataaccctgataaatgcttc | 23 |
| GeneArt | aataatattgaaaaaggaagagtATGAGCCATATTCAACGGGAAACGTCTTGCTCGAGGCCG | |
| Partial | CGATTAAATTCCAACATGGATGCTGATTTATATGGGTATAAATGGGCTCG | |
| Codon | CGATAATGTCGGGCAATCAGGTGCGACAATCTATCGATTGTATGGGAAGC | |
| Optimized | CCGATGCGCCAGAGTTGTTTCTGAAACATGGCAAAGGTAGCGTTGCCAAT | |
| | GATGTTACAGATGAGATGGTCAGACTAAACTGGCTGACGGAATTTATGCC | |
| | TCTTCCGACCATCAAGCATTTTATCCGTACTCCTGATGATGCATGGTTACT | |
| | CACCACTGCGATCCCCGGGAAAACAGCATTCCAGGTATTAGAAGAATATC | |
| | CTGATTCAGGTGAAAATATTGTTGATGCGCTGGCAGTGTTCCTGCGCCGG | |
| | TTGCATTCGATTCCTGTTTGTAATTGTCCTTTTAACAGCGATCGCGTATTTC | |
| | GTCTCGCTCAGGCGCAATCACGAATGAATAACGGTTTGGTTGATGCGAGT | |
| | GATTTTGATGACGAGCGTAATGGCTGGCCTGTTGAACAAGTCTGGAAAGA | |
| | AATGCATAAGCTTTTGCCATTCTCACCGGATTCAGTCGTCACTCATGGTGA | |
| | TTTCTCACTTGATAACCTTATTTTTGACGAGGGGAAATTAATAGGTTGTAT | |
| | TGATGTTGGACGAGTCGGAATCGCAGACCGATACCAGGATCTTGCCATCC | |
| | TATGGAACTGCCTCGGTGAGTTTTCTCCTTCATTACAGAAACGGCTTTTTC | |
| | AAAAATATGGTATTGATAATCCTGATATGAATAAATTGCAGTTTCATTTG | |
| | ATGCTCGATGAGTTTTTCTAActgtcagaccaagtttactcatatatactttagattgatttaaaacttcatttttaattt | |
| | aaaaggatctaggtgaagatcctttttgataatctcatgaccaaaatcccttaacgtgagttttcgttccactgagcgtca | |
| | gaccccgtagaaaagatcaaaggatcttcttgagatcctttttttctgcgcgtaatctgctgcttgcaaacaaaaaaacca | |
| | ccgctaccagcggtggtttgtttgccggatcaagagctaccaactctttttccgaaggtaactggcttcagcagagcgcaga | |
| | taccaaatactgttcttctagtgtagccgtagttaggccaccacttcaagaactctgtagcaccgcctacatacctcgctct | |
| | gctaatcctgttaccagtggctgctgccagtggcgataagtcgtgtcttaccgggttggactcaagacgatagttaccggat | |
| | aaggcgcagcggtcgggctgaacggggggttcgtgcacacagcccagcttggagcgaacgacctacaccgaactgagat | |
| | acctacagcgtgagctatgagaaagcgccacgcttcccgaagggagaaaggcggacaggtatccggtaagcggcagg | |
| | gtcggaacaggagagcgcacgagggagcttccagggggaaacgcctggtatctttatagtcctgtcgggtttcgccacct | |
| | ctgacttgagcgtcgatttttgtgatgctcgtcaggggggggagcctatggaaaaacgccagcaacgcggcctttttacg | |
| | gttcctggccttttgctggccttttgctcacatgttctttcctgcgttatcccctgattctgtggataaccgtattaccgcc | |
| | tttgagtgagctgataccgctcgccgcagccgaacgaccgagcgcagcgagtcagtgagcgaggaagcggaagagcgcccaa | |
| | tacgcaaaccgcctctccccgcgcgttggccgattcattaatgcagctggcacgacaggtttcccgactggaaagcgggcag | |
| | tgagcgcaacgcaattaatgtgagttagctcactcattaggcaccccaggctttacactttatgcttccggctcgtatgttg | |
| | tgtggaattgtgagcggataacaatttcacacaggaaacagctatgaccatgattacgaattgcctgcaggcagctgcgcg | |
| | ctcgctcgctcactgaggccgcccgggcaaagcccgggcgtcgggcgacctttggtcgcccggcctcagtgagcgagc | |
| | gagcgcgcagagagggagtggccaactccatcactaggggttcctatcgatggcgcgcctccagatatcatacactcgagca | |
| | actttgtatagaaaagttgggtgctatagagcacaagattttatttggtgagatggtgctttcatgaattcccccaacagag | |
| | ccaagctctccatctagtggacagggaagctagcagcaaaccttcccttcactacaaaacttcattgcttggccaaaaagag | |
| | agttaattcaatgtagacatctatgtaggcaattaaaaacctattgatgtataaaacagtttgcattcatggagggcaacta | |
| | aatacattctaggactttataaaagatcactttttatttatgcacagggtggaacaagatggattatcaagtgtcaagtcca | |
| | atctatgacatcaattattatacatcggagccctgccaaaaaatcaatgtgaagcaaatcgcagcccgcctcctgcctccgc | |
| | tctactcactggtgttcatctttggttttgtgggcaacatgctggtcatcctcatcctgataaactgcaaaaggctgaagag | |
| | catgactgacatctacctgctcaacctggccatctctgacctgtttttccttcttactgtccccttctgggctcactatgct | |
| | gccgcccagtgggactttggaaatacaatgtgtcaactcttgacagaacagttggaacagcagaatatgggccaaacaggat | |
| | atctgtggtaagcagttcctgccccggctcagggccaagaacagttggaacagcagaatatgggccaaacaggatatctgtg | |
| | gtaagcagttcctgccccggctcagggccaagaacagatggtccccagatgcggtcccgccctcagcagtttctagagaacc | |
| | atcagatgtttccagggtgccccaaggacctgaaatgaccctgtgccttatttgaactaaccaatcagttcgcttctcgctt | |
| | ctgttcgcgcgcttctgctccccgagctctatataagcagagctcgtttagtgaaccgtcagatccaagtttgtacaaaaaa | |
| | gcaggctgccaccATGCCCCGCTACGGAGCGTCACTCCGCCAGAGCTGCCCCAGGTCC | |
| | GGCAGGGAGCAGGGACAAGACGGGACCGCCGGAGCTCCTGGACTCCTTT | |
| | GGATGGGACTGGTGCTGGCGCTGGCGCTTGCTCTGGCGCTGGCCCTGGCA | |
| | CTGTCTGACTCTCGGGTTCTCTGGGCTCCGGCAGAAGCTCACCCTCTTTCT | |
| | CCTCAAGGCCATCCTGCCAGGTTACATCGCATAGTGCCCCGGCTCCGAGA | |
| | TGTCTTTGGGTGGGGGAACCTCACCTGCCCAATCTGCAAAGGTCTATTCA | |
| | CCGCCATCAACCTCGGGCTGAAGAAGGAACCCAATGTGGCTCGCGTGGG | |
| | CTCCGTGGCCATCAAGCTGTGCAATCTGCTGAAGATAGCACCACCTGCCG | |
| | TGTGCCAATCCATTGTCCACCTCTTTGAGGATGACATGGTGGAGGTGTGG | |
| | AGACGCTCAGTGCTGAGCCCATCTGAGGCCTGTGGCCTGCTCCTGGGCTC | |
| | CACCTGTGGGCACTGGGACATTTTCTCATCTTGGAACATCTCTTTGCCTAC | |
| | TGTGCCGAAGCCGCCCCCCAAACCCCCTAGCCCTCCAGCCCCAGGTGCTC | |
| | CTGTCAGCCGCATCCTCTTCCTCACTGACCTGCACTGGGATCATGACTACC | |
| | TGGAGGGCACGGACCCTGACTGTGCAGACCCACTGTGCTGCCGCCGGGGT | |
| | TCTGGCCTGCCGCCTGCATCCAGGCCAGGTGCCGGATACTGGGGCGAATA | |
| | CAGCAAGTGTGACCTGCCCCTGAGGACCCTGGAGAGCCTGTTGAGTGGGC | |
| | TGGGCCCAGCCGGACCTTTTGATATGGTGTACTGGACAGGAGACATCCCC | |
| | GCACATGATGTCTGGCACCAGACTCGTCAGGACCAACTGCGGGCCCTGAC | |
| | CACCGTCACAGCACTTGTGAGGAAGTTCCTGGGGCCAGTGCCAGTGTACC | |
| | CTGCTGTGGGTAACCATGAAAGCACACCTGTCAATAGCTTCCCTCCCCCC | |
| | TTCATTGAGGGCAACCACTCCTCCCGCTGGCTCTATGAAGCGATGGCCAA | |
| | GGCTTGGGAGCCCTGGCTGCCTGCCGAAGCCCTGCGCACCCTCAGAATTG | |
| | GGGGGTTCTATGCTCTTTCCCCATACCCCGGTCTCCGCCTCATCTCTCTCA | |
| | ATATGAATTTTTGTTCCCGTGAGAACTTCTGGCTCTTGATCAACTCCACGG | |
| | ATCCCGCAGGACAGCTCCAGTGGCTGGTGGGGGAGCTTCAGGCTGCTGAG | |
| | GATCGAGGAGACAAAGTGCATATAATTGGCCACATTCCCCCAGGGCACTG | |
| | TCTGAAGAGCTGGAGCTGGAATTATTACCGAATTGTAGCCAGGTATGAGA | |
| | ACACCCTGGCTGCTCAGTTCTTTGGCCACACTCATGTGGATGAATTTGAG | |
| | GTCTTCTATGATGAAGAGACTCTGAGCCGGCCGCTGGCTGTAGCCTTCCT | |
| | GGCACCCAGTGCAACTACCTACATCGGCCTTAATCCTGGTTACCGTGTGT | |
| | ACCAAATAGATGGAAACTACTCCGGGAGCTCTCACGTGGTCCTGGACCAT | |
| | GAGACCTACATCCTGAATCTGACCCAGGCAAACATACCGGGAGCCATAC | |
| | CGCACTGGCAGCTTCTCTACAGGGCTCGAGAAACCTATGGGCTGCCCAAC | |
| | ACACTGCCTACCGCCTGGCACAACCTGGTATATCGCATGCGGGGCGACAT | |
| | GCAACTTTTCCAGACCTTCTGGTTTCTCTACCATAAGGGCCACCCACCCTC | |
| | GGAGCCCTGTGGCACGCCCTGTCGTCTGGCTACTCTTTGTGCCCAGCTCTC | |
| | TGCCCGTGCTGACAGCCCTGCTCTGTGCCGCCACCTGATGCCAGATGGGA | |
| | GCCTCCCAGAGGCCCAGAGCCTGTGGCCAAGGCCACTGTTTTGCTAGacccagctttcttgta | |
| | caaagtggctgtgccttctagttgccagccatctgttgtttgcccctcccccgtgccttccttgaccctggaaggtgccact | |
| | cccactgtcctttcctaataaaatgaggaaattgcatcgcattgtctgagtaggtgtcattctattctggggggtggggtg | |
| | gggcaggacagcaagggggaggattgggaagagaatagcaggcatgctggggactctattttataggcttcttctctggaa | |
| | tcttcttcatcatcctcctgacaatcgataggtacctggctgtcgtccatgctgtgtttgctttaaaagccaggacggtcac | |
| | ctttggggtggtgacaagtgtgatcacttgggtggtggctgtgtttgcgtctctcccaggaatcatctttaccagatctca | |
| | aaaagaaggtcttcattacacctgcagctctcattttccatacagtcagtatcaattctggaagaatttccagacattaaag | |
| | atagtcatcttggggctggtcctgccgctgcttgtcatggtcatctgctactcgggaatcctaaaaactctgcttcggtgtc | |
| | gaaatgagaagaagaggcacagggctgtgaggcttatcttcaccatcatgattgtttattttctcttctgggctccctacaa | |
| | cattgtccttctcctgaacaccttccaggaattctttggcctgaataattgcagtagctctaacaggttggaccaagctatg | |
| | caggtgacagagactcttgggatgacgcactgctgcatcaaccccatcatctatgcctttgtcggggagaagttcagaaacc | |
| | aactttattatacatagttgatggccggccgcttcgagttaattaatccaaccggttaccgcctaggatcgatagatctagg | |
| | aacccctagtgatggagttggccactccctctctgcgcgctcgctcgctcactgaggccgggcgaccaaaggtcgcccgacg | |
| | cccgggctttgcccgggcggcctcagtgagcgagcgagcgcgcagctgcctgcaggcagcttggcactggccgtcgttttac | |
| | aacgtcgtgactgggaaaaccctggcgttacccaacttaatcgccttgcagcacatccccctttcgccagctggcgtaatag | |
| | cgaagaggcccgcaccgatcgcccttcccaacagttgcgcagcctgaatggcgaatggcgcctgatgcggtattttctcctt | |
| | acgcatctgtgcggtatttcacaccgcatacgtcaaagcaaccatagtacgcgccctgtagcggcgcattaagcgcggcggg | |
| | tgtggtggttacgcgcagcgtgaccgctacacttgccagcgccctagcgcccgctcctttcgctttcttcccttcctttctc | |
| | gccacgttcgccggctttccccgtcaagctctaaatcgggggctccctttagggttccgatttagtgctttacggcacctcg | |
| | accccaaaaaacttgatttgggtgatggttcacgtagtgggccatcgccctgatagacggtttttcgccctttgacgttgga | |
| | gtccacgttctttaatagtggactcttgttccaaactggaacaacactcaaccctatctcgggctattcttttgatttataa | |
| | gggattttgccgatttcggcctattggttaaaaaatgagctgatttaacaaaaatttaacgcgaattttaacaaaatattaa | |
| | cgtttacaattttatggtgcactctcagtacaatctgctctgatgccgcatagttaagccagccccgacacccgccaacacc | |
| | cgctgacgcgccctgacgggcttgtctgctcccggcatccgcttacagacaagctgtgaccgtctccgggagctgcatgtgt | |
| | cagaggttttcaccgtcatcaccgaaacgcgcgagacgaaagggcctcgtgatacgcctatttttataggttaatgtcatga | |
| | taataatggtttcttagacgtcaggtggcacttttcggggaaatgtg | |
| p00691_SMPD1 | cgcggaacccctatttgtttatttttctaaatacattcaaatatgtatccgctcatgagacaataaccctgataaatgcttc | 24 |
| C629S GeneArt | aataatattgaaaaaggaagagtATGAGCCATATTCAACGGGAAACGTCTTGCTCGAGGCCG | |
| Partial Codon | CGATTAAATTCCAACATGGATGCTGATTTATATGGGTATAAATGGGCTCG | |
| Optimized | CGATAATGTCGGGCAATCAGGTGCGACAATCTATCGATTGTATGGGAAGC | |
| | CCGATGCGCCAGAGTTGTTTCTGAAACATGGCAAAGGTAGCGTTGCCAAT | |
| | GATGTTACAGATGAGATGGTCAGACTAAACTGGCTGACGGAATTTATGCC | |
| | TCTTCCGACCATCAAGCATTTTATCCGTACTCCTGATGATGCATGGTTACT | |
| | CACCACTGCGATCCCCGGGAAAACAGCATTCCAGGTATTAGAAGAATATC | |
| | CTGATTCAGGTGAAAATATTGTTGATGCGCTGGCAGTGTTCCTGCGCCGG | |
| | TTGCATTCGATTCCTGTTTGTAATTGTCCTTTTAACAGCGATCGCGTATTTC | |
| | GTCTCGCTCAGGCGCAATCACGAATGAATAACGGTTTGGTTGATGCGAGT | |
| | GATTTTGATGACGAGCGTAATGGCTGGCCTGTTGAACAAGTCTGGAAAGA | |
| | AATGCATAAGCTTTTGCCATTCTCACCGGATTCAGTCGTCACTCATGGTGA | |
| | TTTCTCACTTGATAACCTTATTTTTGACGAGGGGAAATTAATAGGTTGTAT | |
| | TGATGTTGGACGAGTCGGAATCGCAGACCGATACCAGGATCTTGCCATCC | |
| | TATGGAACTGCCTCGGTGAGTTTTCTCCTTCATTACAGAAACGGCTTTTTC | |
| | AAAAATATGGTATTGATAATCCTGATATGAATAAATTGCAGTTTCATTTG | |
| | ATGCTCGATGAGTTTTTCTAActgtcagaccaagtttactcatatatactttagattgatttaaaacttcatttttaatt | |
| | taaaaggatctaggtgaagatcctttttgataatctcatgaccaaaatcccttaacgtgagttttcgttccactgagcgtc | |
| | agaccccgtagaaaagatcaaaggatcttcttgagatcctttttttctgcgcgtaatctgctgcttgcaaacaaaaaaacca | |
| | ccgctaccagcggtggtttgtttgccggatcaagagctaccaactctttttccgaaggtaactggcttcagcagagcgcaga | |
| | taccaaatactgttcttctagtgtagccgtagttaggccaccacttcaagaactctgtagcaccgcctacatacctcgctct | |
| | gctaatcctgttaccagtggctgctgccagtggcgataagtcgtgtcttaccgggttggactcaagacgatagttaccgga | |
| | taaggcgcagcggtcgggctgaacggggggttcgtgcacacagcccagcttggagcgaacgacctacaccgaactgagat | |
| | acctacagcgtgagctatgagaaagcgccacgcttcccgaagggagaaaggcggacaggtatccggtaagcggcagg | |
| | gtcggaacaggagagcgcacgagggagcttccagggggaaacgcctggtatctttatagtcctgtcgggtttcgccacct | |
| | ctgacttgagcgtcgatttttgtgatgctcgtcaggggggcggagcctatggaaaaacgccagcaacgcggcctttttacg | |
| | gttcctggccttttgctggccttttgctcacatgttctttcctgcgttatcccctgattctgtggataaccgtattaccgcc | |
| | tttgagtgagctgataccgctcgccgcagccgaacgaccgagcgcagcgagtcagtgagcgaggaagcggaagagcgcccaa | |
| | tacgcaaaccgcctctccccgcgcgttggccgattcattaatgcagctggcacgacaggtttcccgactggaaagcgggcag | |
| | tgagcgcaacgcaattaatgtgagttagctcactcattaggcaccccaggctttacactttatgcttccggctcgtatgttg | |
| | tgtggaattgtgagcggataacaatttcacacaggaaacagctatgaccatgattacgaattgcctgcaggcagctgcgcg | |
| | ctcgctcgctcactgaggccgcccgggcaaagcccgggcgtcgggcgacctttggtcgcccggcctcagtgagcgagc | |
| | gagcgcgcagagagggagtggccaactccatcactaggggttcctatcgatggcgcgcctccagatatcatacactcgagc | |
| | aactttgtatagaaaagttgggtgctatagagcacaagattttatttggtgagatggtgctttcatgaattcccccaacaga | |
| | gccaagctctccatctagtggacagggaagctagcagcaaaccttcccttcactacaaaacttcattgcttggccaaaaag | |
| | agagttaattcaatgtagacatctatgtaggcaattaaaaacctattgatgtataaaacagtttgcattcatggagggcaac | |
| | taaatacattctaggactttataaaagatcactttttatttatgcacagggtggaacaagatggattatcaagtgtcaagtc | |
| | caatctatgacatcaattattatacatcggagccctgccaaaaaatcaatgtgaagcaaatcgcagcccgcctcctgcctcc | |
| | gctctactcactggtgttcatctttggttttgtgggcaacatgctggtcatcctcatcctgataaactgcaaaaggctgaag | |
| | agcatgactgacatctacctgctcaacctggccatctctgacctgtttttccttcttactgtccccttctgggctcactatg | |
| | ctgccgcccagtgggactttggaaatacaatgtgtcaactcttgacagaacagttggaacagcagaatatgggccaaacagg | |
| | atatctgtggtaagcagttcctgccccggctcagggccaagaacagttggaacagcagaatatgggccaaacaggatatctg | |
| | tggtaagcagttcctgccccggctcagggccaagaacagatggtccccagatgcggtcccgccctcagcagtttctagagaa | |
| | ccatcagatgtttccagggtgccccaaggacctgaaatgaccctgtgccttatttgaactaaccaatcagttcgcttctcgc | |
| | ttctgttcgcgcgcttctgctccccgagctctatataagcagagctcgtttagtgaaccgtcagatccaagtttgtacaaaa | |
| | aagcaggctgccaccATGCCCCGCTACGGAGCGTCACTCCGCCAGAGCTGCCCCAGGTCC | |
| | GGCAGGGAGCAGGGACAAGACGGGACCGCCGGAGCTCCTGGACTCCTTT | |
| | GGATGGGACTGGTGCTGGCGCTGGCGCTTGCTCTGGCGCTGGCCCTGGCA | |
| | CTGTCTGACTCTCGGGTTCTCTGGGCTCCGGCAGAAGCTCACCCTCTTTCT | |
| | CCTCAAGGCCATCCTGCCAGGTTACATCGCATAGTGCCCCGGCTCCGAGA | |
| | TGTCTTTGGGTGGGGGAACCTCACCTGCCCAATCTGCAAAGGTCTATTCA | |
| | CCGCCATCAACCTCGGGCTGAAGAAGGAACCCAATGTGGCTCGCGTGGG | |
| | CTCCGTGGCCATCAAGCTGTGCAATCTGCTGAAGATAGCACCACCTGCCG | |
| | TGTGCCAATCCATTGTCCACCTCTTTGAGGATGACATGGTGGAGGTGTGG | |
| | AGACGCTCAGTGCTGAGCCCATCTGAGGCCTGTGGCCTGCTCCTGGGCTC | |
| | CACCTGTGGGCACTGGGACATTTTCTCATCTTGGAACATCTCTTTGCCTAC | |
| | TGTGCCGAAGCCGCCCCCCAAACCCCCTAGCCCTCCAGCCCCAGGTGCTC | |
| | CTGTCAGCCGCATCCTCTTCCTCACTGACCTGCACTGGGATCATGACTACC | |
| | TGGAGGGCACGGACCCTGACTGTGCAGACCCACTGTGCTGCCGCCGGGGT | |
| | TCTGGCCTGCCGCCTGCATCCAGGCCAGGTGCCGGATACTGGGGCGAATA | |
| | CAGCAAGTGTGACCTGCCCCTGAGGACCCTGGAGAGCCTGTTGAGTGGGC | |
| | TGGGCCCAGCCGGACCTTTTGATATGGTGTACTGGACAGGAGACATCCCC | |
| | GCACATGATGTCTGGCACCAGACTCGTCAGGACCAACTGCGGGCCCTGAC | |
| | CACCGTCACAGCACTTGTGAGGAAGTTCCTGGGGCCAGTGCCAGTGTACC | |
| | CTGCTGTGGGTAACCATGAAAGCACACCTGTCAATAGCTTCCCTCCCCCC | |
| | TTCATTGAGGGCAACCACTCCTCCCGCTGGCTCTATGAAGCGATGGCCAA | |
| | GGCTTGGGAGCCCTGGCTGCCTGCCGAAGCCCTGCGCACCCTCAGAATTG | |
| | GGGGGTTCTATGCTCTTTCCCCATACCCCGGTCTCCGCCTCATCTCTCTCA | |
| | ATATGAATTTTTGTTCCCGTGAGAACTTCTGGCTCTTGATCAACTCCACGG | |
| | ATCCCGCAGGACAGCTCCAGTGGCTGGTGGGGGAGCTTCAGGCTGCTGAG | |
| | GATCGAGGAGACAAAGTGCATATAATTGGCCACATTCCCCCAGGGCACTG | |
| | TCTGAAGAGCTGGAGCTGGAATTATTACCGAATTGTAGCCAGGTATGAGA | |
| | ACACCCTGGCTGCTCAGTTCTTTGGCCACACTCATGTGGATGAATTTGAG | |
| | GTCTTCTATGATGAAGAGACTCTGAGCCGGCCGCTGGCTGTAGCCTTCCT | |
| | GGCACCCAGTGCAACTACCTACATCGGCCTTAATCCTGGTTACCGTGTGT | |
| | ACCAAATAGATGGAAACTACTCCGGGAGCTCTCACGTGGTCCTGGACCAT | |
| | GAGACCTACATCCTGAATCTGACCCAGGCAAACATACCGGGAGCCATAC | |
| | CGCACTGGCAGCTTCTCTACAGGGCTCGAGAAACCTATGGGCTGCCCAAC | |
| | ACACTGCCTACCGCCTGGCACAACCTGGTATATCGCATGCGGGGCGACAT | |
| | GCAACTTTTCCAGACCTTCTGGTTTCTCTACCATAAGGGCCACCCACCCTC | |
| | GGAGCCCTGTGGCACGCCCTGTCGTCTGGCTACTCTTTGTGCCCAGCTCTC | |
| | TGCCCGTGCTGACAGCCCTGCTCTGTGCCGCCACCTGATGCCAGATGGGA | |
| | GCCTCCCAGAGGCCCAGAGCCTGTGGCCAAGGCCACTGTTTTCCTAGacccagctttcttgtacaaagtgg | |
| | ctgtgccttctagttgccagccatctgttgtttgcccctcccccgtgccttccttgaccctggaaggtgccactcc | |
| | cactgtcctttcctaataaaatgaggaaattgcatcgcattgtctgagtaggtgtcattctattctggggggtg | |
| | gggtggggcaggacagcaagggggaggattgggaagagaatagcaggcatgctggggactctattttataggcttcttct | |
| | ctggaatcttcttcatcatcctcctgacaatcgataggtacctggctgtcgtccatgctgtgtttgctttaaaagccaggac | |
| | ggtcacctttggggtggtgacaagtgtgatcacttgggtggtggctgtgtttgcgtctctcccaggaatcatctttaccaga | |
| | tctcaaaaagaaggtcttcattacacctgcagctctcattttccatacagtcagtatcaattctggaagaatttccagacat | |
| | taaagatagtcatcttggggctggtcctgccgctgcttgtcatggtcatctgctactcgggaatcctaaaaactctgcttcg | |
| | gagaagaagaggcacagggctgtgaggcttatcttcaccatcatgattgtttattttctcttctgggctccctacaacattg | |
| | gtgtcgaaattccttctcctgaacaccttccaggaattctttggcctgaataattgcagtagctctaacaggttggaccaag | |
| | ctatgcaggtgacagagactcttgggatgacgcactgctgcatcaaccccatcatctatgcctttgtcggggagaagttcag | |
| | aaaccaactttattatacatagttgatggccggccgcttcgagttaattaatccaaccggttaccgcctaggatcgatagat | |
| | ctaggaacccctagtgatggagttggccactccctctctgcgcgctcgctcgctcactgaggccgggcgaccaaaggtcgcc | |
| | cgacgcccgggctttgcccgggcggcctcagtgagcgagcgagcgcgcagctgcctgcaggcagcttggcactggccgtcgt | |
| | tttacaacgtcgtgactgggaaaaccctggcgttacccaacttaatcgccttgcagcacatccccctttcgccagctggcgt | |
| | aatagcgaagaggcccgcaccgatcgcccttcccaacagttgcgcagcctgaatggcgaatggcgcctgatgcggtattttc | |
| | tccttacgcatctgtgcggtatttcacaccgcatacgtcaaagcaaccatagtacgcgccctgtagcggcgcattaagcgcg | |
| | gcgggtgtggtggttacgcgcagcgtgaccgctacacttgccagcgccctagcgcccgctcctttcgctttcttcccttcct | |
| | ttctcgccacgttcgccggctttccccgtcaagctctaaatcgggggctccctttagggttccgatttagtgctttacggca | |
| | cctcgaccccaaaaaacttgatttgggtgatggttcacgtagtgggccatcgccctgatagacggtttttcgccctttgacg | |
| | ttggagtccacgttctttaatagtggactcttgttccaaactggaacaacactcaaccctatctcgggctattcttttgatt | |
| | tataagggattttgccgatttcggcctattggttaaaaaatgagctgatttaacaaaaatttaacgcgaattttaacaaaat | |
| | attaacgtttacaattttatggtgcactctcagtacaatctgctctgatgccgcatagttaagccagccccgacacccgcca | |
| | acacccgctgacgcgccctgacgggcttgtctgctcccggcatccgcttacagacaagctgtgaccgtctccgggagctgca | |
| | tgtgtcagaggttttcaccgtcatcaccgaaacgcgcgagacgaaagggcctcgtgatacgcctatttttataggttaatgt | |
| | catgataataatggtttcttagacgtcaggtggcacttttcggggaaatgtg | |
| p00695 SMPD1 | cgcggaacccctatttgtttatttttctaaatacattcaaatatgtatccgctcatgagacaataaccctgataaatgcttc | 25 |
| GeneArt Full | aataatattgaaaaaggaagagtATGAGCCATATTCAACGGGAAACGTCTTGCTCGAGGCCG | |
| Codon | CGATTAAATTCCAACATGGATGCTGATTTATATGGGTATAAATGGGCTCG | |
| Optimized | CGATAATGTCGGGCAATCAGGTGCGACAATCTATCGATTGTATGGGAAGC | |
| | CCGATGCGCCAGAGTTGTTTCTGAAACATGGCAAAGGTAGCGTTGCCAAT | |
| | GATGTTACAGATGAGATGGTCAGACTAAACTGGCTGACGGAATTTATGCC | |
| | TCTTCCGACCATCAAGCATTTTATCCGTACTCCTGATGATGCATGGTTACT | |
| | CACCACTGCGATCCCCGGGAAAACAGCATTCCAGGTATTAGAAGAATATC | |
| | CTGATTCAGGTGAAAATATTGTTGATGCGCTGGCAGTGTTCCTGCGCCGG | |
| | TTGCATTCGATTCCTGTTTGTAATTGTCCTTTTAACAGCGATCGCGTATTTC | |
| | GTCTCGCTCAGGCGCAATCACGAATGAATAACGGTTTGGTTGATGCGAGT | |
| | GATTTTGATGACGAGCGTAATGGCTGGCCTGTTGAACAAGTCTGGAAAGA | |
| | AATGCATAAGCTTTTGCCATTCTCACCGGATTCAGTCGTCACTCATGGTGA | |
| | TTTCTCACTTGATAACCTTATTTTTGACGAGGGGAAATTAATAGGTTGTAT | |
| | TGATGTTGGACGAGTCGGAATCGCAGACCGATACCAGGATCTTGCCATCC | |
| | TATGGAACTGCCTCGGTGAGTTTTCTCCTTCATTACAGAAACGGCTTTTTC | |
| | AAAAATATGGTATTGATAATCCTGATATGAATAAATTGCAGTTTCATTTG | |
| | ATGCTCGATGAGTTTTTCTAActgtcagaccaagtttactcatatatactttagattgatttaaaacttcatttttaattta | |
| | aaaggatctaggtgaagatcctttttgataatctcatgaccaaaatcccttaacgtgagttttcgttccactgagcgtcaga | |
| | ccccgtagaaaagatcaaaggatcttcttgagatcctttttttctgcgcgtaatctgctgcttgcaaacaaaaaaaccaccg | |
| | ctaccagcggtggtttgtttgccggatcaagagctaccaactctttttccgaaggtaactggcttcagcagagcgcagatac | |
| | caaatactgttcttctagtgtagccgtagttaggccaccacttcaagaactctgtagcaccgcctacatacctcgctctgct | |
| | aatcctgttaccagtggctgctgccagtggcgataagtcgtgtcttaccgggttggactcaagacgatagttaccggataag | |
| | gcgcagcggtcgggctgaacggggggttcgtgcacacagcccagcttggagcgaacgacctacaccgaactgagatacctac | |
| | agcgtgagctatgagaaagcgccacgcttcccgaagggagaaaggcggacaggtatccggtaagcggcagggtcggaacagg | |
| | agagcgcacgagggagcttccagggggaaacgcctggtatctttatagtcctgtcgggtttcgccacctctgacttgagcgt | |
| | cgatttttgtgatgctcgtcaggggggcggagcctatggaaaaacgccagcaacgcggcctttttacggttcctggcctttt | |
| | gctggccttttgctcacatgttctttcctgcgttatcccctgattctgtggataaccgtattaccgcctttgagtgagctga | |
| | taccgctcgccgcagccgaacgaccgagcgcagcgagtcagtgagcgaggaagcggaagagcgcccaatacgcaaaccgcct | |
| | ctccccgcgcgttggccgattcattaatgcagctggcacgacaggtttcccgactggaaagcgggcagtgagcgcaacgcaa | |
| | ttaatgtgagttagctcactcattaggcaccccaggctttacactttatgcttccggctcgtatgttgtgtggaattgtgag | |
| | cggataacaatttcacacaggaaacagctatgaccatgattacgaattgcctgcaggcagctgcgcgctcgctcgctcactg | |
| | aggccgcccgggcaaagcccgggcgtcggggacctttggtcgcccggcctcagtgagcgagcgagcgcgcagagagggagtg | |
| | gccaactccatcactaggggttcctatcgatggcgcgcctccagatatcatacactcgagcaactttgtatagaaaagttgg | |
| | gtgctatagagcacaagattttatttggtgagatggtgctttcatgaattcccccaacagagccaagctctccatctagtgg | |
| | acagggaagctagcagcaaaccttcccttcactacaaaacttcattgcttggccaaaaagagagttaattcaatgtagacat | |
| | ctatgtaggcaattaaaaacctattgatgtataaaacagtttgcattcatggagggcaactaaatacattctaggactttat | |
| | aaaagatcactttttatttatgcacagggtggaacaagatggattatcaagtgtcaagtccaatctatgacatcaattatta | |
| | tacatcggagccctgccaaaaaatcaatgtgaagcaaatcgcagcccgcctcctgcctccgctctactcactggtgttcatc | |
| | tttggttttgtgggcaacatgctggtcatcctcatcctgataaactgcaaaaggctgaagagcatgactgacatctacctgc | |
| | tcaacctggccatctctgacctgtttttccttcttactgtccccttctgggctcactatgctgccgcccagtgggactttgg | |
| | aaatacaatgtgtcaactcttgacagaacagttggaacagcagaatatgggccaaacaggatatctgtggtaagcagttcct | |
| | gccccggctcagggccaagaacagttggaacagcagaatatgggccaaacaggatatctgtggtaagcagttcctgccccgg | |
| | ctcagggccaagaacagatggtccccagatgcggtcccgccctcagcagtttctagagaaccatcagatgtttccagggtgc | |
| | cccaaggacctgaaatgaccctgtgccttatttgaactaaccaatcagttcgcttctcgcttctgttcgcgcgcttctgctc | |
| | cccgagctctatataagcagagctcgtttagtgaaccgtcagatccaagtttgtacaaaaaagcaggc | |
| | tgccaccATGCCTAGATACGGCGCCAGCCTGAGACAGAGCTGTCCTAGATCT | |
| | GGCAGAGAGCAGGGCCAAGACGGAACAGCTGGTGCTCCTGGACTGCTGT | |
| | GGATGGGACTTGTTCTGGCTCTGGCCCTGGCTCTTGCATTGGCACTGGCTC | |
| | TGAGCGACAGCAGAGTTCTGTGGGCTCCTGCCGAAGCTCATCCTCTGAGT | |
| | CCTCAGGGACATCCCGCCAGACTGCACAGAATCGTGCCCAGACTGAGAG | |
| | ATGTGTTCGGCTGGGGCAACCTGACATGCCCTATCTGCAAGGGCCTGTTC | |
| | ACCGCCATCAACCTGGGCCTGAAGAAAGAACCCAACGTGGCCAGAGTGG | |
| | GCAGCGTGGCCATCAAGCTGTGCAACCTGCTGAAGATTGCCCCTCCTGCC | |
| | GTGTGCCAGTCTATCGTGCACCTGTTCGAGGACGACATGGTGGAAGTGTG | |
| | GCGGAGAAGCGTGCTGTCTCCATCTGAAGCTTGTGGCCTGCTGCTGGGCT | |
| | CTACATGTGGCCACTGGGACATCTTTAGCAGCTGGAACATCAGCCTGCCT | |
| | ACCGTGCCTAAGCCTCCTCCAAAGCCTCCATCTCCTCCAGCACCTGGCGC | |
| | TCCAGTGTCCAGAATCCTGTTCCTGACCGACCTGCACTGGGACCACGATT | |
| | ACCTGGAAGGCACCGATCCTGACTGCGCCGATCCTCTGTGTTGCAGAAGA | |
| | GGCTCTGGACTGCCTCCTGCCTCTAGACCAGGTGCCGGATATTGGGGCGA | |
| | GTACAGCAAGTGCGACCTGCCTCTGAGAACCCTGGAAAGCCTGCTGTCTG | |
| | GACTGGGACCTGCCGGACCTTTCGATATGGTGTACTGGACCGGCGACATC | |
| | CCCGCTCACGATGTGTGGCATCAGACCAGACAGGACCAGCTGAGAGCCC | |
| | TGACAACAGTGACAGCCCTCGTGCGGAAGTTTCTGGGACCAGTGCCTGTG | |
| | TATCCCGCCGTGGGAAATCACGAGAGCACCCCTGTGAACAGCTTCCCTCC | |
| | ACCTTTCATCGAGGGCAACCACAGCAGCAGATGGCTGTACGAAGCCATG | |
| | GCCAAGGCCTGGGAACCTTGGCTTCCAGCTGAAGCACTGCGGACCCTGAG | |
| | AATCGGCGGCTTTTATGCTCTGAGCCCCTATCCTGGCCTGAGACTGATCA | |
| | GCCTGAACATGAATTTCTGCAGCCGCGAGAACTTCTGGCTGCTGATCAAC | |
| | TCCACAGATCCTGCCGGCCAGCTGCAGTGGCTTGTTGGAGAATTGCAGGC | |
| | CGCCGAGGACAGAGGCGATAAGGTGCACATCATCGGACACATCCCTCCA | |
| | GGCCACTGCCTGAAGTCTTGGAGCTGGAACTACTACCGGATTGTGGCCAG | |
| | ATACGAGAACACCCTGGCCGCTCAGTTCTTCGGCCACACACACGTGGACG | |
| | AGTTCGAGGTGTTCTACGACGAGGAAACCCTGAGCAGACCTCTGGCCGTG | |
| | GCATTTCTGGCCCCAAGCGCCACCACATATATCGGACTGAACCCCGGCTA | |
| | CCGGGTGTACCAGATCGACGGCAATTACAGCGGCAGCTCCCACGTGGTGC | |
| | TGGACCACGAGACTTACATCCTGAATCTGACCCAGGCCAACATTCCCGGC | |
| | GCTATCCCTCATTGGCAGCTGCTGTATCGGGCCAGAGAGACATACGGCCT | |
| | GCCTAACACACTGCCAACCGCCTGGCACAACCTGGTGTACAGAATGAGA | |
| | GGCGACATGCAGCTGTTTCAGACCTTCTGGTTCCTGTACCACAAGGGACA | |
| | CCCTCCAAGCGAGCCTTGTGGCACCCCTTGTAGACTGGCCACTCTGTGTG | |
| | CTCAGCTGTCCGCCAGAGCTGATAGCCCCGCTCTGTGCAGACACCTGATG | |
| | CCTGATGGAAGCCTGCCTGAGGCTCAGTCCCTTTGGCCTAGACCTCTGTTC | |
| | TGCTGAacccagctttcttgtacaaagtggctgtgccttctagttgccagccatctgttgtttgcccctcccccgtgcctt | |
| | ccttgaccctggaaggtgccactcccactgtcctttcctaataaaatgaggaaattgcatcgcattgtctgagtaggtgtca | |
| | ttctattctggggggtggggggggcaggacagcaagggggaggattgggaagagaatagcaggcatgctggggactctattt | |
| | tataggcttcttctctggaatcttcttcatcatcctcctgacaatcgataggtacctggctgtcgtccatgctgtgtttgct | |
| | ttaaaagccaggacggtcacctttggggtggtgacaagtgtgatcacttgggtggtggctgtgtttgcgtctctcccaggaa | |
| | tcatctttaccagatctcaaaaagaaggtcttcattacacctgcagctctcattttccatacagtcagtatcaattctggaa | |
| | gaatttccagacattaaagatagtcatcttggggctggtcctgccgctgcttgtcatggtcatctgctactcgggaatccta | |
| | aaaactctgcttcggtgtcgaaatgagaagaagaggcacagggctgtgaggcttatcttcaccatcatgattgtttattttc | |
| | tcttctgggctccctacaacattgtccttctcctgaacaccttccaggaattctttggcctgaataattgcagtagctctaa | |
| | caggttggaccaagctatgcaggtgacagagactcttgggatgacgcactgctgcatcaaccccatcatctatgcctttgtc | |
| | ggggagaagttcagaaaccaactttattatacatagttgatggccggccgcttcgagttaattaatccaaccggttaccgcc | |
| | taggatcgatagatctaggaacccctagtgatggagttggccactccctctctgcgcgctcgctcgctcactgagg | |
| | ccgggcgaccaaaggtcgcccgacgcccgggctttgcccgggcggcctcagtgagcgagcgagcgcgcagctgc | |
| | ctgcaggcagcttggcactggccgtcgttttacaacgtcgtgactgggaaaaccctggcgttacccaacttaatcgcctt | |
| | gcagcacatccccctttcgccagctggcgtaatagcgaagaggcccgcaccgatcgcccttcccaacagtt | |
| | gcgcagcctgaatggcgaatggcgcctgatgcggtattttctccttacgcatctgtgcggtatttcacac | |
| | cgcatacgtcaaagcaaccatagtacgcgccctgtagcggcgcattaagcgcggcgggtgtggtggttacgcgca | |
| | gcgtgaccgctacacttgccagcgccctagcgcccgctcctttcgctttcttcccttcctttctcgccacgttcgccg | |
| | gctttccccgtcaagctctaaatcgggggctccctttagggttccgatttagtgctttacggcacctcgaccccaaaaaa | |
| | cttgatttgggtgatggttcacgtagtgggccatcgccctgatagacggtttttcgccctttgacgttggagtcca | |
| | cgttctttaatagtggactcttgttccaaactggaacaacactcaaccctatctcgggctattcttttgatttataaggg | |
| | attttgccgatttcggcctattggttaaaaaatgagctgatttaacaaaaatttaacgcgaattttaacaaaatattaa | |
| | cgtttacaattttatggtgcactctcagtacaatctgctctgatgccgcatagttaagccagccccgacacccgccaacac | |
| | ccgctgacgcgccctgacgggcttgtctgctcccggcatccgcttacagacaagctgtgaccgtctccgggagctgcatgtg | |
| | tcagaggttttcaccgtcatcaccgaaacgcgcgagacgaaagggcctcgtgatacgcctatttttataggttaatgtcat | |
| | gataataatggtttcttagacgtcaggtggcacttttcggggaaatgtg | |
| p00696_SMPD1 | cgcggaacccctatttgtttatttttctaaatacattcaaatatgtatccgctcatgagacaataaccctgataaatgcttc | 26 |
| C629S GeneArt | aataatattgaaaaaggaagagtATGAGCCATATTCAACGGGAAACGTCTTGCTCGAGGCCG | |
| Full Codon | CGATTAAATTCCAACATGGATGCTGATTTATATGGGTATAAATGGGCTCG | |
| Optimized | CGATAATGTCGGGCAATCAGGTGCGACAATCTATCGATTGTATGGGAAGC | |
| | CCGATGCGCCAGAGTTGTTTCTGAAACATGGCAAAGGTAGCGTTGCCAAT | |
| | GATGTTACAGATGAGATGGTCAGACTAAACTGGCTGACGGAATTTATGCC | |
| | TCTTCCGACCATCAAGCATTTTATCCGTACTCCTGATGATGCATGGTTACT | |
| | CACCACTGCGATCCCCGGGAAAACAGCATTCCAGGTATTAGAAGAATATC | |
| | CTGATTCAGGTGAAAATATTGTTGATGCGCTGGCAGTGTTCCTGCGCCGG | |
| | TTGCATTCGATTCCTGTTTGTAATTGTCCTTTTAACAGCGATCGCGTATTTC | |
| | GTCTCGCTCAGGCGCAATCACGAATGAATAACGGTTTGGTTGATGCGAGT | |
| | GATTTTGATGACGAGCGTAATGGCTGGCCTGTTGAACAAGTCTGGAAAGA | |
| | AATGCATAAGCTTTTGCCATTCTCACCGGATTCAGTCGTCACTCATGGTGA | |
| | TTTCTCACTTGATAACCTTATTTTTGACGAGGGGAAATTAATAGGTTGTAT | |
| | TGATGTTGGACGAGTCGGAATCGCAGACCGATACCAGGATCTTGCCATCC | |
| | TATGGAACTGCCTCGGTGAGTTTTCTCCTTCATTACAGAAACGGCTTTTTC | |
| | AAAAATATGGTATTGATAATCCTGATATGAATAAATTGCAGTTTCATTTG | |
| | ATGCTCGATGAGTTTTTCTAActgtcagaccaagtttactcatatatactttagattgatttaaaacttcatttttaatt | |
| | taaaaggatctaggtgaagatcctttttgataatctcatgaccaaaatcccttaacgtgagttttcgttccactgagcgtc | |
| | agaccccgtagaaaagatcaaaggatcttcttgagatcctttttttctgcgcgtaatctgctgcttgcaaacaaaaaaacca | |
| | ccgctaccagcggtggtttgtttgccggatcaagagctaccaactctttttccgaaggtaactggcttcagcagagcgcaga | |
| | taccaaatactgttcttctagtgtagccgtagttaggccaccacttcaagaactctgtagcaccgcctacatacctcgctct | |
| | gctaatcctgttaccagtggctgctgccagtggcgataagtcgtgtcttaccgggttggactcaagacgatagttaccggat | |
| | aaggcgcagcggtcgggctgaacggggggttcgtgcacacagcccagcttggagcgaacgacctacaccgaactgagat | |
| | acctacagcgtgagctatgagaaagcgccacgcttcccgaagggagaaaggcggacaggtatccggtaagcggcagg | |
| | gtcggaacaggagagcgcacgagggagcttccagggggaaacgcctggtatctttatagtcctgtcgggtttcgccacct | |
| | ctgacttgagcgtcgatttttgtgatgctcgtcaggggggggagcctatggaaaaacgccagcaacgcggcctttttacg | |
| | gttcctggccttttgctggccttttgctcacatgttctttcctgcgttatcccctgattctgtggataaccgtattaccgcc | |
| | tttgagtgagctgataccgctcgccgcagccgaacgaccgagcgcagcgagtcagtgagcgaggaagcggaagagcgcccaa | |
| | tacgcaaaccgcctctccccgcgcgttggccgattcattaatgcagctggcacgacaggtttcccgactggaaagcgggc | |
| | agtgagcgcaacgcaattaatgtgagttagctcactcattaggcaccccaggctttacactttatgcttccggctcgtatgt | |
| | tgtgtggaattgtgagcggataacaatttcacacaggaaacagctatgaccatgattacgaattgcctgcaggcagctgcgc | |
| | gctcgctcgctcactgaggccgcccgggcaaagcccgggcgtcgggcgacctttggtcgcccggcctcagtgagcgagc | |
| | gagcgcgcagagagggagtggccaactccatcactaggggttcctatcgatggcgcgcctccagatatcatacactcgagc | |
| | aactttgtatagaaaagttgggtgctatagagcacaagattttatttggtgagatggtgctttcatgaattcccccaacaga | |
| | gccaagctctccatctagtggacagggaagctagcagcaaaccttcccttcactacaaaacttcattgcttggccaaaaag | |
| | agagttaattcaatgtagacatctatgtaggcaattaaaaacctattgatgtataaaacagtttgcattcatggagggcaac | |
| | taaatacattctaggactttataaaagatcactttttatttatgcacagggtggaacaagatggattatcaagtgtcaagtc | |
| | caatctatgacatcaattattatacatcggagccctgccaaaaaatcaatgtgaagcaaatcgcagcccgcctcctgcctcc | |
| | gctctactcactggtgttcatctttggttttgtgggcaacatgctggtcatcctcatcctgataaactgcaaaaggctgaag | |
| | agcatgactgacatctacctgctcaacctggccatctctgacctgtttttccttcttactgtccccttctgggctcactatg | |
| | ctgccgcccagtgggactttggaaatacaatgtgtcaactcttgacagaacagttggaacagcagaatatgggccaaacagg | |
| | atatctgtggtaagcagttcctgccccggctcagggccaagaacagttggaacagcagaatatgggccaaacaggatatctg | |
| | tggtaagcagttcctgccccggctcagggccaagaacagatggtccccagatgcggtcccgccctcagcagtttctagagaa | |
| | ccatcagatgtttccagggtgccccaaggacctgaaatgaccctgtgccttatttgaactaaccaatcagttcgcttctcgc | |
| | ttctgttcgcgcgcttctgctccccgagctctatataagcagagctcgtttagtgaaccgtcagatccaagtttgtacaaaa | |
| | aagcaggctgccaccATGCCTAGATACGGCGCCAGCCTGAGACAGAGCTGTCCTAGATCT | |
| | GGCAGAGAGCAGGGCCAAGACGGAACAGCTGGTGCTCCTGGACTGCTGT | |
| | GGATGGGACTTGTTCTGGCTCTGGCCCTGGCTCTTGCATTGGCACTGGCTC | |
| | TGAGCGACAGCAGAGTTCTGTGGGCTCCTGCCGAAGCTCATCCTCTGAGT | |
| | CCTCAGGGACATCCCGCCAGACTGCACAGAATCGTGCCCAGACTGAGAG | |
| | ATGTGTTCGGCTGGGGCAACCTGACATGCCCTATCTGCAAGGGCCTGTTC | |
| | ACCGCCATCAACCTGGGCCTGAAGAAAGAACCCAACGTGGCCAGAGTGG | |
| | GCAGCGTGGCCATCAAGCTGTGCAACCTGCTGAAGATTGCCCCTCCTGCC | |
| | GTGTGCCAGTCTATCGTGCACCTGTTCGAGGACGACATGGTGGAAGTGTG | |
| | GCGGAGAAGCGTGCTGTCTCCATCTGAAGCTTGTGGCCTGCTGCTGGGCT | |
| | CTACATGTGGCCACTGGGACATCTTTAGCAGCTGGAACATCAGCCTGCCT | |
| | ACCGTGCCTAAGCCTCCTCCAAAGCCTCCATCTCCTCCAGCACCTGGCGC | |
| | TCCAGTGTCCAGAATCCTGTTCCTGACCGACCTGCACTGGGACCACGATT | |
| | ACCTGGAAGGCACCGATCCTGACTGCGCCGATCCTCTGTGTTGCAGAAGA | |
| | GGCTCTGGACTGCCTCCTGCCTCTAGACCAGGTGCCGGATATTGGGGCGA | |
| | GTACAGCAAGTGCGACCTGCCTCTGAGAACCCTGGAAAGCCTGCTGTCTG | |
| | GACTGGGACCTGCCGGACCTTTCGATATGGTGTACTGGACCGGCGACATC | |
| | CCCGCTCACGATGTGTGGCATCAGACCAGACAGGACCAGCTGAGAGCCC | |
| | TGACAACAGTGACAGCCCTCGTGCGGAAGTTTCTGGGACCAGTGCCTGTG | |
| | TATCCCGCCGTGGGAAATCACGAGAGCACCCCTGTGAACAGCTTCCCTCC | |
| | ACCTTTCATCGAGGGCAACCACAGCAGCAGATGGCTGTACGAAGCCATG | |
| | GCCAAGGCCTGGGAACCTTGGCTTCCAGCTGAAGCACTGCGGACCCTGAG | |
| | AATCGGCGGCTTTTATGCTCTGAGCCCCTATCCTGGCCTGAGACTGATCA | |
| | GCCTGAACATGAATTTCTGCAGCCGCGAGAACTTCTGGCTGCTGATCAAC | |
| | TCCACAGATCCTGCCGGCCAGCTGCAGTGGCTTGTTGGAGAATTGCAGGC | |
| | CGCCGAGGACAGAGGCGATAAGGTGCACATCATCGGACACATCCCTCCA | |
| | GGCCACTGCCTGAAGTCTTGGAGCTGGAACTACTACCGGATTGTGGCCAG | |
| | ATACGAGAACACCCTGGCCGCTCAGTTCTTCGGCCACACACACGTGGACG | |
| | AGTTCGAGGTGTTCTACGACGAGGAAACCCTGAGCAGACCTCTGGCCGTG | |
| | GCATTTCTGGCCCCAAGCGCCACCACATATATCGGACTGAACCCCGGCTA | |
| | CCGGGTGTACCAGATCGACGGCAATTACAGCGGCAGCTCCCACGTGGTGC | |
| | TGGACCACGAGACTTACATCCTGAATCTGACCCAGGCCAACATTCCCGGC | |
| | GCTATCCCTCATTGGCAGCTGCTGTATCGGGCCAGAGAGACATACGGCCT | |
| | GCCTAACACACTGCCAACCGCCTGGCACAACCTGGTGTACAGAATGAGA | |
| | GGCGACATGCAGCTGTTTCAGACCTTCTGGTTCCTGTACCACAAGGGACA | |
| | CCCTCCAAGCGAGCCTTGTGGCACCCCTTGTAGACTGGCCACTCTGTGTG | |
| | CTCAGCTGTCCGCCAGAGCTGATAGCCCCGCTCTGTGCAGACACCTGATG | |
| | CCTGATGGAAGCCTGCCTGAGGCTCAGTCCCTTTGGCCTAGACCTCTGTTC | |
| | TCCTGAacccagctttcttgtacaaagtggctgtgccttctagttgccagccatctgttgtttgcccctcccccgtgcctt | |
| | ccttgaccctggaaggtgccactcccactgtcctttcctaataaaatgaggaaattgcatcgcattgtctgagtaggtgtca | |
| | ttctattctggggggtggggggggcaggacagcaagggggaggattgggaagagaatagcaggcatgctggggactct | |
| | attttataggcttcttctctggaatcttcttcatcatcctcctgacaatcgataggtacctggctgtcgtccatgctgtgtt | |
| | tgctttaaaagccaggacggtcacctttggggtggtgacaagtgtgatcacttgggtggtggctgtgtttgcgtctctccca | |
| | ggaatcatctttaccagatctcaaaaagaaggtcttcattacacctgcagctctcattttccatacagtcagtatcaattct | |
| | ggaagaatttccagacattaaagatagtcatcttggggctggtcctgccgctgcttgtcatggtcatctgctactcgggaat | |
| | cctaaaaactctgcttcggtgtcgaaatgagaagaagaggcacagggctgtgaggcttatcttcaccatcatgattgtttat | |
| | tttctcttctgggctccctacaacattgtccttctcctgaacaccttccaggaattctttggcctgaataattgcagtagct | |
| | ctaacaggttggaccaagctatgcaggtgacagagactcttgggatgacgcactgctgcatcaaccccatcatctatgcctt | |
| | tgtcggggagaagttcagaaaccaactttattatacatagttgatggccggccgcttcgagttaattaatccaaccggttac | |
| | cgcctaggatcgatagatctaggaacccctagtgatggagttggccactccctctctgcgcgctcgctcgctcactgaggcc | |
| | gggcgaccaaaggtcgcccgacgcccgggctttgcccgggcggcctcagtgagcgagcgagcgcgcagctgcctgcaggcag | |
| | cttggcactggccgtcgttttacaacgtcgtgactgggaaaaccctggcgttacccaacttaatcgccttgcagcacatccc | |
| | cctttcgccagctggcgtaatagcgaagaggcccgcaccgatcgcccttcccaacagttgcgcagcctgaatggcgaatggc | |
| | gcctgatgcggtattttctccttacgcatctgtgcggtatttcacaccgcatacgtcaaagcaaccatagtacgcgccctgt | |
| | agcggcgcattaagcgcggcgggtgtggtggttacgcgcagcgtgaccgctacacttgccagcgccctagcgcccgctcctt | |
| | tcgctttcttcccttcctttctcgccacgttcgccggctttccccgtcaagctctaaatcgggggctccctttagggttccg | |
| | atttagtgctttacggcacctcgaccccaaaaaacttgatttgggtgatggttcacgtagtgggccatcgccctgatagacg | |
| | gtttttcgccctttgacgttggagtccacgttctttaatagtggactcttgttccaaactggaacaacactcaaccctatct | |
| | cgggctattcttttgatttataagggattttgccgatttcggcctattggttaaaaaatgagctgatttaacaaaaatttaa | |
| | cgcgaattttaacaaaatattaacgtttacaattttatggtgcactctcagtacaatctgctctgatgccgcatagttaagc | |
| | cagccccgacacccgccaacacccgctgacgcgccctgacgggcttgtctgctcccggcatccgcttacagacaagctgtga | |
| | ccgtctccgggagctgcatgtgtcagaggttttcaccgtcatcaccgaaacgcgcgagacgaaagggcctcgtgatacgcct | |
| | atttttataggttaatgtcatgataataatggtttcttagacgtcaggtggcacttttcggggaaatgtg | |
| SMPD1 wild- | gaacagttggaacagcagaatatgggccaaacaggatatctgtggtaagcagttcctgccccggctcagggccaagaac | 27 |
| type | agttggaacagcagaatatgggccaaacaggatatctgtggtaagcagttcctgccccggctcagggccaagaacagat | |
| expression | ggtccccagatgcggtcccgccctcagcagtttctagagaaccatcagatgtttccagggtgccccaaggacctgaaatg | |
| cassette | accctgtgccttatttgaactaaccaatcagttcgcttctcgcttctgttcgcgcgcttctgctccccgagctctatataag | |
| only | cagagctcgtttagtgaaccgtcagatccaagtttgtacaaaaaagcaggctgccaccatgccccgctacggagcgtcactc | |
| (No HAs or | cgccagagctgccccaggtccggccgggagcagggacaagacgggaccgccggagcccccggactcctttggatgggc | |
| plasmid | ctggtgctggcgctggcgctggcgctggcgctggcgctggctctgtctgactctcgggttctctgggctccggcagaggc | |
| backbone) | tcaccctctttctccccaaggccatcctgccaggttacatcgcatagtgccccggctccgagatgtctttggggggggaac | |
| | ctcacctgcccaatctgcaaaggtctattcaccgccatcaacctcgggctgaagaaggaacccaatgtggctcgcgtggg | |
| | ctccgtggccatcaagctgtgcaatctgctgaagatagcaccacctgccgtgtgccaatccattgtccacctctttgaggat | |
| | gacatggtggaggtgtggagacgctcagtgctgagcccatctgaggcctgtggcctgctcctgggctccacctgtgggca | |
| | ctgggacattttctcatcttggaacatctctttgcctactgtgccgaagccgccccccaaaccccctagccccccagcccca | |
| | ggtgcccctgtcagccgcatcctcttcctcactgacctgcactgggatcatgactacctggagggcacggaccctgactgt | |
| | gcagacccactgtgctgccgccggggttctggcctgccgcccgcatcccggccaggtgccggatactggggcgaatac | |
| | agcaagtgtgacctgcccctgaggaccctggagagcctgttgagtgggctgggcccagccggcccttttgatatggtgta | |
| | ctggacaggagacatccccgcacatgatgtctggcaccagactcgtcaggaccaactgcgggccctgaccaccgtcaca | |
| | gcacttgtgaggaagttcctggggccagtgccagtgtaccctgctgtgggtaaccatgaaagcacacctgtcaatagcttc | |
| | cctccccccttcattgagggcaaccactcctcccgctggctctatgaagcgatggccaaggcttgggagccctggctgcctg | |
| | ccgaagccctgcgcaccctcagaattggggggttctatgctctttccccataccccggtctccgcctcatctctctcaatat | |
| | gaatttttgttcccgtgagaacttctggctcttgatcaactccacggatcccgcaggacagctccagtggctggtgggggag | |
| | cttcaggctgctgaggatcgaggagacaaagtgcatataattggccacattcccccagggcactgtctgaagagctggagct | |
| | ggaattattaccgaattgtagccaggtatgagaacaccctggctgctcagttctttggccacactcatgtggatgaatttga | |
| | ggtcttctatgatgaagagactctgagccggccgctggctgtagccttcctggcacccagtgcaactacctacatcggcctt | |
| | aatcctggttaccgtgtgtaccaaatagatggaaactactccgggagctctcacgtggtcctggaccatgagacctacatcc | |
| | tgaatctgacccaggcaaacataccgggagccataccgcactggcagcttctctacagggctcgagaaacctatgggctg | |
| | cccaacacactgcctaccgcctggcacaacctggtatatcgcatgcggggcgacatgcaacttttccagaccttctggtttc | |
| | tctaccataagggccacccaccctcggagccctgtggcacgccctgccgtctggctactctttgtgcccagctctctgccc | |
| | gtgctgacagccctgctctgtgccgccacctgatgccagatgggagcctcccagaggcccagagcctgtggccaaggccact | |
| | gttttgctagacccagctttcttgtacaaagtggctgtgccttctagttgccagccatctgttgtttgcccctcccccgtgc | |
| | cttccttgaccctggaaggtgccactcccactgtcctttcctaataaaatgaggaaattgcatcgcattgtctgagtaggtg | |
| | tcattctattctggggggtggggtggggcaggacagcaagggggaggattgggaagagaatagcaggcatgctgggga | |
| SMPD1 | gaacagttggaacagcagaatatgggccaaacaggatatctgtggtaagcagttcctgccccggctcagggccaagaac | 28 |
| GeneArt | agttggaacagcagaatatgggccaaacaggatatctgtggtaagcagttcctgccccggctcagggccaagaacagat | |
| Partial | ggtccccagatgcggtcccgccctcagcagtttctagagaaccatcagatgtttccagggtgccccaaggacctgaaatg | |
| Codon | accctgtgccttatttgaactaaccaatcagttcgcttctcgcttctgttcgcgcgcttctgctccccgagctctatataag | |
| Optimized | cagagctcgtttagtgaaccgtcagatccaagtttgtacaaaaaagcaggctgccaccATGCCCCGCTACGGAG | |
| expression | CGTCACTCCGCCAGAGCTGCCCCAGGTCCGGCAGGGAGCAGGGACAAGA | |
| cassette | CGGGACCGCCGGAGCTCCTGGACTCCTTTGGATGGGACTGGTGCTGGCGC | |
| only | TGGCGCTTGCTCTGGCGCTGGCCCTGGCACTGTCTGACTCTCGGGTTCTCT | |
| (No HAs or | GGGCTCCGGCAGAAGCTCACCCTCTTTCTCCTCAAGGCCATCCTGCCAGG | |
| plasmid | TTACATCGCATAGTGCCCCGGCTCCGAGATGTCTTTGGGTGGGGGAACCT | |
| backbone) | CACCTGCCCAATCTGCAAAGGTCTATTCACCGCCATCAACCTCGGGCTGA | |
| | AGAAGGAACCCAATGTGGCTCGCGTGGGCTCCGTGGCCATCAAGCTGTGC | |
| | AATCTGCTGAAGATAGCACCACCTGCCGTGTGCCAATCCATTGTCCACCT | |
| | CTTTGAGGATGACATGGTGGAGGTGTGGAGACGCTCAGTGCTGAGCCCAT | |
| | CTGAGGCCTGTGGCCTGCTCCTGGGCTCCACCTGTGGGCACTGGGACATT | |
| | TTCTCATCTTGGAACATCTCTTTGCCTACTGTGCCGAAGCCGCCCCCCAAA | |
| | CCCCCTAGCCCTCCAGCCCCAGGTGCTCCTGTCAGCCGCATCCTCTTCCTC | |
| | ACTGACCTGCACTGGGATCATGACTACCTGGAGGGCACGGACCCTGACTG | |
| | TGCAGACCCACTGTGCTGCCGCCGGGGTTCTGGCCTGCCGCCTGCATCCA | |
| | GGCCAGGTGCCGGATACTGGGGCGAATACAGCAAGTGTGACCTGCCCCT | |
| | GAGGACCCTGGAGAGCCTGTTGAGTGGGCTGGGCCCAGCCGGACCTTTTG | |
| | ATATGGTGTACTGGACAGGAGACATCCCCGCACATGATGTCTGGCACCAG | |
| | ACTCGTCAGGACCAACTGCGGGCCCTGACCACCGTCACAGCACTTGTGAG | |
| | GAAGTTCCTGGGGCCAGTGCCAGTGTACCCTGCTGTGGGTAACCATGAAA | |
| | GCACACCTGTCAATAGCTTCCCTCCCCCCTTCATTGAGGGCAACCACTCCT | |
| | CCCGCTGGCTCTATGAAGCGATGGCCAAGGCTTGGGAGCCCTGGCTGCCT | |
| | GCCGAAGCCCTGCGCACCCTCAGAATTGGGGGGTTCTATGCTCTTTCCCC | |
| | ATACCCCGGTCTCCGCCTCATCTCTCTCAATATGAATTTTTGTTCCCGTGA | |
| | GAACTTCTGGCTCTTGATCAACTCCACGGATCCCGCAGGACAGCTCCAGT | |
| | GGCTGGTGGGGGAGCTTCAGGCTGCTGAGGATCGAGGAGACAAAGTGCA | |
| | TATAATTGGCCACATTCCCCCAGGGCACTGTCTGAAGAGCTGGAGCTGGA | |
| | ATTATTACCGAATTGTAGCCAGGTATGAGAACACCCTGGCTGCTCAGTTC | |
| | TTTGGCCACACTCATGTGGATGAATTTGAGGTCTTCTATGATGAAGAGAC | |
| | TCTGAGCCGGCCGCTGGCTGTAGCCTTCCTGGCACCCAGTGCAACTACCT | |
| | ACATCGGCCTTAATCCTGGTTACCGTGTGTACCAAATAGATGGAAACTAC | |
| | TCCGGGAGCTCTCACGTGGTCCTGGACCATGAGACCTACATCCTGAATCT | |
| | GACCCAGGCAAACATACCGGGAGCCATACCGCACTGGCAGCTTCTCTACA | |
| | GGGCTCGAGAAACCTATGGGCTGCCCAACACACTGCCTACCGCCTGGCAC | |
| | AACCTGGTATATCGCATGCGGGGCGACATGCAACTTTTCCAGACCTTCTG | |
| | GTTTCTCTACCATAAGGGCCACCCACCCTCGGAGCCCTGTGGCACGCCCT | |
| | GTCGTCTGGCTACTCTTTGTGCCCAGCTCTCTGCCCGTGCTGACAGCCCTG | |
| | CTCTGTGCCGCCACCTGATGCCAGATGGGAGCCTCCCAGAGGCCCAGAGC | |
| | CTGTGGCCAAGGCCACTGTTTTGCTAGacccagctttcttgtacaaagtggctgtgccttctagttgcc | |
| | agccatctgttgtttgcccctcccccgtgccttccttgaccctggaaggtgccactcccactgtcctttcctaataaaatga | |
| | ggaaattgcatcgcattgtctgagtaggtgtcattctattctggggggggggtggggcaggacagcaagggggaggatt | |
| | gggaagagaatagcaggcatgctgggga | |
| SMPD1 C629S | gaacagttggaacagcagaatatgggccaaacaggatatctgtggtaagcagttcctgccccggctcagggccaagaac | 29 |
| GeneArt | agttggaacagcagaatatgggccaaacaggatatctgtggtaagcagttcctgccccggctcagggccaagaacagat | |
| Partial | ggtccccagatgcggtcccgccctcagcagtttctagagaaccatcagatgtttccagggtgccccaaggacctgaaatg | |
| Codon | accctgtgccttatttgaactaaccaatcagttcgcttctcgcttctgttcgcgcgcttctgctccccgagctctatataag | |
| Optimized | cagagctcgtttagtgaaccgtcagatccaagtttgtacaaaaaagcaggctgccaccATGCCCCGCTACGGAG | |
| expression | CGTCACTCCGCCAGAGCTGCCCCAGGTCCGGCAGGGAGCAGGGACAAGA | |
| cassette | CGGGACCGCCGGAGCTCCTGGACTCCTTTGGATGGGACTGGTGCTGGCGC | |
| only | TGGCGCTTGCTCTGGCGCTGGCCCTGGCACTGTCTGACTCTCGGGTTCTCT | |
| (No HAs or | GGGCTCCGGCAGAAGCTCACCCTCTTTCTCCTCAAGGCCATCCTGCCAGG | |
| plasmid | TTACATCGCATAGTGCCCCGGCTCCGAGATGTCTTTGGGTGGGGGAACCT | |
| backbone) | CACCTGCCCAATCTGCAAAGGTCTATTCACCGCCATCAACCTCGGGCTGA | |
| | AGAAGGAACCCAATGTGGCTCGCGTGGGCTCCGTGGCCATCAAGCTGTGC | |
| | AATCTGCTGAAGATAGCACCACCTGCCGTGTGCCAATCCATTGTCCACCT | |
| | CTTTGAGGATGACATGGTGGAGGTGTGGAGACGCTCAGTGCTGAGCCCAT | |
| | CTGAGGCCTGTGGCCTGCTCCTGGGCTCCACCTGTGGGCACTGGGACATT | |
| | TTCTCATCTTGGAACATCTCTTTGCCTACTGTGCCGAAGCCGCCCCCCAAA | |
| | CCCCCTAGCCCTCCAGCCCCAGGTGCTCCTGTCAGCCGCATCCTCTTCCTC | |
| | ACTGACCTGCACTGGGATCATGACTACCTGGAGGGCACGGACCCTGACTG | |
| | TGCAGACCCACTGTGCTGCCGCCGGGGTTCTGGCCTGCCGCCTGCATCCA | |
| | GGCCAGGTGCCGGATACTGGGGCGAATACAGCAAGTGTGACCTGCCCCT | |
| | GAGGACCCTGGAGAGCCTGTTGAGTGGGCTGGGCCCAGCCGGACCTTTTG | |
| | ATATGGTGTACTGGACAGGAGACATCCCCGCACATGATGTCTGGCACCAG | |
| | ACTCGTCAGGACCAACTGCGGGCCCTGACCACCGTCACAGCACTTGTGAG | |
| | GAAGTTCCTGGGGCCAGTGCCAGTGTACCCTGCTGTGGGTAACCATGAAA | |
| | GCACACCTGTCAATAGCTTCCCTCCCCCCTTCATTGAGGGCAACCACTCCT | |
| | CCCGCTGGCTCTATGAAGCGATGGCCAAGGCTTGGGAGCCCTGGCTGCCT | |
| | GCCGAAGCCCTGCGCACCCTCAGAATTGGGGGGTTCTATGCTCTTTCCCC | |
| | ATACCCCGGTCTCCGCCTCATCTCTCTCAATATGAATTTTTGTTCCCGTGA | |
| | GAACTTCTGGCTCTTGATCAACTCCACGGATCCCGCAGGACAGCTCCAGT | |
| | GGCTGGTGGGGGAGCTTCAGGCTGCTGAGGATCGAGGAGACAAAGTGCA | |
| | TATAATTGGCCACATTCCCCCAGGGCACTGTCTGAAGAGCTGGAGCTGGA | |
| | ATTATTACCGAATTGTAGCCAGGTATGAGAACACCCTGGCTGCTCAGTTC | |
| | TTTGGCCACACTCATGTGGATGAATTTGAGGTCTTCTATGATGAAGAGAC | |
| | TCTGAGCCGGCCGCTGGCTGTAGCCTTCCTGGCACCCAGTGCAACTACCT | |
| | ACATCGGCCTTAATCCTGGTTACCGTGTGTACCAAATAGATGGAAACTAC | |
| | TCCGGGAGCTCTCACGTGGTCCTGGACCATGAGACCTACATCCTGAATCT | |
| | GACCCAGGCAAACATACCGGGAGCCATACCGCACTGGCAGCTTCTCTACA | |
| | GGGCTCGAGAAACCTATGGGCTGCCCAACACACTGCCTACCGCCTGGCAC | |
| | AACCTGGTATATCGCATGCGGGGCGACATGCAACTTTTCCAGACCTTCTG | |
| | GTTTCTCTACCATAAGGGCCACCCACCCTCGGAGCCCTGTGGCACGCCCT | |
| | GTCGTCTGGCTACTCTTTGTGCCCAGCTCTCTGCCCGTGCTGACAGCCCTG | |
| | CTCTGTGCCGCCACCTGATGCCAGATGGGAGCCTCCCAGAGGCCCAGAGC | |
| | CTGTGGCCAAGGCCACTGTTTTCCTAGacccagctttcttgtacaaagtggctgtgccttctagttgcca | |
| | gccatctgttgtttgcccctcccccgtgccttccttgaccctggaaggtgccactcccactgtcctttcctaataaaatga | |
| | ggaaattgcatcgcattgtctgagtaggtgtcattctattctggggggtggggggggcaggacagcaagggggaggatt | |
| | gggaagagaatagcaggcatgctgggga | |
| SMPD1 | gaacagttggaacagcagaatatgggccaaacaggatatctgtggtaagcagttcctgccccggctcagggccaagaac | 30 |
| GeneArt Full | agttggaacagcagaatatgggccaaacaggatatctgtggtaagcagttcctgccccggctcagggccaagaacagat | |
| Codon | ggtccccagatgcggtcccgccctcagcagtttctagagaaccatcagatgtttccagggtgccccaaggacctgaaatg | |
| Optimized | accctgtgccttatttgaactaaccaatcagttcgcttctcgcttctgttcgcgcgcttctgctccccgagctctatataag | |
| expression | cagagctcgtttagtgaaccgtcagatccaagtttgtacaaaaaagcaggctgccaccATGCCTAGATACGGCG | |
| cassette only | CCAGCCTGAGACAGAGCTGTCCTAGATCTGGCAGAGAGCAGGGCCAAGA | |
| (No HAs or | CGGAACAGCTGGTGCTCCTGGACTGCTGTGGATGGGACTTGTTCTGGCTC | |
| plasmid | TGGCCCTGGCTCTTGCATTGGCACTGGCTCTGAGCGACAGCAGAGTTCTG | |
| backbone) | TGGGCTCCTGCCGAAGCTCATCCTCTGAGTCCTCAGGGACATCCCGCCAG | |
| | ACTGCACAGAATCGTGCCCAGACTGAGAGATGTGTTCGGCTGGGGCAACC | |
| | TGACATGCCCTATCTGCAAGGGCCTGTTCACCGCCATCAACCTGGGCCTG | |
| | AAGAAAGAACCCAACGTGGCCAGAGTGGGCAGCGTGGCCATCAAGCTGT | |
| | GCAACCTGCTGAAGATTGCCCCTCCTGCCGTGTGCCAGTCTATCGTGCAC | |
| | CTGTTCGAGGACGACATGGTGGAAGTGTGGCGGAGAAGCGTGCTGTCTCC | |
| | ATCTGAAGCTTGTGGCCTGCTGCTGGGCTCTACATGTGGCCACTGGGACA | |
| | TCTTTAGCAGCTGGAACATCAGCCTGCCTACCGTGCCTAAGCCTCCTCCA | |
| | AAGCCTCCATCTCCTCCAGCACCTGGCGCTCCAGTGTCCAGAATCCTGTTC | |
| | CTGACCGACCTGCACTGGGACCACGATTACCTGGAAGGCACCGATCCTGA | |
| | CTGCGCCGATCCTCTGTGTTGCAGAAGAGGCTCTGGACTGCCTCCTGCCT | |
| | CTAGACCAGGTGCCGGATATTGGGGCGAGTACAGCAAGTGCGACCTGCCT | |
| | CTGAGAACCCTGGAAAGCCTGCTGTCTGGACTGGGACCTGCCGGACCTTT | |
| | CGATATGGTGTACTGGACCGGCGACATCCCCGCTCACGATGTGTGGCATC | |
| | AGACCAGACAGGACCAGCTGAGAGCCCTGACAACAGTGACAGCCCTCGT | |
| | GCGGAAGTTTCTGGGACCAGTGCCTGTGTATCCCGCCGTGGGAAATCACG | |
| | AGAGCACCCCTGTGAACAGCTTCCCTCCACCTTTCATCGAGGGCAACCAC | |
| | AGCAGCAGATGGCTGTACGAAGCCATGGCCAAGGCCTGGGAACCTTGGC | |
| | TTCCAGCTGAAGCACTGCGGACCCTGAGAATCGGCGGCTTTTATGCTCTG | |
| | AGCCCCTATCCTGGCCTGAGACTGATCAGCCTGAACATGAATTTCTGCAG | |
| | CCGCGAGAACTTCTGGCTGCTGATCAACTCCACAGATCCTGCCGGCCAGC | |
| | TGCAGTGGCTTGTTGGAGAATTGCAGGCCGCCGAGGACAGAGGCGATAA | |
| | GGTGCACATCATCGGACACATCCCTCCAGGCCACTGCCTGAAGTCTTGGA | |
| | GCTGGAACTACTACCGGATTGTGGCCAGATACGAGAACACCCTGGCCGCT | |
| | CAGTTCTTCGGCCACACACACGTGGACGAGTTCGAGGTGTTCTACGACGA | |
| | GGAAACCCTGAGCAGACCTCTGGCCGTGGCATTTCTGGCCCCAAGCGCCA | |
| | CCACATATATCGGACTGAACCCCGGCTACCGGGTGTACCAGATCGACGGC | |
| | AATTACAGCGGCAGCTCCCACGTGGTGCTGGACCACGAGACTTACATCCT | |
| | GAATCTGACCCAGGCCAACATTCCCGGCGCTATCCCTCATTGGCAGCTGC | |
| | TGTATCGGGCCAGAGAGACATACGGCCTGCCTAACACACTGCCAACCGCC | |
| | TGGCACAACCTGGTGTACAGAATGAGAGGCGACATGCAGCTGTTTCAGAC | |
| | CTTCTGGTTCCTGTACCACAAGGGACACCCTCCAAGCGAGCCTTGTGGCA | |
| | CCCCTTGTAGACTGGCCACTCTGTGTGCTCAGCTGTCCGCCAGAGCTGAT | |
| | AGCCCCGCTCTGTGCAGACACCTGATGCCTGATGGAAGCCTGCCTGAGGC | |
| | TCAGTCCCTTTGGCCTAGACCTCTGTTCTGCTGAacccagctttcttgtacaaagtggctgtgcctt | |
| | ctagttgccagccatctgttgtttgcccctcccccgtgccttccttgaccctggaaggtgccactcccactgtcctttcct | |
| | aataaaatgaggaaattgcatcgcattgtctgagtaggtgtcattctattctggggggtggggggggcaggacagcaagg | |
| | gggaggattgggaagagaatagcaggcatgctgggga | |
| SMPD1 C629S | gaacagttggaacagcagaatatgggccaaacaggatatctgtggtaagcagttcctgccccggctcagggccaagaac | 31 |
| GeneArt Full | agttggaacagcagaatatgggccaaacaggatatctgtggtaagcagttcctgccccggctcagggccaagaacagat | |
| Codon | ggtccccagatgcggtcccgccctcagcagtttctagagaaccatcagatgtttccagggtgccccaaggacctgaaatg | |
| Optimized | accctgtgccttatttgaactaaccaatcagttcgcttctcgcttctgttcgcgcgcttctgctccccgagctctatataag | |
| expression | cagagctcgtttagtgaaccgtcagatccaagtttgtacaaaaaagcaggctgccaccATGCCTAGATACGGCG | |
| cassette only | CCAGCCTGAGACAGAGCTGTCCTAGATCTGGCAGAGAGCAGGGCCAAGA | |
| (No HAs or | CGGAACAGCTGGTGCTCCTGGACTGCTGTGGATGGGACTTGTTCTGGCTC | |
| plasmid | TGGCCCTGGCTCTTGCATTGGCACTGGCTCTGAGCGACAGCAGAGTTCTG | |
| backbone) | TGGGCTCCTGCCGAAGCTCATCCTCTGAGTCCTCAGGGACATCCCGCCAG | |
| | ACTGCACAGAATCGTGCCCAGACTGAGAGATGTGTTCGGCTGGGGCAACC | |
| | TGACATGCCCTATCTGCAAGGGCCTGTTCACCGCCATCAACCTGGGCCTG | |
| | AAGAAAGAACCCAACGTGGCCAGAGTGGGCAGCGTGGCCATCAAGCTGT | |
| | GCAACCTGCTGAAGATTGCCCCTCCTGCCGTGTGCCAGTCTATCGTGCAC | |
| | CTGTTCGAGGACGACATGGTGGAAGTGTGGCGGAGAAGCGTGCTGTCTCC | |
| | ATCTGAAGCTTGTGGCCTGCTGCTGGGCTCTACATGTGGCCACTGGGACA | |
| | TCTTTAGCAGCTGGAACATCAGCCTGCCTACCGTGCCTAAGCCTCCTCCA | |
| | AAGCCTCCATCTCCTCCAGCACCTGGCGCTCCAGTGTCCAGAATCCTGTTC | |
| | CTGACCGACCTGCACTGGGACCACGATTACCTGGAAGGCACCGATCCTGA | |
| | CTGCGCCGATCCTCTGTGTTGCAGAAGAGGCTCTGGACTGCCTCCTGCCT | |
| | CTAGACCAGGTGCCGGATATTGGGGCGAGTACAGCAAGTGCGACCTGCCT | |
| | CTGAGAACCCTGGAAAGCCTGCTGTCTGGACTGGGACCTGCCGGACCTTT | |
| | CGATATGGTGTACTGGACCGGCGACATCCCCGCTCACGATGTGTGGCATC | |
| | AGACCAGACAGGACCAGCTGAGAGCCCTGACAACAGTGACAGCCCTCGT | |
| | GCGGAAGTTTCTGGGACCAGTGCCTGTGTATCCCGCCGTGGGAAATCACG | |
| | AGAGCACCCCTGTGAACAGCTTCCCTCCACCTTTCATCGAGGGCAACCAC | |
| | AGCAGCAGATGGCTGTACGAAGCCATGGCCAAGGCCTGGGAACCTTGGC | |
| | TTCCAGCTGAAGCACTGCGGACCCTGAGAATCGGCGGCTTTTATGCTCTG | |
| | AGCCCCTATCCTGGCCTGAGACTGATCAGCCTGAACATGAATTTCTGCAG | |
| | CCGCGAGAACTTCTGGCTGCTGATCAACTCCACAGATCCTGCCGGCCAGC | |
| | TGCAGTGGCTTGTTGGAGAATTGCAGGCCGCCGAGGACAGAGGCGATAA | |
| | GGTGCACATCATCGGACACATCCCTCCAGGCCACTGCCTGAAGTCTTGGA | |
| | GCTGGAACTACTACCGGATTGTGGCCAGATACGAGAACACCCTGGCCGCT | |
| | CAGTTCTTCGGCCACACACACGTGGACGAGTTCGAGGTGTTCTACGACGA | |
| | GGAAACCCTGAGCAGACCTCTGGCCGTGGCATTTCTGGCCCCAAGCGCCA | |
| | CCACATATATCGGACTGAACCCCGGCTACCGGGTGTACCAGATCGACGGC | |
| | AATTACAGCGGCAGCTCCCACGTGGTGCTGGACCACGAGACTTACATCCT | |
| | GAATCTGACCCAGGCCAACATTCCCGGCGCTATCCCTCATTGGCAGCTGC | |
| | TGTATCGGGCCAGAGAGACATACGGCCTGCCTAACACACTGCCAACCGCC | |
| | TGGCACAACCTGGTGTACAGAATGAGAGGCGACATGCAGCTGTTTCAGAC | |
| | CTTCTGGTTCCTGTACCACAAGGGACACCCTCCAAGCGAGCCTTGTGGCA | |
| | CCCCTTGTAGACTGGCCACTCTGTGTGCTCAGCTGTCCGCCAGAGCTGAT | |
| | AGCCCCGCTCTGTGCAGACACCTGATGCCTGATGGAAGCCTGCCTGAGGC | |
| | TCAGTCCCTTTGGCCTAGACCTCTGTTCTCCTGAacccagctttcttgtacaaagtggctgtgcctt | |
| | ctagttgccagccatctgttgtttgcccctcccccgtgccttccttgaccctggaaggtgccactcccactgtcctttcct | |
| | aataaaatgaggaaattgcatcgcattgtctgagtaggtgtcattctattctggggggtggggggggcaggacagcaagg | |
| | gggaggattgggaagagaatagcaggcatgctgggga | |

TABLE 3
Exemplary Promoters

| | | SEQ ID |
|---|---|---|
| Name | Nucleotide Sequence | NO |
| MND | gaacagttggaacagcagaatatgggccaaacaggatatctgtggtaagcagttcctgccccggctcagggccaagaa | 32 |
| | cagttggaacagcagaatatgggccaaacaggatatctgtggtaagcagttcctgccccggctcagggccaagaacag | |
| | atggtccccagatgcggtcccgccctcagcagtttctagagaaccatcagatgtttccagggtgccccaaggacctga | |
| | aatgaccctgtgccttatttgaactaaccaatcagttcgcttctcgcttctgttcgcgcgcttctgctccccgagctc | |
| | tatataagcagagctcgtttagtgaaccgtcagatc | |
| EF-1a | gggcagagcgcacatcgcccacagtccccgagaagttggggggaggggtcggcaattgatccggtgcctagagaag | 33 |
| | gtggcgcggggtaaactgggaaagtgatgtcgtgtactggctccgcctttttcccgaggggggggagaaccgtatata | |
| | agtgcagtagtcgccgtgaacgttctttttcgcaacgggtttgccgccagaacacagg | |

TABLE 4
Exemplary Transgenes

| | | SEQ |
|---|---|---|
| | | ID |
| Name | Nucleotide Sequence | NO |
| Factor IX | ATGCAGCGCGTGAACATGATCATGGCAGAATCACCAGGCCTCATCACCA | 34 |
| CDS | TCTGCCTTTTAGGATATCTACTCAGTGCTGAATGTACAGTTTTTCTTGATC | |
| | ATGAAAACGCCAACAAAATTCTGAATCGGCCAAAGAGGTATAATTCAGG | |
| | TAAATTGGAAGAGTTTGTTCAAGGGAACCTTGAGAGAGAATGTATGGAA | |
| | GAAAAGTGTAGTTTTGAAGAAGCACGAGAAGTTTTTGAAAACACTGAAA | |
| | GAACAACTGAATTTTGGAAGCAGTATGTTGATGTAACATGTAACATTAA | |
| | GAATGGCAGATGCGAGCAGTTTTGTAAAAATAGTGCTGATAACAAGGTG | |
| | GTTTGCTCCTGTACTGAGGGATATCGACTTGCAGAAAACCAGAAGTCCT | |
| | GTGAACCAGCAGTGCCATTTCCATGTGGAAGAGTTTCTGTTTCACAAACT | |
| | TCTAAGCTCACCCGTGCTGAGACTGTTTTTCCTGATGTGGACTATGTAAA | |
| | TTCTACTGAAGCTGAAACCATTTTGGATAACATCACTCAAAGCACCCAA | |
| | TCATTTAATGACTTCACTCGGGTTGTTGGTGGAGAAGATGCCAAACCAG | |
| | GTCAATTCCCTTGGCAGGTTGTTTTGAATGGTAAAGTTGATGCATTCTGT | |
| | GGAGGCTCTATCGTTAATGAAAAATGGATTGTAACTGCTGCCCACTGTG | |
| | TTGAAACTGGTGTTAAAATTACAGTIGTCGCAGGTGAACATAATATTGA | |
| | GGAGACAGAACATACAGAGCAAAAGCGAAATGTGATTCGAATTATTCCT | |
| | CACCACAACTACAATGCAGCTATTAATAAGTACAACCATGACATTGCCC | |
| | TTCTGGAACTGGACGAACCCTTAGTGCTAAACAGCTACGTTACACCTATT | |
| | TGCATTGCTGACAAGGAATACACGAACATCTTCCTCAAATTTGGATCTG | |
| | GCTATGTAAGTGGCTGGGGAAGAGTCTTCCACAAAGGGAGATCAGCTTT | |
| | AGTTCTTCAGTACCTTAGAGTTCCACTTGTTGACCGAGCCACATGTCTTC | |
| | GATCTACAAAGTTCACCATCTATAACAACATGTTCTGTGCTGGCTTCCAT | |
| | GAAGGAGGTAGAGATTCATGTCAAGGAGATAGTGGGGGACCCCATGTT | |
| | ACTGAAGTGGAAGGGACCAGTTTCTTAACTGGAATTATTAGCTGGGGTG | |
| | AAGAGTGTGCAATGAAAGGCAAATATGGAATATATACCAAGGTATCCCG | |
| | GTATGTCAACTGGATTAAGGAAAAAACAAAGCTCACTTAA | |
| SMPD1 wild- | atgccccgctacggagcgtcactccgccagagctgccccaggtccggccgggagcagggacaagacgggaccgcc | 35 |
| type | ggagcccccggactcctttggatgggcctggtgctggcgctggcgctggcgctggcgctggcgctggctctgtctgact | |
| | ctcgggttctctgggctccggcagaggctcaccctctttctccccaaggccatcctgccaggttacatcgcatagtgcccc | |
| | ggctccgagatgtctttggggggggaacctcacctgcccaatctgcaaaggtctattcaccgccatcaacctcgggctg | |
| | aagaaggaacccaatgtggctcgcgtgggctccgtggccatcaagctgtgcaatctgctgaagatagcaccacctgcc | |
| | gtgtgccaatccattgtccacctctttgaggatgacatggtggaggtgtggagacgctcagtgctgagcccatctgaggc | |
| | ctgtggcctgctcctgggctccacctgtgggcactgggacattttctcatcttggaacatctctttgcctactgtgccgaagc | |
| | cgccccccaaaccccctagccccccagccccaggtgcccctgtcagccgcatcctcttcctcactgacctgcactggga | |
| | tcatgactacctggagggcacggaccctgactgtgcagacccactgtgctgccgccggggttctggcctgccgcccgc | |
| | atcccggccaggtgccggatactggggcgaatacagcaagtgtgacctgcccctgaggaccctggagagcctgttga | |
| | gtgggctgggcccagccggcccttttgatatggtgtactggacaggagacatccccgcacatgatgtctggcaccagac | |
| | tcgtcaggaccaactgcgggccctgaccaccgtcacagcacttgtgaggaagttcctggggccagtgccagtgtaccct | |
| | gctgtgggtaaccatgaaagcacacctgtcaatagcttccctccccccttcattgagggcaaccactcctcccgctggctc | |
| | tatgaagcgatggccaaggcttgggagccctggctgcctgccgaagccctgcgcaccctcagaattggggggttctatg | |
| | ctctttccccataccccggtctccgcctcatctctctcaatatgaatttttgttcccgtgagaacttctggctcttgatca | |
| | actccacggatcccgcaggacagctccagtggctggtgggggagcttcaggctgctgaggatcgaggagacaaagtgcatat | |
| | aattggccacattcccccagggcactgtctgaagagctggagctggaattattaccgaattgtagccaggtatgagaaca | |
| | ccctggctgctcagttctttggccacactcatgtggatgaatttgaggtcttctatgatgaagagactctgagccggccgct | |
| | ggctgtagccttcctggcacccagtgcaactacctacatcggccttaatcctggttaccgtgtgtaccaaatagatggaaa | |
| | ctactccgggagctctcacgtggtcctggaccatgagacctacatcctgaatctgacccaggcaaacataccgggagcc | |
| | ataccgcactggcagcttctctacagggctcgagaaacctatgggctgcccaacacactgcctaccgcctggcacaacc | |
| | tggtatatcgcatgcggggcgacatgcaacttttccagaccttctggtttctctaccataagggccacccaccctcggagc | |
| | cctgtggcacgccctgccgtctggctactctttgtgcccagctctctgcccgtgctgacagccctgctctgtgccgccacc | |
| | tgatgccagatgggagcctcccagaggcccagagcctgtggccaaggccactgttttgctag | |
| SMPD1 | ATGCCCCGCTACGGAGCGTCACTCCGCCAGAGCTGCCCCAGGTCCGGCA | 36 |
| GeneArt | GGGAGCAGGGACAAGACGGGACCGCCGGAGCTCCTGGACTCCTTTGGAT | |
| Partial | GGGACTGGTGCTGGCGCTGGCGCTTGCTCTGGCGCTGGCCCTGGCACTG | |
| Codon | TCTGACTCTCGGGTTCTCTGGGCTCCGGCAGAAGCTCACCCTCTTTCTCC | |
| Optimized | TCAAGGCCATCCTGCCAGGTTACATCGCATAGTGCCCCGGCTCCGAGAT | |
| | GTCTTTGGGTGGGGGAACCTCACCTGCCCAATCTGCAAAGGTCTATTCAC | |
| | CGCCATCAACCTCGGGCTGAAGAAGGAACCCAATGTGGCTCGCGTGGGC | |
| | TCCGTGGCCATCAAGCTGTGCAATCTGCTGAAGATAGCACCACCTGCCG | |
| | TGTGCCAATCCATTGTCCACCTCTTTGAGGATGACATGGTGGAGGTGTGG | |
| | AGACGCTCAGTGCTGAGCCCATCTGAGGCCTGTGGCCTGCTCCTGGGCT | |
| | CCACCTGTGGGCACTGGGACATTTTCTCATCTTGGAACATCTCTTTGCCT | |
| | ACTGTGCCGAAGCCGCCCCCCAAACCCCCTAGCCCTCCAGCCCCAGGTG | |
| | CTCCTGTCAGCCGCATCCTCTTCCTCACTGACCTGCACTGGGATCATGAC | |
| | TACCTGGAGGGCACGGACCCTGACTGTGCAGACCCACTGTGCTGCCGCC | |
| | GGGGTTCTGGCCTGCCGCCTGCATCCAGGCCAGGTGCCGGATACTGGGG | |
| | CGAATACAGCAAGTGTGACCTGCCCCTGAGGACCCTGGAGAGCCTGTTG | |
| | AGTGGGCTGGGCCCAGCCGGACCTTTTGATATGGTGTACTGGACAGGAG | |
| | ACATCCCCGCACATGATGTCTGGCACCAGACTCGTCAGGACCAACTGCG | |
| | GGCCCTGACCACCGTCACAGCACTTGTGAGGAAGTTCCTGGGGCCAGTG | |
| | CCAGTGTACCCTGCTGTGGGTAACCATGAAAGCACACCTGTCAATAGCT | |
| | TCCCTCCCCCCTTCATTGAGGGCAACCACTCCTCCCGCTGGCTCTATGAA | |
| | GCGATGGCCAAGGCTTGGGAGCCCTGGCTGCCTGCCGAAGCCCTGCGCA | |
| | CCCTCAGAATTGGGGGGTTCTATGCTCTTTCCCCATACCCCGGTCTCCGC | |
| | CTCATCTCTCTCAATATGAATTTTTGTTCCCGTGAGAACTTCTGGCTCTTG | |
| | ATCAACTCCACGGATCCCGCAGGACAGCTCCAGTGGCTGGTGGGGGAGC | |
| | TTCAGGCTGCTGAGGATCGAGGAGACAAAGTGCATATAATTGGCCACAT | |
| | TCCCCCAGGGCACTGTCTGAAGAGCTGGAGCTGGAATTATTACCGAATT | |
| | GTAGCCAGGTATGAGAACACCCTGGCTGCTCAGTTCTTTGGCCACACTC | |
| | ATGTGGATGAATTTGAGGTCTTCTATGATGAAGAGACTCTGAGCCGGCC | |
| | GCTGGCTGTAGCCTTCCTGGCACCCAGTGCAACTACCTACATCGGCCTTA | |
| | ATCCTGGTTACCGTGTGTACCAAATAGATGGAAACTACTCCGGGAGCTC | |
| | TCACGTGGTCCTGGACCATGAGACCTACATCCTGAATCTGACCCAGGCA | |
| | AACATACCGGGAGCCATACCGCACTGGCAGCTTCTCTACAGGGCTCGAG | |
| | AAACCTATGGGCTGCCCAACACACTGCCTACCGCCTGGCACAACCTGGT | |
| | ATATCGCATGCGGGGCGACATGCAACTTTTCCAGACCTTCTGGTTTCTCT | |
| | ACCATAAGGGCCACCCACCCTCGGAGCCCTGTGGCACGCCCTGTCGTCT | |
| | GGCTACTCTTTGTGCCCAGCTCTCTGCCCGTGCTGACAGCCCTGCTCTGT | |
| | GCCGCCACCTGATGCCAGATGGGAGCCTCCCAGAGGCCCAGAGCCTGTG | |
| | GCCAAGGCCACTGTTTTGCTAG | |
| SMPD1 C629S | ATGCCCCGCTACGGAGCGTCACTCCGCCAGAGCTGCCCCAGGTCCGGCA | 37 |
| GeneArt | GGGAGCAGGGACAAGACGGGACCGCCGGAGCTCCTGGACTCCTTTGGAT | |
| Partial | GGGACTGGTGCTGGCGCTGGCGCTTGCTCTGGCGCTGGCCCTGGCACTG | |
| Codon | TCTGACTCTCGGGTTCTCTGGGCTCCGGCAGAAGCTCACCCTCTTTCTCC | |
| Optimized | TCAAGGCCATCCTGCCAGGTTACATCGCATAGTGCCCCGGCTCCGAGAT | |
| | GTCTTTGGGTGGGGGAACCTCACCTGCCCAATCTGCAAAGGTCTATTCAC | |
| | CGCCATCAACCTCGGGCTGAAGAAGGAACCCAATGTGGCTCGCGTGGGC | |
| | TCCGTGGCCATCAAGCTGTGCAATCTGCTGAAGATAGCACCACCTGCCG | |
| | TGTGCCAATCCATTGTCCACCTCTTTGAGGATGACATGGTGGAGGTGTGG | |
| | AGACGCTCAGTGCTGAGCCCATCTGAGGCCTGTGGCCTGCTCCTGGGCT | |
| | CCACCTGTGGGCACTGGGACATTTTCTCATCTTGGAACATCTCTTTGCCT | |
| | ACTGTGCCGAAGCCGCCCCCCAAACCCCCTAGCCCTCCAGCCCCAGGTG | |
| | CTCCTGTCAGCCGCATCCTCTTCCTCACTGACCTGCACTGGGATCATGAC | |
| | TACCTGGAGGGCACGGACCCTGACTGTGCAGACCCACTGTGCTGCCGCC | |
| | GGGGTTCTGGCCTGCCGCCTGCATCCAGGCCAGGTGCCGGATACTGGGG | |
| | CGAATACAGCAAGTGTGACCTGCCCCTGAGGACCCTGGAGAGCCTGTTG | |
| | AGTGGGCTGGGCCCAGCCGGACCTTTTGATATGGTGTACTGGACAGGAG | |
| | ACATCCCCGCACATGATGTCTGGCACCAGACTCGTCAGGACCAACTGCG | |
| | GGCCCTGACCACCGTCACAGCACTTGTGAGGAAGTTCCTGGGGCCAGTG | |
| | CCAGTGTACCCTGCTGTGGGTAACCATGAAAGCACACCTGTCAATAGCT | |
| | TCCCTCCCCCCTTCATTGAGGGCAACCACTCCTCCCGCTGGCTCTATGAA | |
| | GCGATGGCCAAGGCTTGGGAGCCCTGGCTGCCTGCCGAAGCCCTGCGCA | |
| | CCCTCAGAATTGGGGGGTTCTATGCTCTTTCCCCATACCCCGGTCTCCGC | |
| | CTCATCTCTCTCAATATGAATTTTTGTTCCCGTGAGAACTTCTGGCTCTTG | |
| | ATCAACTCCACGGATCCCGCAGGACAGCTCCAGTGGCTGGTGGGGGAGC | |
| | TTCAGGCTGCTGAGGATCGAGGAGACAAAGTGCATATAATTGGCCACAT | |
| | TCCCCCAGGGCACTGTCTGAAGAGCTGGAGCTGGAATTATTACCGAATT | |
| | GTAGCCAGGTATGAGAACACCCTGGCTGCTCAGTTCTTTGGCCACACTC | |
| | ATGTGGATGAATTTGAGGTCTTCTATGATGAAGAGACTCTGAGCCGGCC | |
| | GCTGGCTGTAGCCTTCCTGGCACCCAGTGCAACTACCTACATCGGCCTTA | |
| | ATCCTGGTTACCGTGTGTACCAAATAGATGGAAACTACTCCGGGAGCTC | |
| | TCACGTGGTCCTGGACCATGAGACCTACATCCTGAATCTGACCCAGGCA | |
| | AACATACCGGGAGCCATACCGCACTGGCAGCTTCTCTACAGGGCTCGAG | |
| | AAACCTATGGGCTGCCCAACACACTGCCTACCGCCTGGCACAACCTGGT | |
| | ATATCGCATGCGGGGCGACATGCAACTTTTCCAGACCTTCTGGTTTCTCT | |
| | ACCATAAGGGCCACCCACCCTCGGAGCCCTGTGGCACGCCCTGTCGTCT | |
| | GGCTACTCTTTGTGCCCAGCTCTCTGCCCGTGCTGACAGCCCTGCTCTGT | |
| | GCCGCCACCTGATGCCAGATGGGAGCCTCCCAGAGGCCCAGAGCCTGTG | |
| | GCCAAGGCCACTGTTTTCCTAG | |
| SMPD1 | ATGCCTAGATACGGCGCCAGCCTGAGACAGAGCTGTCCTAGATCTGGCA | 38 |
| GeneArt Full | GAGAGCAGGGCCAAGACGGAACAGCTGGTGCTCCTGGACTGCTGTGGAT | |
| Codon | GGGACTTGTTCTGGCTCTGGCCCTGGCTCTTGCATTGGCACTGGCTCTGA | |
| Optimized | GCGACAGCAGAGTTCTGTGGGCTCCTGCCGAAGCTCATCCTCTGAGTCCT | |
| | CAGGGACATCCCGCCAGACTGCACAGAATCGTGCCCAGACTGAGAGATG | |
| | TGTTCGGCTGGGGCAACCTGACATGCCCTATCTGCAAGGGCCTGTTCACC | |
| | GCCATCAACCTGGGCCTGAAGAAAGAACCCAACGTGGCCAGAGTGGGC | |
| | AGCGTGGCCATCAAGCTGTGCAACCTGCTGAAGATTGCCCCTCCTGCCG | |
| | TGTGCCAGTCTATCGTGCACCTGTTCGAGGACGACATGGTGGAAGTGTG | |
| | GCGGAGAAGCGTGCTGTCTCCATCTGAAGCTTGTGGCCTGCTGCTGGGC | |
| | TCTACATGTGGCCACTGGGACATCTTTAGCAGCTGGAACATCAGCCTGC | |
| | CTACCGTGCCTAAGCCTCCTCCAAAGCCTCCATCTCCTCCAGCACCTGGC | |
| | GCTCCAGTGTCCAGAATCCTGTTCCTGACCGACCTGCACTGGGACCACG | |
| | ATTACCTGGAAGGCACCGATCCTGACTGCGCCGATCCTCTGTGTTGCAG | |
| | AAGAGGCTCTGGACTGCCTCCTGCCTCTAGACCAGGTGCCGGATATTGG | |
| | GGCGAGTACAGCAAGTGCGACCTGCCTCTGAGAACCCTGGAAAGCCTGC | |
| | TGTCTGGACTGGGACCTGCCGGACCTTTCGATATGGTGTACTGGACCGG | |
| | CGACATCCCCGCTCACGATGTGTGGCATCAGACCAGACAGGACCAGCTG | |
| | AGAGCCCTGACAACAGTGACAGCCCTCGTGCGGAAGTTTCTGGGACCAG | |
| | TGCCTGTGTATCCCGCCGTGGGAAATCACGAGAGCACCCCTGTGAACAG | |
| | CTTCCCTCCACCTTTCATCGAGGGCAACCACAGCAGCAGATGGCTGTAC | |
| | GAAGCCATGGCCAAGGCCTGGGAACCTTGGCTTCCAGCTGAAGCACTGC | |
| | GGACCCTGAGAATCGGCGGCTTTTATGCTCTGAGCCCCTATCCTGGCCTG | |
| | AGACTGATCAGCCTGAACATGAATTTCTGCAGCCGCGAGAACTTCTGGC | |
| | TGCTGATCAACTCCACAGATCCTGCCGGCCAGCTGCAGTGGCTTGTTGG | |
| | AGAATTGCAGGCCGCCGAGGACAGAGGCGATAAGGTGCACATCATCGG | |
| | ACACATCCCTCCAGGCCACTGCCTGAAGTCTTGGAGCTGGAACTACTAC | |
| | CGGATTGTGGCCAGATACGAGAACACCCTGGCCGCTCAGTTCTTCGGCC | |
| | ACACACACGTGGACGAGTTCGAGGTGTTCTACGACGAGGAAACCCTGAG | |
| | CAGACCTCTGGCCGTGGCATTTCTGGCCCCAAGCGCCACCACATATATC | |
| | GGACTGAACCCCGGCTACCGGGTGTACCAGATCGACGGCAATTACAGCG | |
| | GCAGCTCCCACGTGGTGCTGGACCACGAGACTTACATCCTGAATCTGAC | |
| | CCAGGCCAACATTCCCGGCGCTATCCCTCATTGGCAGCTGCTGTATCGGG | |
| | CCAGAGAGACATACGGCCTGCCTAACACACTGCCAACCGCCTGGCACAA | |
| | CCTGGTGTACAGAATGAGAGGCGACATGCAGCTGTTTCAGACCTTCTGG | |
| | TTCCTGTACCACAAGGGACACCCTCCAAGCGAGCCTTGTGGCACCCCTT | |
| | GTAGACTGGCCACTCTGTGTGCTCAGCTGTCCGCCAGAGCTGATAGCCC | |
| | CGCTCTGTGCAGACACCTGATGCCTGATGGAAGCCTGCCTGAGGCTCAG | |
| | TCCCTTTGGCCTAGACCTCTGTTCTGCTGA | |
| SMPD1 C629S | ATGCCTAGATACGGCGCCAGCCTGAGACAGAGCTGTCCTAGATCTGGCA | 39 |
| GeneArt Full | GAGAGCAGGGCCAAGACGGAACAGCTGGTGCTCCTGGACTGCTGTGGAT | |
| Codon | GGGACTTGTTCTGGCTCTGGCCCTGGCTCTTGCATTGGCACTGGCTCTGA | |
| Optimized | GCGACAGCAGAGTTCTGTGGGCTCCTGCCGAAGCTCATCCTCTGAGTCCT | |
| | CAGGGACATCCCGCCAGACTGCACAGAATCGTGCCCAGACTGAGAGATG | |
| | TGTTCGGCTGGGGCAACCTGACATGCCCTATCTGCAAGGGCCTGTTCACC | |
| | GCCATCAACCTGGGCCTGAAGAAAGAACCCAACGTGGCCAGAGTGGGC | |
| | AGCGTGGCCATCAAGCTGTGCAACCTGCTGAAGATTGCCCCTCCTGCCG | |
| | TGTGCCAGTCTATCGTGCACCTGTTCGAGGACGACATGGTGGAAGTGTG | |
| | GCGGAGAAGCGTGCTGTCTCCATCTGAAGCTTGTGGCCTGCTGCTGGGC | |
| | TCTACATGTGGCCACTGGGACATCTTTAGCAGCTGGAACATCAGCCTGC | |
| | CTACCGTGCCTAAGCCTCCTCCAAAGCCTCCATCTCCTCCAGCACCTGGC | |
| | GCTCCAGTGTCCAGAATCCTGTTCCTGACCGACCTGCACTGGGACCACG | |
| | ATTACCTGGAAGGCACCGATCCTGACTGCGCCGATCCTCTGTGTTGCAG | |
| | AAGAGGCTCTGGACTGCCTCCTGCCTCTAGACCAGGTGCCGGATATTGG | |
| | GGCGAGTACAGCAAGTGCGACCTGCCTCTGAGAACCCTGGAAAGCCTGC | |
| | TGTCTGGACTGGGACCTGCCGGACCTTTCGATATGGTGTACTGGACCGG | |
| | CGACATCCCCGCTCACGATGTGTGGCATCAGACCAGACAGGACCAGCTG | |
| | AGAGCCCTGACAACAGTGACAGCCCTCGTGCGGAAGTTTCTGGGACCAG | |
| | TGCCTGTGTATCCCGCCGTGGGAAATCACGAGAGCACCCCTGTGAACAG | |
| | CTTCCCTCCACCTTTCATCGAGGGCAACCACAGCAGCAGATGGCTGTAC | |
| | GAAGCCATGGCCAAGGCCTGGGAACCTTGGCTTCCAGCTGAAGCACTGC | |
| | GGACCCTGAGAATCGGCGGCTTTTATGCTCTGAGCCCCTATCCTGGCCTG | |
| | AGACTGATCAGCCTGAACATGAATTTCTGCAGCCGCGAGAACTTCTGGC | |
| | TGCTGATCAACTCCACAGATCCTGCCGGCCAGCTGCAGTGGCTTGTTGG | |
| | AGAATTGCAGGCCGCCGAGGACAGAGGCGATAAGGTGCACATCATCGG | |
| | ACACATCCCTCCAGGCCACTGCCTGAAGTCTTGGAGCTGGAACTACTAC | |
| | CGGATTGTGGCCAGATACGAGAACACCCTGGCCGCTCAGTTCTTCGGCC | |
| | ACACACACGTGGACGAGTTCGAGGTGTTCTACGACGAGGAAACCCTGAG | |
| | CAGACCTCTGGCCGTGGCATTTCTGGCCCCAAGCGCCACCACATATATC | |
| | GGACTGAACCCCGGCTACCGGGTGTACCAGATCGACGGCAATTACAGCG | |
| | GCAGCTCCCACGTGGTGCTGGACCACGAGACTTACATCCTGAATCTGAC | |
| | CCAGGCCAACATTCCCGGCGCTATCCCTCATTGGCAGCTGCTGTATCGGG | |
| | CCAGAGAGACATACGGCCTGCCTAACACACTGCCAACCGCCTGGCACAA | |
| | CCTGGTGTACAGAATGAGAGGCGACATGCAGCTGTTTCAGACCTTCTGG | |
| | TTCCTGTACCACAAGGGACACCCTCCAAGCGAGCCTTGTGGCACCCCTT | |
| | GTAGACTGGCCACTCTGTGTGCTCAGCTGTCCGCCAGAGCTGATAGCCC | |
| | CGCTCTGTGCAGACACCTGATGCCTGATGGAAGCCTGCCTGAGGCTCAG | |
| | TCCCTTTGGCCTAGACCTCTGTTCTCCTGA | |

TABLE 5
Exemplary Terminator Sequences

| | | SEQ |
|---|---|---|
| Name | Nucleotide Sequence | ID NO |
| BGH polyA | ctgtgccttctagttgccagccatctgttgtttgcccctccccc | 40 |
| | gtgccttccttgaccctggaaggtgccactcccactgtc | |
| | ctttcctaataaaatgaggaaattgcatcgcattgtctgagtag | |
| | gtgtcattctattctggggggtggggggggcaggac | |
| | agcaagggggaggattgggaagagaatagcaggcatgctgggga | |
| SV40 polyA | CAGACATGATAAGATACATTGATGAGTTTGGACAAACCACAACTAGAAT | 41 |
| | GCAGTGAAAAAAATGCTTTATTTGTGAAATTTGTGATGCTATTGCTTTAT | |
| | TTGTAACCATTATAAGCTGCAATAAACAAGTTAACAACAACAATTGCAT | |
| | TCATTTTATGTTTCAGGTTCAGGGGGAGGTGTGGGAGGTTTTTT | |

TABLE 6
Exemplary Guide RNA Sequences

| | | SEQ |
|---|---|---|
| Name | Nucleotide Sequence | ID NO |
| gCCR5_232 | CAATGTGTCAACTCTTGACA | 42 |
| gJCHAIN_2 | GTTAAGGTGTGTGATCTATA | 43 |
| gJCHAIN_31 | AAGAACCATTTGCTTTTCTG | 44 |
| gJCHAIN_54 | TCTCACCACCATATACGAGT | 45 |
| gJtolGHM_23 | GTCTCAGGAGCGGTGTCTGT | 46 |
| gNLRC5_26 | ACTGCCAGGTGTCCGAACCC | 47 |
| gNLRC5_28 | GAAGTTCTTCCTCCCCAACA | 48 |
| gNLRC5_75 | AGCGCCCACATCAGAGCTGT | 49 |
| gNLRC5_89 | CTTTGGTCCGCTGTACCCAG | 50 |
| gNLRC5_93 | ATTTGTCTGTAACTCCACCA | 51 |
| gNLRC5_94 | ACGTCTCCGAAGCCTGTGTG | 52 |
| gNLRC5_95 | TGATGGCTACCTCCCGTCCA | 53 |
| gNLRC5_99 | CAGGGTTAACAAGGGCCCGA | 54 |
| gNLRC5_105 | ACAGGCGACTTGGCACAGTG | 55 |
| gNLRC5_578 | GGGACAAACCGTTCTCACTG | 56 |
| gB2M_3 | GAGTAGCGCGAGCACAGCTA | 57 |
| gCD58_5 | CGCGTCGCTCCCAGCAACCA | 58 |
| gRFX5_4 | GTACTTACGAAATGGTACCT | 59 |

### REFERENCES

Hung et. Al., Molecular Therapy, 2018: https://doi.org/10.1016/j.ymthe.2017.11.012.
Maruo et. Al., Trends in Molecular Medicine, 2014, 10.1016/j.molmed.2014.09.003.
NIH, Niemann Pick Disease, Accessed: 2023, https://www.ninds.nih.gov/health-information/disorders/niemann-pick-disease.
Qui et. Al., Journal of Biological Chemistry, 2003, 10.1074/jbc.M303022200.
Schuchman, E. H., and Desnick, R. J., 2001. Niemann-Pick disease types A and B: acid sphingomyelinase deficiencies. In The Metabolic and Molecular Bases of Inherited Disease, 8^{th} edn. (Scriver, C.R., Beaudet, A.L., Sly, W. S., and Valle, D., eds; Childs, B., Kinzler, K. W., and Vogelstein, B., assoc. eds.), pp. 3589. McGraw-Hill, New York
Schuchman, E. H., FEBS Letters, 2009: https://doi.org/10.1016/j.febslet.2009.11.083.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference in their entireties.

1. A method of genetically modifying a B lineage cell population, the method comprising steps of:
(a) isolating primary B cells so that a primary B cell population is obtained;
(b) activating the primary B cell population; and
(c) during or after the activating step, multiplex engineering the primary B cell population such that a first expression cassette comprising a first transgene integrates into a first target locus; and either:
(i) a second expression cassette comprising a second transgene integrates into a second target locus; or
(ii) endogenous gene expression at the second target locus is disrupted without integration of a transgene;
thereby generating a genetically modified B lineage cell population.

2. The method of claim 1, further comprising a step of expanding the genetically modified B lineage cell population.

3. The method of claim 1 or 2, further comprising a step of differentiating the genetically modified B lineage cell population into a population of plasmablasts.

4. The method of claim 3, wherein the differentiation step comprises contacting the genetically modified B lineage cell population with culture media comprising:
(a) IL-2;
(b) IL-6;
(c) IL-10; and/or
(d) IL-15.

5. The method of claim 3 or 4, further comprising a step of differentiating the population of plasmablasts into a population of plasma cells.

6. The method of claim 5, wherein the differentiation step comprises contacting the population of plasmablasts with culture media comprising:
(a) IL-6;
(b) IL-15; and/or
(c) IFN-alpha-2-beta (IFNα-2β).

7. The method of any one of the above claims, wherein the first and second expression cassette arc contacted with the primary B lineage cell population simultaneously.

8. The method of any one of the above claims, wherein the first and second expression cassette are contacted with the primary B lineage cell population sequentially.

9. The method of claim 8, wherein the first expression cassette is contacted with the primary B lineage cell population on day 3 of the culture process, and the second expression cassette is contacted with the primary B lineage cell population on day 4 of the culture process, wherein the culture process has a day 1 start date.

10. The method of claim 9, wherein the time range of contact of the first expression cassette and second expression cassette is about 24 hours to about 144 hours.

11. The method of claim 7, wherein the expression cassette further comprises:
(a) a 5′ homology arm that is at least 95% identical to a sequence 5′ to the double-stranded break site; and
(b) a 3′ homology arm that is at least 95% identical to a sequence 3′ to the double-stranded break site.

12. The method of claim 11, wherein the 5′ homology arm comprises or is:
(i) a length about 350-850 base pairs in size,
(ii) a PAM site or absence of a PAM site, and/or
(iii) symmetrical or asymmetrical in length with the 3′ homology arm.

13. The method of claim 11 or 12, wherein the 3′ homology arm comprises or is:
(i) a length about 350-850 base pairs in size,
(ii) a PAM site or absence of PAM site, and/or
(iii) symmetrical or asymmetrical in length with the 5′ homology arm.

14. The method of any one of claims 7-13, further comprising an adeno-associated viral (AAV) vector.

15. The method of any one of the above claims, wherein the step of engineering comprises contacting the primary B cell population with a targeted nuclease capable of introducing double-stranded breaks.

16. The method of claim 15, wherein the targeted nuclease is or comprises a CRISPR-associated (Cas) protein, zinc finger nuclease (ZFN), transcription activator-like effector-based nuclease (TALEN), or meganuclease.

17. The method of claim 15, wherein the Cas protein is or comprises Cas9, Cas 12a, or Cas13a, or a variant thereof.

18. The method of claim 15, wherein the Cas protein is complexed with a guide RNA (gRNA).

19. The method of claim 18, wherein the gRNA is a single guide RNA (sgRNA).

20. The method of any one of the above claims, wherein the step of engineering comprises:
(a) electroporation of the primary B cell population, and/or
(b) transduction of the primary B cell population with one or more expression cassettes.

21. The method of claim 20, wherein the electroporation is performed on a composition comprising:
(a) the Cas protein complexed with the gRNA; and
(b) the primary B cell population.

22. The method of claim 20 or 21, wherein or after the step of engineering, the primary B cell population may undergo an additional step of engineering comprising:
(a) electroporation of the primary B cell population, and/or
(b) transduction of the primary B cell population with one or more expression cassettes.

23. The method of any one of the above claims, wherein the B lineage cell population is contacted with media that further comprises one or more of the following: XL413, M3814, nocodazole, and/or LAH4.

24. A population of genetically modified B lineage cells comprising a transgene, wherein the transgene is expressed from an endogenous CCR5 locus.

25. The population of genetically modified B lineage cells of claim 24, wherein the cells express a transgene and at least partially disrupt expression of endogenous CCR5.

26. A population of genetically modified B lineage cells comprising a transgene, wherein the transgene is expressed from an endogenous JCHAIN locus.

27. The population of genetically modified B lineage cells of claim 26, wherein the cells express a transgene without disrupting expression of endogenous JCHAIN.

28. A population of genetically modified B lineage cells comprising a transgene, wherein a transgene is expressed from an endogenous IgH locus.

29. The population of genetically modified B lineage cells of claim 28, wherein the cells express a transgene without disrupting expression of endogenous IgH.

30. A population of genetically modified B lineage cells comprising a transgene, wherein the transgene is expressed from an endogenous B2M locus.

31. The population of genetically modified B lineage cells of claim 30, wherein the cells express a transgene and at least partially disrupt expression of endogenous B2M.

32. The population of any one of claims 26-31, wherein the population of genetically modified B lineage cells is or comprises a population of genetically modified plasma cells.

33. The population of any one of claims 26-31, wherein the population of genetically modified B lineage cells is or comprises a population of plasmablasts.

34. The population of claim 33, wherein the population of genetically modified B lineage cells is or comprises a population of plasma cell precursors.

35. The method of any one of the above claims, further comprising a step of assessing one or more of (a)-(c) using an assay:
(a) percent of homology-directed repair;
(b) percent of indels; and/or
(c) amount of a reporter protein

36. The method of claim 35, wherein the reporter protein is or comprises of: green fluorescent protein (GFP), blue fluorescent protein (BFP), red fluorescent protein (RFP), and/or luciferase.

37. The method of claim 35, wherein the assay comprises or is one or more of: fluorescence-activated cell sorting (FAC-sort), Western Blot, flow cytometry, enzyme-linked immunosorbent spot assay (ELISpot), enzyme-linked immunosorbent assay (ELISA), droplet digital PCR (ddPCR), and inference of CRISPR editing (ICE).

38. A population of engineered B lineage cells comprising a transgene sequence, wherein the transgene is integrated in at least 10% of the cells.

39. The population of claim 38, wherein the transgene is expressed from an endogenous CCR5 locus.

40. A pharmaceutical composition comprising one or more B lineage cells selected from the population of genetically modified B lincage cells of any of the above claims, and one or more pharmaceutically acceptable excipients.

41. A method of administration of a pharmaceutical composition, the method comprising a step of: administering a pharmaceutical composition comprising:
(a) a population of B lineage cells selected from the population of genetically modified B lineage cells of any one of the above claims; and
(b) one or more pharmaceutically acceptable excipients;
wherein the pharmaceutical composition is administered to a subject.

42. The method of claim 41, wherein step (a) further comprises selecting a population of B lineage cells with high percentage of integration.

43. The method of claim 41, wherein the population of B lineage cells are characterized with at least 10% integration of a transgene.

44. A method of treating a disease, disorder, or condition in a subject, the method comprising administering to the subject a therapeutically effective amount of a pharmaceutical composition of claim 41, thereby treating the disease, disorder, or condition in the subject.

45. The method of claim 44, wherein the pharmaceutical composition is administered intravenously.

46. The method of any one of claims 44-45, wherein the pharmaceutical composition is administered to an adult subject.

47. The method of any one of claims 44-45, wherein the pharmaceutical composition is administered to a pediatric subject.

48. A method of characterizing a population of engineered B lineage cells of any one of the above claims, comprising assessing one or more of (a)-(f) using an assay:
(a) presence of a CD38 marker;
(b) presence of a CD138 marker;
(c) presence of a CD27 marker in at least 50% of live, singlet cells;
(d) secretion of at least 0.5 pg/cell/day of IgG;
(e) secretion of at least 2.5 pg/cell/day of IgM; and
(f) secretion of at least 0.5 pg/cell/day of IgD;

49. The method of claim 48, wherein the assay comprises or is one or more of: fluorescence-activated cell sorting (FAC-sort), Western Blot, flow cytometry, enzyme-linked immunosorbent spot assay (ELISpot), quantitative polymerase chain reaction (qPCR), reverse transcriptase PCR (RT-PCR), RT-qPCR, Meso Scale Discovery (MSD) and enzyme-linked immunosorbent assay (ELISA).

50. The method of claim 48 or 49, wherein one or more B lineage cells selected from the population of engraftable engineered B lineage cells engraft within the bone marrow of the subject.

51. A method of monitoring engraftment of engraftable engineered B lineage cells within a subject, comprising one or more of: bioluminescence, ELISpot, flow cytometry, qPCR, RT-PCR, RT-qPCR, and enzyme-linked immunosorbent assay.

52. The method of claim 1, wherein the first target locus is selected from CCR5, JCHAIN, and IGH.

53. The method of claim 1 or 52, wherein the second target locus is selected from B2M, BCMA, CD19, and CD20.

54. The method of claim 1, wherein the first target locus is CCR5 and the second target locus is CD19, wherein endogenous gene expression at the second target locus is disrupted without integration of a transgene.

55. The method of claim 54, wherein the first transgene is a BiTE.

56. The method of claim 1, wherein the first target locus is CCR5 and the second target locus is B2M, wherein endogenous gene expression at the second target locus is disrupted without integration of a transgene.

57. The method of claim 1, wherein the first target locus is JCHAIN and the second target locus is B2M, wherein endogenous gene expression at the second target locus is disrupted without integration of a transgene.

58. The method of claim 1, wherein the first target locus is IGH and the second target locus is B2M, wherein endogenous gene expression at the second target locus is disrupted without integration of a transgene.

59. The method of claim 1, wherein the first target locus is CCR5 and the second target locus is B2M, wherein a second expression cassette comprising a second transgene integrates into the second target locus.

60. The method of claim 1, wherein the first target locus is JCHAIN and the second target locus is B2M, wherein a second expression cassette comprising a second transgene integrates into the second target locus.

61. The method of claim 1, wherein the first target locus is IGH and the second target locus is B2M, wherein a second expression cassette comprising a second transgene integrates into the second target locus.

62. The method of any one of claims 54-61, wherein the first transgene integrating into the first target locus is a BiTE.

63. The method of any one of claims 54-61, wherein the first transgene integrating into the first target locus is selected from Factor IX, SMPD1, and blinatumomab.

64. The method of any one of claims 59-63, wherein the second transgene integrating into the second target locus is selected from HLA-A, HLA-B, HLA-C, HLA-E, and HLA-G.

65. A method of genetically modifying a B lineage cell population, the method comprising the steps of:
(a) isolating primary B cells so that a primary B cell population is obtained;
(b) activating the primary B cell population; and
(c) during or after the activating step, engineering the primary B cell population to reduce expression of one or more endogenous target genes;
thereby generating a genetically modified B lineage cell population.

66. The method of claim 65, wherein the endogenous target gene is NLRC5.

67. The method of claim 65, wherein the endogenous target gene is CD58.

68. The method of claim 65, wherein the endogenous target gene is RFX5.

69. The method of claim 65, wherein the endogenous target gene is TAP2.

70. The method of claim 65, wherein the endogenous target gene is TAPBP.

71. The method of claim 65, wherein the endogenous target genes are B2M and CD58.

72. The method of claim 65, wherein the endogenous target genes are NLRC5 and CD58.

73. The method of claim 65, wherein the endogenous target genes are RFX5 and CD58.

74. The method of any one of the above claims, wherein the method for reducing expression comprises knockout of the one or more endogenous target genes.

75. The method of any one of the above claims, wherein the engineering step comprises integrating one or more transgenes at one or more target loci.

76. The method of claim 75, wherein integration of the one or more transgenes reduces expression of the one or more endogenous target genes.

77. The method of claim 75 or 76, wherein the transgene comprises a sequence encoding HLA-E.

78. The method of claim 75 or 76, wherein the transgene comprises a sequence encoding HLA-G.

79. The method of claim 75 or 76, wherein the transgene comprises a sequence encoding CD47.

80. The method of claim 75 or 76, wherein the transgene comprises a sequence encoding PDL1.

81. The method of claim 75, wherein the transgene comprises Factor IX (FIX).

82. The method of claim 75, wherein the transgene comprises sphingomyelin phosphodiesterase 1 (SMPD1).

83. The method of claim 75, wherein the transgene comprises a bispecific dual-scFv.

84. The method of any one of claims 75-83, wherein the engineering step comprises integrating the transgene into an endogenous CCR5 locus.

85. The method of any one of claims 75-83, wherein the engineering step comprises integrating a transgene into an endogenous B2M locus.

86. The method of any one of claims 75-83, wherein the engineering step comprises integrating a transgene into an endogenous CD19 locus.

87. The method of any one of claims 75-83, wherein the engineering step comprises integrating a transgene into an endogenous JCHAIN locus.

88. The method of any one of claims 75-83, wherein the engineering step comprises integrating a transgene into an endogenous AAVS1 locus.

89. The method of any one of claims 75-83, wherein the engineering step comprises integrating a transgene into an endogenous TRAC locus.

90. The method of any one of claims 75-83, wherein the engineering step comprises integrating a transgene into an endogenous GSH locus.

91. The method of any one of the above claims, further comprising a step of transferring the genetically modified B lineage cell population to cell culture media without human or bovine serum for about 24 hours.

92. The method of any one of the above claims, wherein the step of engineering further comprises introducing a donor construct comprising the transgene into the primary B cell population.

93. The method of claim 92, wherein the donor construct comprises:
(a) a 5′ homology arm that is at least 95% identical to a sequence 5′ to the double-stranded break site; and
(b) a 3′ homology arm that is at least 95% identical to a sequence 3′ to the double-stranded break site.

94. The method of claim 95, wherein the donor construct comprises a transgene sequence of any one of SEQ ID NOs: 34-39.

95. The method of claim 93 or 94, wherein the donor construct comprises homology arm sequences of any one of SEQ ID NOs: 10-18.

96. A population of genetically modified B lineage cells comprising a knockout modification at one or more endogenous gene loci.

97. The population of claim 96, wherein the knockout modification is at an endogenous NLRC5 gene locus.

98. The population of claim 96, wherein the knockout modification is at an endogenous B2M gene locus.

99. The population of claim 96, wherein the knockout modification is at an endogenous CD58 gene locus.

100. The population of claim 96, wherein the knockout modification is at an endogenous RFX5 gene locus.

101. The population of claim 96, wherein the knockout modification is at an endogenous TAP2 gene locus.

102. The population of claim 96, wherein the knockout modification is at an endogenous TAPBP gene locus.

103. The population of claim 96, wherein the knockout modification is at an endogenous B2M gene locus and an endogenous CD58 locus.

104. The population of claim 96, wherein the knockout modification is at an endogenous NLRC5 gene locus and an endogenous CD58 locus.

105. The population of claim 96, wherein the knockout modification is at an endogenous RFX5 gene locus and an endogenous CD58 locus.

106. The population of any one of the above claims, wherein the population further comprises a transgene sequence expressed from one or more target loci.

107. The population of claim 106, wherein the population comprises a knockout modification at an endogenous B2M gene locus and a transgene expressed from a CCR5 locus.

108. The population of claim 107, wherein the transgene comprises a sequence encoding a HLA-E or HLA-G protein.

109. The population of claim 108, wherein the transgene further comprises a 2A peptide or IRES sequence 3′ of the sequence encoding a HLA-E protein.

110. The population of claim 109, wherein the transgene further comprises a sequence encoding a second protein 3′ of the 2A peptide or IRES sequence.


SEQUENCE LISTING (62 sequences)

SEQ ID NO: 1 - DNA, 23 bp, synthetic construct
  source 1..23: mol_type = other DNA
gcagcaaaccttcccttcactac

SEQ ID NO: 2 - DNA, 23 bp, synthetic construct
  source 1..23: mol_type = other DNA
aggattcccgagtagcagatgac

SEQ ID NO: 3 - DNA, 22 bp, synthetic construct
  source 1..22: mol_type = other DNA
gggtggaacaagatggattatc

SEQ ID NO: 4 - DNA, 20 bp, synthetic construct
  source 1..20: mol_type = other DNA
catcgcattgtctgagtagg

SEQ ID NO: 5 - DNA, 19 bp, synthetic construct
  source 1..19: mol_type = other DNA
cagtggatcgggtgtaaac

SEQ ID NO: 6 - DNA, 27 bp, synthetic construct
  source 1..27: mol_type = other DNA
tcgggagcctcttgctggaaaatagaa

SEQ ID NO: 7 - DNA, 21 bp, synthetic construct
  source 1..21: mol_type = other DNA
ccacatcagaaggaagactac

SEQ ID NO: 8 - DNA, 19 bp, synthetic construct
  source 1..19: mol_type = other DNA
gctgtatgaatccaggtcc

SEQ ID NO: 9 - DNA, 26 bp, synthetic construct
  source 1..26: mol_type = other DNA
tgtttcctccaggataaggcagctgt

SEQ ID NO: 10 - DNA, 600 bp, synthetic construct
  source 1..600: mol_type = other DNA
ggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgaca

SEQ ID NO: 11 - DNA, 600 bp, synthetic construct
  source 1..600: mol_type = other DNA
tacaaaatacagattttgtgagatatatacaaaatacagattttgtgagatatatacaaaatacagattttgtgagatatatacaaaatacatattttgtgagatatatacaaaatatatatttaaatctggtagcttggttaattaaggactagtataaaaaattaacataagatacacttgggaccttaaaggactttcatttctctccccttccttcatctctcctccttcacttaccttcatttattattctaggcatgacaagtgtcagatgtctgatttgaagactaatatgaatatatctaggtaataaaatggaaaatccctatttcaacattcttaatggaatccttccttgaaattattattagtcttctggtttaaacatttatatagtgtaaatgcaaaataaaaatgattgtatcaaattatggtttttatttggatgctttgcaagaaaatacttaaatgtgagtcatacatttttccagttcagcatttaaacttcagacttcaaccacagttgtgattgtttttagtttgttagctgcctggagtgttattttaagaaagcagaagcaccatcatttgcacact

SEQ ID NO: 12 - DNA, 600 bp, synthetic construct
  source 1..600: mol_type = other DNA
atacatattttgtgagatatatacaaaatatatatttaaatctggtagcttggttaattaaggactagtataaaaaattaacataagatacacttgggaccttaaaggactttcatttctctccccttccttcatctctcctccttcacttaccttcatttattattctaggcatgacaagtgtcagatgtctgatttgaagactaatatgaatatatctaggtaataaaatggaaaatccctatttcaacattcttaatggaatccttccttgaaattattattagtcttctggtttaaacatttatatagtgtaaatgcaaaataaaaatgattgtatcaaattatggtttttatttggatgctttgcaagaaaatacttaaatgtgagtcatacatttttccagttcagcatttaaacttcagacttcaaccacagttgtgattgtttttagtttgttagctgcctggagtgttattttaagaaagcagaagcaccatcatttgcacactccttatagatcacacaccttaaccctgactttttttgctccagtttttcagaagaagtgaagtcaagatgaagaaccatttgctttt

SEQ ID NO: 13 - DNA, 600 bp, synthetic construct
  source 1..600: mol_type = other DNA
ttttctaaagaaaaaatcattggcattcatagaaaggccaatttctcttaatagttcaataagtggatttgatcttataaaaaggcaggtgtttctttggaaatgacagactccaacatcaatttttttaaaaattctccctttcttgtcactataaataacttgtttagacagatatacagttgggaataagcctaacacagtagaaattgctgtatggtgtagataaaacaatcatattatcatatcattaattatattgcttactttcaactaatatatattaaagattggaaaatcccataagctattctgtattgtagagctgcttatgtctgaaaggagtcatcccttgctgtcatgtcagagctgcaagaactaattgattttggattgaaatgtgtagtcacattttgagacagcatttgaggggattgtctaatacatatatttgcttttcagctgtaaaaaatgtgatcctacagaagtggagctggataatcagatagttactgctacccagagcaatatctgtgatgaagacagtgctacagagacctgctacacttatgacagaaacaagtgctacacagctgtggt

SEQ ID NO: 14 - DNA, 450 bp, synthetic construct
  source 1..450: mol_type = other DNA
atgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtc

SEQ ID NO: 15 - DNA, 600 bp, synthetic construct
  source 1..600: mol_type = other DNA
ctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaac

SEQ ID NO: 16 - DNA, 600 bp, synthetic construct
  source 1..600: mol_type = other DNA
agatcacacaccttaaccctgactttttttgctccagtttttcagaagaagtgaagtcaagatgaagaaccatttgcttttctggggagtcctggcggtttttattaaggctgttcatgtgaaaggtatgtgatatttagaaaatgatcccagatcaaaggaaaaatataggacagatctgtttttcagttttaaacatttatgcctatgctttggttggccagttagccagctactttttcctaacatgctttcatctttctatggcagttgtctttacatagataagactctttttattcttttttctcttatcttacttacttgtttgcctactacaaactagagaataagaaattagctaactacagatagtcctgcttatatgaatgtccatattgaatcttaaaaatctataatccttcccttgagtctcttaaattaatattctggagaaatgaaataataattactgtaagtacaacatggaatgcattgggattttatttccccaaagaggctggtattggttttaacagctttcctggatctgatcccacagatttattatgaccctttaacaatgtttaatgttataca

SEQ ID NO: 17 - DNA, 600 bp, synthetic construct
  source 1..600: mol_type = other DNA
cgtatatggtggtgagaccaaaatggtggaaacagccttaaccccagatgcctgctatcctgactaatttaagtcattgctgactgcatagctctttttcttgagaggctctccattttgattcagaaagttagcatatttattaccaatgaatttgaaaccagggcttttttttttttttgggtgatgtaaaaccaactccctgccaccaaaataattaaaatagtcacattgttatctttattaggtaatcacttcttaattatatgttcatactctaagtatcaaaatcttccaattatcatgctcacctgaaagaggtatgctctcttaggaatacagtttctagcattaaacaaataaacaaggggagaaaataaaactcaaggactgaaaatcaggaggtgtaataaaatgttcctcgcattcccccccgcttttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctaga

SEQ ID NO: 18 - DNA, 450 bp, synthetic construct
  source 1..450: mol_type = other DNA
tgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccat

SEQ ID NO: 19 - DNA, 1392 bp, synthetic construct
  source 1..1392: mol_type = other DNA
gccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgctgagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctga

SEQ ID NO: 20 - DNA, 1392 bp, synthetic construct
  source 1..1392: mol_type = other DNA
gccaccatgcagagggtgaacatgatcatggctgagagccctggcctgatcaccatctgcctgctgggctacctgctgagtgctgagtgcacagtgttcctggaccatgagaatgccaacaagatcctgaacagacccaagagatacaacagtggcaagctggaggagtttgtgcagggcaacctggagagggagtgcatggaggagaagtgcagctttgaggaggccagggaagtgtttgagaacacagagagaaccacagagttctggaagcagtatgtggatggagaccagtgtgagagcaacccttgcctgaatggaggcagctgcaaggatgacatcaacagctatgagtgctggtgcccttttggctttgagggcaagaactgtgagctggatgtgacctgcaacatcaagaatggaaggtgtgagcagttctgcaagaactctgctgacaacaaagtggtgtgtagctgcacagagggctacagactggctgagaaccagaagtcttgtgagccagctgtgccttttccctgtggcagagtcagtgtgtcccagaccagcaagctgaccagagctgagacagtgttccctgatgtggactatgtgaatagcacagaggctgagaccatcctggacaacatcacccagagcacccagtccttcaatgacttcaccagagttgtgggaggggaggatgccaagcctggccagttcccctggcaggtggtgctgaatggcaaagtggatgccttctgtggaggcagcattgtgaatgagaagtggattgtgacagctgcccactgtgtggagacaggagtgaagatcacagtggtggctggagaacacaatattgaggagacagagcacactgagcagaagaggaatgtcatcagaattatcccccaccacaactacaatgctgccatcaacaagtacaaccatgacattgccctgctggagctggatgagcctctggtgctgaatagctatgtgacccccatctgcattgctgacaaggagtacaccaacatcttcctgaagtttggcagtggctatgtcagtggctggggcagagtgttccacaagggcagatctgccctggtgctgcagtacctgagagtgcccctggtggacagagccacctgcctgttgagcaccaagttcaccatctacaacaacatgttctgtgctggcttccatgagggaggcagagacagctgccagggagattctggagggccccatgtgacagaagtggagggcaccagcttcctgacaggcatcatcagctggggggaggagtgtgccatgaagggcaagtatggcatctacaccaaagtcagcagatatgtgaactggatcaaggagaaaaccaagctgacctga

SEQ ID NO: 21 - DNA, 1392 bp, synthetic construct
  source 1..1392: mol_type = other DNA
gccaccatgcagagggtgaacatgatcatggcagagagccctggcctgatcaccatctgcctgctgggctacctgctgtctgcagaatgtacagtgtttctggaccatgagaatgccaacaagatcctgaacaggcccaagagatacaactctggcaagctggaggagtttgtgcagggcaacctggagagggagtgcatggaggagaagtgcagctttgaggaggccagggaagtgtttgagaacactgagaggaccactgagttctggaagcagtatgtggatggagaccagtgtgagagcaacccttgcctgaatggaggcagctgcaaggatgacatcaacagctatgagtgctggtgcccatttggctttgagggcaagaactgtgagctggatgtgacctgcaacatcaagaatggcagatgtgagcagttctgcaagaactctgctgacaacaaagtggtgtgtagctgcactgagggctacagactggcagagaatcagaagtcctgtgaacctgctgtgccttttccctgtggcagagtctctgtgtcccagaccagcaagctgaccagagcagagactgtgttcccagatgtggactatgtgaatagcactgaggcagagaccatcctggacaacatcacccagagcacccagtccttcaatgacttcaccagagttgtgggaggagaggatgccaagccaggccagttcccctggcaggtggtgctgaatggcaaagtggatgccttctgtggaggcagcattgtgaatgagaagtggattgtgacagcagcccactgtgtggagactggagtgaagatcactgtggtggcaggagaacacaatattgaggagactgagcacacagagcagaagagaaatgtcatcaggattatcccccaccacaactacaatgcagccatcaacaagtacaaccatgacattgccctgctggagctggatgagcctctggtgctgaatagctatgtgacccccatctgcattgcagacaaggagtacaccaacatcttcctgaagtttggctctggctatgtcagtggctggggcagagtgttccacaagggcagatctgccctggtgctgcagtacctgagagtgcccctggtggacagagccacctgcctgttgagcaccaagttcaccatctacaacaacatgttctgtgcaggcttccatgagggaggcagagacagctgccagggagactctggaggaccccatgtgactgaagtggagggcaccagcttcctgactggcatcatcagctggggagaggagtgtgccatgaagggcaagtatggcatctacaccaaagtcagcagatatgtgaactggatcaaggagaaaaccaagctgacctga

SEQ ID NO: 22 - DNA, 7196 bp, synthetic construct
  source 1..7196: mol_type = other DNA
cgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttgggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaaccaactttattatacatagttgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 23 - DNA, 7196 bp, synthetic construct
  source 1..7196: mol_type = other DNA
cgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttgggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttgctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaaccaactttattatacatagttgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 24 - DNA, 7196 bp, synthetic construct
  source 1..7196: mol_type = other DNA
cgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttgggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttcctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaaccaactttattatacatagttgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 25 - DNA, 7196 bp, synthetic construct
  source 1..7196: mol_type = other DNA
cgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttgggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctgctgaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaaccaactttattatacatagttgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 26 - DNA, 7196 bp, synthetic construct
  source 1..7196: mol_type = other DNA
cgcggaacccctatttgtttatttttctaaatacattcaaatatgtatccgctcatgagacaataaccctgataaatgcttcaataatattgaaaaaggaagagtatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttgggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctcctgaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaaccaactttattatacatagttgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtg

SEQ ID NO: 27 - DNA, 2506 bp, synthetic construct
  source 1..2506: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 28 - DNA, 2506 bp, synthetic construct
  source 1..2506: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttgctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 29 - DNA, 2506 bp, synthetic construct
  source 1..2506: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttcctagacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 30 - DNA, 2506 bp, synthetic construct
  source 1..2506: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctgctgaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 31 - DNA, 2506 bp, synthetic construct
  source 1..2506: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatccaagtttgtacaaaaaagcaggctgccaccatgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctcctgaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 32 - DNA, 348 bp, synthetic construct
  source 1..348: mol_type = other DNA
gaacagttggaacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 33 - DNA, 213 bp, synthetic construct
  source 1..213: mol_type = other DNA
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 34 - DNA, 1272 bp, synthetic construct
  source 1..1272: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 35 - DNA, 1896 bp, synthetic construct
  source 1..1896: mol_type = other DNA
atgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctag

SEQ ID NO: 36 - DNA, 1896 bp, synthetic construct
  source 1..1896: mol_type = other DNA
atgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttgctag

SEQ ID NO: 37 - DNA, 1896 bp, synthetic construct
  source 1..1896: mol_type = other DNA
atgccccgctacggagcgtcactccgccagagctgccccaggtccggcagggagcagggacaagacgggaccgccggagctcctggactcctttggatgggactggtgctggcgctggcgcttgctctggcgctggccctggcactgtctgactctcgggttctctgggctccggcagaagctcaccctctttctcctcaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccctccagccccaggtgctcctgtcagccgcatcctcttcctcactgacctgcactgggatcatgactacctggagggcacggaccctgactgtgcagacccactgtgctgccgccggggttctggcctgccgcctgcatccaggccaggtgccggatactggggcgaatacagcaagtgtgacctgcccctgaggaccctggagagcctgttgagtgggctgggcccagccggaccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgtcgtctggctactctttgtgcccagctctctgcccgtgctgacagccctgctctgtgccgccacctgatgccagatgggagcctcccagaggcccagagcctgtggccaaggccactgttttcctag

SEQ ID NO: 38 - DNA, 1896 bp, synthetic construct
  source 1..1896: mol_type = other DNA
atgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctgctga

SEQ ID NO: 39 - DNA, 1896 bp, synthetic construct
  source 1..1896: mol_type = other DNA
atgcctagatacggcgccagcctgagacagagctgtcctagatctggcagagagcagggccaagacggaacagctggtgctcctggactgctgtggatgggacttgttctggctctggccctggctcttgcattggcactggctctgagcgacagcagagttctgtgggctcctgccgaagctcatcctctgagtcctcagggacatcccgccagactgcacagaatcgtgcccagactgagagatgtgttcggctggggcaacctgacatgccctatctgcaagggcctgttcaccgccatcaacctgggcctgaagaaagaacccaacgtggccagagtgggcagcgtggccatcaagctgtgcaacctgctgaagattgcccctcctgccgtgtgccagtctatcgtgcacctgttcgaggacgacatggtggaagtgtggcggagaagcgtgctgtctccatctgaagcttgtggcctgctgctgggctctacatgtggccactgggacatctttagcagctggaacatcagcctgcctaccgtgcctaagcctcctccaaagcctccatctcctccagcacctggcgctccagtgtccagaatcctgttcctgaccgacctgcactgggaccacgattacctggaaggcaccgatcctgactgcgccgatcctctgtgttgcagaagaggctctggactgcctcctgcctctagaccaggtgccggatattggggcgagtacagcaagtgcgacctgcctctgagaaccctggaaagcctgctgtctggactgggacctgccggacctttcgatatggtgtactggaccggcgacatccccgctcacgatgtgtggcatcagaccagacaggaccagctgagagccctgacaacagtgacagccctcgtgcggaagtttctgggaccagtgcctgtgtatcccgccgtgggaaatcacgagagcacccctgtgaacagcttccctccacctttcatcgagggcaaccacagcagcagatggctgtacgaagccatggccaaggcctgggaaccttggcttccagctgaagcactgcggaccctgagaatcggcggcttttatgctctgagcccctatcctggcctgagactgatcagcctgaacatgaatttctgcagccgcgagaacttctggctgctgatcaactccacagatcctgccggccagctgcagtggcttgttggagaattgcaggccgccgaggacagaggcgataaggtgcacatcatcggacacatccctccaggccactgcctgaagtcttggagctggaactactaccggattgtggccagatacgagaacaccctggccgctcagttcttcggccacacacacgtggacgagttcgaggtgttctacgacgaggaaaccctgagcagacctctggccgtggcatttctggccccaagcgccaccacatatatcggactgaaccccggctaccgggtgtaccagatcgacggcaattacagcggcagctcccacgtggtgctggaccacgagacttacatcctgaatctgacccaggccaacattcccggcgctatccctcattggcagctgctgtatcgggccagagagacatacggcctgcctaacacactgccaaccgcctggcacaacctggtgtacagaatgagaggcgacatgcagctgtttcagaccttctggttcctgtaccacaagggacaccctccaagcgagccttgtggcaccccttgtagactggccactctgtgtgctcagctgtccgccagagctgatagccccgctctgtgcagacacctgatgcctgatggaagcctgcctgaggctcagtccctttggcctagacctctgttctcctga

SEQ ID NO: 40 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 41 - DNA, 192 bp, synthetic construct
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 42 - DNA, 20 bp, synthetic construct
  source 1..20: mol_type = other DNA
caatgtgtcaactcttgaca

SEQ ID NO: 43 - DNA, 20 bp, synthetic construct
  source 1..20: mol_type = other DNA
gttaaggtgtgtgatctata

SEQ ID NO: 44 - DNA, 20 bp, synthetic construct
  source 1..20: mol_type = other DNA
aagaaccatttgcttttctg

SEQ ID NO: 45 - DNA, 20 bp, synthetic construct
  source 1..20: mol_type = other DNA
tctcaccaccatatacgagt

SEQ ID NO: 46 - DNA, 20 bp, synthetic construct
  source 1..20: mol_type = other DNA
gtctcaggagcggtgtctgt

SEQ ID NO: 47 - DNA, 20 bp, synthetic construct
  source 1..20: mol_type = other DNA
actgccaggtgtccgaaccc

SEQ ID NO: 48 - DNA, 20 bp, synthetic construct
  source 1..20: mol_type = other DNA
gaagttcttcctccccaaca

SEQ ID NO: 49 - DNA, 20 bp, synthetic construct
  source 1..20: mol_type = other DNA
agcgcccacatcagagctgt

SEQ ID NO: 50 - DNA, 20 bp, synthetic construct
  source 1..20: mol_type = other DNA
ctttggtccgctgtacccag

SEQ ID NO: 51 - DNA, 20 bp, synthetic construct
  source 1..20: mol_type = other DNA
atttgtctgtaactccacca

SEQ ID NO: 52 - DNA, 20 bp, synthetic construct
  source 1..20: mol_type = other DNA
acgtctccgaagcctgtgtg

SEQ ID NO: 53 - DNA, 20 bp, synthetic construct
  source 1..20: mol_type = other DNA
tgatggctacctcccgtcca

SEQ ID NO: 54 - DNA, 20 bp, synthetic construct
  source 1..20: mol_type = other DNA
cagggttaacaagggcccga

SEQ ID NO: 55 - DNA, 20 bp, synthetic construct
  source 1..20: mol_type = other DNA
acaggcgacttggcacagtg

SEQ ID NO: 56 - DNA, 20 bp, synthetic construct
  source 1..20: mol_type = other DNA
gggacaaaccgttctcactg

SEQ ID NO: 57 - DNA, 20 bp, synthetic construct
  source 1..20: mol_type = other DNA
gagtagcgcgagcacagcta

SEQ ID NO: 58 - DNA, 20 bp, synthetic construct
  source 1..20: mol_type = other DNA
cgcgtcgctcccagcaacca

SEQ ID NO: 59 - DNA, 20 bp, synthetic construct
  source 1..20: mol_type = other DNA
gtacttacgaaatggtacct

SEQ ID NO: 60 - DNA, 600 bp, synthetic construct
  source 1..600: mol_type = other DNA
agtcctggcggtttttattaaggctgttcatgtgaaaggtatgtgatatttagaaaatgatcccagatcaaaggaaaaatataggacagatctgtttttcagttttaaacatttatgcctatgctttggttggccagttagccagctactttttcctaacatgctttcatctttctatggcagttgtctttacatagataagactctttttattcttttttctcttatcttacttacttgtttgcctactacaaactagagaataagaaattagctaactacagatagtcctgcttatatgaatgtccatattgaatcttaaaaatctataatccttcccttgagtctcttaaattaatattctggagaaatgaaataataattactgtaagtacaacatggaatgcattgggattttatttccccaaagaggctggtattggttttaacagctttcctggatctgatcccacagatttattatgaccctttaacaatgtttaatgttatacaggttgcctataaattgatgactgaacttttgttattttgtttactgatctttcaaacaacttcagaaaatggaatacattctgaatt

SEQ ID NO: 61 - DNA, 20 bp, synthetic construct
  source 1..20: mol_type = other DNA
aggaccttctctgagctgtc

SEQ ID NO: 62 - DNA, 20 bp, synthetic construct
  source 1..20: mol_type = other DNA
gccctaaactttgtcccgac